(19) United States
(12) Patent Application Publication
(10) Pub. No.: US 2024/0308998 A1
(43) Pub. Date: Sep. 19, 2024

(54) PYRROLOPYRIDONE DERIVATIVES USEFUL IN THE TREATMENT OF CANCER

(71) Applicant: Tay Therapeutics Limited, Dudee (GB)

(72) Inventors: Christopher Andrew WOODLAND, Dundee (GB); Mark BELL, Dundee (GB)

(21) Appl. No.: 18/575,006

(22) PCT Filed: Jun. 29, 2022
(86) PCT No.: PCT/GB2022/051667

§ 371 Date: Dec. 28, 2023

(30) Foreign Application Priority Data
Jun. 29, 2021 (GB) 2109324.0
Jun. 1, 2022 (GB) 2208160.8

(51) Int. Cl. C07D 471/04; A61K 31/444; A61K 31/506

(52) U.S. Cl. CPC C07D 471/04; A61K 31/444; A61K 31/506

(57) ABSTRACT

This invention relates to compounds comprising a pyrrolopyridone core, pharmaceutically-acceptable salts thereof, and compositions comprising the same. The compounds and salts herein are useful as anti-inflammatory and/or other therapies.

This invention relates to compounds comprising a pyrrolopyridone core, and pharmaceutically-acceptable salts and compositions of such compounds. The compounds herein are useful as anti-inflammatory and/or other therapies. Therefore, the present disclosure also concerns compounds for use as medicaments, particularly for the treatment of inflammatory diseases.

### CROSS REFERENCES TO RELATED APPLICATIONS

This application claims priority from GB Patent Application No. 2109324.0, filed Jun. 29, 2021, and GB Patent Application No. 2208160.8, filed Jun. 1, 2022, which are hereby incorporated by reference in their entirety.

### BACKGROUND

Bromodomain and Extra-Terminal (BET) proteins are a family of four bromodomain-containing (BRD) proteins (BRD2, BRD3, BRD4 and BRDT). All four members contain two BRDs (located next to each other toward the N-terminal of the proteins) and an extra-terminal domain (Shi, J. et al. Cancer Cell 25(2):210-225 (2014)). The two BRDs in each BET protein are designated bromodomain I (BDI) and bromodomain II (BDII). The BRD is a functional protein domain that contains a defined and predominantly hydrophobic pocket that binds to acetylated lysine residues, typically those found on transcription factors (Shi, J. et al. Cancer Cell 25(2):210-225 (2014)) or on the N-terminal tails of histone proteins. BRDs function as epigenetic regulators, i.e., they functionally alter gene activity and expression without altering the DNA sequence. For example, BRD4 recruits the transcription factor P-TEFb to promoters leading to altered expression of genes involved in the cell cycle (Yang et al., Mol. Cell Biol. 28: 967-976 (2008)). BRD2 and BRD3 also regulate growth promoting genes (LeRoy et al., Mol Cell 30:51-60 (2008)). Therefore, BRDs are responsible for transducing the signals carried by acetylated lysine residues into various phenotypes. BETs are considered in the art to be ubiquitously expressed in humans except for BRDT, which is normally expressed in the testes but is also expressed by some cancers (Ekaterina B. F. et al. Cell J. 19 (Suppl 1): 1-8 (2017)).

BET proteins have roles in the regulation of biochemical pathways such as MYC, BCL2, FOSL1, P-TEFb, NFkB, Glucocorticoid signalling and others (Shi J. et al. Mol Cell. June 5; 54(5):728-36 (2014)), (Hajmirza A. Biomedicines. February 6; 6(1). pii: E16 (2018)), (Shan N. Elife. September 11; 6. pii: e27861. (2017)), (Huang B. Mol Cell Biol. March; 29(5):1375-87 (2009)). As such, BET inhibitors are considered to have potential uses in a range of inflammatory diseases, cancers, infections, metabolic diseases, CNS disorders, fibrotic diseases, and cardiac diseases (Deanna A. M. et al. J Exp Med. Oct 21; 210(11): 2181-2190 (2013)), (Rab K. P. et al. Trends Pharmacol. Sci. March; 33(3):146-53 (2012)), (Anna C. B. et al. J Immunol. April 1; 190(7): 3670-3678 (2013)), (Zuber J. et al. Nature. August 3; 478(7370):524-8. (2011)), (Montserrat P. S. et al. Epigenetics.; 12(5): 323-339 (2017)), (Qiming D. et al. Sci Transl Med. May 17; 9(390): eaah5084. (2017)), (Kristin M. K et al. J Biol Chem. August 11; 292 (32): 13284-13295 (2017)), (Ning D. et al. PNAS December 22, 112 (51) 15713-15718 (2015)).

The inhibition of BDII domain of BET proteins has been shown to effect inflammatory diseases, metabolic disease, cancers, and fibrotic diseases (Gilan et. al., Science 368, 387-394 (2020)), (L. M Tsujikawa et. al. Clin Epigenetics. 2019; 11(1):102), (E. Faivre et al. Nature 578, 306-310 (2020)), (M. Zhang, et. al. Cellular Signalling 61 (2019) 20-29).

Compounds that can inhibit or affect the function of BET proteins have the potential to modulate gene expression and treat diseases that are at least in part caused by abnormal regulation of BET protein activity. Several small molecules have been reported to be effective in BET inhibition, including diazepine-, 3,5-dimethylisoxazole-, thiazol-2-one-, diazobenzene-, and 4-acylpyrrole-based compounds (see M. Brand et al, ACS Chem. Biol. 2015, 10, 22-39, WO2011054553, WO2011054845). Compounds that can selectively inhibit the function of BDII over BDI have the potential to modulate gene expression and treat diseases that are at least in part caused by abnormal regulation of BET protein activity, while offering the potential of an improved therapeutic index. Several small molecules have been reported to be effective in selectively inhibiting the function of BET BDII over BDI, including (BY27, RVX-297, ABBV744, GSK046, GSK620, GSK549 (Chen D. et. al. Eur J Med Chem 182, 2019, 111633), (Wells P. S. et. al. Proc. Natl. Acad. Sci. U.S.A. 2013, 110, 19754-19759) (Sheppard G. S. et. al. J. Med. Chem. 2020, 63, 10, 5585-5623), (Preston A. et. al. J. Med. Chem. 2020, 63, 17, 9070-9092), (Seal J. T. et. al. J. Med. Chem. 2020, 63, 17, 9093-9126). Improved therapeutic index and pre-clinical safety of BDII selective BET inhibitors verses pan-BET inhibitors have been demonstrated (E. Faivre et al. Nature 578, 306-310 (2020)).

Compounds comprising 6-methyl-1,6-dihydro-7H-pyrrolo[2,3-c]pyridin-7-one moieties, substituted at the 4- and/or 2-positions are described in patent applications WO 2017177955, WO 2015081280, WO 2014206150, WO 2014206345, WO 2013097601, WO 2013097052 and WO 2018130174 as useful for the inhibition of BET proteins.

WO2020216779A1 discloses compounds useful in anti-inflammatory and anti-cancer therapies.

WO 2021068755 discloses compounds having BRD4 inhibitory activity, and a preparation method thereof.

WO 2018195155 discloses compounds for the treatment of diseases mediated by aberrant cell signalling, such as inflammatory disorders, cancer and neoplastic disease.

Particular compounds described exhibit selective inhibitory activity against CBP compared with BRD4.

WO 2021003310 discloses novel bromodomain and extraterminal domain (BET) inhibitors and therapeutic methods of treating conditions and diseases using the BET inhibitors disclosed. The present disclosure provides novel BET protein inhibitors, their use as medicaments, compositions containing them and processes for their preparation.

### BRIEF SUMMARY OF THE DISCLOSURE

In accordance with a first aspect, the present disclosure provides a compound of formula (I), or a pharmaceutically acceptable salt or N-oxide thereof:

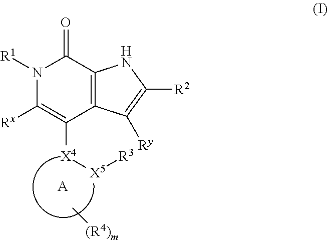

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is independently selected from 5-membered heterocyclyl, 6-membered heterocyclyl and phenyl, each optionally substituted with from 1 to 4 R^{2a} groups;
R^{2a} is independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₆ cycloalkyl and 4- to 6-membered heterocyclyl;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₃-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₃-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, cyclopropyl, cyclobutyl, and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₃-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₃-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, NR⁵R⁶, OR⁶, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R^{x} and R^{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂-C₁-C₄-alkyl.

In one or more embodiments, the compounds of formula (I) may be an enantiomer, a mixture of enantiomers, a racemate, a diastereoisomer, a mixture of diastereoisomers, a geometric isomer, a mixture of geometric isomers, a tautomer or a mixture of tautomers. The compound of formula (I) may also be in the form of a solvate or hydrate.

In an embodiment, the compound of formula (I) is a compound of formula (II):

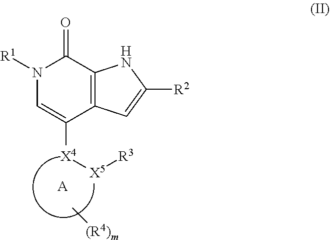

wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is independently selected from 5-membered heterocyclyl, 6-membered heterocyclyl and phenyl, each optionally substituted with from 1 to 4 R^{2a} groups;
R^{2a} is independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₆ cycloalkyl and 4- to 6-membered heterocyclyl;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₃-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₃-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, cyclopropyl, cyclobutyl, and 4- to 6-membered heterocycloalkyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₃-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₃-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.

In an embodiment, the compound of formula (I) is a compound of formula (III):

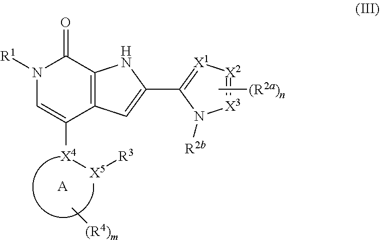

wherein X⁴, X⁵, Ring A, R^{2a}, R³, R⁴ and m are as described above for compounds of formula (I) and wherein [symbol] is independently selected from a single bond and a double bond;
R^{2b} is independently selected from H, C₁-C₄-alkyl, C₃-C₆ cycloalkyl, and 4- to 6-membered heterocyclyl;
X¹ is independently selected from carbon and nitrogen;
X² and X³ are each independently selected from carbon, nitrogen, oxygen and sulfur; wherein where one of X² or X³ is oxygen or sulfur, the other of X² and X³ must be carbon; and where [symbol] is a double bond, X² and X³ are each independently selected from carbon and nitrogen; and
n is independently an integer selected from 0, 1, 2, 3 and 4. R^{2b} may be H.

In an embodiment, the compound of formula (I) is a compound of formula (IV):

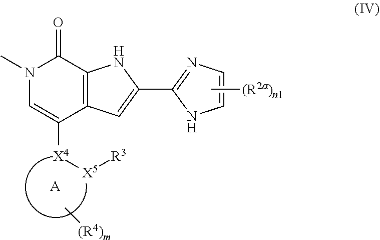

wherein X⁴, X⁵, Ring A, R^{2a}, R³, R⁴, and m are as described above for compounds of formula (I) and wherein n1 is independently an integer selected from 0, 1 and 2.

In an embodiment, the compound of formula (I) is a compound of formula (V):

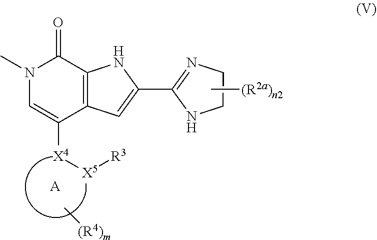

wherein X⁴, X⁵, Ring A, R^{2a}, R³, R⁴, and m are as described above for compounds of formula (I) and n2 is independently an integer selected from 0, 1, 2, 3 and 4.

In an embodiment, the compound of formula (I) is a compound of formula (VI):

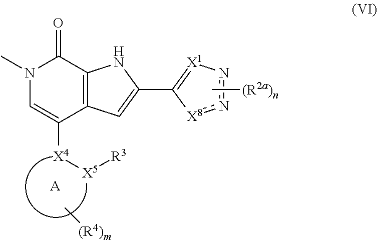

wherein X⁴, X⁵, Ring A, R^{2a}, R³, R⁴ and m are as described above for compounds of formula (I) and wherein [symbol] is independently selected from a single bond and a double bond;
X¹ and X⁸ are each independently selected from carbon and nitrogen; and
n is independently an integer selected from 0, 1, 2, 3 and 4.

In an embodiment, the compound of formula (I) is a compound of formula (VII):

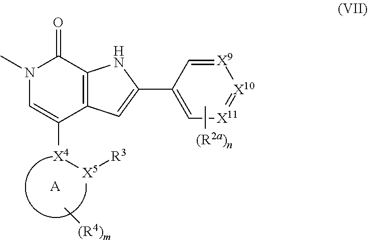

wherein X⁴, X⁵, Ring A, R^{2a}, R³, R⁴ and m are as described above for compounds of formula (I);
X⁹, X¹⁰, and X¹¹ are each independently selected from carbon and nitrogen; and
n is independently an integer selected from 0, 1, 2, 3, 4, and 5.

In an embodiment, the compound of formula (I) is a compound of formula (VIII):

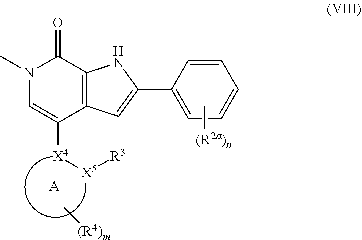

wherein X⁴, X⁵, Ring A, R¹, R^{2a}, R³, R⁴ and m are as described above for compounds of formula (I); and
n is independently an integer selected from 0, 1, 2, 3, 4, and 5.

In an embodiment, the compound of formula (I) is a compound of formula (IX):

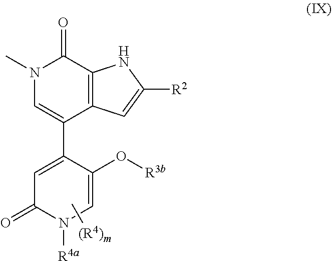

wherein R², R^{3b}, and R⁴ are as described above for compounds of formula (I); and wherein:
m is an integer selected from 0, 1, or 2; and
R^{4a} is independently selected from H, C₁-C₄-alkyl, cyclopropyl, cyclobutyl, and 4- to 6-membered heterocycloalkyl. In an embodiment, R^{4a} is independently selected from H, C₁-C₄-alkyl, and cyclopropyl.

In an embodiment, the compound of formula (I) is a compound of formula (X):

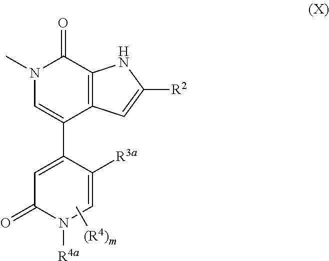

wherein R², R^{3a}, and R⁴ are as described above for compounds of formula (I); and wherein:
m is an integer selected from 0, 1, or 2; and
R^{4a} is independently selected from H, C₁-C₄-alkyl, cyclopropyl, cyclobutyl, and 4- to 6-membered heterocycloalkyl. R^{4a} may be independently selected from H, C₁-C₄-alkyl, and cyclopropyl.

In an embodiment, the compound of formula (I) is a compound of formula (XI):

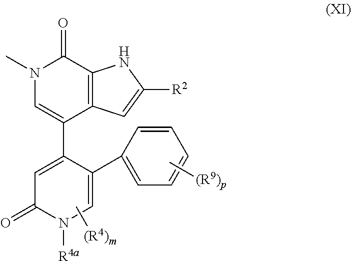

wherein R² and R⁴ are as described above for compounds of formula (I); and wherein:
m is an integer selected from 0, 1, or 2;
p is an integer selected from 0, 1, 2, 3, 4 and 5; and
R^{4a} is independently selected from H, C₁-C₄-alkyl, cyclopropyl, cyclobutyl, and 4- to 6-membered heterocycloalkyl. R^{4a} may be independently selected from H, C₁-C₄-alkyl, and cyclopropyl.

In an embodiment, the compound of formula (I) is a compound of formula (XII):

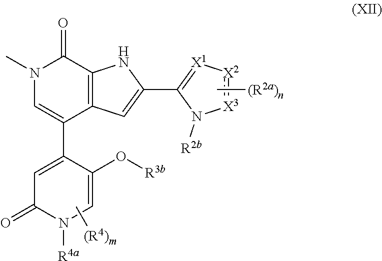

wherein R^{2a}, R^{3b}, and R⁴ are as described above for compounds of formula (I); and [symbol]
R^{2b}, X¹, X², X³ and n are as described above for compounds of formula (III); and wherein:
m is an integer selected from 0, 1, or 2; and
R^{4a} is independently selected from H, C₁-C₄-alkyl, cyclopropyl, cyclobutyl, and 4- to 6-membered heterocycloalkyl. R^{2b} may be H. R^{4a} may be independently selected from H, C₁-C₄-alkyl, and cyclopropyl.

In an embodiment, the compound of formula (I) is a compound of formula (XIII):

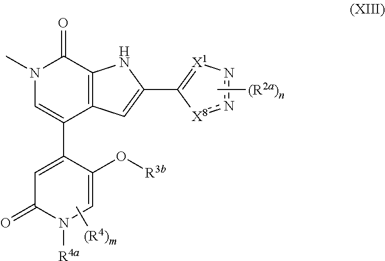

wherein R^{2a}, R^{3b}, R⁴ and m are as described above for compounds of formula (I) and wherein [symbol] is independently selected from a single bond and a double bond;
X¹ and X³ are each independently selected from carbon and nitrogen;
n is independently an integer selected from 0, 1, 2, 3 and 4; and
R^{4a} is independently selected from H, C₁-C₄-alkyl, cyclopropyl, cyclobutyl, and 4- to 6-membered heterocycloalkyl. R^{4a} may be independently selected from H, C₁-C₄-alkyl, and cyclopropyl.

In an embodiment, the compound of formula (I) is a compound of formula (XIV):

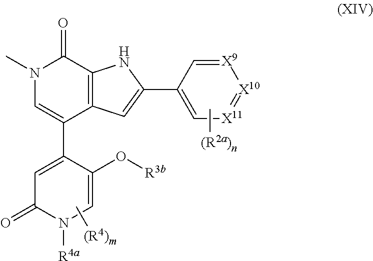

wherein R^{2a}, R^{3b}, R⁴ and m are as described above for compounds of formula (I);
X⁹, X¹⁰, and X¹¹ are each independently selected from carbon and nitrogen;
n is independently an integer selected from 0, 1, 2, 3, 4, and 5; and
R^{4a} is independently selected from H, C₁-C₄-alkyl, cyclopropyl, cyclobutyl, and 4- to 6-membered heterocycloalkyl. R^{4a} may be independently selected from H, C₁-C₄-alkyl, and cyclopropyl.

In an embodiment, the compound of formula (I) is a compound of formula (XV):

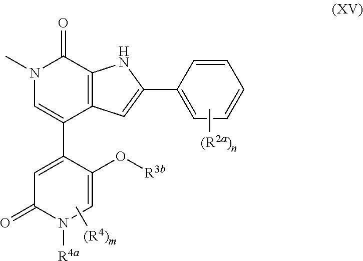

wherein R^{2a}, R^{3b}, R⁴ and m are as described above for compounds of formula (I);
n is independently an integer selected from 0, 1, 2, 3, 4, and 5; and
R^{4a} is independently selected from H, C₁-C₄-alkyl, cyclopropyl, cyclobutyl, and 4- to 6-membered heterocycloalkyl. R^{4a} may be independently selected from H, C₁-C₄-alkyl, and cyclopropyl.

In an embodiment, the compound of formula (I) is a compound of formula (XVI):

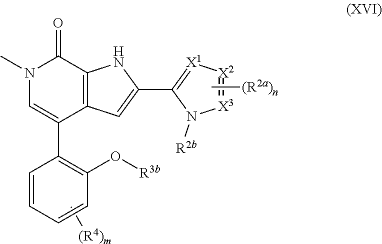

wherein R^{2a}, R^{3b}, R⁴, and m are as described above for compounds of formula (I); and [symbol] , R², X¹, X², X³ and n are as described above for compounds of formula (III). R^{2b} may be H.

In an embodiment, the compound of formula (I) is a compound of formula (XVII):

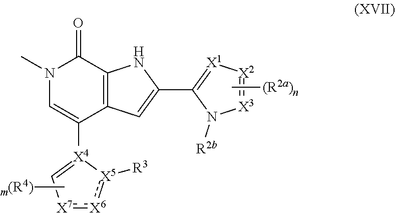

wherein X⁴, X⁵, R^{2a}, R³ and R⁴ are as described above for compounds of formula (I); and [symbol] R^{2b}, X¹, X², X³ and n are as described above for compounds of formula (III); and wherein
each [symbol] is independently selected from a single bond and a double bond;
X⁶ is independently selected from carbon and nitrogen;
X⁷ is independently selected from carbon and nitrogen; and
m is an integer selected from 0, 1, 2, or 3. R^{2b} may be H.

In an embodiment, the compound of formula (I) is a compound of formula (XVIII):

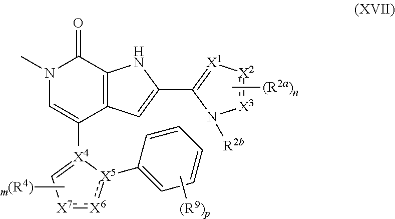

wherein X⁴, X⁵, R^{2a} and R⁴ are as described above for compounds of formula (I); and [symbol] , R^{2b}, X¹, X², X³ and n are as described above for compounds of formula (III); and wherein
each [symbol] is independently selected from a single bond and a double bond;
X⁶ is independently selected from carbon and nitrogen;
X⁷ is independently selected from carbon and nitrogen;
m is an integer selected from 0, 1, 2, or 3; and
p is an integer selected from 0, 1, 2, 3, 4 and 5.

In an embodiment, the compound of formula (I) is a compound of formula (XIX):

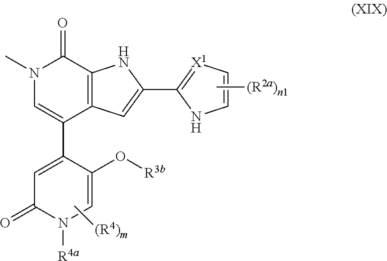

wherein R^{2a}, R^{3b} and R⁴ are as described above for compounds of formula (I), and wherein:
n1 is independently an integer selected from 0, 1 or 2;
m is an integer selected from 0, 1, or 2; and
R^{4a} is independently selected from H, methyl, cyclopropyl, and oxetan-3-yl. R^{4a} may be selected from H, methyl, and cyclopropyl.

In an embodiment, the compound of formula (I) is a compound of formula (XX):

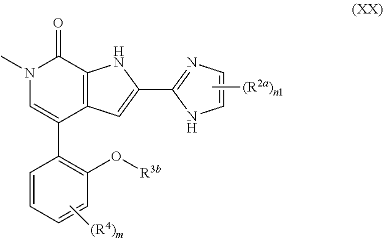

wherein R^{2a}, R^{3b}, R⁴, and m are as described above for compounds of formula (I); and wherein n1 is independently an integer selected from 0, 1 and 2.

In an embodiment, the compound of formula (I) is a compound of formula (XXI):

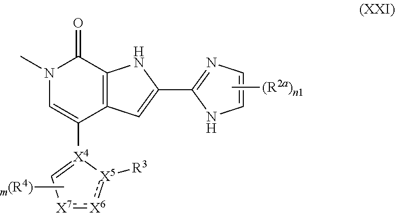

wherein X⁴, X⁵, R²¹, R³, R⁴ are as described above for compounds of formula (I); and wherein
each [symbol] is independently selected from a single bond and a double bond;
X⁶ is independently selected from carbon and nitrogen;
X⁷ is independently selected from carbon and nitrogen;
n1 is independently an integer selected from 0, 1 and 2; and m is an integer selected from 0, 1, 2, or 3.

In an embodiment, the compound of formula (I) is a compound of formula (XXII):

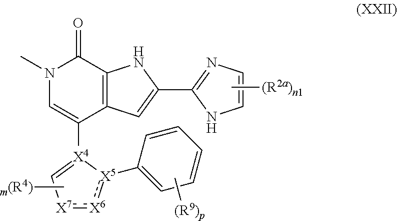

wherein X⁴, X⁵, R^{2a}, R⁴ and R⁹, are as described above for compounds of formula (I); and wherein
[symbol] is independently selected from a single bond and a double bond;
X⁶ is independently selected from carbon and nitrogen;
X⁷ is independently selected from carbon and nitrogen;
n1 is an integer selected from 0, 1 and 2;
m is an integer selected from 0, 1, 2, or 3; and
p is an integer selected from 0, 1, 2, 3, 4, and 5.

In an embodiment, the compound of formula (I) is a compound of formula (XXIII):

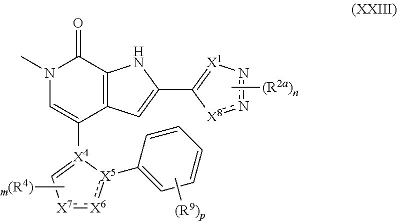

wherein X⁴, X⁵, R^{2a}, R⁴, and R⁹ are as described above for compounds of formula (I) and wherein [symbol] is independently selected from a single bond and a double bond;
X¹, X⁶, X⁷, and X⁸ are each independently selected from carbon and nitrogen;
n is an integer selected from 0, 1, 2, 3, or 4;
m is an integer selected from 0, 1, 2, or 3; and
p is an integer selected from 0, 1, 2, 3, 4, and 5.

In an embodiment, the compound of formula (I) is a compound of formula (XXIV):

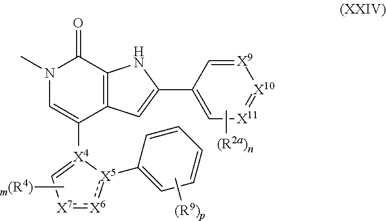

wherein X⁴, X⁵, R^{2a}, R⁴, and R⁹ are as described above for compounds of formula (I) and wherein [symbol] is independently selected from a single bond and a double bond;
X⁶, X⁷, X⁹, X¹⁰, and X¹¹ are each independently selected from carbon and nitrogen;
n is an integer selected from 0, 1, 2, 3, 4, or 5;
m is an integer selected from 0, 1, 2, or 3; and
p is an integer selected from 0, 1, 2, 3, 4, and 5.

In an embodiment, the compound of formula (I) is a compound of formula (XXV):

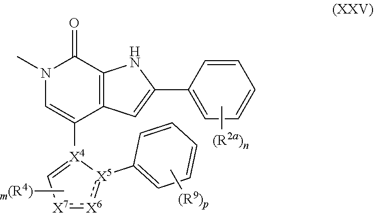

wherein X⁴, X⁵, R^{2a}, R⁴, and R⁹ are as described above for compounds of formula (I) and wherein [symbol] is independently selected from a single bond and a double bond;
X⁶ and X⁷ are each independently selected from carbon and nitrogen;
n is an integer selected from 0, 1, 2, 3, 4, or 5;
m is an integer selected from 0, 1, 2, or 3; and
p is an integer selected from 0, 1, 2, 3, 4, and 5.

In an embodiment, the compound of formula (I) is a compound of formula (XXVI):

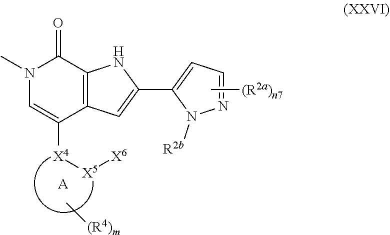

wherein X⁴, X⁵, Ring A, R^{2a}, R³, R⁴, and m are as described above for compounds of formula (I); wherein R^{2b} is as described above for compounds of formula (III); and wherein n7 is independently an integer selected from 0, 1 and 2.

In an embodiment, the compound of formula (I) is a compound of formula (XXVII):

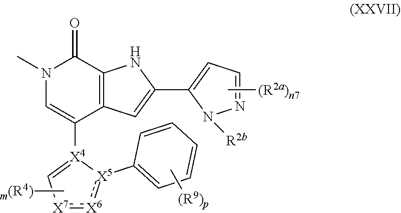

wherein X⁴, X⁵, R^{2a}, R⁴ and R⁹, are as described above for compounds of formula (I); wherein R^{2b} is as described above for compounds of formula (III); and wherein
[symbol] is independently selected from a single bond and a double bond;
X⁶ is independently selected from carbon and nitrogen;
X⁷ is independently selected from carbon and nitrogen;
n7 is an integer selected from 0, 1 and 2;
m is an integer selected from 0, 1, 2, or 3; and
p is an integer selected from 0, 1, 2, 3, 4, and 5.

In an embodiment, the compound of formula (I) is a compound of formula (XXVIII):

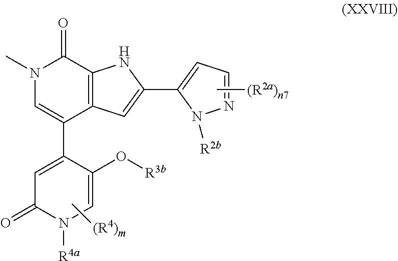

wherein R^{2a}, R^{3b} and R⁴ are as described above for compounds of formula (I); wherein R^{2b} is as described above for compounds of formula (III); and wherein:
n7 is independently an integer selected from 0, 1 or 2;
m is an integer selected from 0, 1, or 2; and
R^{4a} is independently selected from H, methyl, cyclopropyl, and oxetan-3-yl.

In an embodiment, the compound of formula (I) is a compound of formula (XXIX):

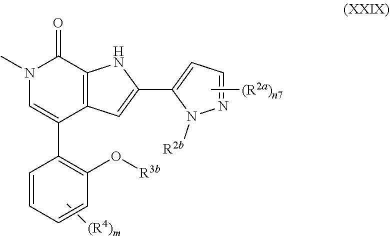

wherein R^{2a}, R^{3b}, R⁴, and m are as described above for compounds of formula (I); wherein R^{2b} is as described above for compounds of formula (III); and wherein n7 is independently an integer selected from 0, 1 and 2.

In an embodiment, the compound of formula (I) is a compound of formula (XXX):

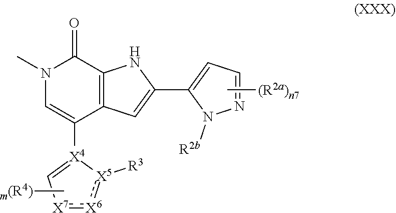

wherein X⁴, X⁵, R^{2a}, R³, R⁴ are as described above for compounds of formula (I); wherein R^{2b} is as described above for compounds of formula (III); and wherein
each [symbol] is independently selected from a single bond and a double bond;
X⁶ is independently selected from carbon and nitrogen;
X⁷ is independently selected from carbon and nitrogen;
n7 is independently an integer selected from 0, 1 and 2; and
m is an integer selected from 0, 1, 2, or 3.

In an embodiment, the compound of formula (I) is a compound of formula (XXXI):

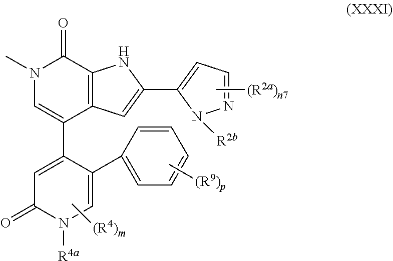

wherein R^{2a}, R⁴, R⁹ are as described above for compounds of formula (I); wherein R^{2b} is as described above for compounds of formula (III);
m is an integer selected from 0, 1 and 2; and
p is an integer selected from 0, 1, 2, 3, 4, and 5;
n7 is independently an integer selected from 0, 1 and 2;
and wherein R^{4a} is independently selected from H, C₁-C₄-alkyl, cyclopropyl, cyclobutyl, and 4- to 6-membered heterocycloalkyl.

In an embodiment, the compound of formula (I) is a compound of formula (XXXII):

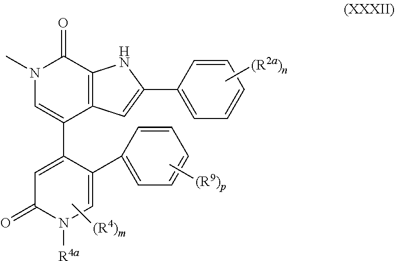

wherein R^{2a}, R⁴, R⁹ are as described above for compounds of formula (I);
m is an integer selected from 0, 1 and 2; and
p is an integer selected from 0, 1, 2, 3, 4, and 5;
n is independently an integer selected from 0, 1, 2, 3, 4, and 5;
and wherein R^{4a} is independently selected from H, C₁-C₄-alkyl, cyclopropyl, cyclobutyl, and 4- to 6-membered heterocycloalkyl.

In an embodiment, the compound of formula (I) is a compound of formula (XXXIII):

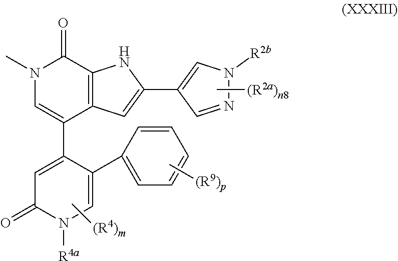

wherein R^{2a}, R⁴, R⁹ are as described above for compounds of formula (I); wherein R^{2b} is as described above for compounds of formula (III);
m is an integer selected from 0, 1 and 2; and
p is an integer selected from 0, 1, 2, 3, 4, and 5;
n8 is independently an integer selected from 0, 1 and 2;
and wherein R^{4a} is independently selected from H, C₁-C₄-alkyl, cyclopropyl, cyclobutyl, and 4- to 6-membered heterocycloalkyl.

In an embodiment, the compound of formula (I) is a compound of formula (XXXIV):

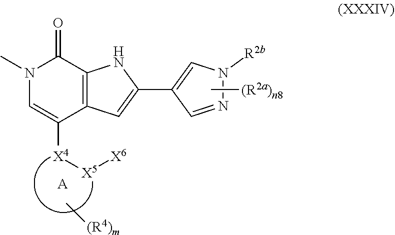

wherein X⁴, X⁵, Ring A, R^{2a}, R³, R⁴, and m are as described above for compounds of formula (I); wherein R^{2b} is as described above for compounds of formula (III); and wherein n8 is independently an integer selected from 0, 1 and 2.

In an embodiment, the compound of formula (I) is a compound of formula (XXXV):

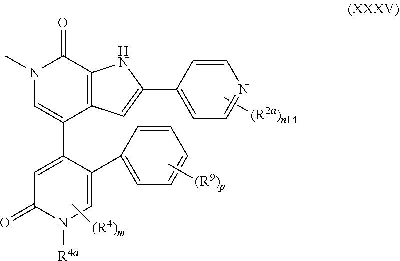

wherein R^{2a}, R⁴, R⁹ are as described above for compounds of formula (I);
m is an integer selected from 0, 1 and 2; and
p is an integer selected from 0, 1, 2, 3, 4, and 5;
n14 is independently an integer selected from 0, 1, 2, 3 and 4;
and wherein R^{4a} is independently selected from H, C₁-C₄-alkyl, cyclopropyl, cyclobutyl, and 4- to 6-membered heterocycloalkyl.

In an embodiment, the compound of formula (I) is a compound of formula (XXXVI):

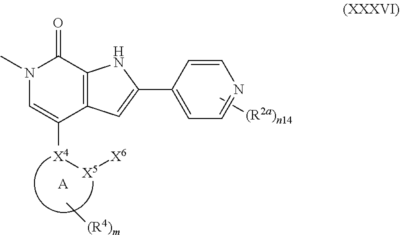

wherein X⁴, X⁵, Ring A, R^{2a}, R³, R⁴, and m are as described above for compounds of formula (I); and wherein n14 is independently an integer selected from 0, 1, 2, 3 and 4.

In an embodiment, the compound of formula (I) is a compound of formula (XXXVII):

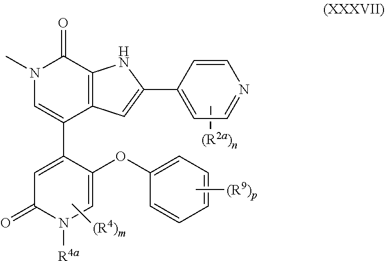

wherein R^{2a}, R⁴, R⁹ are as described above for compounds of formula (I);
m is an integer selected from 0, 1 and 2; and
p is an integer selected from 0, 1, 2, 3, 4, and 5;
n is independently an integer selected from 0, 1, 2, 3, 4 and 5;
and wherein R^{4a} is independently selected from H, C₁-C₄-alkyl, cyclopropyl, cyclobutyl, and 4- to 6-membered heterocycloalkyl.

In an embodiment, the compound of formula (I) is a compound of formula (XXXVIII):

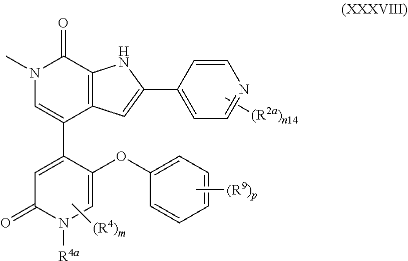

wherein R^{2a}, R⁴, R⁹ are as described above for compounds of formula (I);
m is an integer selected from 0, 1 and 2; and
p is an integer selected from 0, 1, 2, 3, 4, and 5;
n14 is independently an integer selected from 0, 1, 2, 3 and 4;
and wherein R^{4a} is independently selected from H, C₁-C₄-alkyl, cyclopropyl, cyclobutyl, and 4- to 6-membered heterocycloalkyl.

The following embodiments apply to compounds of any of formulae (I)-(XXXVIII). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

In an embodiment, R¹ is C₁-C₃-alkyl. In an embodiment, R¹ is C₁-C₃-fluoroalkyl. In an embodiment, R¹ is C₃-cycloalkyl. In an embodiment, R¹ is independently selected from C₁-alkyl, C₁-fluoroalkyl, and C₃-cycloalkyl. Preferably, R¹ is C₁-alkyl, i.e., methyl.

In an embodiment, R² is

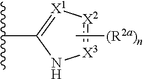

wherein [symbol] is independently selected from a single bond and a double bond;
X¹ is independently selected from carbon and nitrogen;
X² and X³ are each independently selected from carbon, nitrogen, oxygen and sulfur; wherein where one of X² or X³ is oxygen or sulfur, the other of X² and X³ must be carbon; and where [symbol] is a bond, X² and X³ are each independently selected from carbon and nitrogen; and
n is independently an integer selected from 0, 1, 2, 3 and 4.

In an embodiment, R² is

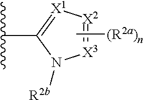

wherein [symbol] is independently selected from a single bond and a double bond; and R^{2b} is independently selected from H, C₁-C₄-alkyl, C₃-C₆ cycloalkyl, and 4- to 6-membered heterocyclyl.

In an embodiment, R² is

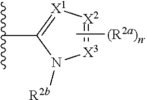

In an embodiment, X¹ is carbon. In an embodiment, X¹ is nitrogen.

In an embodiment, X² and X³ are each independently selected from carbon and nitrogen. In an embodiment, X² and X³ are each independently selected from carbon and oxygen. In an embodiment, X² and X³ are each carbon.

In an embodiment, R² is a 5-membered heterocyclyl group; optionally substituted with from 1 to 4 R^{2a} groups.

In an embodiment, R² is

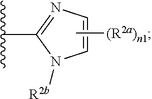

wherein n1 is independently an integer selected from 0, 1 and 2.

In an embodiment, R² is

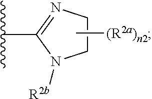

wherein n2 is independently an integer selected from 0, 1, 2 and 3.

In an embodiment, R² is

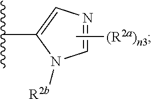

wherein n3 is independently an integer selected from 0, 1 and 2.

In an embodiment, R² is

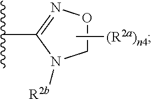

wherein n4 is independently an integer selected from 0, 1 and 2.

In an embodiment, R² is

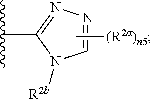

wherein n5 is independently an integer selected from 0 and 1.

In an embodiment, R² is

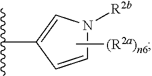

wherein n6 is independently an integer selected from 0, 1, 2, 3, and 4.

In an embodiment, R² is

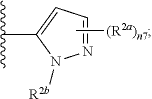

wherein n7 is independently an integer selected from 0, 1, 2, and 3.

In an embodiment, R² is

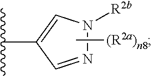

wherein n8 is independently an integer selected from 0, 1, 2, and 3.

In an embodiment, R² is

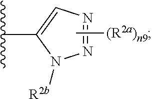

wherein n9 is independently an integer selected from 0, 1, and 2.

In an embodiment, R² is

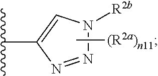

wherein n11 is independently an integer selected from 0, 1, and 2.

R^{2b} is independently selected from H, C₁-C₄-alkyl, C₃-C₅ cycloalkyl, and 4- to 6-membered heterocyclyl. R^{2b} may be H. R^{2b} may be selected from H, C₁-C₄-alkyl and cyclopropyl. R^{2b} may be selected from C₁-C₄-alkyl and cyclopropyl. R^{2b} may be C₁-C₄-alkyl, e.g., methyl. R^{2b} may be 4- to 6-membered heterocyclyl. R^{2b} may be oxetanyl or azetidinyl. In an embodiment, R^{2b} is oxetanyl. In an embodiment, R^{2b} is oxetan-3-yl.

In an embodiment, R² is

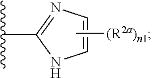

wherein n1 is independently an integer selected from 0, and 2.

In an embodiment, R² is

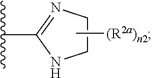

wherein n2 is independently an integer selected from 0, 1, 2 and 3.

In an embodiment, R² is

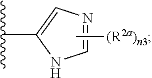

wherein n3 is independently an integer selected from 0, 1 and 2.

In an embodiment, R² is

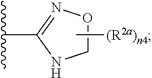

wherein n4 is independently an integer selected from 0, 1 and 2.

In an embodiment, R² is

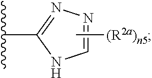

wherein n5 is independently an integer selected from 0 and 1.

In an embodiment, R² is

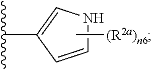

wherein n6 is independently an integer selected from 0, 1, 2, 3, and 4.

In an embodiment, R² is

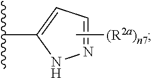

wherein n7 is independently an integer selected from 0, 1, 2, and 3.

In an embodiment, R² is

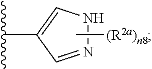

wherein n8 is independently an integer selected from 0, 1, 2, and 3. In an embodiment R^{2a} is not C₁-C₄-alkyl. In an embodiment R^{2a} is not methyl.

In an embodiment, R² is

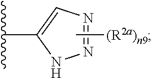

wherein n9 is independently an integer selected from 0, 1, and 2.

In an embodiment, R² is

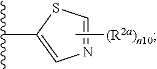

wherein n10 is independently an integer selected from 0, 1, and 2.

In an embodiment, R² is

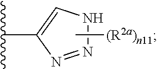

wherein n11 is independently an integer selected from 0, 1, and 2.

In an embodiment, R² is

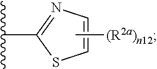

wherein n12 is independently an integer selected from 0, 1, 2, 3, and 4.

In an embodiment, R² is

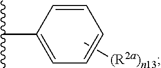

wherein n13 is independently an integer selected from 0, 1, 2, 3, 4, and 5.

In an embodiment, R² is

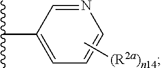

wherein n14 is independently an integer selected from 0, 1, 2, 3, and 4.

In an embodiment, R² is

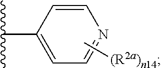

wherein n14 is independently an integer selected from 0, 1, 2, 3, and 4.

In an embodiment, R² is

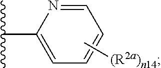

wherein n14 is independently an integer selected from 0, 1, 2, 3, and 4.

In an embodiment, R² is

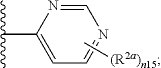

wherein n15 is independently an integer selected from 0, 1, 2, and 3.

In an embodiment, R² is

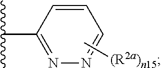

wherein n15 is independently an integer selected from 0, 1, 2, and 3.

In an embodiment, R² is

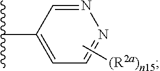

wherein n15 is independently an integer selected from 0, 1, 2, and 3.

In an embodiment, R² is

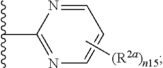

wherein n15 is independently an integer selected from 0, 1, 2, and 3.

In an embodiment, R² is

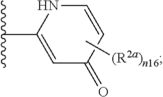

wherein n16 is independently an integer selected from 0, 1, 2, 3, and 4.

In an embodiment, R² is a substituted or unsubstituted imidazolidine or a substituted or unsubstituted imidazoline.

In an embodiment, R² is a substituted or unsubstituted thiazole or a substituted or unsubstituted isothiazole.

In an embodiment, R² is a substituted or unsubstituted triazole.

In an embodiment, R² is a substituted or unsubstituted pyridazine (unsaturated or saturated), a substituted or unsubstituted pyrimidine (unsaturated or saturated), a substituted or unsubstituted pyrazine, or a substituted or unsubstituted piperazine.

In an embodiment, R² is a substituted or unsubstituted triazine or a substituted or unsubstituted triazinane.

In an embodiment, R² is a substituted or unsubstituted pyridone.

In an embodiment, R² is a substituted or unsubstituted pyrazolidine or a substituted or unsubstituted pyrazoline.

In embodiments where R² is depicted as comprising an NH group within the ring, it is to be understood that the nitrogen atom may be substituted with an R^{2a} group defined herein, where chemically possible, to give an NR^{2a} group within the ring.

In an embodiment, R^{2a} is independently at each occurrence selected from ═O, halo, nitro, cyano, NR⁵R⁶, OR⁷, SR⁶, S(O)₂R⁶, SO₂NR⁶R⁶, CO₂R⁶, C(O)R⁶, CONR⁶R⁶, C₁-C₄-alkyl, C₁-C₄-haloalkyl, C₃-C₆ cycloalkyl, and 4- to 6-membered heterocyclyl.

In an embodiment, R^{2a} is independently at each occurrence selected from ═O, halo, cyano, CO₂R⁶, C(O)R⁶, C₁-C₄-alkyl, C₁-C₄-haloalkyl, C₃-C₆ cycloalkyl, and 4- to 6-membered heterocyclyl.

In an embodiment, R^{2a} is independently at each occurrence selected from ═O, halo, cyano, S(O)₂R⁶, CO₂R⁶, CONR⁶R⁶, C₁-C₄-alkyl, C₁-C₄-haloalkyl, C₃-C₆ cycloalkyl, and 4- to 6-membered heterocyclyl.

In an embodiment, R^{2a} is independently at each occurrence selected from ═O, halo, cyano, S(O)₂R⁶, CO₂R⁶, CONR⁶R⁶, C₁-C₂-alkyl, C₁-C₂-haloalkyl, C₃-C₄ cycloalkyl, and 4- to 6-membered heterocyclyl.

In an embodiment, R^{2a} is independently at each occurrence selected from ═O, halo, cyano, S(O)₂R⁶, CO₂R⁶, CONR⁶R⁶, C₁-C₂-alkyl, C₁-C₂-haloalkyl, cyclopropyl, cyclobutyl, and 4-membered heterocyclyl.

In an embodiment, R^{2a} is independently at each occurrence selected from ═O, halo, cyano, S(O)₂R⁶, CO₂R⁶, CONR⁶R⁶, C₁-C₂-alkyl, C₁-C₂-haloalkyl, and 4-membered heterocyclyl.

In an embodiment, R^{2a} is independently at each occurrence selected from ═O, halo, OR⁷, C₁-C₄-alkyl, and C₁-C₄-haloalkyl.

In an embodiment, R^{2a} is independently at each occurrence selected from halo, OR⁷, C₁-C₄-alkyl, and C₁-C₄-haloalkyl. In an embodiment, R^{2a} is independently at each occurrence selected from halo, C₁-C₄-alkyl, and C₁-C₄-haloalkyl. In an embodiment, R^{2a} is independently at each occurrence selected from C₁-alkyl and C₁-haloalkyl. In an embodiment, R^{2a} is C₁-C₄-alkyl, e.g., methyl.

In an embodiment, n is an integer selected from 0, 1, and 2. In an embodiment, n is 2. In an embodiment, n is 0. Preferably, n is 1. In an embodiment, where n is 1, R² is attached to X³.

In an embodiment, n1 is 0. Preferably, n1 is 1. In an embodiment, where n1 is 1, R^{2a} is attached to X³.

In an embodiment, n is an integer selected from 0, 1, and 2. In an embodiment, n2 is 2. In an embodiment, n2 is 0. In an embodiment, n2 is 1.

In an embodiment, n3 is 0. Preferably, n3 is 1. In an embodiment, where n3 is 1, R^{2a} is attached to X³.

In an embodiment, n4 is 2. In an embodiment, n4 is 0. In an embodiment, n4 is 1.

In an embodiment, n5 is 0. In an embodiment, n5 is 1.

In an embodiment, n6 is 0. In an embodiment, n6 is 1. In an embodiment, n6 is 2.

In an embodiment, n6 is 3.

In an embodiment, n7 is 0. In an embodiment, n7 is 1. In an embodiment, n7 is 2.

In an embodiment, n7 is 3.

In an embodiment, n8 is 0. In an embodiment, n8 is 1. In an embodiment, n8 is 2.

In an embodiment, n8 is 3.

In an embodiment, n9 is 0. In an embodiment, n9 is 1. In an embodiment, n9 is 2.

In an embodiment, n10 is 0. In an embodiment, n10 is 1. In an embodiment, n10 is 2.

In an embodiment, n11 is 0. In an embodiment, n11 is 1. In an embodiment, n11 is 2.

In an embodiment, n12 is 0. In an embodiment, n12 is 1. In an embodiment, n12 is 2.

In an embodiment, n13 is 0. In an embodiment, n13 is 1. In an embodiment, n13 is 2. In an embodiment, n13 is 3.

In an embodiment, n14 is 0. In an embodiment, n14 is 1. In an embodiment, n14 is 2. In an embodiment, n14 is 3.

In an embodiment, n15 is 0. In an embodiment, n15 is 1. In an embodiment, n15 is 2. In an embodiment, n15 is 3.

In an embodiment, n16 is 0. In an embodiment, n16 is 1. In an embodiment, n16 is 2.

In an embodiment, R² is selected from:

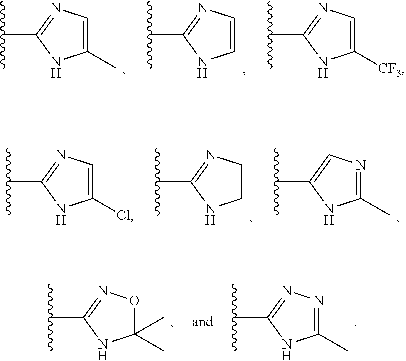

In an embodiment, R² is selected from:

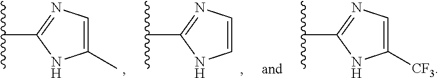

In an embodiment, R² is

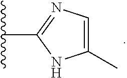 [SMILES: *c1ncc(C)[nH]1]

In an embodiment, R² is selected from:

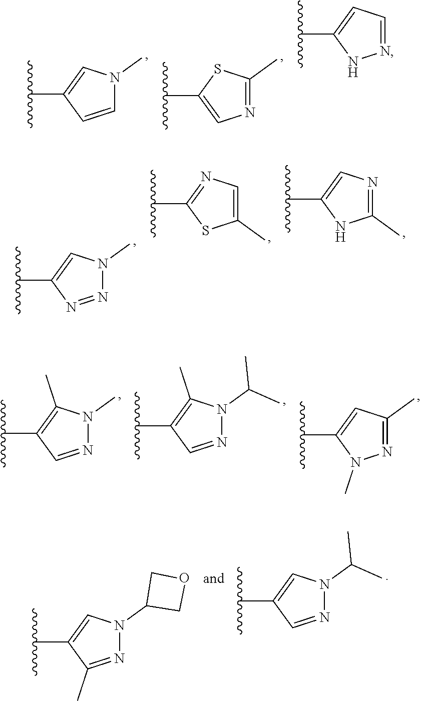

In an embodiment, R² is selected from:

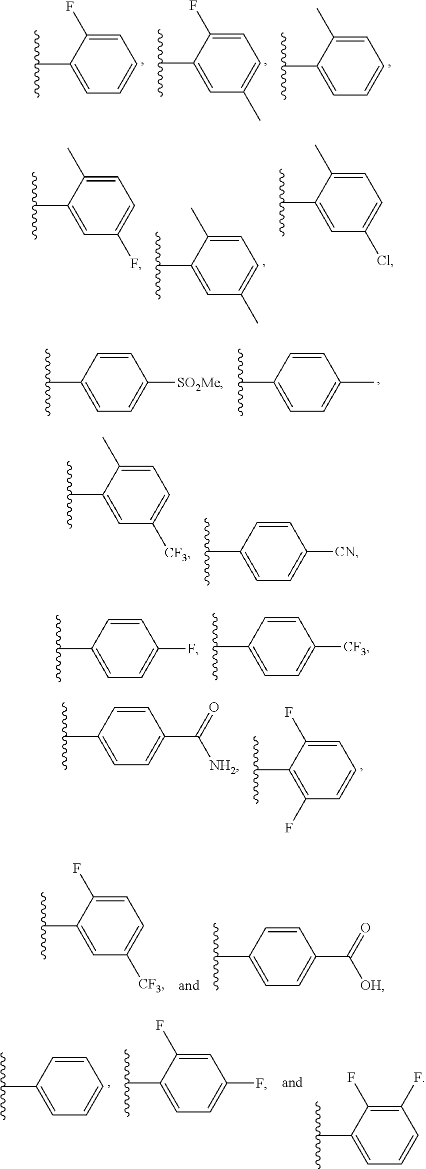

In an embodiment, R² is selected from:

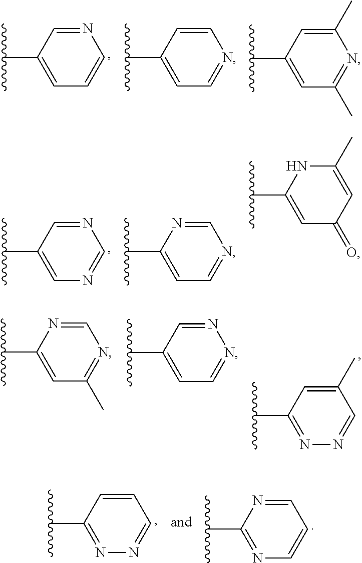

In an embodiment, X⁴ is carbon. In an embodiment, X⁴ is nitrogen.

In an embodiment, X⁵ is carbon. In an embodiment, X⁵ is nitrogen.

In an embodiment, Ring A is a phenyl ring. In an embodiment, Ring A is a 5- or 6-membered heterocyclyl. In an embodiment, Ring A is a 5- or 6-membered heteroaryl. In an embodiment, Ring A is a 5-membered heteroaryl ring. In an embodiment, Ring A is a 6-membered heterocyclyl ring. In an embodiment, Ring A is a 6-membered heteroaryl ring.

In an embodiment, when Ring A is a 5 membered heterocyclyl it is not a pyrrolidone.

In an embodiment, Ring A is phenyl. In an embodiment, Ring A is pyridone. Said pyridine may be substituted on the nitrogen with a C₁-C₄-alkyl group, a cyclopropyl, a cyclobutyl, or a 4-membered heterocycloalkyl group. Said pyridone may be substituted on the nitrogen with either a C₁-C₄-alkyl group or a cyclopropyl group. In an embodiment, Ring A is N—C₁-C₄-alkyl pyridone. In an embodiment, Ring A is pyridine. In an embodiment, Ring A is pyrrole. In an embodiment, Ring A is imidazole. In an embodiment, Ring A is pyrazole.

In an embodiment, Ring A is triazole. In an embodiment, Ring A is tetrazole.

In an embodiment,

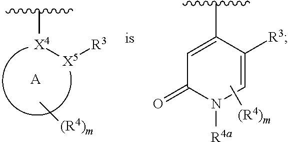

wherein R^{4a} is selected from H, C₁-C₄-alkyl (e.g., methyl), cyclopropyl, cyclobutyl, 4- to 6-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}. In an embodiment the heterocycloalkyl is oxetanyl or an azetidinyl.

In an embodiment,

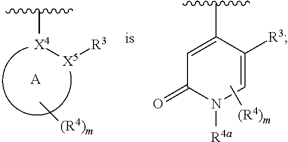

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

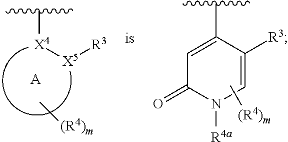

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl), cyclopropyl, and oxetan-3-yl.

In an embodiment,

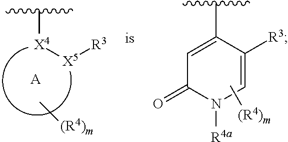

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In an embodiment,

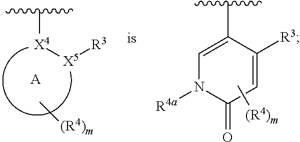

wherein R^{4a} is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

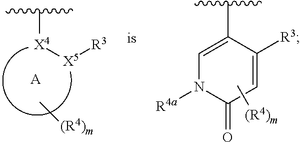

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

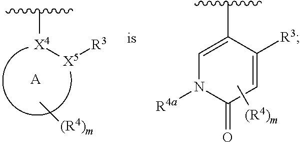

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl), cyclopropyl, and oxetan-3-yl.

In an embodiment,

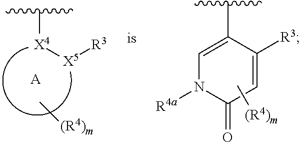

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In an embodiment,

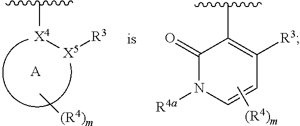

wherein R⁴ is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

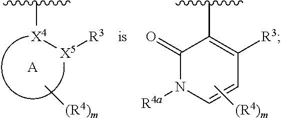

wherein R^{4a} is selected from H, C₁-C₄-alkyl (e.g., methyl), cyclopropyl, SOR⁶, S(O)₂R^{b}, SO₂NR⁶R⁶, C₀-C₄-alkylene-CO₂R, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

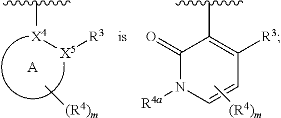

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e. g., methyl), cyclopropyl, and oxetan-3-yl.

In an embodiment,

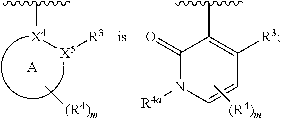

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In an embodiment,

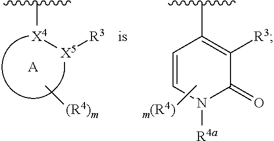

wherein R^{4a} is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

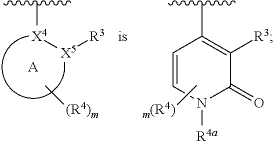

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

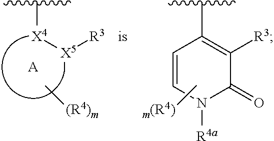

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl) cyclopropyl, and oxetan-3-yl.

In an embodiment,

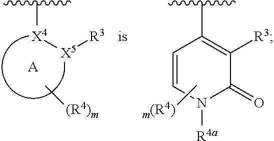

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In an embodiment,

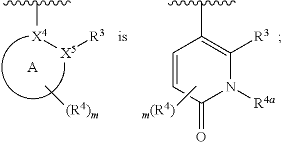

wherein R^{4a} is selected from H, C₁-C₄-alkyl (e.g., methyl), cyclopropyl, cyclobutyl, 4-membered heterocycloalkyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

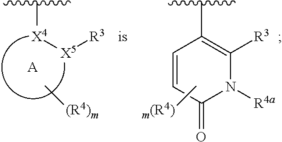

wherein R^{4a} is selected from H, C₁-C₄-alkyl (e.g., methyl), cyclopropyl, SOR⁶, S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl-S(O)₂R⁶, C₂-C₄-alkylene-NR⁵R⁶, C₂-C₄-alkylene-OR⁷, and cyclopropyl-OR^{a}; optionally wherein R³ is OR^{3b}.

In an embodiment,

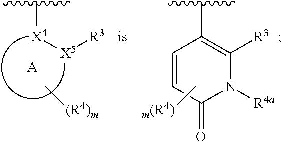

wherein R^{4a} is selected from H, C₁-C₄-alkyl (e.g., methyl), cyclopropyl, cyclobutyl, and 4-membered heterocycloalkyl; optionally wherein R³ is OR^{3b}. R^{4a} may be selected from C₁-C₄-alkyl (e.g., methyl) cyclopropyl, and oxetan-3-yl.

In an embodiment,

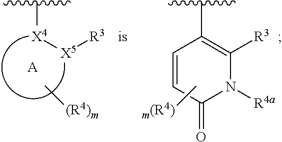

wherein R^{4a} is selected from H, C₁-C₄-alkyl (e.g., methyl), and cyclopropyl; optionally wherein R³ is OR^{3b}. R^{4a} may be selected from C₁-C₄-alkyl (e.g., methyl) and cyclopropyl.

In an embodiment,

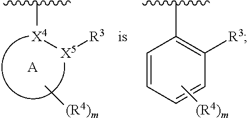

optionally wherein R³ is OR^{3b}.

In an embodiment,

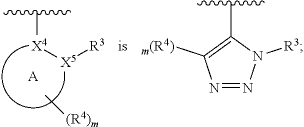

optionally wherein R³ is R³.

In an embodiment,

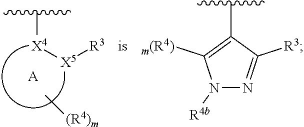

wherein R^{4b} is selected from S(O)₂R⁶, C₁-C₄-alkyl, C₁-C₄-alkyl-S(O)₂R⁶, C₁-C₄-haloalkyl, cyclopropyl, and cyclobutyl; optionally wherein R³ is R^{3a}.

In an embodiment,

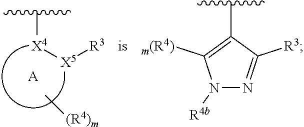

wherein R^{4b} is selected from S(O)₂R⁶, C₁-C₄-alkyl, C₁-C₄-alkyl-S(O)₂R⁶, C₁-C₄-haloalkyl, and cyclopropyl; optionally wherein R³ is R^{3a}.

In an embodiment,

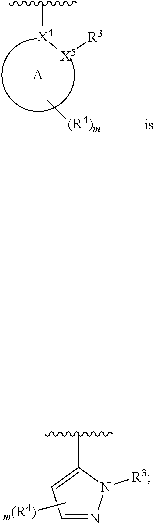

optionally wherein R³ is R^{3a}.

In an embodiment,

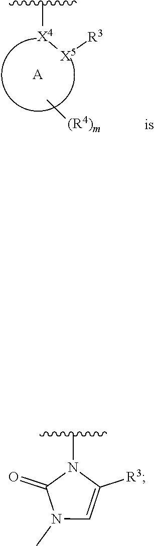

optionally wherein R³ is R^{3a}.

In an embodiment,

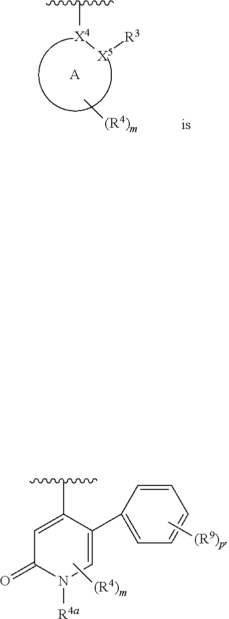

wherein
m is an integer selected from 0, 1 and 2; and
p is an integer selected from 0, 1, 2, 3, 4, and 5;
and R^{4a} is independently selected from H, C₁-C₄-alkyl, cyclopropyl, cyclobutyl, and 4- to 6-membered heterocycloalkyl.

In an embodiment, R³ is independently selected from R^{3a} and OR^{3b}. In an embodiment, R³ is R^{3a}. In an embodiment, R³ is OR^{3b}. It may be that, where Ring A is a 5-membered heteroaryl group, R³ is R^{3a}. It may be that, where Ring A is pyridone group, R³ is R^{3a}. It may be that, where Ring A is phenyl or pyridone, R³ is OR^{3b}.

In an embodiment, R^{3a} is independently selected from H, CN, C₁-C₄-alkyl, C₂-C₄-alkenyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₃-cycloalkenyl, 3- to 8-membered heterocycloalkyl, phenyl and 5- or 6-membered heteroaryl; wherein where R^{3c} is cycloalkyl or heterocycloalkyl, R^{3c} is optionally substituted with from 1 to 4 R³ groups and where R^{3c} is phenyl or heteroaryl, R^{3c} is optionally substituted with from 1 to 5 R⁹ groups;

In an embodiment, R^{3a} is independently selected from CN, C₁-C₄-alkyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₃-cycloalkenyl, 5- to 8-membered heterocycloalkenyl, 3- to 8-membered heterocycloalkyl, and phenyl; wherein where R^{3c} is cycloalkyl, heterocycloalkyl, cycloalkenyl, or heterocycloalkenyl, R^{3c} is optionally substituted with from 1 to 4 R³ groups and where R^{3c} is phenyl, R^{3c} is optionally substituted with from 1 to 5 R⁹ groups.

In an embodiment, R^{3a} is independently selected from CN, C₁-C₄-alkyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₃-cycloalkenyl, 3- to 8-membered heterocycloalkyl, and phenyl; wherein where R^{3c} is cycloalkyl or heterocycloalkyl, R^{3c} is optionally substituted with from 1 to 4 R³ groups and where R^{3c} is phenyl, R^{3c} is optionally substituted with from 1 to 5 R⁹ groups.

In an embodiment, R^{3a} is independently selected from C₃-C₄-alkyl, C₃-C₄-haloalkyl, C₃-haloalkenyl, and C₀-C₃-alkylene-R^{3c}; wherein R^{3c} is independently at each occurrence selected from C₃-C₆-cycloalkyl, C₅-C₆-cycloalkenyl, 5- to 6-membered heterocycloalkenyl, 4- to 6-membered heterocycloalkyl, and phenyl; wherein where R^{3c} is cycloalkyl, heterocycloalkyl, cycloalkenyl, or heterocycloalkenyl, R^{3c} is optionally substituted with from 1 to 4 R³ groups and where R^{3c} is phenyl, R^{3c} is optionally substituted with from 1 to 5 R⁹ groups.

In an embodiment, R^{3a} is independently selected from C₃-C₄-alkyl, C₃-C₄-haloalkyl, C₃-haloalkenyl, and C₀-C₃-alkylene-R^{3c}; wherein R^{3c} is independently at each occurrence selected from C₃-C₆-cycloalkyl, C₅-C₆-cycloalkenyl, 4- to 6-membered heterocycloalkyl, and phenyl; wherein where R^{3c} is cycloalkyl or heterocycloalkyl, R^{3c} is optionally substituted with from 1 to 4 R³ groups and where R^{3c} is phenyl, R^{3c} is optionally substituted with from 1 to 5 R⁹ groups.

In an embodiment, R^{3a} is C₀-C₃-alkylene-R^{3c}; wherein R^{3c} is independently at each occurrence selected from C₅-cycloalkyl, C₅-cycloalkenyl, 6-membered heterocycloalkenyl, 6-membered heterocycloalkyl, and phenyl; wherein where R^{3c} is cycloalkyl, cycloalkenyl, heterocycloalkenyl or heterocycloalkyl, R^{3c} is optionally substituted with from 1 to 4 R⁸ groups and where R^{3c} is phenyl, R^{3c} is optionally substituted with from 1 to 5 R⁹ groups.

In an embodiment, R^{3a} is C₀-C₃-alkylene-R^{3c}; wherein R^{3c} is independently at each occurrence selected from C₅-cycloalkyl, C₅-cycloalkenyl, 6-membered heterocycloalkyl, and phenyl; wherein where R^{3c} is cycloalkyl, cycloalkenyl or heterocycloalkyl, R^{3c} is optionally substituted with from 1 to 4 R³ groups and where R^{3c} is phenyl, R^{3c} is optionally substituted with from 1 to 5 R⁹ groups.

In an embodiment, R^{3a} is C₀-C₃-alkylene-R^{3c}; wherein R^{3c} is phenyl; and wherein R^{3c} is optionally substituted with from 1 to 5 R⁹ groups.

In an embodiment, R^{3a} is phenyl, optionally substituted with from 1 to 3 R⁹ groups. Where R^{3c}, R^{3a} or R³ are phenyl, it may be that the phenyl is substituted with from 1 to 3 R⁹ groups.

In an embodiment, R^{3a} is R^{3c}; wherein R^{3c} is phenyl; wherein R^{3c} is optionally substituted with from 1 or 2 R⁹ groups; and wherein the para-position on the phenyl group is unsubstituted.

In an embodiment, R^{3a} is C₃-C₄-alkyl. In an embodiment, R^{3a} is C₃-C₄-haloalkyl. In an embodiment, R^{3a} is C₂-C₄-haloalkenyl. In an embodiment, R^{3a} is and C₀-C₃-alkylene-R^{3c}; wherein R^{3c} is independently at each occurrence selected from C₃-C₆-cycloalkyl, C₅-C₆-cycloalkenyl, 5- to 6-membered heterocycloalkenyl, and 4- to 6-membered heterocycloalkyl.

In an embodiment, R^{3a} is C₃-C₆-cycloalkyl. In an embodiment, R^{3a} is C₅-C₆-cycloalkenyl. In an embodiment, R^{3a} is 5- to 6-membered heterocycloalkenyl, In an embodiment, R^{3a} is 4-membered heterocycloalkyl. In an embodiment, R^{3a} is 5-membered heterocycloalkyl. In an embodiment, R^{3a} is 6-membered heterocycloalkyl. It may be that where R^{3a} or R^{3c} is cycloalkyl or heterocycloalkyl, R^{3c} is substituted with from 1 to 4 R⁸ groups.

In an embodiment, R^{3a} is C₃-C₄-alkyl. In an embodiment, R^{3a} is C₃-C₄-haloalkyl. In an embodiment, R^{3a} is C₂-C₄-haloalkenyl. In an embodiment, R^{3a} is and C₀-C₃-alkylene-R^{3c}; wherein R^{3c} is independently at each occurrence selected from C₃-C₆-cycloalkyl, C₅-C₆-cycloalkenyl, and 4- to 6-membered heterocycloalkyl. In an embodiment, R^{3a} is C₃-C₆-cycloalkyl. In an embodiment, R^{3a} C₅-C₆-cycloalkenyl. In an embodiment, R^{3a} is 4-membered heterocycloalkyl. In an embodiment, R^{3a} is 5-membered heterocycloalkyl. In an embodiment, R^{3a} is 6-membered heterocycloalkyl. It may be that where R^{3a} or R^{3c} is cycloalkyl or heterocycloalkyl, R^{3c} is substituted with from 1 to 4 R⁸ groups.

In an embodiment, R³ is selected from phenyl or —O-phenyl, wherein R³ is optionally substituted with from 1 to 5 R⁹ groups. In an embodiment, R³ is unsubstituted phenyl. In an embodiment, R³ is —O-phenyl, wherein R³ is substituted with 2 R⁹ groups.

In an embodiment, R³ is

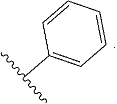 [SMILES: *c1ccccc1]

In an embodiment, R³ is

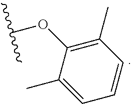 [SMILES: *Oc1c(C)cccc1C]

It may be that where Ring A is 5-membered heteroaryl, R^{3a} is optionally substituted phenyl. It may be that where Ring A is 5-membered heteroaryl, R^{3a} is optionally substituted 6-membered heteroaryl.

In an embodiment, R^{3a} is selected from:

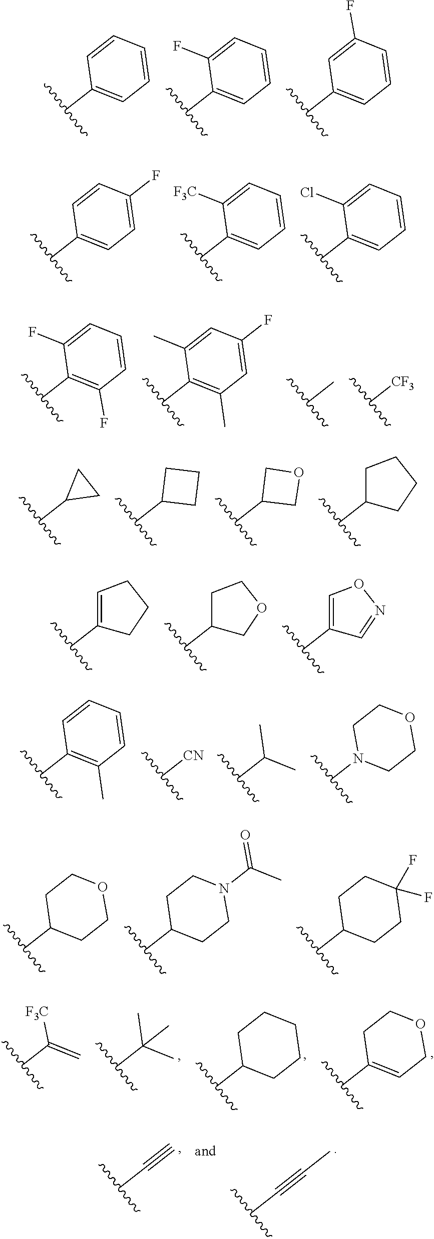

In an embodiment, R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₃-cycloalkyl, 3- to 8-membered heterocycloalkyl, and phenyl; wherein where R^{3d} is cycloalkyl or heterocycloalkyl, R^{3d} is optionally substituted with from 1 to 4 R⁸ groups and where R^{3d} is phenyl, R^{3d} is optionally substituted with from 1 to 5 R⁹ groups.

In an embodiment, R^{3b} is independently selected from C₄-alkyl, C₂-C₄-alkylene-O—C₁, C₄-haloalkyl and C₀-C₃-alkylene-R^{3d}; wherein R^{3d} is independently at each occurrence selected from C₃-C₅-cycloalkyl, 4- to 6-membered heterocycloalkyl, and phenyl; wherein where R^{3d} is cycloalkyl or heterocycloalkyl, R^{3d} is optionally substituted with from 1 to 4 R⁸ groups and where R^{3d} is phenyl, R^{3d} is optionally substituted with from 1 to 5 R⁹ groups.

In an embodiment, R^{3b} is C₀-C₃-alkylene-R^{3d}; wherein R^{3d} is independently at each occurrence selected from C₃-C₆-cycloalkyl, 4- to 6-membered heterocycloalkyl, and phenyl; wherein where R^{3d} is cycloalkyl or heterocycloalkyl, R^{3d} is optionally substituted with from 1 to 4 R⁸ groups and where R^{3d} is phenyl, R^{3d} is optionally substituted with from 1 to 5 R⁹ groups.

In an embodiment, R^{3b} is C₀-C₃-alkylene-R^{3d}; wherein R^{3d} is independently at each occurrence selected from C₅-cycloalkyl, 6-membered heterocycloalkyl, and phenyl; wherein where R^{3d} is cycloalkyl or heterocycloalkyl, R^{3d} is optionally substituted with from 1 to 4 R⁸ groups and where R^{3d} is phenyl, R^{3d} is optionally substituted with from 1 to 5 R⁹ groups.

In an embodiment, R^{3b} is C₀-C₃-alkylene-R^{3d}; wherein R^{3d} is phenyl; and wherein R^{3d} is optionally substituted with from 1 to 5 R⁹ groups.

In an embodiment, R^{3b} is phenyl; optionally substituted with from 1 to 3 R⁹ groups.

In an embodiment, R^{3b} is C₀-C₃-alkylene-R^{3d}; wherein R^{3d} is independently at each occurrence selected from C₃-C₆-cycloalkyl, and 4- to 6-membered heterocycloalkyl; wherein R^{3d} is optionally substituted with from 1 to 4 R⁸ groups.

In an embodiment, R^{3b} is C₀-C₃-alkylene-R^{3d}; wherein R^{3d} is C₃-C₆-cycloalkyl; wherein R^{3d} is optionally substituted with from 1 to 4 R⁸ groups.

In an embodiment, R^{3b} is C₀-C₃-alkylene-R^{3d}; wherein R^{3d} is 4- to 6-membered heterocycloalkyl; wherein R^{3d} is optionally substituted with from 1 to 4 R⁸ groups.

In an embodiment, R^{3b} is 4- to 6-membered heterocycloalkyl; optionally substituted with from 1 to 4 R⁸ groups. In an embodiment, R^{3b} is 4-membered heterocycloalkyl; optionally substituted with from 1 to 2 R⁸ groups. In an embodiment, R^{3b} is 5-membered heterocycloalkyl; optionally substituted with from 1 to 3 R⁸ groups. In an embodiment, R^{3b} is 6-membered heterocycloalkyl; optionally substituted with from 1 to 4 R⁸ groups.

In an embodiment, R^{3b} is C₁-C₄-alkyl. In an embodiment, R^{3b} is C₂-C₄-alkylene-O—C₁-C₄-alkyl. In an embodiment, R^{3b} is C₁-C₄-haloalkyl.

In an embodiment, R^{3b} is C₃-C₄-alkyl. In an embodiment, R^{3b} is C₂-C₄-alkylene-O—C₁-C₄-alkyl. In an embodiment, R^{3b} is C₃-C₄-haloalkyl.

It may be that where Ring A is 5-membered heteroaryl, R^{3b} is optionally substituted C₅-cycloalkyl. It may be that where Ring A is 5-membered heteroaryl, R^{3b} is optionally substituted 6-membered heterocycloalkyl. It may be that where Ring A is 5-membered heteroaryl, R^{3b} is substituted or unsubstituted phenyl. It may be that where Ring A is 5-membered heteroaryl, R^{3b} is optionally substituted 6-membered heteroaryl.

In an embodiment, R^{3b} is selected from:

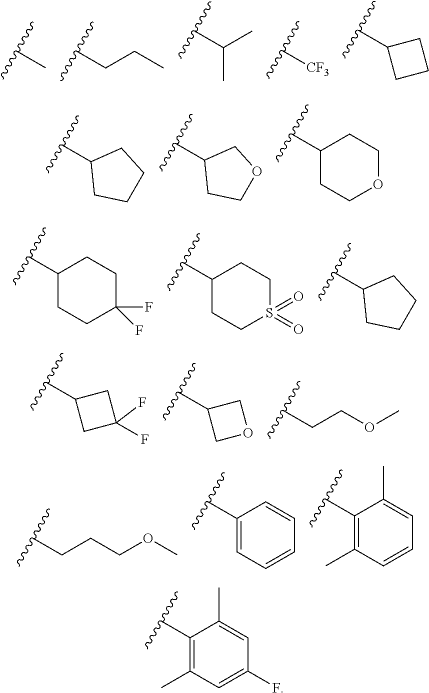

In an embodiment, R⁴ is independently at each occurrence selected from cyano, C₀-C₄-alkylene-NR⁵R⁶, C₀-C₄-alkylene-OR⁷, S(O)₂R⁶, C₁-C₄-alkyl, 4- to 6-membered heterocycloalkyl, C₁-C₄-alkyl-S(O)₂R⁶, and C₁-C₄-haloalkyl.

In an embodiment, R⁴ is independently at each occurrence selected from cyano, C₀-C₄-alkylene-NR⁵R⁶, C₀-C₄-alkylene-OR⁷, S(O)₂R⁶, C₁-C₄-alkyl, C₁-C₄-alkyl-S(O)₂R⁶, and C₁-C₄-haloalkyl.

In an embodiment, R⁴ is independently at each occurrence selected from cyano, C₀-C₄-alkylene-NR⁵R⁶, C₀-C₄-alkylene-OR⁷, S(O)₂R⁶, C₁-C₂-alkyl, 4-membered heterocycloalkyl, C(CH₃)₂OH, C₁-C₂-alkyl-S(O)₂R⁶, and C₁-C₂-haloalkyl.

In an embodiment, R⁴ is independently at each occurrence selected from cyano, C₀-C₄-alkylene-NR⁵R⁶, C₀-C₄-alkylene-OR⁷, S(O)₂R⁶, C₁-C₂-alkyl, C(CH₃)₂OH, C₁-C₂-alkyl-S(O)₂R⁶, and C₁-C₂-haloalkyl.

In an embodiment, R⁴ is independently at each occurrence selected from C₀-C₄-alkylene-NR⁵R⁶, S(O)₂R⁶, C₁-alkyl, 4-membered heterocycloalkyl, C(CH₃)₂OH, C₁-alkyl-S(O)₂R⁶, and C₁-haloalkyl.

In an embodiment, R⁴ is independently at each occurrence selected from C₀-C₄-alkylene-NR⁵R⁶, S(O)₂R⁶, C₁-alkyl, C(CH₃)₂OH, C₁-alkyl-S(O)₂R⁶, and C₁-haloalkyl.

In an embodiment, R⁴ is independently at each occurrence selected from cyano, NR⁵R⁶, OR⁷, S(O)₂R⁶, C₁-C₂-alkyl, 4-membered heterocycloalkyl, C(CH₃)₂OH, C₁-C₂-alkyl-S(O)₂R⁶, and C₁-C₂-haloalkyl.

In an embodiment, R⁴ is independently at each occurrence selected from cyano, NR⁵R⁶, OR⁷, S(O)₂R⁶, C₁-C₂-alkyl, C(CH₃)₂OH, C₁-C₂-alkyl-S(O)₂R⁶, and C₁-C₂-haloalkyl.

In an embodiment, R⁴ is independently at each occurrence selected from NR⁵R⁶, S(O)₂R⁶, C₁-alkyl, oxetanyl (e.g., oxetan-3-yl), C(CH₃)₂OH, C₁-alkyl-S(O)₂R⁶, and C₁-haloalkyl.

In an embodiment, R⁴ is independently at each occurrence selected from NR⁵R⁶, S(O)₂R⁶, C₁-alkyl, C(CH₃)₂OH, C₁-alkyl-S(O)₂R⁶, and C₁-haloalkyl.

In an embodiment, R⁴ is independently at each occurrence selected from N(H)S(O)₂Me, S(O)₂MeR⁶, C(CH₃)₂OH, C₁-alkyl-S(O)₂Me.

In an embodiment, m is an integer selected from 0, 1, and 2. In an embodiment, m is 2. In an embodiment, m is 1. In an embodiment, m is 0.

In an embodiment, R^{4a} is H. In an embodiment, R^{4a} is methyl. In an embodiment, R^{4a} is cyclopropyl. In an embodiment, R^{4a} is a 4-membered heterocycloalkyl. In an embodiment, R^{4a} is oxetanyl. In an embodiment, R^{4a} is oxetan-3-yl. In an embodiment, R^{4a} is oxetanyl or azetidinyl. In an embodiment, R^{4a} is independently selected from H, C₁-C₄-alkyl and cyclopropyl. In an embodiment, R^{4a} is independently selected from C₁-C₄-alkyl, cyclopropyl and cyclobutyl. In an embodiment R^{4a} is cyclopropyl.

In an embodiment, R^{4b} is selected from S(O)₂R⁶. In an embodiment, R^{4b} is C₁-C₄-alkyl. In an embodiment, R^{4b} is C₁-C₄-alkyl-S(O)₂R⁶. In an embodiment, R^{4b} is C₁-C₄-haloalkyl. In an embodiment, R^{4b} is cyclopropyl.

In an embodiment, R^{4b} is selected from S(O)₂—C₁-C₃-alkyl, e.g., S(O)₂Me. In an embodiment, R^{4b} is C₁-C₄-alkyl, e.g., methyl. In an embodiment, R^{4b} is C₁-C₄-alkyl-S(O)₂-C₁-C₄-alkyl, e.g., —CH₂—S(O)₂-Me.

In an embodiment, R⁵ is independently at each occurrence selected from H, C₁-C₄-alkyl, and S(O)₂—C₁-C₄-alkyl.

In an embodiment, R⁵ is S(O)₂—C₁-C₄-alkyl; optionally wherein R⁵ is S(O)₂—C₁-alkyl.

In an embodiment, R⁵ is H. In an embodiment, R⁵ is methyl.

In an embodiment, R⁶ is independently at each occurrence selected from H and C₁-C₄-alkyl. In an embodiment, R⁶ is H. In an embodiment, R⁶ is methyl.

In an embodiment, R⁷ is independently at each occurrence selected from H, C₁-C₄-alkyl, and C₁-C₄-haloalkyl.

In an embodiment, R⁷ is independently at each occurrence selected from H, and C₁-C₄-alkyl.

In an embodiment, R⁷ is independently at each occurrence selected from H, C₁-C₂-alkyl, and C₁-C₂-haloalkyl.

In an embodiment, R⁷ is independently at each occurrence selected from H, and C₁-C₂-alkyl.

In an embodiment, R⁷ is independently at each occurrence H.

In an embodiment, R⁸ is independently at each occurrence selected from ═O, fluoro, nitro, cyano, NR⁵R⁶, OR⁷, C(O)R⁶, C₁-C₄-alkyl, C₁-C₄-haloalkyl and cyclopropyl.

In an embodiment, R⁸ is independently at each occurrence selected from ═O, fluoro, C(O)R⁶, C₁-C₂-alkyl, and C₁-C₂-haloalkyl.

In an embodiment, R⁸ is independently at each occurrence selected from ═O, fluoro, and C(O)R⁶. In an embodiment, R⁸ is independently at each occurrence selected from ═O, fluoro, and C(O)Me.

In an embodiment, R⁹ is independently at each occurrence selected from halo, nitro, cyano, NR⁵R⁶, OR⁷, C(O)R⁶, C₁-C₄-alkyl, C₁-C₄-haloalkyl and cyclopropyl.

In an embodiment, R⁹ is independently at each occurrence selected from halo, C₁-C₄-alkyl, and C₁-C₄-haloalkyl.

In an embodiment, R⁹ is independently at each occurrence selected from halo, C₁-C₂-alkyl, and C₁-C₂-haloalkyl.

In an embodiment, R⁹ is independently at each occurrence selected from halo, and C₁-C₂-alkyl. In an embodiment, R⁹ is independently at each occurrence selected from fluoro and methyl.

In an embodiment, R^{x} and R^{y} are each independently selected from H, halo, nitro, cyano, NR⁵R⁶, OR⁷, SR⁶, C₁-C₄-alkyl, C₁-C₄-haloalkyl and C₃-C₄-cycloalkyl.

In an embodiment, R^{x} and R^{y} are each independently selected from H, halo, cyano, C₁-C₂-alkyl, C₁-C₂-haloalkyl and C₃-cycloalkyl and.

In an embodiment, R^{x} is H. In an embodiment, R^{y} is H. In an embodiment, R^{x} and R^{y} are each H.

In an embodiment, any of the alkyl or alkenyl groups are optionally substituted, where chemically possible, by 1 to 5 substituents which are each independently at each occurrence selected from the group consisting of: oxo, fluoro, NR^{a}R^{b}, OR^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂-C₁-C₄-alkyl.

In an embodiment, X⁶ is carbon. In an embodiment, X⁶ is nitrogen.

In an embodiment, X⁷ is carbon. In an embodiment, X⁷ is nitrogen.

In an embodiment, p is an integer selected from 0, 1, 2, and 3. In an embodiment, p is 3. In an embodiment, p is 2. In an embodiment, p is 1. In an embodiment, p is 0.

In an embodiment, the compound according to formula (I) is selected from:

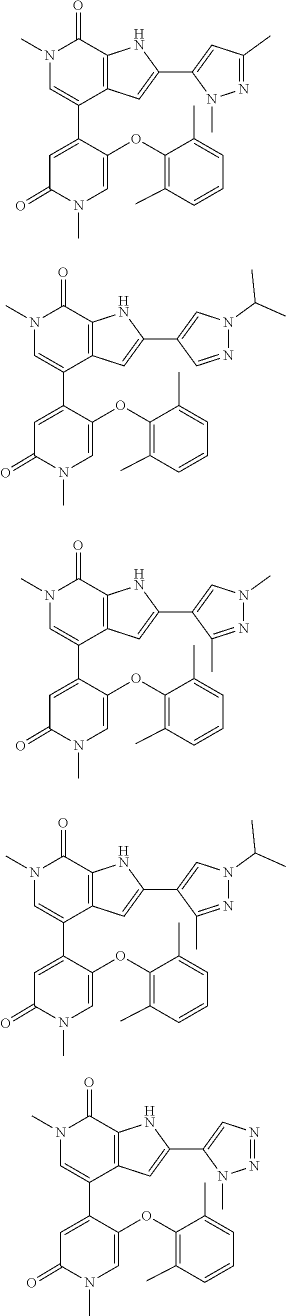

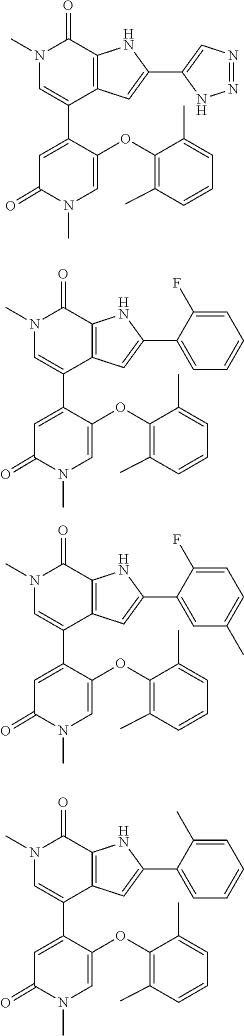

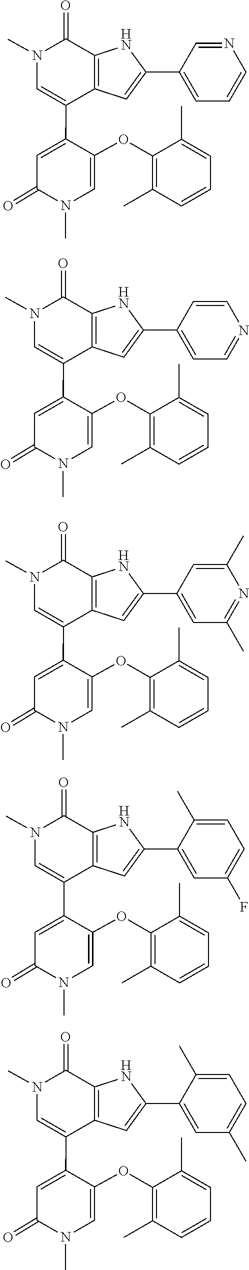

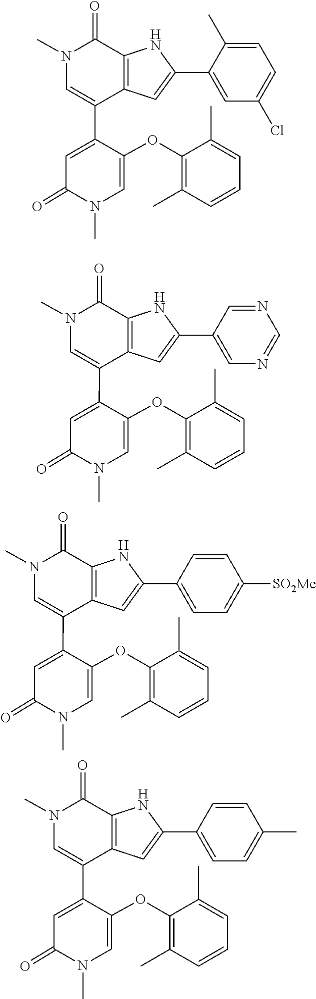

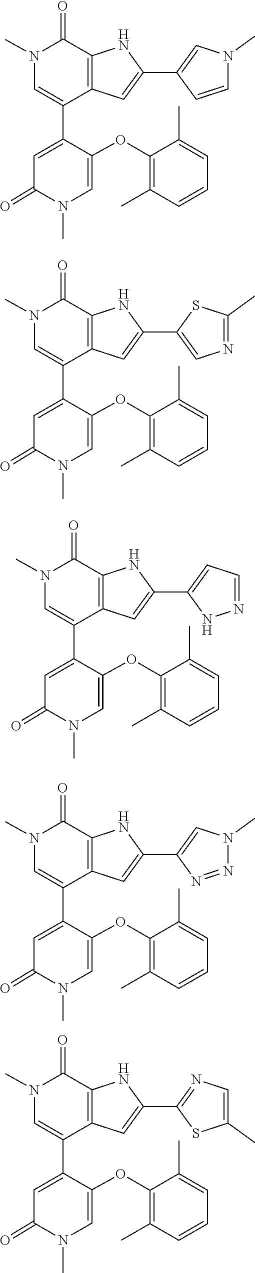

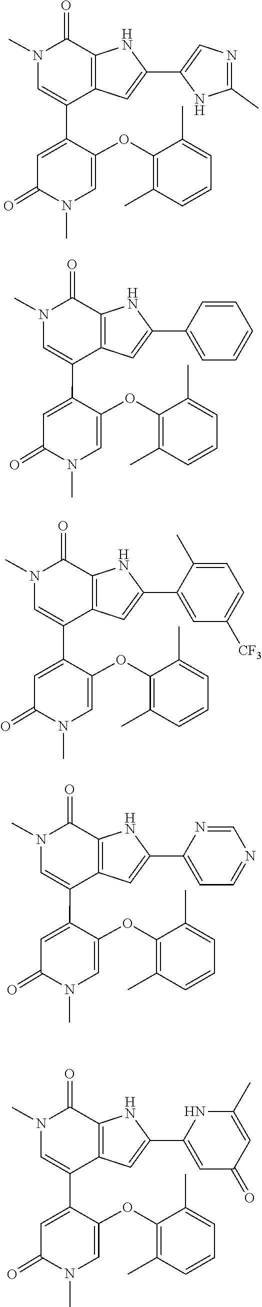

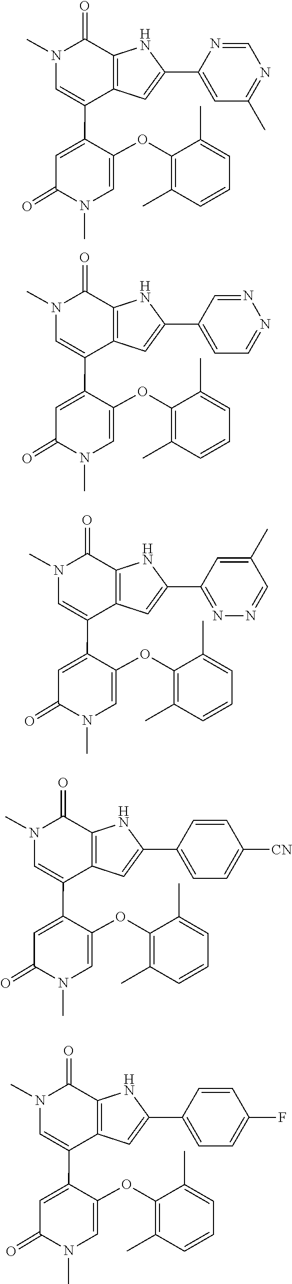

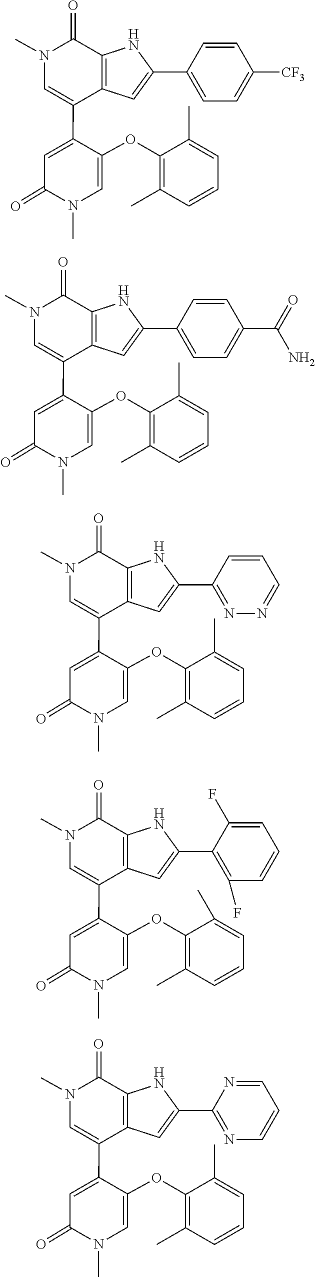

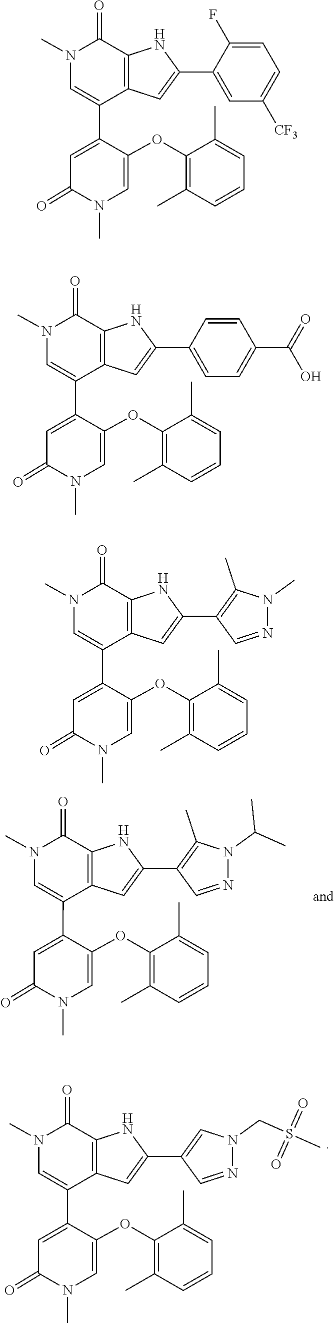

In an embodiment, the compound according to formula (I) is selected from:

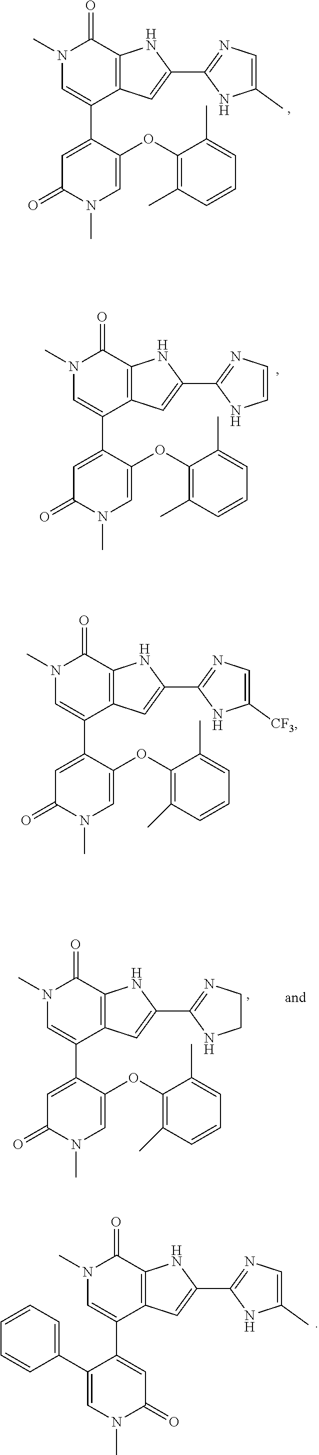 [SMILES: Cc1cnc(-c2cc3c(-c4cc(=O)n(C)cc4Oc4c(C)cccc4C)cn(C)c(=O)c3[nH]2)[nH]1 ; Cc1cccc(C)c1Oc1cn(C)c(=O)cc1-c1cn(C)c(=O)c2[nH]c(-c3ncc[nH]3)cc12 ; Cc1cccc(C)c1Oc1cn(C)c(=O)cc1-c1cn(C)c(=O)c2[nH]c(-c3ncc(C(F)(F)F)[nH]3)cc12 ; Cc1cccc(C)c1Oc1cn(C)c(=O)cc1-c1cn(C)c(=O)c2[nH]c(C3=NCCN3)cc12 ; Cc1cnc(-c2cc3c(-c4cc(=O)n(C)cc4-c4ccccc4)cn(C)c(=O)c3[nH]2)[nH]1]

In an embodiment, the compound according to formula (I) is selected from:

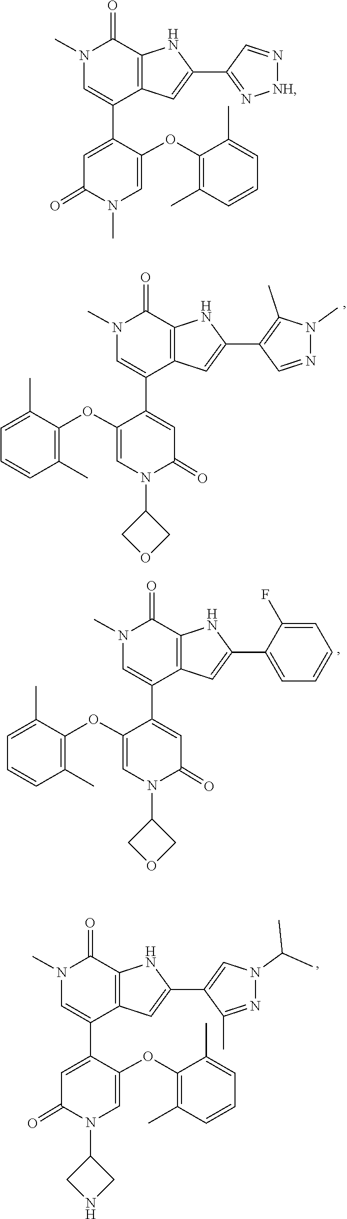

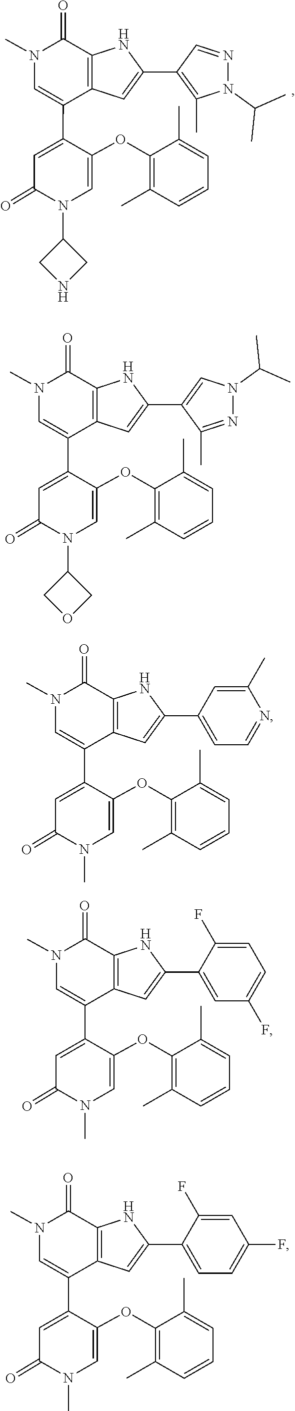

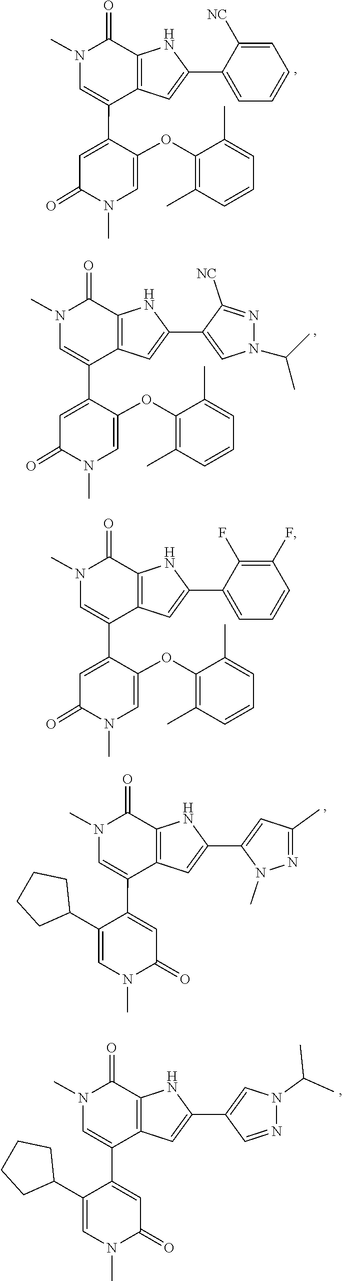

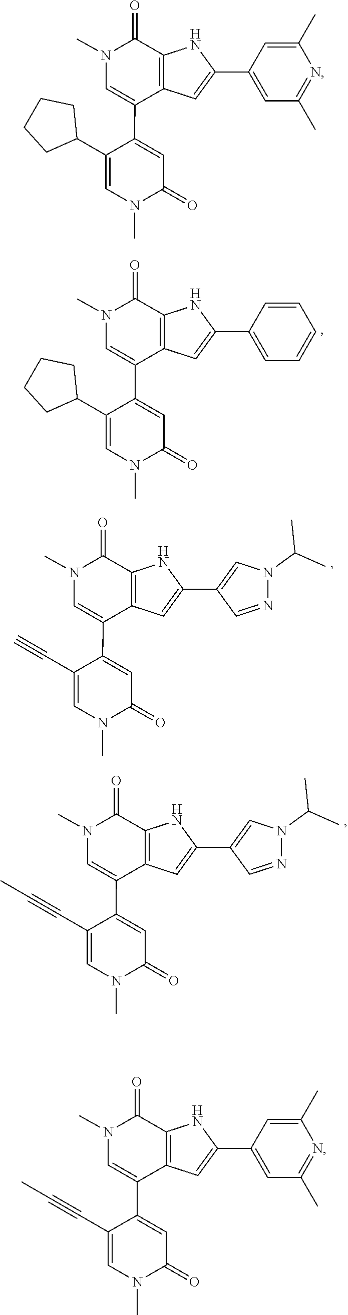

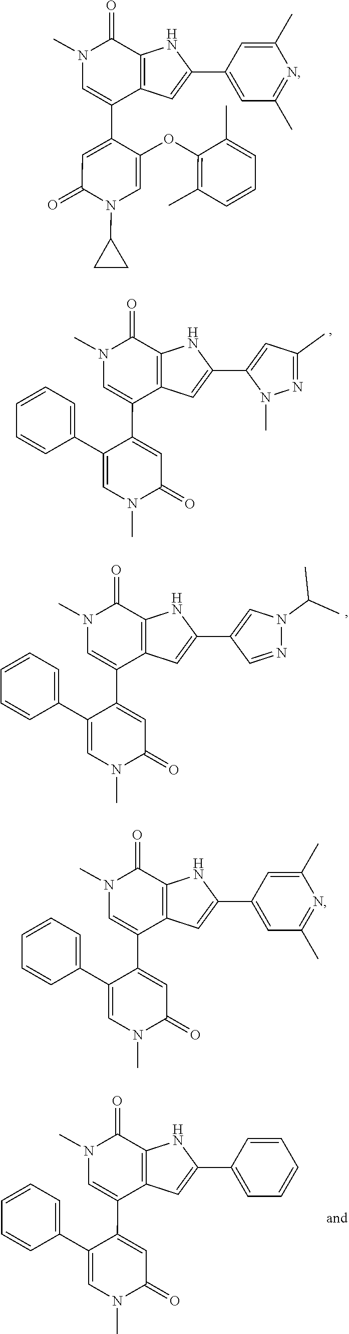

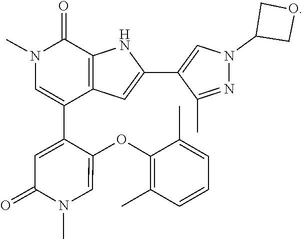 [SMILES: Cc1cccc(C)c1Oc1cn(C)c(=O)cc1-c1cn(C)c(=O)c2[nH]c(-c3cn(C4COC4)nc3C)cc12]

In an embodiment, the compound according to formula (I) is selected from structures where R² is a saturated or unsaturated six membered heterocyclic ring with one nitrogen atom that may at any position be substituted or unsubstituted as described herein and may include a pyridone.

In an embodiment, the compound according to formula (I) is selected from structures where R² is a saturated or unsaturated six membered heterocyclic ring with two nitrogen atoms that may at any position be substituted or unsubstituted as described herein.

In an embodiment, the compound according to formula (I) is selected from structures where R² is a saturated or unsaturated six membered heterocyclic ring with three nitrogen atoms that may at any position be substituted or unsubstituted as described herein.

In an embodiment, the compound according to formula (I) is selected from structures where R² is a saturated or unsaturated five membered heterocyclic ring with one nitrogen atom that may at any position be substituted or unsubstituted as described herein.

In an embodiment, the compound according to formula (I) is selected from structures where R² is a saturated or unsaturated five membered heterocyclic ring with two nitrogen atoms that may at any position be substituted or unsubstituted as described herein.

In an embodiment, the compound according to formula (I) is selected from structures where R² is a saturated or unsaturated five membered heterocyclic ring with three nitrogen atoms that may at any position be substituted or unsubstituted as described herein.

In an embodiment, the compound according to formula (I) is selected from structures where R² is a saturated or unsaturated five membered heterocyclic ring with one nitrogen atom and one sulphur that may at any position be substituted or unsubstituted as described herein.

In an embodiment, the compound according to formula (I) is not

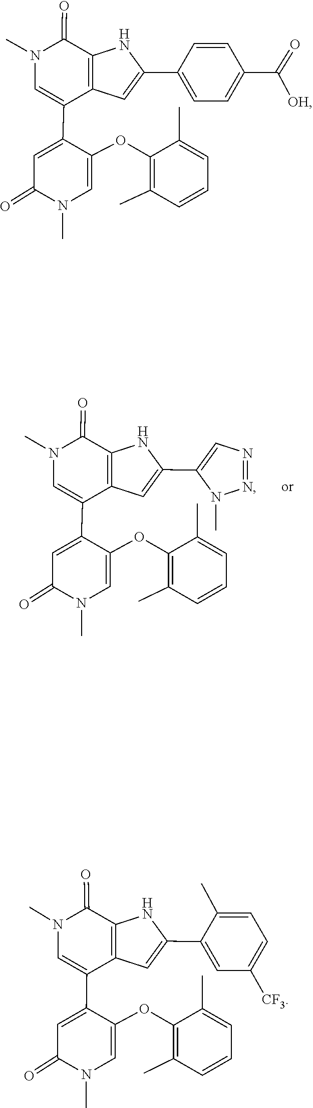 [SMILES: Cc1cccc(C)c1Oc1cn(C)c(=O)cc1-c1cn(C)c(=O)c2[nH]c(-c3ccc(C(=O)O)cc3)cc12 ; Cc1cccc(C)c1Oc1cn(C)c(=O)cc1-c1cn(C)c(=O)c2[nH]c(-c3cnnn3C)cc12 ; Cc1ccc(C(F)(F)F)cc1-c1cc2c(-c3cc(=O)n(C)cc3Oc3c(C)cccc3C)cn(C)c(=O)c2[nH]1]

In an embodiment, the compound according to formula (I) is not

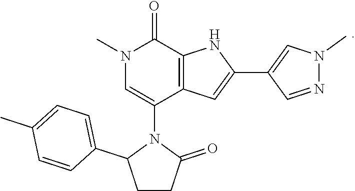

The inventors have found that certain compounds of the disclosure may have increased metabolic stability. The inventors have also found that certain compounds of the disclosure may have increased activity against BRD4 BD2. The inventors have also found that certain compounds of the disclosure may have increased selectivity for BRD4 BD2 over BRD4 BD1. The inventors have also found that certain compounds of the disclosure may have increased bioavailability.

In accordance with a second aspect, the present disclosure provides a pharmaceutical composition comprising a compound defined in the first aspect, and one or more pharmaceutically acceptable excipients.

In accordance with a third aspect, the present disclosure provides a compound as defined in the first aspect or a pharmaceutical composition as defined in the second aspect, for use as a medicament.

In accordance with a fourth aspect, the present disclosure provides the use of a compound as defined in the first aspect or a pharmaceutical composition as defined in the second aspect, for the manufacture of a medicament.

In accordance with a fifth aspect, the present disclosure provides a compound as defined in the first aspect, or a pharmaceutical composition as defined in the second aspect, for use in a method of treatment or prophylaxis of an inflammatory disease, e.g., inflammatory skin disorders, respiratory diseases, gastrointestinal diseases, eye diseases, cancers, rheumatic diseases, demyelinating diseases and fibrotic diseases.

In accordance with a sixth aspect, the present disclosure provides a method for the treatment or prophylaxis of an inflammatory disease, e.g., inflammatory skin disorders, respiratory diseases, gastrointestinal diseases, eye diseases, cancers, rheumatic diseases, demyelinating diseases, and fibrotic diseases, said method comprising administering to a subject, an effective amount of a compound as defined in the first aspect, or a pharmaceutical composition as defined in the second aspect.

In accordance with a seventh aspect, the present disclosure provides the use of a compound as defined in the first aspect, or a pharmaceutical composition as defined in the second aspect for the manufacture of a medicament for the treatment or prophylaxis of an inflammatory disease, e.g., inflammatory skin disorders, respiratory diseases, gastrointestinal diseases, eye diseases, cancers, rheumatic diseases, demyelinating diseases, and fibrotic diseases, said method comprising administering to a subject, an effective amount of a compound as defined in the first aspect, or a pharmaceutical composition as defined in the second aspect.

In accordance with an eighth aspect, the present disclosure provides a method of inhibiting Bromodomain and Extra-Terminal protein activity in a subject, said method comprising administering to a subject an effective amount of a compound as defined in the first aspect, or a pharmaceutical composition as defined in the second aspect.

In accordance with a ninth aspect, the present disclosure provides a method of treating a disorder associated with Bromodomain and Extra-Terminal protein activity in a subject, said method comprising administering to a subject an effective amount of a compound as defined in the first aspect, or a pharmaceutical composition as defined in the second aspect.

Selective BET BDII inhibitors, such as the compounds disclosed herein, may in one or more embodiments, be of value and used in the treatment or amelioration of the following non-limiting examples of disorders and diseases.

Selective BET BDII inhibitors, such as the compounds disclosed herein, may in one or more embodiments, be of value and used in the treatment or amelioration of inflammatory disorders, immune disorders, and autoimmune disorders, which include diseases that have or may have an inflammatory or autoimmune component.

The inflammatory disorder, immune disorder, or autoimmune disorder may be a skin disorder selected from acne, inflammatory acne, acne fulminans, angiofibroma, nodular papulopustular acne, acne conglobata, acute erysipelas, alopecia, alopecia areata, alopecia totalis, atopic dermatitis, alopecia universalis, autoimmune bullous skin disorder such as pemphigus vulgaris (PV) or bullous pemphigoid (BP), bacterial skin infections, viral skin infections, bullous diseases, cellulitis, cutaneous abscesses, carbuncles, chronic hand eczema, cutaneous mastocytosis, Dercum disease, dermatological pain, dermatological inflammation, contact dermatitis, dermatitis, dermatitis herpetiformis, dermatomyositis, chronic atypical neutrophilic dermatosis with lipodystrophy and elevated temperature (CANDLE), neutrophilic dermatoses, such as pyoderma gangrenosum and Sweets syndrome, paronychial infections, pustulosis palmoplantaris edematous, erythema multiforme, erythema nodosum, granuloma annulare, pemphigus, epidermal necrolysis pemphigus, paraneoplastic pemphigus, erythrasma, ecthyma, eczema, folliculitis, furuncles, gustatory sweating, hyperhidrosis, Hailey-Hailey disease, hives, hidradenitis suppurativa, hypertrophic scars, impetigo, ichthyosis, ischemic necrosis, keloids, necrotizing subcutaneous infections, actinic keratosis, keratosis pilaris, miliaria, molluscum contagiosum, lichen planus, netherton syndrome, pityriasis rubra pilaris, psoriasis, pruritus, prurigo nodularis, rashes, rosacea, pediculosis, pityriasis rosea, scleroderma, scalded skin syndrome, skin rash, skin irritation, skin sensitization (e.g., contact dermatitis or allergic contact dermatitis), trauma or injury to the skin, post-operative or post-surgical skin conditions, wounds, burns (including chemical, electrical fire, friction, radiation, temperature related, thermal and cold), sunburn, scarring, scabies, skin ulcers, urticaria pigmentosa, urticarial and chronic idiopathic pruritus, vitiligo, warts, and xerosis.

The inflammatory disorder, immune disorder, or autoimmune disorder may be a respiratory disease selected from asthma, bronchiectasis, bronchiolitis, byssinosis, chronic obstructive pulmonary disease (COPD), cystic fibrosis, hypersensitivity pneumonitis, mesothelioma, pneumoconiosis, (idiopathic) pulmonary fibrosis, rhinitis, rhinosinusitis and sarcoidosis.

The inflammatory disorder, immune disorder, or autoimmune disorder may be a gastrointestinal disease selected from celiac disease, Crohn's disease, eosinophilic esophagitis, inflammatory bowel disease, retroperitoneal fibrosis, and ulcerative colitis.

The inflammatory disorder, immune disorder, or autoimmune disorder may be an eye disease selected from conjunctivitis, dry eye syndrome, iritis, keratitis, macular degeneration, myasthenia gravis, scleritis, Sjögran's syndrome, and uveitis.

The inflammatory disorder, immune disorder, or autoimmune disorder may be a cardiovascular disease or associated disorder, selected from cerebrovascular disease, aorta disease, arrhythmias, atherosclerosis, aneurysm, angina, stroke, carditis, cardiac hypertrophy, cardiomyopathy, endocarditis, coronary artery disease, deep vein thrombosis, heart attack, heart disease, heart failure, Marfan syndrome, myocarditis, peripheral artery disease, pericarditis. pulmonary embolism, rheumatic heart disease, thrombosis, valvular heart disease, ventricular heart disease, ventricle dysfunction, and vascular diseases.

The inflammatory disorder, immune disorder, or autoimmune disorder may be a systemic indication selected from Addison's disease, AIDS, ankylosing spondylitis, atherosclerosis, arthritis, Behcet's disease, cryopyrin-associated periodic syndromes (CAPS), chronic kidney diseases (including, but not limited to nephritis, nephropathy, hypertensive nephropathy, HIV-associated nephropathy, IgA nephropathy, familial Mediterranean fever, focal segmental glomerulosclerosis, Grave's disease, juvenile arthritis, lymphangitis, lymphadenitis, lupus nephritis, minimal change disease, neurofibromatoses, polycystic kidney disease and tubular interstitial nephritis), acute kidney injury disease or condition (including, but are not limited to ischemia-reperfusion induced, cardiac and major surgery induced, percutaneous coronary intervention induced, radio-contrast agent induced, sepsis induced, pneumonia induced, and drug toxicity induced), giant cell arthritis, glomerulonephritis, gout, hepatitis, hepatitis B, hepatitis C, hypophysitis, Kawasaki disease, liver fibrosis, multiple sclerosis, myositis, osteoarthritis, pancreatitis, pneumonitis, polyarteritis nodosa, primary biliary cirrhosis, prostate disease, prostatitis, benign prostatic hyperplasia (BPH), psoriatic arthritis, rheumatoid arthritis, scleritis, scleroderma (cutaneous or systemic), sclerosing cholangitis, sepsis, systemic lupus erythematosus, systemic mastocytosis, Takayasu's arteritis, thyroiditis, toxic shock, vasculitis, warm autoimmune hemolytic anemia, and Wegener's granulomatosis.

The inflammatory disorder, immune disorder, or autoimmune disorder may be an autoimmune disease or indication where immunosuppression would be desirable, for instance, to avoid organ transplant rejection and graft versus host disease (chronic or acute).

Selective BET BDII inhibitors, such as the compounds disclosed herein, may in one or more embodiments, be of value and used in the treatment or amelioration of cancers.

The cancer may be a skin or systemic cancer, selected from acoustic neuroma, anal cancer, bladder cancer, Bowen's disease, brain cancer, breast cancer, carcinomas including basal cell carcinoma, bile duct carcinoma, bronchogenic carcinoma, choriocarcinoma, embryonal carcinoma, cystadenocarcinoma, epithelial carcinoma, medullary carcinoma, NUT midline carcinoma (NMC), papillary carcinoma, papillary adenocarcinomas, renal cell carcinoma, sebaceous gland carcinoma, small cell lung carcinoma, squamous cell carcinoma, and sweat gland carcinoma, cervical cancer, chordoma, colon cancer, colorectal cancer, craniopharyngioma, dysproliferative changes (dysplasias and metaplasias), endometrial cancer, ependymoma, esophageal cancer, essential thrombocythemia, estrogen-receptor positive breast cancer, Ewing's tumour, genital cancer, cancer of the cervix, cancer of the vulva, vulvar intraepithelial neoplasia (VIN), cancer of the vagina, germ cell testicular cancer, gastrointestinal cancers, gastric cancer, glioblastoma, glioma, heavy chain disease, hemangioblastoma, hepatocellular cancer, hepatoma, hormone insensitive prostate cancer, keratinocyte carcinomas, kidney cancer, leukaemias including acute leukaemia, acute lymphocytic leukaemia, acute myeloid leukaemia, acute myelocytic leukaemia (monocyctic, myeloblastic, adenocarcinoma, angiosarcoma, astrocytoma, myelomonocytic and promyelocytic), acute t-cell leukaemia, chronic leukaemia, chronic lymphocytic leukaemia, chronic myelocytic (granulocytic) leukaemia, chronic myelogenous leukaemia, erythroleukemia, lymphoblastic leukaemia, and myelogenous leukaemia, liver cancer, lung cancer, lymphoid malignancies of T-cell or B-cell origin, lymphomas (Hodgkin's and non-Hodgkin's) including cutaneous T-cell lymphoma, diffuse large B-cell lymphoma, and follicular lymphoma, cutaneous (skin) lymphomas, malignancies and hyperproliferative disorders including of the bladder, breast, colon, lung, ovaries, pancreas, prostate, skin and uterus, advanced malignancies, medulloblastoma, melanoma, meningioma, Merkel cell cancer mesothelioma, metastatic cancer, multiple myeloma, myeloma, pancreatic cancer, myelofibrosis, myeloproliferative neoplasms, neuroblastoma, non-small cell lung cancer, head and neck cancer, oligodendroglioma, oral cancer, ovarian cancer, pancreatic cancer, pinealoma, polycythemia vera, prostate cancer, rectal cancer, retinoblastoma, sarcomas including chondrosarcoma, endotheliosarcoma, fibrosarcoma, gliosarcoma, leiomyosarcoma, liposarcoma, lymphagioendotheliosarcoma, lymphangiosarcoma, myxosarcoma, Castleman's disease and Kaposi's sarcoma, osteogenic sarcoma, and rhabdomyosarcoma, seminoma, skin cancer, skin adnexal tumors, and sarcomas, small cell lung cancer, solid tumors, stomach cancer, synovioma, testicular tumours, thyroid cancer, uterine cancer, Waldenstrom's macroglobulinemia, and Wilms' tumour.

Selective BET BDII inhibitors, such as the compounds disclosed herein, may in one or more embodiments, be used to provide male contraception.

Selective BET BDII inhibitors, such as the compounds disclosed herein, may in one or more embodiments, be of use in the treatment or amelioration of obesity, dyslipidaemia, cholesteatoma, hypercholesterolemia, Alzheimer's disease, metabolic syndrome, hepatic steatosis, type I diabetes, type II diabetes, and complications from diabetes, insulin resistance, and diabetic retinopathy or diabetic neuropathy.

Selective BET BDII inhibitors, such as the compounds disclosed herein, may in one or more embodiments, be of use in the treatment or amelioration of an immune system dysfunction, a viral disease, a bacterial disease, a yeast disease, non-inflammatory acne, an allergic disease, asthma, food allergy, rhinitis, an IL-6 pathway-related disease, an immune response, and a hyperproliferative disorder;

Selective BET BDII inhibitors, such as the compounds disclosed herein, may in one or more embodiments, be of use in the treatment or amelioration of Aicardi-Goutieres syndrome, chilblain lupus, stimulator of interferon genes-Associated Vasculopathy with onset in Infancy (SAVI), Singleton-Merten syndrome, retinal vasculopathy with cerebral leukodystrophy, autoimmune uveitis, lupus, systemic sclerosis, an autoimmune thyroid disease, an allograft rejection, a graft-versus-host disease, an allograft rejection reaction, and a graft-versus-host reaction.

Selective BET BDII inhibitors, such as the compounds disclosed herein, may in one or more embodiments, be of use in the treatment or amelioration of disorders caused by a virus, such as Epstein-Barr virus (EBV), HIV, HTLV 1, chickenpox, herpes simplex virus infections, herpes zoster virus (VZV), and human papillomavirus (HPV) disease.

Selective BET BDII inhibitors, such as the compounds disclosed herein, may in one or more embodiments, be of use in the treatment or amelioration of mucopurulent cervicitis (MPC), urethritis, nongonococcal urethritis (NGU), vulvar disorders, vulvodynia, vulvar pain, vulvar dystrophy, pelvic inflammation, endometritis, salpingitis, oophoritis, dyspareunia, anal and rectal disease, anal abscess/fistula, anal fissure, anal warts, hemorrhoids, anal itch, pruritus ani, fecal incontinence, constipation, and polyps of the colon and rectum.

Selective BET BDII inhibitors, such as the compounds disclosed herein, may in one or more embodiments, be of use in the restoration of integrity or acceleration of the restoration of the integrity of an area of broken or damaged tissue, skin or mucosa, and in the reduction and amelioration of scar formation or scars.

Selective BET BDII inhibitors, such as the compounds disclosed herein, may in one or more embodiments, be of use in the treatment or amelioration of pyoderma gangrenosum (PG), palmar plantar pustulosis (PPP), and generalized pustular psoriasis (GPP).

Selective BET BDII inhibitors, such as the compounds disclosed herein, may in one or more embodiments, be of use in the treatment or amelioration of Crohn's disease, multiple sclerosis, rheumatoid arthritis, rhinosinusitis, and ulcerative colitis.

Selective BET BDII inhibitors, such as the compounds disclosed herein, may in one or more embodiments, be of use in the treatment or amelioration of cryopyrin-associated periodic syndromes (CAPS), cardiovascular disease, cerebrovascular disease, familial mediterranean fever, Grave's disease, liver fibrosis, neurofibromatoses, myocarditis, pericarditis, prostate disease, prostatitis, benign prostatic hyperplasia (BPH), systemic mastocytosis, and warm autoimmune hemolytic anemia.

Selective BET BDII inhibitors, such as the compounds disclosed herein, may in one or more embodiments, be of use in the treatment or amelioration of angiofibroma, chronic hand eczema, cutaneous mastocytosis, urticaria pigmentosa, neutrophilic dermatoses such as pyoderma gangrenosum and Sweets syndrome, chronic atypical neutrophilic dermatosis with lipodystrophy and elevated temperature (CANDLE), ichthyosis, keloids, scars, hypertrophic scars, netherton syndrome, pruritus, prurigo nodularis, and urticaria pigmentosa.

Selective BET BDII inhibitors, such as the compounds disclosed herein, may in one or more embodiments, also be of value and used in the palliation, diagnosis or prevention of any disease, disorder or condition in humans of one or more of the aforesaid non-limiting examples of disorders and diseases.

Treatment or amelioration with selective BET BDII inhibitors, such as compositions comprising the compounds disclosed herein or salts thereof (or combinations thereof), in some embodiments may be effective if applied orally, in some other embodiments may be effective if applied by injection, in some other embodiments may be effective if applied topically, and in some further embodiments may be effective if applied topically and orally or by injection and topically or by orally and injection. In one or more embodiments treatment or amelioration with selective BET BDII inhibitors, such as compositions comprising the compounds disclosed herein or salts thereof (or combinations thereof), may be effective orally where the compounds have good bioavailability e.g., >about 25%.

In one or more embodiments compounds disclosed herein are active against BRD4 BD2 and selective over BRD4 BD1. In one or more embodiments BET BDII selective protein inhibitors exhibit greater than about 100 Fold selectivity, greater than about 200 Fold selectivity, greater than about 250 Fold selectivity, greater than about 300 Fold selectivity, greater than about 350 Fold selectivity greater than about 400 Fold selectivity, greater than about 500 Fold selectivity, greater than about 600 Fold selectivity, greater than about 700 Fold selectivity, greater than about 800 Fold selectivity, greater than about 900 Fold selectivity, or greater than about 1000 Fold selectivity for BDII over BDI depending e.g., on the structure. In an embodiment BET BDII selective protein inhibitors exhibit greater than about 200 Fold selectivity. In one or more embodiments BET BDII selective protein inhibitors exhibit an IC50 of <about 0.2 μM, <about 0.15 μM, <about 0.1 μM, or <about 0.05 μM for BRD4 BDII. In one or more embodiments BET BDII selective protein inhibitors exhibit an IC50 ranging from <about 0.2 μM to <about 0.05 μM.

In addition to the compounds showing activity and selectivity other factors in selecting promising drug candidates can include for example, plasma stability, clearance, pK, and bioavailability. For drug candidates for oral delivery a higher bioavailability can translate into a lower dosage and potentially fewer side effects e.g., in the alimentary canal.

In one or more embodiments, BET BDII selective protein inhibitors exhibit a mouse plasma stability of greater than about 70%, greater than about 75%, greater than about 80%, greater than about 85%, greater than about 90%, or greater than about 95% at 120 minutes.

In an embodiment, BET BDII selective protein inhibitors exhibit a mouse plasma stability of about 90% or greater at 120 minutes.

In some embodiments BET BDII selective protein inhibitors with a mouse plasma stability of about 90% or greater than 90% at 120 minutes are promising drug candidates, but compounds having a lower mouse plasma stability may in some other embodiments be useful in particular contexts.

In one or more embodiments, BET BDII selective protein inhibitors exhibit a mouse microsomal stability of <about 5, <about 4, <about 3, <about 2, or <about 1 ml/min/g liver. In an embodiment, BET BDII selective protein inhibitors exhibit a mouse microsomal stability of <2 about ml/min/g liver.

In some embodiments, BET BDII selective protein inhibitors with a mouse microsomal stability of <about 2 ml/min/g liver are promising drug candidates, but compounds having a lower mouse microsomal stability may in some other embodiments be useful in particular contexts.

In one or more embodiments, BET BDII selective protein inhibitors exhibit a rat microsomal stability of >about 20 minutes, >about 20 minutes, >about 30 minutes, >about 40 minutes, >about 50 minutes, or >about 60 minutes half-life. In an embodiment, BET BDII selective protein inhibitors exhibit a rat microsomal stability of >about 30 minutes half-life.

In some embodiments, BET BDII selective protein inhibitors with a rat microsomal stability of >about 30 minutes half-life are promising drug candidates, but compounds having a lower rat microsomal stability may in some other embodiments be useful in particular contexts.

In one or more embodiments BET BDII selective protein inhibitors exhibit an IL-22 IC50 of <about 250 nM, <about 50 nM, or <about 10 nM and/or an IL-17A IC50 of <about 250 nM, <about 50 nM, or <about 10 nM. In an embodiment, BET BDII selective protein inhibitors exhibit an IL-22 IC50 of <about 20 nM and or an IL-17A IC50 of <about 20 nM.

In some embodiments BET BDII selective protein inhibitors with an IL-22 IC50 of <about 20 nM and or an IL-17A IC50 of <about 20 nM are promising drug candidates, but compounds having a lower activity may in some other embodiments be useful in particular contexts.

In one or more embodiments BET BDII selective protein inhibitors exhibit a bioavailability of >about 12%, >about 20%, >about 25%, >about 30%, >about 40%, >about 50%, >about 60%>about 70%, >about 80%, >about 90%, or >about 95%. In an embodiment, BET BDII selective protein inhibitors exhibit a bioavailability of >about 25%. In an embodiment, BET BDII selective protein inhibitors with a bioavailability of >about 55%.

In one or more embodiments BET BDII selective protein inhibitors with a bioavailability of >about 25% are promising and >about 55% are advantageous drug candidates for oral administration, but compounds having a bioavailability of about 25% or less may in some embodiments be useful in particular contexts.

In one or more embodiments, some compounds have two or more or all of the following characteristics an IL-22 IC50 of <about 20 nM, an IL-17A IC50 of <about 20 nM, a bioavailability of >about 25%, a mouse plasma stability of about 90% or greater than 90% at 120 minutes, a mouse microsomal stability of <about 2 ml/min/g liver, a rat microsomal stability of >about 30 minutes half-life in addition to having good activity and a selectivity of greater than about 200 Fold.

In one or more embodiments, some compounds have two or more or all of the following characteristics an IL-22 IC50 of <about 10 nM, an IL-17A IC50 of <about 10 nM, a bioavailability of >about 25% or >about 55%, a mouse plasma stability of about 90% or greater than 90% at 120 minutes, a mouse microsomal stability of <about 2 ml/min/g liver, a rat microsomal stability of >about 30 minutes half-life in addition to having good activity and a selectivity of greater than about 200 Fold.

In some embodiments when applied topically, the compounds disclosed herein may be effective where the compound is delivered primarily or substantially into the skin with low levels of transdermal penetration. In some embodiments when applied topically the compounds disclosed herein may be effective where the compound is delivered primarily or substantially transdermally. In some embodiments when applied topically the compounds disclosed herein may be effective where the compound is delivered intradermally and transdermally. In some embodiments the penetration of the compound in the epidermis can be higher than that in the dermis. In some embodiments the penetration of the compound in the dermis can be higher than in the epidermis. In some embodiments the penetration of the compound in the dermis is similar to that in the epidermis. In some embodiments the concentration of the compound per unit volume in the epidermis can be higher than that in the dermis. In some embodiments the concentration of the compound per unit volume in the dermis can be higher than in the epidermis. In some embodiments the concentration of the compound per unit volume in the dermis is similar to that in the epidermis.

Compositions comprising a compound disclosed herein or salt thereof (or combinations thereof) may in one or more embodiments be administered buccally, by inhalation (e.g., spray, nebulizer, or powder puff), epidural, by injection (including intraarticular, intravenous, intracoronary, subcutaneous, intramyocardial, intraperitoneal, intramuscular, intravascular or infusion), intradermal, intraperitoneal, intrapulmonary, intraarticular (e.g., injection), nasally, orally, parenterally, rectally, sublingually, topically, transdermally, vaginally, or via an implanted reservoir.

Pharmaceutical compositions of the disclosure may be suitable for topical or transdermal administration.

Examples of dosage forms for topical or transdermal administration of a compound disclosed herein or salt thereof include creams, drops, lotions, emulsions, foams, gels, inhalants, mousses, ointments, pastes, patches, powders, solutions, or sprays.

In some embodiments, compositions comprising a novel compound disclosed herein or salt thereof (or combinations thereof) may be administered to young children. In some embodiments, compositions comprising a compound of the disclosure or salt thereof (or combinations thereof) may be administered to adolescents or teenagers. In some embodiments, compositions comprising a compound of the disclosure or salt thereof (or combinations thereof) may be administered to adults.

The disclosure may also be defined according to any one of the following numbered clauses:

1. A compound of formula (I), or a pharmaceutically acceptable salt or N-oxide thereof:

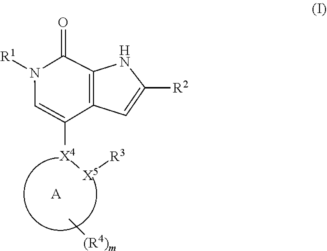

wherein
X⁴ is independently selected from carbon and nitrogen;
X⁵ is independently selected from carbon and nitrogen;
Ring A is independently selected from phenyl ring and 5-, or 6-membered heterocyclyl;
R¹ is independently selected from C₁-C₃-alkyl, C₁-C₃-fluoroalkyl, and C₃-cycloalkyl;
R² is a 5-membered heterocyclyl group optionally substituted with from 1 to 4 R^{2a} groups;
R^{2a} is independently at each occurrence selected from ═O, halo, OR⁷, C₁-C₄-alkyl, and C₁-C₄-haloalkyl;
R³ is independently at each occurrence selected from R^{3a}, OR^{3b}, or NR⁶R^{3b};
R^{3a} is independently selected from H, CN, C₁-C₄-alkyl, C₂-C₄-alkenyl, C₁-C₄-haloalkyl, C₂-C₄-haloalkenyl, and C₀-C₃-alkylene-R^{3c}; wherein R^{3c} is independently at each occurrence selected from C₃-C₃-cycloalkyl, C₅-C₃-cycloalkenyl, 3- to 8-membered heterocycloalkyl, phenyl and 5- or 6-membered heteroaryl; wherein where R^{3c} is cycloalkyl or heterocycloalkyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from, C₃-C₃-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, and cyclopropyl;
R⁵ is independently at each occurrence selected from H, C₁-C₄-alkyl, C(O)—C₁-C₄-alkyl and S(O)₂—C₁-C₄-alkyl; or R⁵ and R⁶, together with the nitrogen atom to which they are attached form a C₅-C₃-heterocycloalkyl group optionally substituted with from 0 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₃-heterocycloalkyl group optionally substituted with from 0 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, NR⁵R⁶, OR⁶, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
n is an integer selected from 0, 1, 2, 3 and 4;
m is an integer selected from 0, 1, 2, 3 and 4;
wherein any of the aforementioned alkyl, alkylene or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.
   2. A compound of clause 1, wherein R¹ is methyl.
   3. A compound of clause 1 or clause 2, wherein R² is

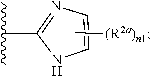

and wherein n1 is independently an integer selected from 0, 1, and 2.
4. A compound of clause 1 or clause 2, wherein R² is

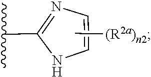

and wherein n2 is independently an integer selected from 0, 1, 2, and 3.
5. A compound of clause 1 or clause 2, wherein R² is

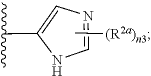

and wherein n3 is independently an integer selected from 0, 1, and 2.
6. A compound of clause 1 or clause 2, wherein R² is

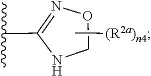

and wherein n4 is independently an integer selected from 0, 1, and 2.
7. A compound of clause 1 or clause 2, wherein R² is

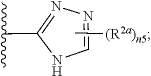

and wherein n5 is independently an integer selected from 0 and 1.
8. A compound of any one of clauses 1 to 7, wherein Ring A is phenyl.
9. A compound of any one of clauses 1 to 7, wherein Ring A is pyridone.
10. A compound of clause 9, wherein the pyridone is substituted on the nitrogen with either a C₁-C₄-alkyl group or a cyclopropyl group.
11. A compound of clause 9 or clause 10, wherein

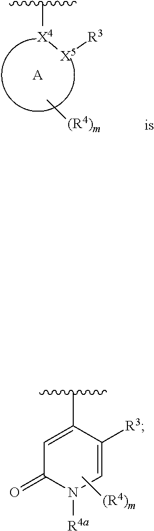

wherein R^{4a} is selected from H, C₁-C₄-alkyl, and cyclopropyl.
12. A compound of any one of clauses 1 to 7, wherein Ring A is 5-membered heteroaryl.
13. A compound of any one of clauses 1 to 12, wherein R³ is R^{3a}.
14. A compound of clause 13, wherein R^{3a} is phenyl, optionally substituted with from 1 to 3 R⁹ groups.
15. A compound of any one of clauses 1 to 12, wherein R³ is OR^{3b}.
16. A compound of clause 15, wherein R^{3b} is phenyl; optionally substituted with from 1 to 3 R⁹ groups.
17. A compound of clause 15, wherein R^{3b} is C₀-C₃-alkylene-R^{3d}; wherein R^{3d} is independently at each occurrence selected from C₃-C₅-cycloalkyl, and 4- to 6-membered heterocycloalkyl; wherein R^{3d} is optionally substituted with from 1 to 4 R⁸ groups.
18. A compound of clause 1, wherein the compound according to formula (I) is selected from:

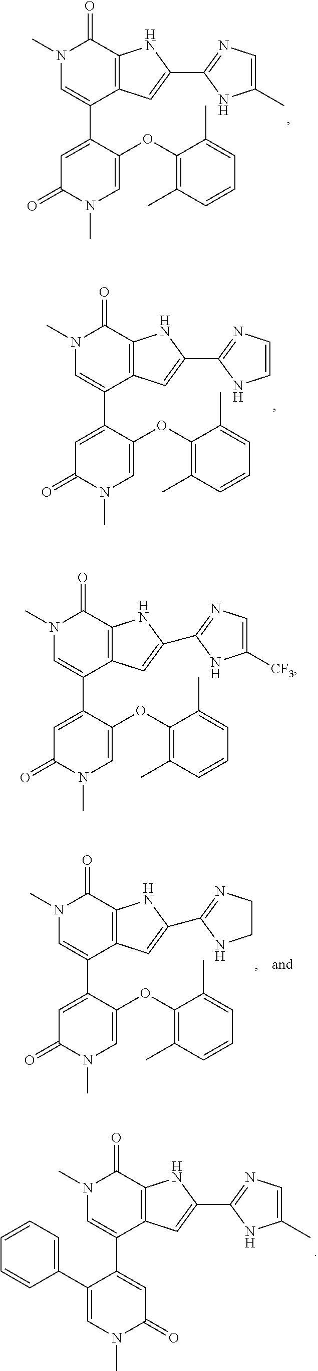

19. A pharmaceutical composition comprising a compound of any one of clauses 1 to 18, and one or more pharmaceutically acceptable excipients.
20. A compound of any one of clauses 1 to 18 for use as a medicament.
21. A compound of any one of clauses 1 to 18 for use in treating a disease selected from inflammatory disorders, immune disorders, and autoimmune disorders.
22. A compound of any one of clauses 1 to 18 for use in treating cancer.

### DETAILED DESCRIPTION

As used herein, the term “about” has its usual meaning in the context of pharmaceutical and cosmetic formulations to allow for reasonable variations in amounts that can achieve the same effect, typically plus or minus up to 30%. For example, if an amount of “about 1” is provided, then the amount can be up to 1.3 or from 0.70. In cases where “about X” will lead to a figure of above 100%, the term in some embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In some embodiments, the examples e.g., amounts of formulation ingredients can be read as if prefixed with the term “about.” In one or more other embodiments, the examples can be read without the term “about.” In some embodiments, the figures can be read with the term “about.” In one or more other embodiments, the figures can be read without the term “about.” In one or more narrower embodiments “about” can be plus or minus up to 15% unless the context indicates otherwise.

Where “about” is used in connection with “>X” or “<X” or a series of such alternatives, it can in some embodiments, include about X. Where “about” is used just at the beginning of a series of alternative amounts of “>about X” or “<about X” or “about >X” or “about <X”, it can in some embodiments be understood to include “about” before all the other alternatives of the series.

The term Cₘ-Cₙ refers to a group with m to n carbon atoms. For example, the term “C₀” refers to a group with 0 carbon atoms.

The term “alkyl” refers to a monovalent linear or branched saturated hydrocarbon chain. For example, C₁-C₆-alkyl may refer to methyl, ethyl, n-propyl, iso-propyl, n-butyl, sec-butyl, tert-butyl, n-pentyl and n-hexyl. The alkyl groups may be unsubstituted or substituted by one or more substituents.

The term “alkylene” refers to a bivalent linear saturated hydrocarbon chain. For example, C₁-C₃-alkylene may refer to methylene, ethylene or propylene. The alkylene groups may be unsubstituted or substituted by one or more substituents. For example, the term “Co-alkylene” refers to a group in which an alkylene chain is absent. For example, “C₀-alkylene-R^{a}” refers to an R^{a}.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g., 1-chloromethyl and 2-chloroethyl, trichloroethyl e.g., 1,2,2-trichloroethyl, 2,2,2-trichloroethyl, fluoroethyl e.g., 1-fluoromethyl and 2-fluoroethyl, trifluoroethyl e.g., 1,2,2-trifluoroethyl and 2,2,2-trifluoroethyl, chloropropyl, trichloropropyl, fluoropropyl, trifluoropropyl. A haloalkyl group may be a fluoroalkyl group, i.e., a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents.

The term “y- to z-membered heterocycloalkyl” refers to a y- to z-membered heterocycloalkyl group. Thus, it may refer to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 6 members. The double bond will typically be between two carbon atoms but may be between a carbon atom and a nitrogen atom. Examples of heterocycloalkyl groups include: oxirane, aziridine, thiirane, oxetane, azetidine, thietane, piperidine, piperazine, morpholine, thiomorpholine, pyrrolidine, tetrahydrofuran, tetrahydrothiophene, dihydrofuran, tetrahydropyran, dihydropyran, dioxane, and azepine. A heterocycloalkyl group may be unsubstituted or substituted by one or more substituents.

Aryl groups may be any aromatic carbocyclic ring system (i.e., a ring system containing 2(2n+1)π electrons). Aryl groups may have from 6 to 10 carbon atoms in the ring system. Aryl groups will typically be phenyl groups. Aryl groups may be naphthyl groups or biphenyl groups.

The term ‘heterocyclyl’ group refers to rings comprising from 1 to 4 heteroatoms independently selected from O, S and N. The rings may be heterocycloalkyl rings (including both saturated and partially saturated rings) or heteroaryl rings. The term “heterocyclyl” also encompasses groups that are tautomers of hydroxy heteroaryl groups, such pyridones, and tautomers of hydroxy heteroaryl groups that are substituted on the nitrogen, such as N-alkyl pyridones.

The term ‘heterocycloalkenyl’ refers to partially saturated rings comprising from 1 to 2 heteroatoms independently selected from O, S and N.

The term “heteroaryl” refers to any aromatic (i.e., a ring system containing 2(2n+1)π electrons) 5 or 6 membered ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g., 1-2) nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine.

For variables which may be selected from “carbon” and “nitrogen” (i.e., X¹, X², X³, X⁴, X⁵, etc.) it is understood that the carbon or nitrogen may additionally comprise hydrogen and/or a designated substituent to the ring system (i.e., —R^{2a}, R⁴).

On ring systems designating an optional substituent (i.e., —R^{2a}, R⁴), it is understood that the substituent, if present, may replace a hydrogen on any carbon or nitrogen of the ring system.

Compounds of the disclosure containing one or more asymmetric carbon atoms can exist as two or more stereoisomers. Certain compounds of the disclosure may exist in particular geometric and/or stereoisomeric forms and the present disclosure contemplates all such compounds, including cis- and trans-isomers, R- and S-enantiomers, diastereomers, the racemic mixtures thereof, and other mixtures thereof, as falling within the scope of the disclosure. Additional asymmetric carbon atoms may be present in a substituent such as an alkyl group. All such isomers, as well as mixtures thereof, are included in this disclosure.

Where a compound of the disclosure contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the disclosure containing, for example, an imino, keto, or oxime group, or so-called valence tautomerism in compounds which contain an aromatic moiety. It follows that a single compound may exhibit more than one type of isomerism.

Included within the scope of the present disclosure are all stereoisomers, geometric isomers and tautomeric forms of the compounds of the disclosure, including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC). Thus, chiral compounds of the disclosure (and chiral precursors thereof) may be obtained in enantiomerically-enriched form using chromatography, typically HPLC, on an asymmetric resin with a mobile phase consisting of a hydrocarbon, typically heptane or hexane, containing from about 0 to about 50% by volume of isopropanol, typically from about 2% to about 20%, and for specific examples, about 0 to about 5% by volume of an alkylamine e.g., about 0.1% diethylamine. Concentration of the eluate affords the enriched mixture.

Alternatively, the racemate (or a racemic precursor) may be reacted with a suitable optically active compound, for example, an alcohol, or, in the case where the compound of the disclosure contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The present disclosure also includes all pharmaceutically acceptable isotopically-labelled compounds of formula (I) and their syntheses, wherein one or more atoms are replaced by atoms having the same atomic number, but an atomic mass or mass number different from the atomic mass or mass number usually found in nature.

Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Isotopically-labelled compounds can generally be prepared by conventional techniques known to those skilled in the art or by processes analogous to those described using an appropriate isotopically-labelled reagent in place of the non-labelled reagent previously employed.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

The activity of the compounds of the present disclosure can be assessed by a variety of in silico, in vitro and in vivo assays. In silico analysis of a variety of compounds has been demonstrated to be predictive of ultimate in vitro and even in vivo activity.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” includes the amount of a compound that, when administered to a mammal for treating a disease, is sufficient to affect such treatment for the disease. The “therapeutically effective amount” will vary depending on the compound, the disease and its severity and the age, weight, etc., of the mammal to be treated.

A compound of the disclosure, or pharmaceutically acceptable salt thereof, may be used on their own but will generally be administered in the form of a pharmaceutical composition in which the compounds of the disclosure, or pharmaceutically acceptable salt thereof, is in association with a pharmaceutically acceptable adjuvant, diluent or carrier.

Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the disclosure, the pharmaceutical composition which is used to administer the compounds of the disclosure will in some embodiments comprise from about 0.005 to about 99% w/w compounds of the disclosure, or comprise from about 0.05 to about 80% w/w compounds of the disclosure, or comprise from about 0.10 to about 70% w/w compounds of the disclosure, or comprise from about 0.10 to about 50% w/w compounds of the disclosure (all percentages by weight being based on total composition). In some embodiments the pharmaceutical composition which is used to administer the compounds of the disclosure will comprise from about 0.005 to about 40% w/w compounds of the disclosure, or comprise from about 0.005 to about 30% w/w compounds of the disclosure, or comprise from about 0.010 to about 20% w/w compounds of the disclosure, or comprise from about 0.010 to about 10% w/w compounds of the disclosure or comprise from about 0.005 to about 5% w/w compounds of the disclosure, or comprise from about 0.005 to about 2% w/w compounds of the disclosure, or comprise from about 0.005 to about 1% w/w compounds of the disclosure, or comprise from about 0.005 to about 0.5% w/w compounds of the disclosure, or comprise from about 0.010 to about 1% w/w compounds of the disclosure, or comprise from about 0.010 to about 0.5% w/w compounds of the disclosure (all percentages by weight being based on total composition).

The pharmaceutical compositions may be administered topically (e.g., to the skin) in the form, e.g., of creams, ointments, gels, lotions, solutions, suspensions; or systemically, e.g., by oral administration in the form of tablets, lozenges, hard or soft capsules, solutions, aqueous or oily suspensions, emulsions, dispersible powders or granules, syrups or elixirs; or by parenteral administration in the form of a sterile aqueous or oily solution, suspension or emulsion for injection (including intraarticular, intravenous, intracoronary, subcutaneous, intramyocardial, intraperitoneal, intramuscular, intravascular or infusion); by rectal administration in the form of suppositories or enemas; by inhalation for example as a finely divided powder or a liquid aerosol or mist; or for administration by insufflation (for example as a finely divided powder).

For oral administration the compounds of the disclosure may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent. Thus, compositions intended for oral use may contain, for example, one or more colouring, sweetening, flavouring and/or preservative agents.

For the preparation of soft gelatine capsules, the compounds of the disclosure may be admixed with, for example, a vegetable oil or polyethylene glycol. Hard gelatine capsules may contain granules of the compound using either the above-mentioned excipients for tablets. Also liquid or semisolid formulations of the compound of the disclosure may be filled into hard gelatine capsules. Liquid preparations for oral application may be in the form of syrups or suspensions, for example, solutions containing the compound of the disclosure, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the disclosure may be administered as a sterile aqueous or oily solution.

The size of the dose for therapeutic or prophylactic purposes of a compound of the disclosure will naturally vary according to the nature and severity of the conditions, the concentration of the compound required for effectiveness in isolated cells, the concentration of the compound required for effectiveness in experimental animals, the age and sex of the animal or patient and the route of administration, according to well known principles of medicine.

Dosage levels, dose frequency, and treatment durations of compounds of the disclosure are expected to differ depending on the formulation and clinical indication, age, and co-morbid medical conditions of the patient.

An effective amount of a compound of the present disclosure for use in therapy of a condition is an amount sufficient to achieve symptomatic relief in a warm-blooded animal, particularly a human of the symptoms of the condition, to mitigate the physical manifestations of the condition, or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from about 0.5 mg to about 0.5 g of active agent (more suitably from about 0.5 to about 100 mg, for example from about 1 to about 30 mg) compounded with an appropriate and convenient amount of excipients which may vary from about 5 to about 98 or about 99 percent by weight of the total composition.

For the above-mentioned compounds of the disclosure the dosage administered will, of course, vary with the compound employed, the mode of administration, the treatment desired and the disorder indicated. In using a compound of the disclosure for therapeutic or prophylactic purposes it will generally be administered so that a daily dose in the range, for example, a daily dose selected from about 0.1 mg/kg to about 100 mg/kg, about 1 mg/kg to about 75 mg/kg, about 1 mg/kg to about 50 mg/kg, about 1 mg/kg to about 20 mg/kg or about 5 mg/kg to about 10 mg/kg body weight is received, given if required in divided doses. In general lower doses will be administered when a parenteral route is employed. Thus, for example, for intravenous or intraperitoneal administration, a dose in the range, for example, about 0.1 mg/kg to about 30 mg/kg body weight will generally be used. Similarly, for administration by intraarticular, a dose in the range, for example, about 0.01 mg/kg to about 30 mg/kg body weight may generally be used. For administration by inhalation, a dose in the range, for example, about 0.05 mg/kg to about 25 mg/kg body weight will be used. Suitably the compound of the disclosure is administered orally, for example in the form of a tablet, or capsule dosage form. The daily dose administered orally may be, for example a total daily dose selected from about 1 mg to about 1000 mg, about 5 mg to about 1000 mg, about 10 mg to about 750 mg or about 25 mg to about 500 mg. Typically, unit dosage forms will contain about 0.5 mg to about 0.5 g of a compound of this disclosure.

The compounds of the disclosure may be administered along with other active compounds as part of a treatment regime. The other active compounds may be administered simultaneously with, subsequently to or previously to the administration of the compounds of the disclosure. It may be that the pharmaceutical formulation comprising the compounds of the disclosure also comprises one or more other active compounds. The other active compounds may be anticancer, anti-inflammatory, antibacterial, antiviral, antiemetic, antithrombotic or compounds that alter the metabolism.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

It will be understood that various modifications may be made to the embodiments disclosed herein. Therefore, the above description should not be construed as limiting, but merely as exemplifications of preferred embodiments. For example, the functions described above and implemented as the best mode for operating the present disclosure are for illustration purposes only. Other arrangements and methods may be implemented by those skilled in the art without departing from the scope and spirit of this disclosure. Moreover, those skilled in the art will envision other modifications within the scope and spirit of the specification appended hereto.

The reader's attention is directed to all papers and documents which are filed concurrently with or previous to this specification in connection with this application and which are open to public inspection with this specification, and the contents of all such papers and documents are incorporated herein by reference.

### EXAMPLES

### Abbreviations

APCI atmospheric pressure chemical ionisation mass spectrum
BD binding domain
BET bromodomain and extra terminal domain
br broad
BRD Bromodomain-containing protein
BRET Bioluminescence resonance energy transfer
c centi
δ delta
d doublet
dd double doublet
DCM dichloromethane
DIBAL-H diisobutylaluminium hydride
Dioxane 1,4-dioxane
DMF dimethyl formamide
DMA dimethyl acetamide
DMSO dimethyl sulfoxide
EA Ethyl acetate
EC effective concentration
ES electrospray
ESI electrospray ionization
FA formic acid
g gram
hr hour
HPLC high performance liquid chromatography
HRMS high resolution mass spectrum
J coupling constant
Kd binding affinity
L litre
LC liquid chromatography
LG leaving group
Luc Luciferase
m multiplet
m milli
m meter
M molar
M+ molecular ion
MHz megahertz
min minutes
mol mole
MS mass spectrometry
m/z mass/charge
n nano
NMR nuclear magnetic resonance
PE petroleum ether
p para
PTSA p-Toluenesulfonic acid
q quartet
Rf retardation factor
rpm revolutions per minute
RT room temperature
s singlet
SM starting material
S_{N}Ar nucleophilic aromatic substitution (addition-elimination)
t triplet
TBAF Tetra-n-butylammonium fluoride
TFA Trifluoroacetic acid
THE tetrahydrofuran
TLC thin layer chromatography
TLR toll-like receptors
TBME methyl tert-butyl ether
t_{R} retention time
S-Phos 2-Dicyclohexylphosphino-2′,6′-dimethoxybiphenyl
X-Phos 2-dicyclohexylphosphino-2′,4′,6′-triisopropylbiphenyl
Ts or Tosyl toluenesulfonyl
DTT dithioreitol
BSA bovine serum albumin
PBS phosphate-buffered saline
MEG monoethylene glycol
NADPH nicotinamide adenine dinucleotide phosphate
p micro
UPLC ultra performance liquid chromatography
UV ultraviolet
vis visible
w/w weight by weight
° C. degree Celsius
% percent

### Equipment

Reactions using microwave irradiation were carried out in a Biotage Initiator microwave.

Normal phase TLCs were carried out on pre-coated silica plates (Kieselgel 60 F₂₅₄, BDH) with visualisation via U.V. light (UV254/365 nm) and/or ninhydrin solution.

Flash chromatography was performed using Combiflash Companion Rf (Teledyne ISCO) and prepacked silica gel columns purchased from Grace Davison Discovery Science or SiliCycle or manually in glass columns using Finar silica mesh size 100-200.

Preparative HPLC separations were performed with either Instrument A) Shimadzu LC-20AP purification system with UV detector, or B) Agilent 1200 series infinity-II purification system with UV detector or a C) Waters 2545 Binary Gradient Module connected to a Waters 2489 UV/visible detector. On all instruments, HPLC chromatographic separations were conducted using either Column A) Xbridge Prep, C18, OBD 19×250 mm, 5 pm, B) VIRDIS PREP SILICA, 2-EP-OBD, 250×19 mm, 5 μm, C) YMC-Actus Triart Prep C18-S, 250×20 mm S-5 μm, 12 nm, D) Sunfire prep C18 column, 30*150 mm, 5 μm, E) Xselect CSH F-Phenyl OBD column, 19*250 mm, 5 μm, F) a XBridge Prep Phenyl OBD Column, 19*150 mm, 5 μm G) Column: Xselect CSH C18 OBD Column 30*150 mm 5 μm using the mobile phase shown.

¹H NMR, ¹³C NMR spectra were recorded on a Bruker AVANCE Ill HD 400 MHz spectrometer (¹H NMR at 400 MHz and ¹³C NMR at 100 MHz) or a Bruker AVANCE III HD 300 MHz (¹H NMR at 300 MHz, ¹³C NMR at 75 MHz) or a Bruker AVANCE NEO 400 MHz spectrometer (¹H NMR at 400 MHz, ¹³C NMR at 100 MHz). Chemical shifts (6) are expressed in ppm recorded using the residual solvent as the internal reference in all cases. Signal splitting patterns are described as singlet (s), doublet (d), triplet (t), quartet (q), multiplet (m), broad (br), or a combination thereof. Coupling constants (J) are quoted to the nearest 0.5 Hz.

LC-MS analysis and chromatographic separation were conducted with a Waters Acquity Ultra performance LC connected to a Waters QDA Mass detector, connected to Waters diode array detector or an Agilent Technologies 1200 series HPLC connected to an Agilent Technologies 6130 quadrupole LC/MS, connected to an Agilent diode array detector. The column used on both was a Waters XBridge column (50 mm×2.1 mm, 2.5 μm particle size,) and the compounds were eluted with, Mobile Phase A: 0.1% Formic acid in Milli Q water (pH=2.70), Mobile Phase B: 0.1% Formic acid in Milli Q water: Acetonitrile (10:90) with a gradient of T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min or a gradient of 5 to 95% acetonitrile/water+0.1% Ammonia. Alternatively a Shimadzu LMCS-2020 was used with A) an Ascentis Express C18 30 mm×3.0 nm column and the compounds were eluted with, Mobile phase A: Water (0.1% FA), Mobile phase B: Acetonitrile (0.1% FA) with a gradient of T=0.01 min (95% A, 5% B) flow rate: 1.50 L/min; T=1.80 min (60% A, 40% B), flow rate: 1.50 L/min; gradient to T=2.00 min (0% A; 100% B) and then held for 0.7 min; flow rate: 1.50 ml/min, end of run at T=2.80 min, flow rate: 1.50 mL/min; B) a HPH C18 50 mm×3.0 mm column and the compounds were eluted with, Mobile phase A: Water (0.04% NH3H2O), Mobile phase B: Acetonitrile with a gradient of T=2.20 min (30% A, 70% B) flow rate: 1.50 L/min; T=2.40 min (5% A, 95% B), and then held for 0.40 min, flow rate: 1.50 L/min; gradient to T=2.85 min (10% A, 90% B), flow rate: 1.50 L/min; flow rate: 1.50 L/min, end of run at T=3.00 min, flow rate: 1.50 L/min; C) a HPH C18 50 mm×3.0 mm column and the compounds were eluted with, Mobile phase A: Water (0.1% FA), Mobile phase B: Acetonitrile (0.1% FA) with a gradient of T=0.01 min (95% A, 5% B) flow rate: 1.50 L/min; T=1.80 min (40% A, 60% B), flow rate: 1.50 L/min; T=2.00 min (0% A; 100% B) and then held for 0.7 min; flow rate: 1.50 ml/min, gradient to T=2.74 min (95% A; 5% B); end of run at T=2.80 min, flow rate: 1.50 mL/min; D) a EVO C18 50 mm×3.0 mm column and the compounds were eluted with, Mobile phase A: Water (5 mM NH4HCO3), Mobile phase B: Acetonitrile with a gradient of T=0.01 min (90% A, 10% B) flow rate: 1.50 L/min; T=2.00 min (30% A, 70% B), flow rate: 1.50 L/min; gradient to T=2.20 min (5% A; 95% B) and then held for 0.40 min; flow rate: 1.50 L/min, T=2.75 min (90% A, 10% B) flow rate: 1.50 L/min; end of run at T=2.80 min, flow rate: 1.50 L/min; E) a Ascentis Express C18 30 mm×3.0 nm column and the compounds were eluted with, Mobile phase A: Water (5 mM NH4HCO3), Mobile phase B: Acetonitrile with a gradient of T=0.01 min (90% A, 10% B) flow rate: 1.50 L/min; T=2.00 min (30% A, 70% B), flow rate: 1.50 L/min; gradient to T=2.20 min (5% A; 95% B) and then held for 0.4 min; T=2.75 min (90% A, 10% B), flow rate: 1.50 L/min; end of run at T=2.80 min, flow rate: 1.50 L/min.

Solvents and reagents were purchased from commercial suppliers and used without further purification. Dry solvents were purchased in sure sealed bottles stored over molecular sieves.

Preparations and compounds have been named using the ChemDraw Professional 19.1 naming application.

### Process for Preparation

Certain compounds of the disclosure may be synthesised according to the general methods disclosed herein. Certain compounds of the disclosure may be synthesised according to or analogously to the syntheses provided in the examples.

The following schemes illustrate methods of synthesising the compounds of the disclosure. Scheme 1-4 illustrates routes for the preparation of general intermediates of the disclosure.

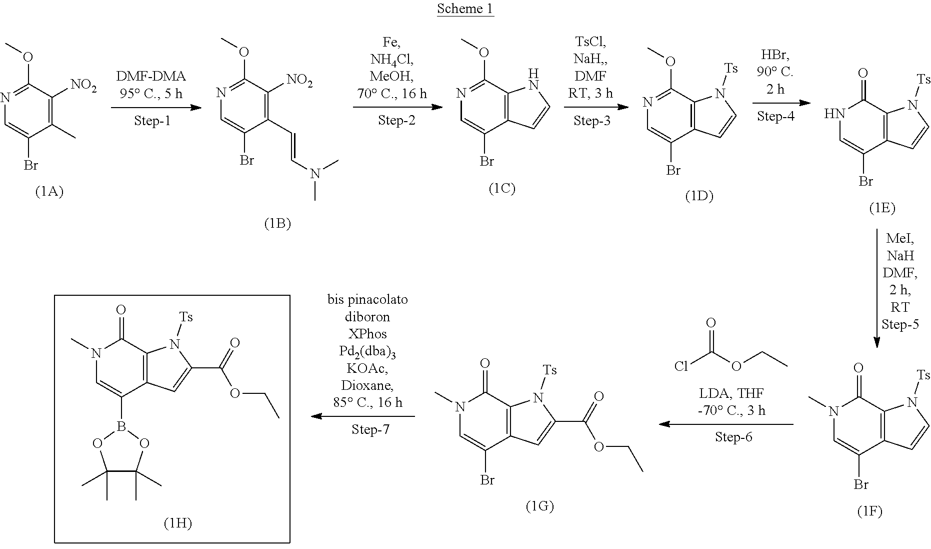

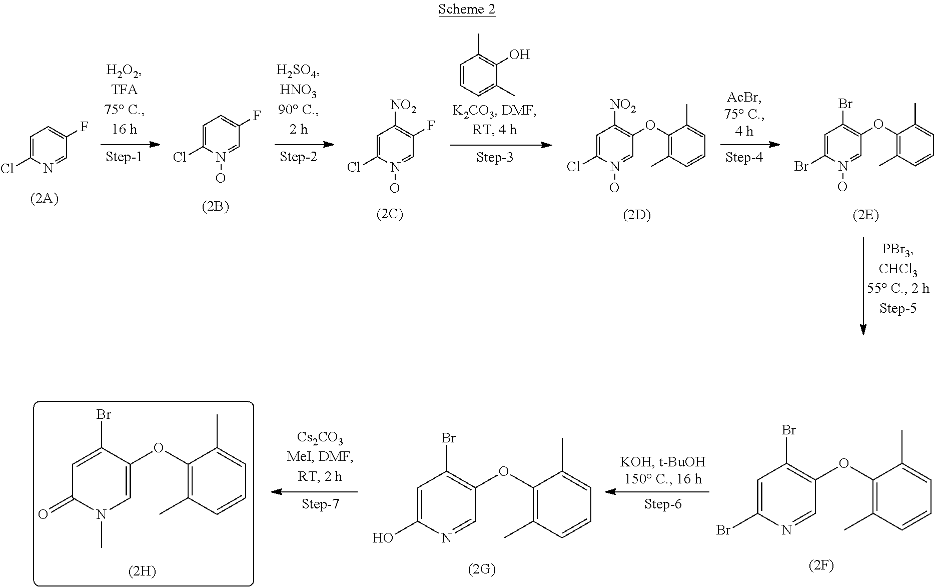

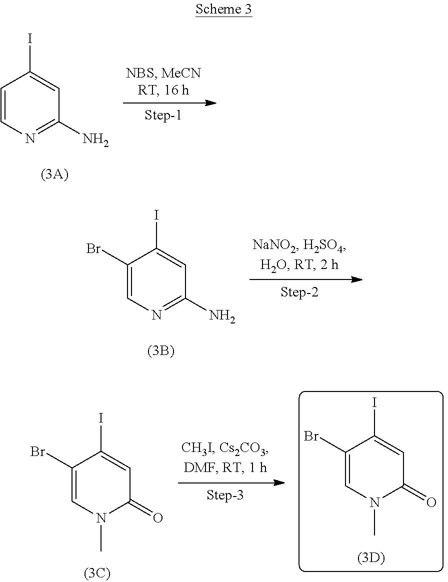

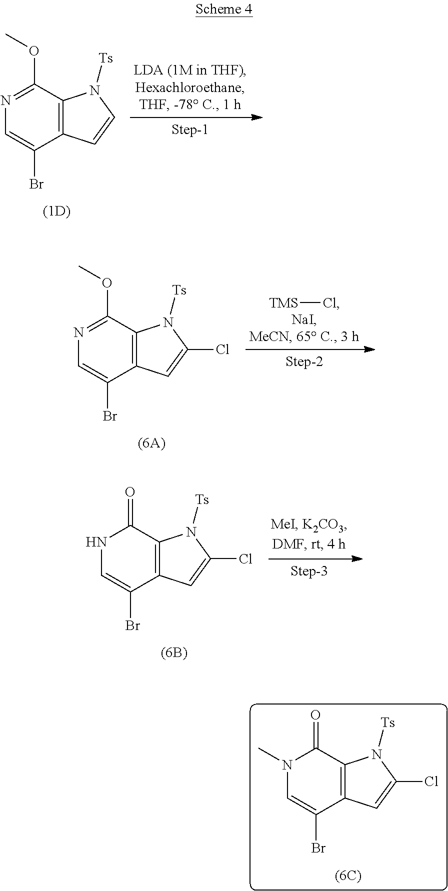 [SMILES: COc1ncc(Br)c2ccn(S(=O)(=O)c3ccc(C)cc3)c12 ; COc1ncc(Br)c2cc(Cl)n(S(=O)(=O)c3ccc(C)cc3)c12 ; C[Si](C)(C)Cl ; Cc1ccc(S(=O)(=O)n2c(Cl)cc3c(Br)c[nH]c(=O)c32)cc1 ; Cc1ccc(S(=O)(=O)n2c(Cl)cc3c(Br)cn(C)c(=O)c32)cc1]

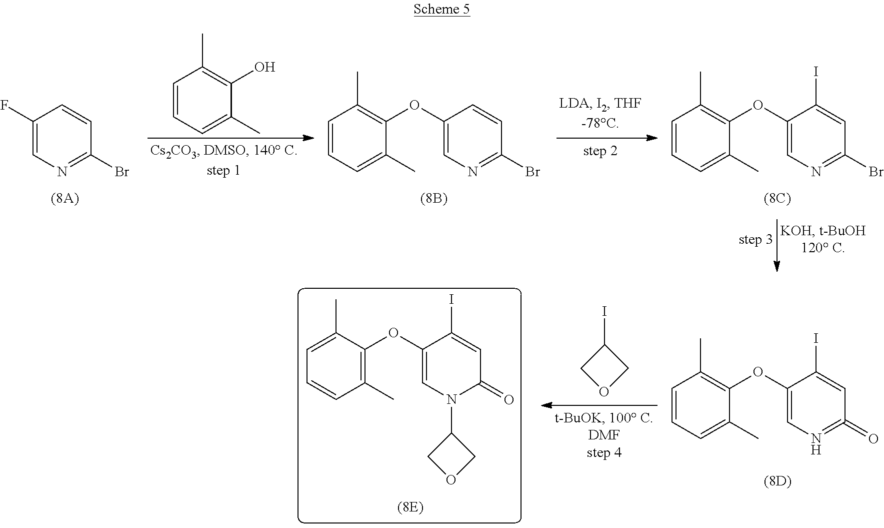

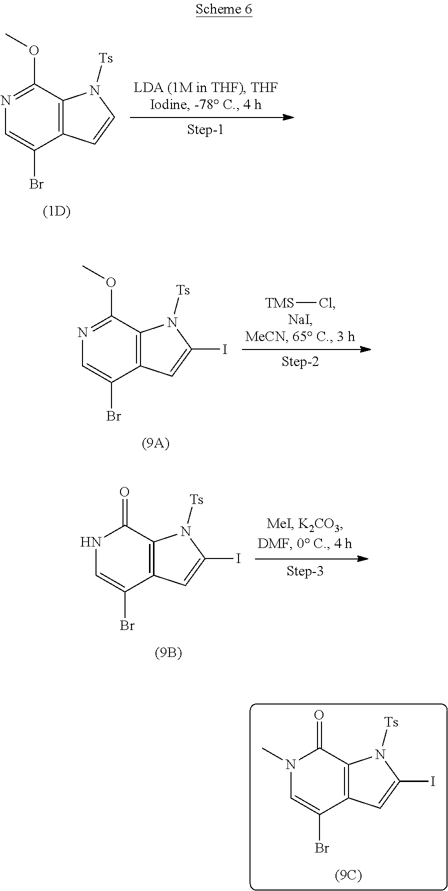 [SMILES: COc1ncc(Br)c2ccn(S(=O)(=O)c3ccc(C)cc3)c12 ; COc1ncc(Br)c2cc(I)n(S(=O)(=O)c3ccc(C)cc3)c12 ; C[Si](C)(C)Cl ; Cc1ccc(S(=O)(=O)n2c(I)cc3c(Br)c[nH]c(=O)c32)cc1 ; Cc1ccc(S(=O)(=O)n2c(I)cc3c(Br)cn(C)c(=O)c32)cc1]

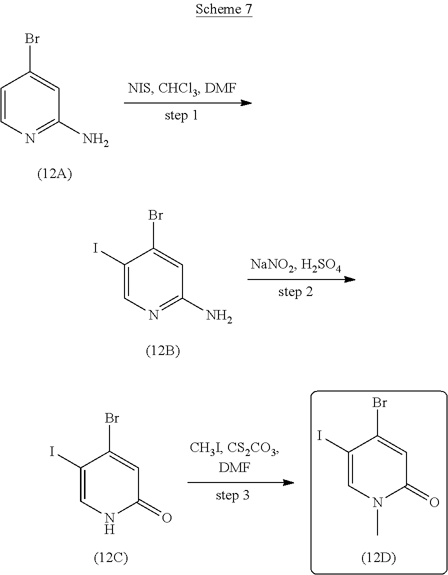

General Scheme 1 illustrates a general route for the preparation of compounds of the disclosure via Suzuki coupling of intermediates followed by reduction, oxidation, ring condensation and deprotection.

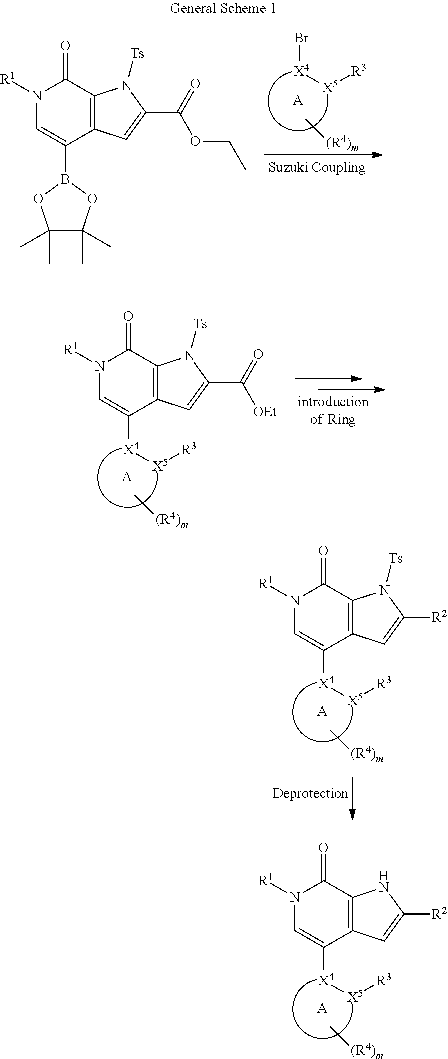

General Scheme 1 a illustrates a general route for the preparation of compounds of the disclosure via Suzuki coupling of intermediates (1H) and (2H) followed by reduction, oxidation, ring condensation and deprotection.

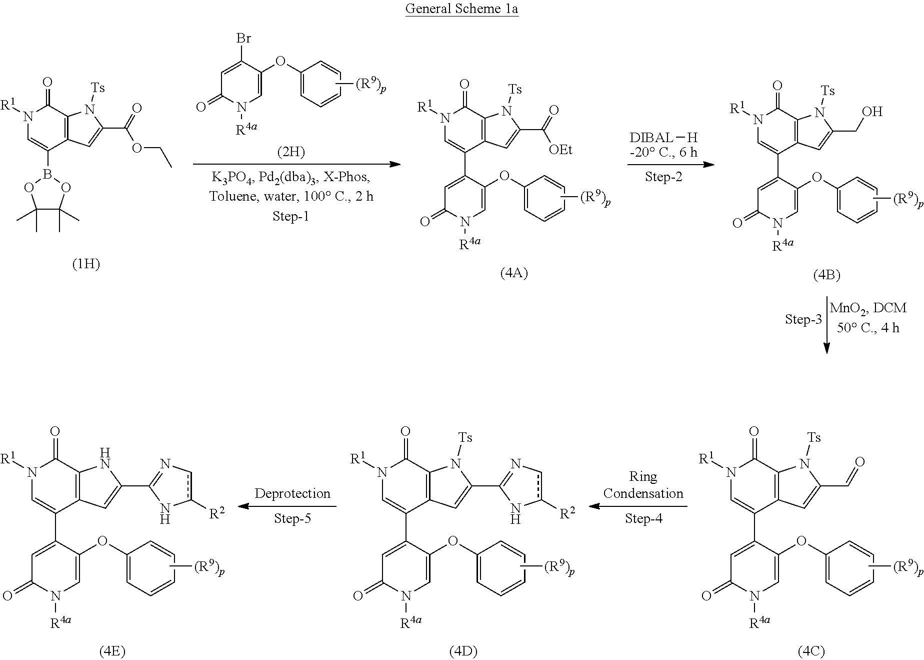

General Scheme 2 illustrates a general route for the preparation of compounds of the disclosure via Suzuki coupling of intermediates followed by Pd coupling, reduction, oxidation, ring condensation and deprotection.

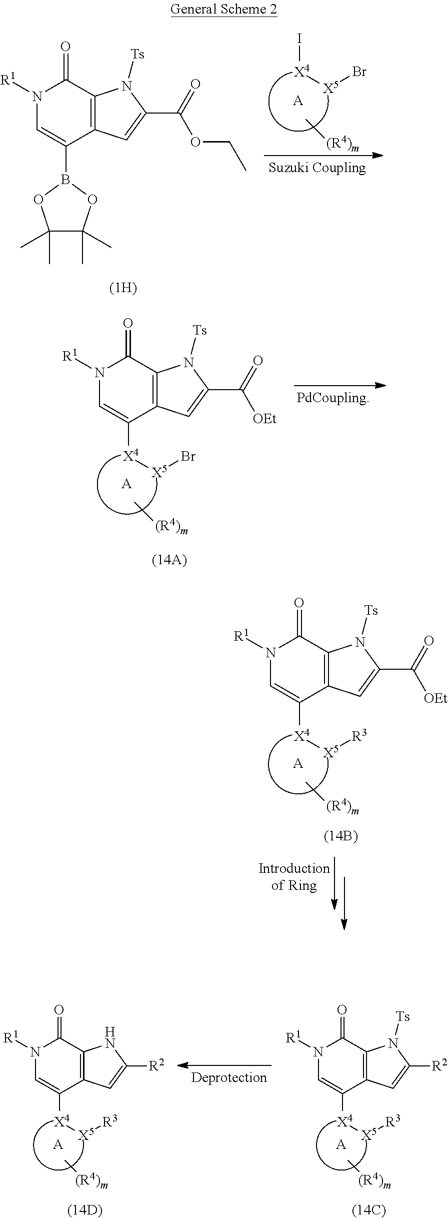

General Scheme 2a illustrates a general route for the preparation of compounds of the disclosure via Suzuki coupling of intermediates (1H) and (3D) followed by Pd coupling, reduction, oxidation, ring condensation and deprotection.

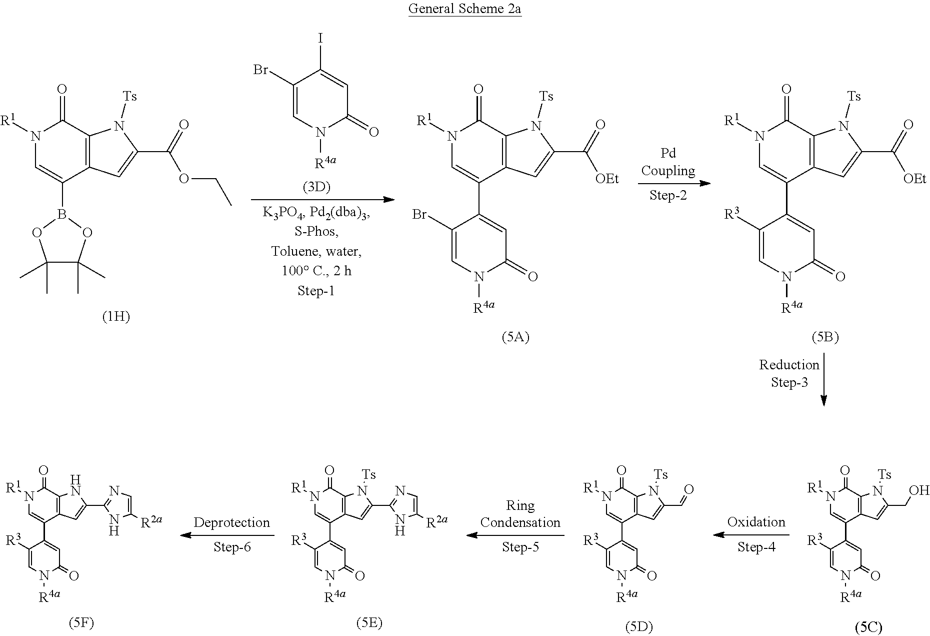

General Scheme 3 illustrates a general route for the preparation of compounds of the disclosure via formation of coupling of partners from (15A), followed by Pd coupling, Pd coupling and deprotection.

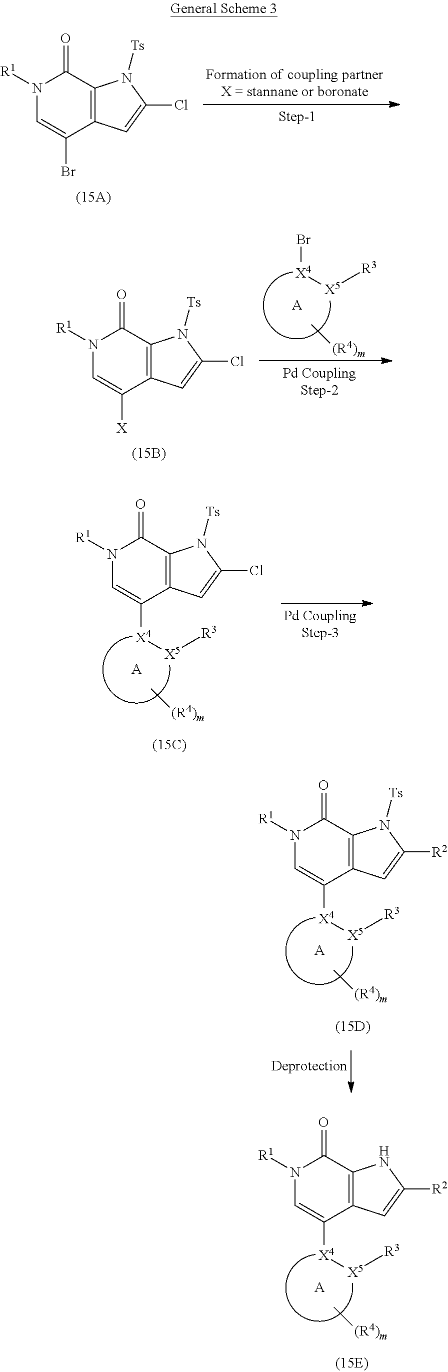

General Scheme 3a illustrates a general route for the preparation of compounds of the disclosure via formation of coupling of partners from (6C), followed by Pd coupling with (2H), Pd coupling and deprotection.

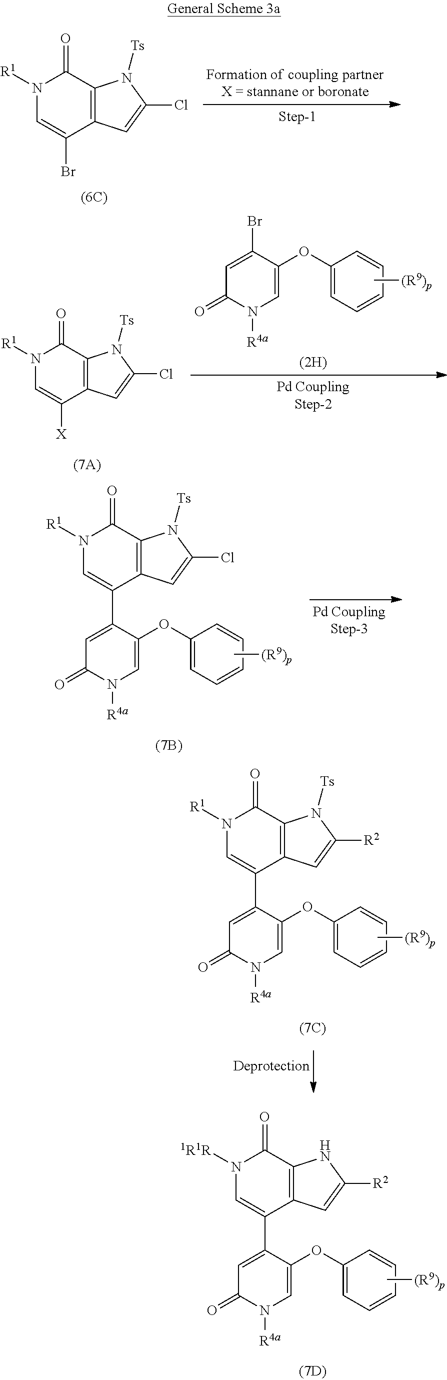

General Scheme 3b illustrates a general route for the preparation of compounds of the disclosure via formation of coupling of partners from (6C), followed by Pd coupling with (2H), deprotection and Pd coupling.

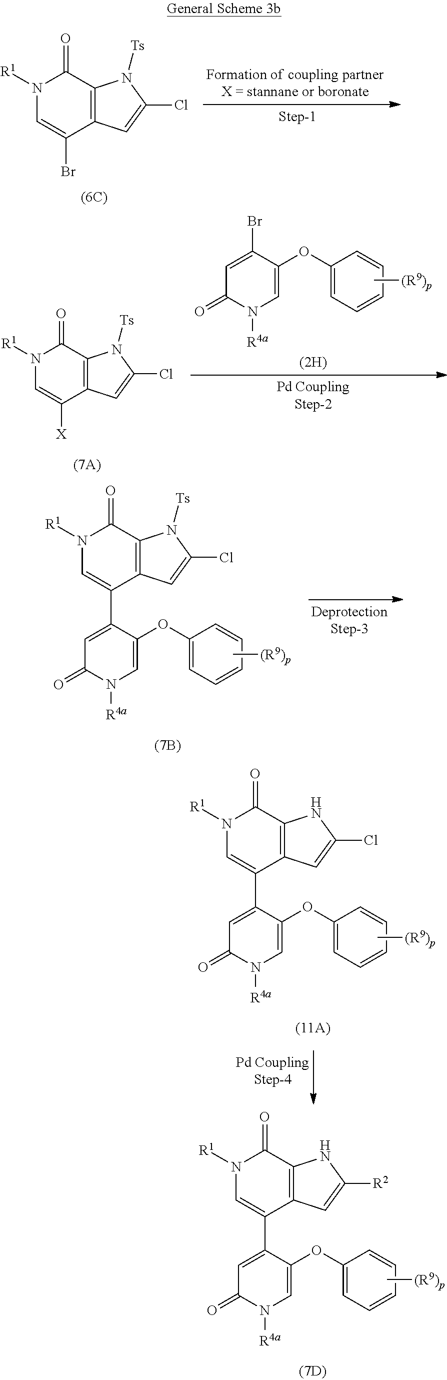

General Scheme 3c illustrates a general route for the preparation of compounds of the disclosure via Pd coupling from (9C), followed by formation of coupling of partners, Pd coupling with (2H) and deprotection.

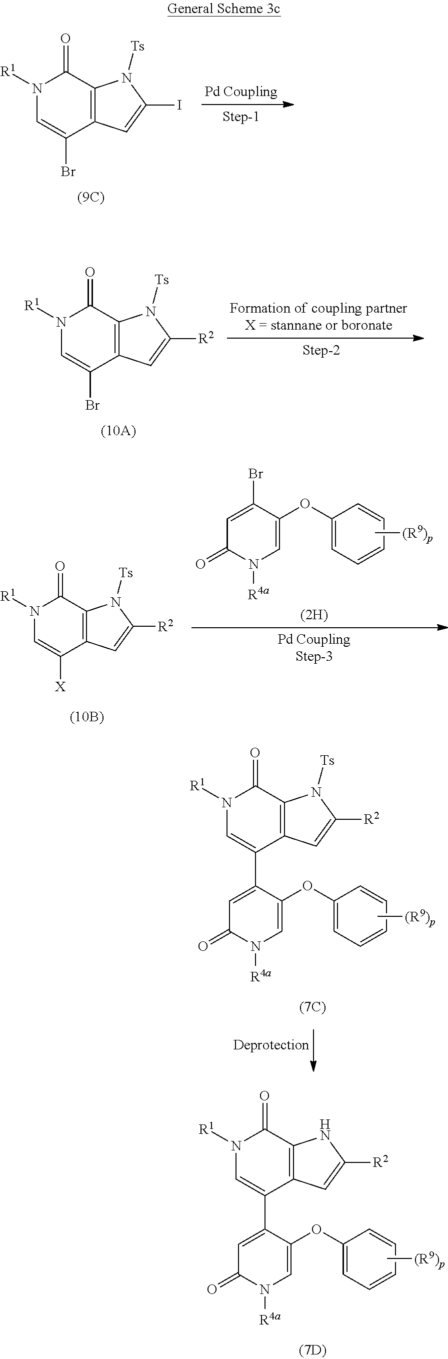

General Scheme 4 illustrates a general route for the preparation of compounds of the disclosure via Pd coupling from (12D), followed by Pd coupling with (7A), Pd coupling and deprotection.

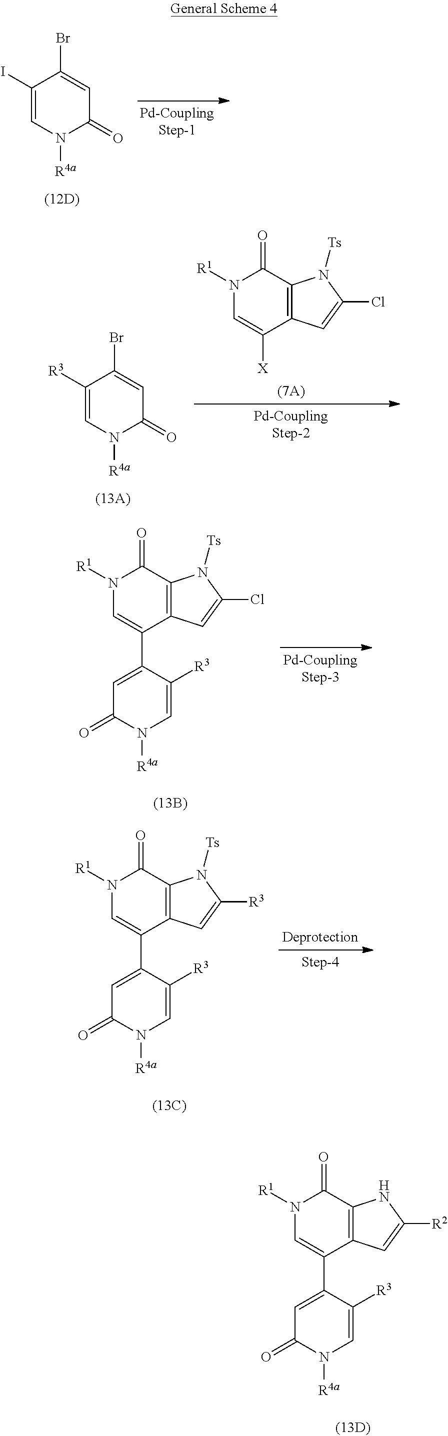

### Example 1: 4-(5-(2,6-dimethylphenoxy)-1-methyl-2-oxo-1,2-dihydropyridin-4-yl)-6-methyl-2-(5-methyl-1H-imidazol-2-yl)-1,6-dihydro-7H-pyrrolo [2,3-c] pyridin-7-one

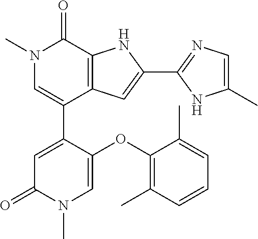

### Preparation 1: (E)-2-(5-bromo-2-methoxy-3-nitropyridin-4-yl)-N,N-dimethylethen-1-amine

5-bromo-2-methoxy-4-methyl-3-nitropyridine (50 g, 202 mmol) was dissolved in DMF (410 mL) under nitrogen and heated to 80° C. DMF-DMA (224 mL, 1.686 mol) was added over a period of 20 min. The resulting dark solution was heated at 95° C. TLC (4:1 heptane/EA) after 5 hr showed no SM remaining. The mixture was cooled to RT and poured into ice water (1100 mL). The resulting suspension was stirred for 15 min then filtered. The collected red solid was washed with water and dried overnight under vacuum at 50° C. (56.6 g, 61%). The material was used directly in preparation 2 without further purification.

¹H NMR (400 MHz, CDCl₃) δ 8.14 (s, 1H), 7.02 (d, J=13.7 Hz, 1H), 4.94 (d, J=13.7 Hz, 1H), 3.97 (s, 3H), 2.94 (s, 6H).

### Preparation 2: 4-bromo-7-methoxy-1H-pyrrolo[2,3-c]pyridine

(E)-2-(5-bromo-2-methoxy-3-nitropyridin-4-yl)-N,N-dimethylethen-1-amine (23.3 g, 77.1 mmol) was partially dissolved in methanol (1100 mL) and ammonium chloride (23.3 g, 436 mmol), followed by water (140 mL). Iron powder (23.3 g, 417 mmol) was added and the mixture heated at reflux. The reaction mixture was stirred using an overhead stirrer. After 5 hr a further aliquot of iron powder (23.3 g, 417 mmol) was added and heating continued overnight. The mixture was cooled and solid Na₂CO₃ was added. The mixture was filtered through a pad of celite and dried under vacuum. The residue was triturated with 4:1 heptane/Ethyl acetate. The mixture was filtered through a pad of silica. The filtrate was evaporated. The residue was purified on silica, eluting with 100:0 to 80:20 heptane/ethyl acetate. Solvent reduction gave an off-white solid (3.7 g, 21%).

HPLC t_{R} (Agilent, acidic, 3.5 min): 1.46 min, MS: m/z 229.0 [M+H]⁺.

### Preparation 3: 4-bromo-7-methoxy-1-tosyl-1H-pyrrolo[2,3-c]pyridine

Sodium hydride (60% w/w, 7.90 g, 198 mmol) was suspended in THF (290 mL) under nitrogen and was cooled to below 4° C. in an ice bath. 4-bromo-7-methoxy-1H-pyrrolo[2,3-c]pyridine (14.0 g, 61.7 mmol) was dissolved in THF (290 mL) and added dropwise over a period of 30 min (evolution of gas was observed and formation of an exotherm raised the reaction temperature to 5° C.). The maroon mixture was stirred at RT for 45 min before cooling to 3° C. 4-Methylbenzenesulfonyl chloride (15.7 g, 82.1 mmol) was dissolved in THF (290 mL) and added dropwise. The resulting grey suspension was stirred 1.5 hr with cooling, and then 1 hr at RT. TLC (3:2 heptane/ethyl acetate) showed no remaining SM. The reaction mixture was quenched by dropwise addition of sat NH₄Cl (300 mL). The mixture was stirred 5 min before separating the phases. The aqueous phase was extracted with ethyl acetate (2×300 mL). The combined organics were washed (brine), dried (MgSO₄), filtered and evaporated to an oil that crystallized on cooling to give a light tan solid (26.2 g 99%). The material was used directly in preparation 4 without further purification.

HPLC t_{R} (Agilent, acidic, 3.5 min): 1.94 min, m/z=383.1 [M+H]⁺.

### Preparation 4: 4-bromo-1-tosyl-1,6-dihydro-7H-pyrrolo[2,3-c]pyridin-7-one

4-bromo-7-methoxy-1-tosyl-1H-pyrrolo[2,3-c]pyridine (26.2 g, 65.3 mmol) was suspended in ethanol (50 mL) and hydrogen bromide (48% w/w, 280 mL) was added in a steady stream. The resulting mixture was heated at 90° C. TLC (3:2 heptane/ethyl acetate) after 2 hr showed no remaining SM. The reaction mixture was cooled to RT and then cooled in an ice bath with stirring for 30 min. The mixture was filtered and the cream coloured solid was collected and washed with water. The solid was dried overnight under vacuum at 50° C. (22.5 g, 94%). The material was used directly in preparation 5 without further purification.

HPLC t_{R} (Agilent, acidic, 3.5 min): 1.59 min, m/z=369.0 [M+H]⁺.

### Preparation 5: 4-bromo-6-methyl-1-tosyl-1,6-dihydro-7H-pyrrolo[2,3-c]pyridin-7-one

4-bromo-1-tosyl-1,6-dihydro-7H-pyrrolo[2,3-c]pyridin-7-one (22.5 g, 61.3 mmol) was dissolved in DMF (225 mL) under nitrogen. The mixture was cooled to 3° C. and sodium hydride (60% w/w, 3.06 g, 76.6 mmol) added in small portions, producing an evolution of gas and exotherm to 5° C. The mixture was stirred for 20 min with cooling where after the evolution of gas had ceased, iodomethane (7.63 mL, 123 mmol) was added dropwise, producing an exotherm which raised the reaction temperature to 10° C. The mixture was stirred for 15 min with cooling, then for 15 min at RT. LCMS after 2 hr showed no SM remaining. The reaction mixture was quenched by dropwise addition of water (100 mL, evolution of gas and exotherm to 39° C.). The mixture was extracted with ethyl acetate (3×300 mL). The combined organics were washed (brine), dried (Na₂SO₄), filtered and evaporated. The crude product was triturated with TBME and filtered. The collected off-white solid was washed with TBME and dried under vacuum (15 g, 64%).

HPLC t_{R} (Agilent, basic, 6.0 min): 4.0 min, m/z=382.9 [M+H]⁺.

### Preparation 6: ethyl 4-bromo-6-methyl-7-oxo-1-tosyl-6,7-dihydro-1H-pyrrolo[2,3-c]pyridine-2-carboxylate

4-bromo-6-methyl-1-tosyl-1,6-dihydro-7H-pyrrolo[2,3-c]pyridin-7-one (1.3 g, 3.4 mmol), in THF (100 mL) was cooled to −78° C. LDA (2.03 mL, 4.06 mmol) was added dropwise and the resulting solution stirred at this temperature for 30 minutes. Ethyl carbonochloridate (0.39 mL, 4.06 mmol) was added and the reaction stirred for 1 hour at −78° C. Ethyl acetate (500 ml) was added and the organics washed with 2×500 ml water then 1×500 ml saturated brine solution. The organics were then separated and dried (MgSO₄) before concentration to dryness. The crude was then purified by flash column chromatography eluting with ethyl acetate/heptane gradient (0-100%). The desired fractions were combined and dried to afford was reacted to give the title compound (770 mg, 50%).

HPLC t_{R} (Agilent, acidic, 3.5 min): 1.85 min, m/z=453.8 [M]⁺.

### Preparation 7: ethyl 6-methyl-7-oxo-4-(4,4,5,5-tetramethyl-1,3,2-dioxaborolan-2-yl)-1-tosyl-6,7-dihydro-1H-pyrrolo[2,3-c]pyridine-2-carboxylate

To a flask containing XPhos (76 mg, 0.16 mmol), ethyl 4-bromo-6-methyl-7-oxo-1-tosyl-6,7-dihydro-1H-pyrrolo[2,3-c]pyridine-2-carboxylate (710 mg, 1.6 mmol), 4,4,5,5-tetramethyl-2-(4,4,5,5-tetramethyl-1,3,2-dioxaborolan-2-yl)-1,3,2-dioxaborolane (813 mg, 3.2 mmol) and potassium acetate (188 mg, 1.92 mmol) was added 1,4-Dioxane (20 mL) and the suspension was degassed for 10 min. Pd₂(dba)₃ (30 mg, 0.032 mmol) was added, and the mixture degassed for 1 min more. The reaction was heated at 80° C. overnight. The reaction was diluted with ethyl acetate and washed with 50% brine. The organics were dried, filtered and concentrated to a yellow/brown oil. The product was purified by flash chromatography on silica gel (20 g) eluting with ethyl acetate/heptane gradient (0-80%). Fractions corresponding to product were combined and concentrated to give the title compound (437 mg, 56%).

HPLC t_{R} (Agilent, acidic, 3.5 min): 2.10 min. m/z=501.1 [M+H]⁺.

### Preparation 8: 2-chloro-5-fluoropyridine 1-oxide

Trifluoroacetic acid (2.4 L, 8v) was charged in 5 L four neck RBF at to 0° C. 2-chloro-5-fluoropyridine (300 g, 229.02 mmol) was dropwise added to the pre-cooled mixture by using additional funnel over 20 min. 30% hydrogen peroxide (450 mL, 39.70 mmol) was slowly added to reaction mixture. The resulting mixture was heated at 75° C. for 16 h. TLC (5.0:5.0 Hexane:Ethyl acetate) showed no SM remaining. Trifluoroacetic acid (2.3 L) was separated by vacuum distillation. The resulting mixture was diluted by using cold water (2000 mL) & added 70% of aqueous ammonia solution (500 mL). The aqueous fraction was extracted with dichloromethane (6×2000 mL). The combined organics were washed (brine), dried (Na2SO4), filtered and evaporated. The residue was triturated by n-pentane. Solvent reduction to give light brown solid (314 g, 93.32%).

LCMS: (Waters Acquity UPLC with QDA mass Detector, Acidic, 4.0 min): 0.453 min, m/z=147.96 [M+H]+

¹H NMR (400 MHz, DMSO d₆) δ 8.82 (m, 1H), 7.88 (m, 1H), 7.44 (m, 1H).

### Preparation 9: 2-chloro-5-fluoro-4-nitropyridine 1-oxide

2-chloro-5-fluoropyridine 1-oxide (100 g, 677.9 mmol) was dissolved in H₂SO₄ (500 mL, 5V) in 5 L four neck RBF at room temperature. The resulting mixture was heated at 90° C. The pre-stirred solution of H₂SO₄ (1000 mL, 10V) and HNO₃ (283 mL, 6777.9 mmol) at 0° C. was dropwise added to the reaction mixture at 90° C. The reaction mixture was allowed to stir at same temperature for 2 h. TLC (5.0:5.0 Hexane:Ethyl acetate) showed no SM remaining. The resulting mixture was cooled at room temperature & ice (5 Kg) was portion wise added under stirring. The aqueous fraction was extracted with ethyl acetate (2×1000 mL), The combined organics were washed (NaHCO₃ solution), dried (Na₂SO₄), filtered and evaporated. The resulting mixture was triturated by hexane (2×50 mL). Solvent reduction to give light yellow solid (68 g, 53.87%).

LCMS: (Waters Acquity UPLC with QDA mass Detector, Acidic, 4.0 min): 0.830 min, m/z=193.12 [M+H]+.

¹H NMR (400 MHz, DMSO d₆) δ 8.82 (d, J=6.8 Hz, 1H), 8.82 (d, J=8.8 Hz, 1H).

### Preparation 10: 2-chloro-5-(2,6-dimethylphenoxy)-4-nitropyridine 1-oxide

2-chloro-5-fluoro-4-nitropyridine 1-oxide (178.5 g, 934.5 mmol) was dissolved in dimethylformamide (892.5 mL, 5V) under nitrogen. Potassium carbonate (768.20 g, 5,607 mmol) was added to the reaction mixture and allowed to stir for 30 min. 2,6-Dimethylphenol (119.71 g, 981.28 mmol) was added to the reaction mixture and stirred for 4 h at room temperature. TLC (7.0:3.0 Hexane:Ethyl acetate) showed no SM remaining. The resulting mixture was quenched with water (1000 mL) and stirred for 30 min. The resulting residue was filtered and triturated by n-hexane. The solid was dried overnight under vacuum at 45° C. (245 g, 89.67%).

¹H NMR (400 MHz, DMSO d₆) δ 8.72 (s, 1H), 7.42 (s, 1H), 7.23 (m, 3H), 2.14 (s, 6H).

### Preparation 11: 2, 4-dibromo-5-(2,6-dimethylphenoxy) pyridine 1-oxide

2-chloro-5-(2,6-dimethylphenoxy)-4-nitropyridine 1-oxide (40 g, 13.5 mmol) was dissolved in acetyl bromide (200 mL, 5V) at room temperature under nitrogen. The resulting mixture was heated at 75° C. & dropwise added acetyl bromide (200 mL, 5V). The reaction mixture was stirred at same temperature for 4 h. TLC (5.0:5.0 Hexane:Ethyl acetate) showed no SM remaining. The resulting mixture was slowly poured in cold water (5000 mL). The aqueous fraction was extracted with ethyl acetate (2×2000 mL). The combined organics were washed (NaHCO₃ solution), dried (Na2SO4), filtered and evaporated. Solvent reduction to give light brown solid (26 g, 51.35%).

LCMS: (Waters Acquity UPLC with QDA mass Detector, Acidic, 4.0 min): 1.97 min, m/z=372.0 [M+H]+.

¹H NMR (400 MHz, DMSO d₆) δ 8.42 (s, 1H), 7.21 (m, 4H), 2.10 (s, 6H).

### Preparation 12: 2,4-dibromo-5-(2,6-dimethylphenoxy) pyridine

2, 4-dibromo-5-(2,6-dimethylphenoxy) pyridine 1-oxide (200 g, 539.08 mmol) was dissolved in chloroform (2000 mL, 10V) under nitrogen. Phosphorus tribromide (200 mL, 1V) was dropwise added to reaction mixture over 30 min and allowed to stir at 55° C. for 2 h. TLC (5.0:5.0 Hexane:Ethyl acetate) showed no SM remaining. The resulting mixture was slowly poured in cold water (5000 mL). The aqueous fraction was extracted with ethyl acetate (2×2000 mL). The combined organics were washed (NaHCO₃ solution), dried (Na2SO4), filtered and evaporated. Solvent reduction to give light brown solid (130 g, 67.91%).

LCMS: (Waters Acquity UPLC with QDA mass Detector, Acidic, 4.0 min): 2.67 min, m/z=356.1 [M+H]+.

¹H NMR (400 MHz, DMSO d₆) δ 8.16 (s, 1H), 7.36 (s, 1H), 7.20-7.18 (m, 3H), 2.07 (s, 6H).

### Preparation 13: 4-bromo-5-(2,6-dimethyl phenoxy) pyridin-2-ol

2,4-dibromo-5-(2,6-dimethylphenoxy) pyridine (10 g, 28.24 mmol) was dissolved in tert-butanol (125 mL, 12.5V) at room temperature in autoclave. Potassium hydroxide (15.8 g, 282.40 mmol) was added to the reaction mixture and allowed to stir at 150° C. for 16 h. TLC (9.0:1.0 DCM:methanol) showed no SM remaining. The reaction mixture was cooled at 0° C. and added ice. The mixture was acidified by 2N HCl to adjust pH 2. The resulting fraction was extracted with DCM (2×700 mL). The combined organics were washed (brine solution), dried (Na₂SO₄), filtered and evaporated. The residue was triturated by ethyl acetate (100 mL). Solvent reduction to give light brown solid. (4 g, 48.55%) (LCMS purity 43.68%).

LCMS: (Waters Acquity UPLC with QDA mass Detector, Acidic, 4.0 min): 1.69 min, m/z=294.0 [M+H]+

¹H NMR (400 MHz, DMSO d₆) δ 11.27 (br s, 1H), 7.18-7.09 (m, 3H), 6.92 (s, 1H), 6.36 (s, 1H), 2.09 (s, 6H).

### Preparation 14: 4-bromo-5-(2,6-dimethylphenoxy)-1-methylpyridin-2(1H)-one

4-bromo-5-(2,6-dimethylphenoxy) pyridin-2-ol (22.78 g, 77.7 mmol) was dissolved in DMF (227.8 mL, 10v) under nitrogen. Cesium carbonate (76.01 g, 233.2 mmol) was added to the reaction mixture & cooled at 0° C. Mel (176.5 g, 1244 mmol) was drop wise added to the reaction mixture & allowed to stir at room temperature for 2 h. TLC (9.0:1.0 DCM:methanol) showed no SM remaining. The reaction mixture was quenched with water (500 mL) and extracted by DCM (3×500 mL). The combined organics were washed (Brine solution), dried (Na₂SO₄), filtered and evaporated. The residue was purified on silica, eluting with 39.0:61.0 acetonitrile: H2O. Solvent reduction to give off white solid (10 g, 41.90%).

LCMS: (Waters Acquity UPLC with QDA mass Detector, Acidic, 4.0 min): 1.81 min, m/z=310.0 [M+H]+.

¹H NMR (400 MHz, DMSO d₆) δ 7.17-7.08 (m, 3H), 6.92 (s, 1H), 6.73 (s, 1H), 3.26 (s, 3H), 2.12 (s, 6H).

### Preparation 15: ethyl 4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c]pyridine-2-carboxylate

4-bromo-5-(2,6-dimethylphenoxy) pyridin-2-ol (3.0 g, 9.7 mmoL) was dissolved in 1,4-Dioxane (80 mL) and water (13.3 mL). ethyl 6-methyl-7-oxo-4-(4,4,5,5-tetramethyl-1,3,2-dioxaborolan-2-yl)-1-tosyl-6,7-dihydro-1H-pyrrolo[2,3-c] pyridine-2-carboxylate (8.30 g, 16.6 mmoL) was added to the reaction mixture followed by K₃PO₄ (5.17 g, 24.4 mmoL). The suspension was degassed by using argon for 30 min. Pd(dba)₃ (0.44 g, 0.48 mmol) and X-Phos (0.22 g, 0.48 mmol) was added to the reaction mixture and resulting dark solution was heated at 100° C. for 2 h. TLC (9:1 Ethyl acetate: MeOH) showed no SM remaining. The mixture was cooled to room temperature and quenched by addition of water (50 mL). The mixture was stirred 5 min before separating the phases. The aqueous phase was extracted with ethyl acetate (3×50 mL). The combined organic and dried (Na2SO4), filtered and evaporated. The residue was purified on silica, eluting with 06:94 Methanol: EtOAc: Solvent reduction to give off white solid (3.1 g, 52.99%).

LCMS: (Waters Acquity UPLC with QDA mass Detector, Acidic, 4.0 min): 2.12 min, m/z=602.15 [M+H]+.

¹H NMR (400 MHz, DMSO d₆) δ 8.34 (d, J=8.4 Hz, 2H), 7.87 (s, 1H), 7.50 (d, J=8.0 Hz, 2H), 7.12-7.02 (m, 4H), 6.70 (s, 1H), 6.50 (s, 1H), 4.38-4.32 (m, 2H), 3.54 (s, 3H), 3.31 (s, 3H), 2.42 (s, 3H), 2.06 (s, 6H), 1.31 (t, J=6.8 Hz, 3H)

### Preparation 16: 4-(5-(2,6-dimethylphenoxy)-1-methyl-2-oxo-1,2-dihydropyridin-4-yl)-2-(hydroxymethyl)-6-methyl-1-tosyl-1,6-dihydro-7H-pyrrolo[2,3-c]pyridin-7-one

Ethyl 4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c]pyridine-2-carboxylate (2.4 g, 3.99 mmol) was dissolved in DCM (240 mL) under argon and cool the reaction mixture to −20° C. DIBAL-H (11.92 g, 83.8 mmol) was added to the reaction mixture at −20° C. dropwise over 6 h. TLC (9:1 DCM: MeOH) showed no SM remaining. The mixture was diluted with DCM (240 ml) and quenched by addition of NaOH solution (300 mL). The resultant mixture was passed through celite filter. The mixture was separated and aqueous layer re-extracted with DCM (240 ml). The combined organics and dried (Na2SO4), filtered and evaporated. The residue was purified on silica, eluting with 3:97 methanol: DCM Solvent reduction to give off light yellow solid (0.8 g, 34%).

LCMS: (Waters Acquity UPLC with QDA mass Detector, Acidic, 4.0 min): 1.768 min, m/z=560.11 [M+H]⁺

¹H NMR: (400 MHz, DMSO) δ 8.16 (d, J=8.4 Hz, 2H), 7.70 (s, 1H), 7.40 (d, J=8.0 Hz, 2H), 7.11-7.02 (m, 3H), 6.66 (d, J=9.2 Hz, 2H), 6.45 (s, 1H), 5.61 (t, J=5.6 Hz, 1H), 4.92 (d, J=5.6 Hz, 2H), 3.45 (s, 3H), 3.31 (s, 3H), 2.37 (s, 3H), 2.06 (s, 6H).

### Preparation 17: 4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c]pyridine-2-carbaldehyde

4-(5-(2,6-dimethylphenoxy)-1-methyl-2-oxo-1,2-dihydropyridin-4-yl)-2-(hydroxymethyl)-6-methyl-1-tosyl-1,6-dihydro-7H-pyrrolo[2,3-c] pyridin-7-one (0.9 g, 1.60 mmol) was dissolved in DCM (9 mL). Manganese oxide (0.84 g, 9.64 mmol) added and reaction mixture heated to 50° C. for 4 hr. TLC (9:1 DCM: MeOH) showed no SM remaining. The mixture was filtered through celite and concentrated. Solvent reduction to give a light yellow solid (0.80 g, 89%).

LCMS: (Waters Acquity UPLC with QDA mass Detector, Acidic, 4.0 min): 2.022 min, m/z=558.16 [M+H]⁺

¹H NMR: (400 MHz, DMSO) δ 10.30 (s, 1H), 8.26 (d, J=8.4 Hz, 2H), 7.84 (s, 1H), 7.47 (d, J=8.4 Hz, 2H), 7.28 (s, 1H), 7.10-7.03 (m, 3H), 6.70 (s, 1H), 6.50 (s, 1H), 3.52 (s, 3H), 3.33 (s, 3H), 2.42 (s, 3H), 2.05 (s, 6H).

### Preparation 18: 4-(5-(2,6-dimethylphenoxy)-1-methyl-2-oxo-1,2-dihydropyridin-4-yl)-6-methyl-2-(5-methyl-1H-imidazol-2-yl)-1,6-dihydro-7H-pyrrolo [2,3-c] pyridin-7-one

4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c] pyridine-2-carbaldehyde (0.12 g, 0.21 mmol) was dissolved in Methanol (2.4 mL) under nitrogen. Ammonium carbonate (0.06 g, 0.63 mmol) was added to the reaction mixture and allowed to stir at room temperature for 30 min. 2-oxopropanal (0.077 g, 1.07 mmol) was added to reaction mixture. The reaction mixture was heated at 50° C. for 5 h. TLC (9.5:0.5 DCM:methanol) showed no SM remaining. The reaction mixture was cooled at 0° C. Sodium hydroxide (0.042 g, 1.05 mmol) was added to the reaction mixture. The dark solution was allowed to stir at 100° C. for 2 h. TLC (9:1 DCM:methanol) showed no SM remaining. The resulting solution was directly concentrated under reduced pressure. The resulting residue was purified by Prep HPLC purification (Instrument A; Column A; eluted with a gradient of 0.05% ammonium hydroxide solution in water and acetonitrile. Lyophilisation to give white solid (0.03 g, 40.12%)

LCMS: (Waters Acquity UPLC with QDA mass Detector, Acidic, 4.0 min): 1.294 min, m/z=456.1 [M+H]+

¹H NMR (400 MHz, DMSO) δ 12.829 (s, 1H), 7.663 (s, 1H), 7.523 (s, 1H), 7.303 (s, 1H), 7.118-7.056 (m, 3H), 6.752 (s, 1H), 6.603 (s, 1H), 3.622 (s, 3H), 3.348 (s, 3H), 2.348 (s, 3H), 2.081 (s, 6H). Note: One exchangeable—NH not observed.

### Example 2: 4-(5-(2,6-dimethylphenoxy)-1-methyl-2-oxo-1,2-dihydropyridin-4-yl)-2-(1H-imidazol-2-yl)-6-methyl-1,6-dihydro-7H-pyrrolo [2,3-c] pyridin-7-one

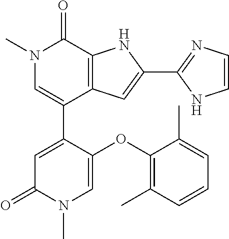 [SMILES: Cc1cccc(C)c1Oc1cn(C)c(=O)cc1-c1cn(C)c(=O)c2[nH]c(-c3ncc[nH]3)cc12]

### Preparation 19: 4-(5-(2,6-dimethylphenoxy)-1-methyl-2-oxo-1,2-dihydropyridin-4-yl)-2-(1H-imidazol-2-yl)-6-methyl-1,6-dihydro-7H-pyrrolo [2,3-c] pyridin-7-one

4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c] pyridine-2-carbaldehyde (0.2 g, 0.35 mmol) was dissolved in Methanol (4 mL) under nitrogen. Ammonium hydroxide (2 ml, 10V) was added to the reaction and the reaction mixture was allowed to stir at room temperature for 30 min. Oxalaldehyde (0.208 g, 3.5 mmol) was added to the reaction mixture at room temperature. The reaction mixture was heated at 50° C. TLC (9.5:0.5 DCM:methanol) after 16 h showed no SM remaining. The reaction mixture was cooled at 0° C. Sodium hydroxide (0.071 g, 1.79 mmol) was added to the reaction mixture. The reaction dark solution was allowed at stir at 100° C. for 3 h. TLC (9:1 DCM:methanol) showed no SM remaining. The resulting solution was directly concentrated to vacuum reduced pressure. The resulting residue was purified by Prep HPLC purification (Instrument B; Column B; eluted with a gradient of 0.1% methanolic ammonia in heptane and acetonitrile. Lyophilisation to give a white solid (0.01 g, 6.2%)

LCMS: (Waters Acquity UPLC with QDA mass Detector, Acidic, 4.0 min): 1.267 min, m/z=442.1 [M+H]+

1H NMR (400 MHz, DMSO) δ 12.364 (br s, 1H), 7.533 (s, 1H), 7.116-7.098 (m, 3H), 7.062-7.046 (m, 2H), 6.833 (s, 1H), 6.676 (s, 1H), 6.573 (s, 1H), 3.592 (s, 3H), 2.095 (s, 6H), 1.293-1.228 (m, 3H). Note: One exchangeable—NH not observed

### Example 3: 4-(5-(2,6-dimethylphenoxy)-1-methyl-2-oxo-1,2-dihydropyridin-4-yl)-6-methyl-2-(5-(trifluoromethyl)-1H-imidazol-2-yl)-1,6-dihydro-7H-pyrrolo[2,3-c] pyridin-7-one

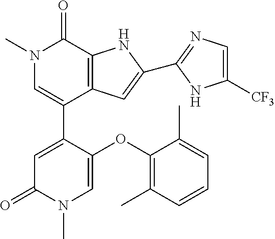 [SMILES: Cc1cccc(C)c1Oc1cn(C)c(=O)cc1-c1cn(C)c(=O)c2[nH]c(-c3ncc(C(F)(F)F)[nH]3)cc12]

### Preparation 20: 3,3,3-trifluoro-2-oxopropanal

3,3-Dibromo-1,1,1-trifluoroacetone (1.0 g, 3.70 mmol) was dissolved in water (5 mL). The resulting solution was allowed to stir for 5 min at room temperature. Sodium acetate (1.2 g, 14.8 mmol) was added to the reaction mixture at room temperature. The resulting solution was allowed to stir at 100° C. for 16 h. TLC (9.5:0.5 DCM/Methanol) showed no SM remaining. The reaction mixture was diluted by water (50 mL). The mixture was stirred 5 min before separating the phases. The aqueous phase was extracted with Ethyl acetate (6×50 mL). The combined organics were washed (brine), dried (Na2SO4), filtered and evaporated to give yellow liquid (0.5 g). The material was used for the next step without any purification.

### Preparation 21: 4-(5-(2,6-dimethylphenoxy)-1-methyl-2-oxo-1,2-dihydropyridin-4-yl)-6-methyl-1-tosyl-2-(5-(trifluoromethyl)-1H-imidazol-2-yl)-1,6-dihydro-7H-pyrrolo[2,3-c] pyridin-7-one

4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c] pyridine-2-carbaldehyde (0.2 g, 0.359 mmol) and 3,3,3-trifluoro-2-oxopropanal (0.362 g, 2.870 mmol) was dissolved in Methanol (4 mL). The resulting solution was allowed to stir for 5 min at 0° C. 25% NH₄OH solution (1 mL) was added to the reaction mixture at 0° C. TLC (9.5:0.5 DCM/Methanol) showed no SM remaining. The reaction mixture was concentrated to under reduced pressure to afford crude material. The residue was purified on silica, eluting with 2.5% Methanol in DCM. Solvent reduction to give Brown solid (0.160 g, 67.22%).

LCMS: (Waters Acquity UPLC with QDA mass Detector, Acidic, 4.0 min): 1.104 min, m/z=664.19 [M+H]+

### Preparation 22: 4-(5-(2,6-dimethylphenoxy)-1-methyl-2-oxo-1,2-dihydropyridin-4-yl)-6-methyl-2-(5-(trifluoromethyl)-1H-imidazol-2-yl)-1,6-dihydro-7H-pyrrolo[2,3-c] pyridin-7-one

4-(5-(2,6-dimethylphenoxy)-1-methyl-2-oxo-1,2-dihydropyridin-4-yl)-6-methyl-1-tosyl-2-(5-(trifluoromethyl)-1H-imidazol-2-yl)-1,6-dihydro-7H-pyrrolo[2,3-c] pyridin-7-one (0.100 g, 0.15 mmol) was dissolved in 1M TBAF solution (5 mL) at room temperature. The resulting solution was allowed to stir at room temperature for 4 h. TLC (9.5:0.5 DCM/Methanol) showed no SM remaining. The reaction mixture was concentrated under reduced pressure to afford crude material. The residue was purified by Prep HPLC purification (Instrument A; Column C; eluted with a gradient of 0.05% ammonium hydroxide solution in water and acetonitrile. Lyophilisation to give white solid (0.006 g, 7.82%).

LCMS: (Waters Acquity UPLC with QDA mass Detector, Acidic, 4.0 min): 1.739 min, m/z=510.12 [M+H]+

1H NMR: (400 MHz, DMSO) δ 12.89 (s, 1H), 12.48 (s, 1H), 7.97 (s, 1H), 7.57 (s, 1H), 7.12-7.03 (m, 3H), 6.96 (s, 1H), 6.69 (s, 1H), 6.58 (s, 1H), 3.60 (s, 3H), 3.33 (s, 3H), 2.09 (s, 6H).

### Example 4: 2-(4,5-dihydro-1H-imidazol-2-yl)-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one

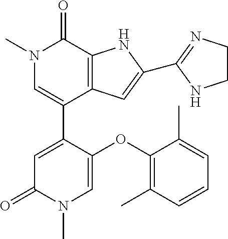 [SMILES: Cc1cccc(C)c1Oc1cn(C)c(=O)cc1-c1cn(C)c(=O)c2[nH]c(C3=NCCN3)cc12]

### Preparation 23: 2-(4,5-dihydro-1H-imidazol-2-yl)-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one

4-(5-(2,6-dimethylphenoxy)-1-methyl-2-oxo-1,2-dihydropyridin-4-yl)-6-methyl-7-oxo-1-tosyl-6,7-dihydro-1H-pyrrolo[2,3-c] pyridine-2-carbaldehyde (0.3 g, 0.538 mmol) and ethane-1,2-diamine (0.033 g, 0.565 mmol) was dissolved in DCM (7.5 mL, 25Vol). The resulting solution was allowed to stir for 15 min at 0° C. NBS (0.1 g, 0.565 mmol) was added to the reaction mixture at 0° C. TLC (9:1 DCM/Methanol) showed no SM remaining. The reaction mixture was quenched by saturated NaHCO₃ (50 mL). The mixture was stirred 5 min before separating the phases. The aqueous phase was extracted with ethyl acetate (2×20 mL). The combined organics were washed (brine), dried (Na₂SO₄), filtered and evaporated to under reduced pressure to give light green solid (0.220 g, 68.42%).

LCMS: (Waters Acquity UPLC with QDA mass Detector, Acidic, 4.0 min): 1.44 min, m/z=598.1 [M+H]+

### Preparation 24: 2-(4,5-dihydro-1H-imidazol-2-yl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

2-(4,5-dihydro-1H-imidazol-2-yl)-4-(5-(2,6-dimethylphenoxy)-1-methyl-2-oxo-1,2-dihydropyridin-4-yl)-6-methyl-1-tosyl-1,6-dihydro-7H-pyrrolo [2,3-c] pyridin-7-one (0.175 g, 0.29 mmol) was dissolved in 1,4 dioxane (2 mL, 22Vol). Sodium hydroxide (0.058 g, 1.46 mmol) was added to the reaction mixture followed by water (0.1 mL) at room temperature. The resulting solution was heated at 100° C. for 2 h. TLC (9.5:0.5 DCM/Methanol) showed no SM remaining. The reaction mixture was concentrated under reduced pressure to give yellow solid crude which was purified by prep-HPLC purification (Instrument A; Column A; eluted with a gradient of 0.05% ammonium hydroxide solution in water and acetonitrile. Lyophilisation to give white solid (0.010 g, 8.47%).

LCMS: (Waters Acquity UPLC with QDA mass Detector, Acidic, 4.0 min): 1.213 min, m/z=444.07 [M+H]⁺

1H NMR: (400 MHz, DMSO) δ 7.45 (s, 1H), 7.11 (d, J=7.2 Hz, 2H), 7.06 (t, J=6.4 Hz, 1H), 6.88 (s, 1H), 6.64 (s, 1H), 6.56 (s, 1H), 3.64 (s, 4H), 3.55 (s, 3H), 3.31 (s, 3H), 2.08 (s, 6H).

### Example 5: 6-methyl-2-(5-methyl-1H-imidazol-2-yl)-4-(1-methyl-2-oxo-5-phenyl-1,2-dihydropyridin-4-yl)-1,6-dihydro-7H-pyrrolo [2,3-c] pyridin-7-one

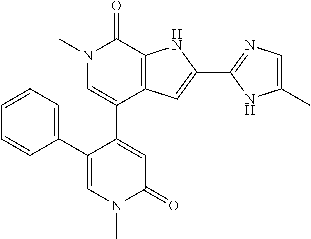 [SMILES: Cc1cnc(-c2cc3c(-c4cc(=O)n(C)cc4-c4ccccc4)cn(C)c(=O)c3[nH]2)[nH]1]

### Preparation 25: 5-bromo-4-iodopyridin-2-amine

4-iodopyridin-2-amine (20.0 g, 90.90 mmol) was dissolved in ACN (800 mL) under nitrogen. N-Bromosuccinamide (16.3 g, 91.8 mmol) was added to the reaction mixture at room temperature. The resulting suspension was allowed to stir at room temperature for 16 h. TLC (3:7 hexane/EA) showed no SM remaining. The resulting solution was directly concentrated under vacuum reduced pressure and added DCM (200 mL). The combined organics washed (brine), dried (Na2SO4), filtered and evaporated to an oil. The Product was purified by flash column chromatography on silica, eluting with 90:10 hexane/ethyl acetate. Fractions corresponding to product were combined and concentrated to give an off white solid (25 g, 92.01%).

LCMS: (Waters Acquity UPLC with QDA mass Detector, Acidic, 4.0 min): 1.22 min, m/z=298.8 [M+H]+

1H NMR: (400 MHz, DMSO) δ 8.01 (s, 1H), 7.05 (s, 1H), 6.27 (s, 2H).

### Preparation 26: 5-bromo-4-iodopyridin-2(1H)-one

5-bromo-4-iodopyridin-2-amine (27.0 g, 90.33 mmol) was dissolved in Sulphuric acid (500 mL). Sodium nitrite (12.50 g, 181.1 mmol) in water (54 mL) was dropwise added to the reaction mixture at 0° C. The reaction mixture was allowed to stir at room temperature for 2 h. TLC (5:5 hexane/ethyl acetate) showed no SM remaining. The resulting mixture was cooled at 0° C. and quenched by dropwise addition of ammonia solution to afford pale yellow precipitate which was filtered. The resulting solid was dried overnight under vacuum at 50° C. (21 g, 77.52%).

LCMS: (Waters Acquity UPLC with QDA mass Detector, Acidic, 4.0 min): 1.19 min, m/z=299.8 [M+H]+

HPLC: (Waters Alliance e2695 with 2998 detector, Basic, 17.0 min): 3.50 min

1H NMR: (400 MHz, DMSO) δ 11.99 (br s, 1H), 7.80 (s, 1H), 7.13 (s, 1H).

### Preparation 27: 5-bromo-4-iodo-1-methylpyridin-2(1H)-one

5-bromo-4-iodopyridin-2(1H)-one (22.0 g, 90.33 mmol) was dissolved in DMF (220 mL) under nitrogen. Cesium carbonate (28.77 g, 181.1 mmol) was added to the reaction mixture followed by dropwise addition of methyl iodide (13.74 mL) at 0° C. The reaction mixture was allowed to stir at room temperature for 1 h. TLC (5:5 hexane/ethyl acetate) showed no SM remaining. The resulting mixture was quenched by addition of water (100 mL). The mixture was stirred 5 min before separating the phases. The aqueous phase extracted with ethyl acetate (3×100 mL). The combined organics were washed with brine (2×50 mL) and dried over Na₂SO₄. Fraction was concentrated to give sticky oil which was triturated by mixture of n-pentane/diethyl ether to give a pale yellow solid (13 g, 56.62%).

LCMS: (Waters Acquity UPLC with QDA mass Detector, Acidic, 4.0 min): 1.31 min, m/z=313.8 [M+H]+

HPLC: (Waters Alliance e2695 with 2998 detector, Basic, 17.0 min): 6.10 min

1H NMR: (400 MHz, DMSO) δ 8.13 (s, 1H), 7.13 (s, 1H), 3.33 (s, 3H).

### Preparation 28: ethyl 4-(5-bromo-1-methyl-2-oxo-1,2-dihydropyridin-4-yl)-6-methyl-7-oxo-1-tosyl-6,7-dihydro-1H-pyrrolo [2,3-c] pyridine-2-carboxylate

5-bromo-4-iodo-1-methylpyridin-2(1H)-one (3.0 g, 9.615 mmol) was dissolved in Dioxane (48 mL) and water (12 mL) at room temperature. ethyl 6-methyl-7-oxo-4-(4,4,5,5-tetramethyl-1,3,2-dioxaborolan-2-yl)-1-tosyl-6,7-dihydro-1H-pyrrolo[2,3-c] pyridine-2-carboxylate (5.76 g, 11.53 mmol) was added to the reaction followed by Sodium carbonate (2.03 g, 19.23 mmol) at room temperature. The reaction mixture was purged by argon for 30 min.

Tetrakis (0.556 g, 0.480 mmol) was added to the reaction mixture and allowed to stir at 95° C. for 16 h. TLC (9.5:0.5 DCM\MeOH) showed no SM remaining. The resulting mixture were slowly poured into cold water to afford white precipitate which was filtered. The solid was dried under vacuum to afford pure product (3.1 g, 92.26%).

LCMS: (Waters Acquity UPLC with QDA mass Detector, Acidic, 4.0 min): 1.879 min, m/z=560.1 [M+H]⁺.

1H NMR: (400 MHz, DMSO) δ 8.33 (d, J=8.4 Hz, 2H), 8.22 (s, 1H), 7.70 (s, 1H), 7.52 (d, J=8.2 Hz, 2H), 6.89 (s, 1H), 6.48 (s, 1H), 4.38-4.32 (m, 2H), 3.50 (s, 3H), 3.47 (s, 3H), 2.44 (s, 3H), 1.30 (t, J=7.2 Hz, 3H).

### Preparation 29: ethyl 6-methyl-4-(1-methyl-2-oxo-5-phenyl-1,2-dihydropyridin-4-yl)-7-oxo-1-tosyl-6,7-dihydro-1H-pyrrolo [2,3-c] pyridine-2-carboxylate

Ethyl 4-(5-bromo-1-methyl-2-oxo-1,2-dihydropyridin-4-yl)-6-methyl-7-oxo-1-tosyl-6,7-dihydro-1H-pyrrolo [2,3-c] pyridine-2-carboxylate (0.5 g, 0.894 mmol) was dissolved in Toluene(8.0 mL) and water (2.0 mL) at room temperature. 4,4,5,5-tetramethyl-2-phenyl-1,3,2-dioxaborolane (0.218 g, 1.788 mmol) was added to the reaction mixture followed by potassium phosphate (0.569 g, 2.683 mmol) at room temperature. The reaction mixture was purged by argon for 30 min. S-Phos (0.036 g, 0.089 mmol) and Pd₂(dba)₃(0.04 g, 0.0447 mmol) were added to the reaction mixture. The resulting mixture was heated at 95° C. for 1 h. TLC (9:1 DCM\Methanol) showed no SM remaining. The reaction mixture was quenched by water extracted by EtOAc (3×50 mL). The combined organic layer was dried (Na₂SO₄), filtered and evaporated. The resulting residue was purified by flash column chromatography using EtOAc\Methanol eluting with zero gradient neat ethyl acetate. Solvent reduction to give a cream solid (0.3 g, 60.30%).

LCMS: (Waters Acquity UPLC with QDA mass Detector, Acidic, 4.0 min):1.937 min, m/z=557.16[M+H]+.

1H NMR: (400 MHz, DMSO) δ 8.22 (d, J=8.4 Hz, 2H), 7.88 (s, 1H), 7.59 (s, 1H), 7.49 (d, J=8.4 Hz, 2H), 7.20-7.12 (m, 5H), 6.46 (d, J=8.7 Hz, 2H), 4.30-4.24 (m, 2H), 3.53 (s, 3H), 3.42 (s, 3H), 2.42 (s, 3H), 1.27 (t, J=14.4 Hz, 3H).

### Preparation 30: 2-(hydroxymethyl)-6-methyl-4-(1-methyl-2-oxo-5-phenyl-1,2-dihydropyridin-4-yl)-1-tosyl-1,6-dihydro-7H-pyrrolo[2,3-c]pyridin-7-one

Ethyl 6-methyl-4-(1-methyl-2-oxo-5-phenyl-1,2-dihydropyridin-4-yl)-7-oxo-1-tosyl-6,7-dihydro-1H-pyrrolo [2,3-c] pyridine-2-carboxylate (0.75 g, 1.34 mmol) was dissolved in DCM (180 mL) under argon and cool the reaction mixture to −20° C. DIBAL-H (28.2 mL, 28.2 mmol) was dropwise added to the reaction mixture at −20° C. dropwise over 6 h. TLC (9:1 DCM: Methanol) showed no SM remaining. The mixture was diluted with DCM (240 ml) and quenched by addition of sodium hydroxide solution (300 mL). The resultant mixture was filtered through celite pad. The mixture was separated and aqueous layer re-extracted with DCM (240 ml). The combined organics and dried (Na2SO4), filtered and evaporated. The residue was purified on silica, eluting with 3:97 methanol: DCM. Solvent reduction to give off light yellow solid (0.2 g, 28%).

LCMS: (Waters Acquity UPLC with QDA mass Detector, Acidic, 4.0 min): 1.537 min, m/z=516.17 [M+H]⁺

1H NMR: (400 MHz, DMSO) δ 7.93 (d, J=8.4 Hz, 2H), 7.86 (s, 1H), 7.49 (d, J=8.4 Hz, 2H), 7.31 (s, 1H), 7.20-7.17 (m, 3H) 7.12-7.10 (m, 2H), 6.40 (s, 1H), 6.18 (s, 1H), 5.45 (t, 1H), 4.75 (d, J=5.2 Hz, 2H), 3.53 (s, 3H), 3.28 (s, 3H), 2.37 (s, 3H).

### Preparation 31: 6-methyl-4-(1-methyl-2-oxo-5-phenyl-1,2-dihydropyridin-4-yl)-7-oxo-1-tosyl-6,7-dihydro-1H-pyrrolo [2,3-c] pyridine-2-carbaldehyde

2-(hydroxymethyl)-6-methyl-4-(1-methyl-2-oxo-5-phenyl-1,2-dihydropyridin-4-yl)-1-tosyl-1,6-dihydro-7H-pyrrolo[2,3-c]pyridin-7-one (0.47 g, 0.91 mmol) was dissolved in DCM (47 mL). Manganese oxide (0.47 g, 5.47 mmol) was added to the reaction mixture and heated to 50° C. for 4 h. TLC (9:1 DCM: MeOH) showed no SM remaining. The mixture was filtered through celite pad and concentrated. Solvent reduction to give a light yellow solid (0.28 g, 59%).

LCMS: (Waters Acquity UPLC with QDA mass Detector, Acidic, 4.0 min): 1.823 min, m/z=514.07 [M+H]⁺

1H NMR: (400 MHz, DMSO) δ 10.08 (s, 1H), 8.12 (d, J=8.4 Hz, 2H), 7.89 (s, 1H), 7.62 (s, 1H), 7.47 (d, J=8.0 Hz, 2H) 7.17-7.13 (m, 5H), 6.63 (s, 1H), 6.48 (s, 1H), 3.53 (s, 3H), 3.42 (s, 3H), 2.42 (s, 3H)

### Preparation 32: 6-methyl-2-(5-methyl-1H-imidazol-2-yl)-4-(1-methyl-2-oxo-5-phenyl-1,2-dihydropyridin-4-yl)-1-tosyl-1,6-dihydro-7H-pyrrolo [2,3-c] pyridin-7-one

6-methyl-4-(1-methyl-2-oxo-5-phenyl-1,2-dihydropyridin-4-yl)-7-oxo-1-tosyl-6,7-dihydro-1H-pyrrolo [2,3-c] pyridine-2-carbaldehyde (0.22 g, 0.428 mmol) was dissolved in Methanol (4.5 mL) under nitrogen. Ammonium carbonate (0.411 g, 4.288 mmol) was added to the reaction mixture and allowed to stir at room temperature for 30 min. 2-oxopropanal (0.15 g, 0.428 mmol) was added to reaction mixture. The reaction mixture was heated at 50° C. for 5 h. TLC (9.5:0.5 DCM:methanol) showed no SM remaining. The resulting solution was directly concentrated to vacuum reduced pressure. The residue was purified by Flash column chromatography and product was eluted at 2% Methanol in DCM. Solvent reduction gave a pure product as white solid (0.05 g, 20%)

LCMS: (Waters Acquity UPLC with QDA mass Detector, Acidic, 4.0 min): 1.256 min, m/z=566.11 [M+H]+

### Preparation 33: 6-methyl-2-(5-methyl-1H-imidazol-2-yl)-4-(1-methyl-2-oxo-5-phenyl-1,2-dihydropyridin-4-yl)-1,6-dihydro-7H-pyrrolo [2,3-c] pyridin-7-one

6-methyl-2-(5-methyl-1H-imidazol-2-yl)-4-(1-methyl-2-oxo-5-phenyl-1,2-dihydropyridin-4-yl)-1-tosyl-1,6-dihydro-7H-pyrrolo[2,3-c] pyridin-7-one (0.200 g, 0.35 mmol) was dissolved in 1M TBAF (5 mL, 7.07Vol) at room temperature. The resulting solution was allowed to stir at room temperature for 16 h. TLC (9.5:0.5 DCM/Methanol) showed no SM remaining. The reaction mixture was concentrated under reduced pressure to give crude material. The residue was purified by prep-HPLC purification (Instrument A; Column A; eluted with a gradient of 0.05% ammonium hydroxide solution in water and acetonitrile. Lyophilisation to give off-white solid (0.0028 g, 1.79%).

LCMS: (Waters Acquity UPLC with QDA mass Detector, Acidic, 4.0 min): 5.772 min, m/z=412.41 [M+H]+

1H NMR: (400 MHz, DMSO) δ 11.98 (s, 1H), 11.84 (s, 1H) 7.87 (s, 1H), 7.39 (t, J=14 Hz, 1H), 7.17-7.12 (m, 3H), 7.06-6.99 (m, 2H), 6.90 (br s, 1H), 6.65 (br s, 1H), 6.48 (br s, 1H), 3.55 (s, 3H), 3.40 (s, 3H), 2.19-2.11 (m, 3H)

### Example 6: 4-(5-(2,6-dimethylphenoxy)-1-methyl-2-oxo-1,2-dihydropyridin-4-yl)-2-(1-isopropyl-3-methyl-1H-pyrazol-4-yl)-6-methyl-1,6-dihydro-7H-pyrrolo[2,3-c]pyridin-7-one

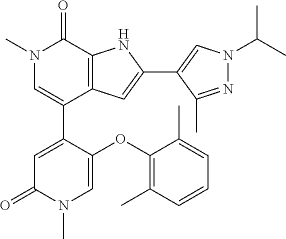 [SMILES: Cc1cccc(C)c1Oc1cn(C)c(=O)cc1-c1cn(C)c(=O)c2[nH]c(-c3cn(C(C)C)nc3C)cc12]

### Preparation 34: 4-bromo-2-chloro-7-methoxy-1-tosyl-1H-pyrrolo [2, 3-c] pyridine

4-bromo-7-methoxy-1-tosyl-1H-pyrrolo[2,3-c] pyridine (80 g, 210.5 mmol) was dissolved in dry THF (1200 mL) under argon. LDA (1M in THF/Heptane) (273.6 mL, 273.6 mmol) was dropwise added to the reaction mixture at −78° C. over a period of 30 min. The orange solution was allowed to stir at ambient temperature for 2 h. Hexachloroethane (82.33 g, 347.33 mmol) in dry Tetrahydrofuran (400 mL) was dropwise added to the reaction mixture at −78° C. TLC (9.5:0.5 Hexane/EtOAc) after 2 h showed no SM remaining. The reaction mixture was quenched by dropwise addition of saturated NH₄Cl (2400 mL). The mixture was stirred for 5 min before separating the phases. The aqueous phase was extracted with ethyl acetate (2×800 mL). The combined organics were washed (brine), dried (Na₂SO₄), filtered and evaporated to an oil that was triturated by EtOAc to give a light yellow solid (76 g, 87%).

LCMS: (Waters, Acidic, 4.0 min): 2.753 min, m/z=414.7 [M+H]+

¹H NMR: (400 MHz, DMSO) δ 8.08 (s, 1H), 7.95 (d, J=8.4, 2H), 7.53 (d, J=8, 2H), 7.05 (s, 1H), 3.87 (s, 3H), 2.42 (s, 3H).

### Preparation 35: 2-chloro-6-methyl-1-tosyl-4-(tributylstannyl)-1,6-dihydro-7H-pyrrolo[2,3-c]pyridin-7-one

1,4 dioxane (50 mL) was degassed with argon for 30 min. 4-bromo-2-chloro-6-methyl-1-tosyl-1,6-dihydro-7H-pyrrolo[2,3-c] pyridin-7-one (5.0 g, 1.20 mmol) and hexabutyllditin (11.6 mL, 22.9 mmol) was added to the reaction mixture followed by tetrakis (1.4 g, 1.20 mmol) at room temperature. The resulting mixture was allowed to stir at 130° C. for 3 h whereupon TLC (3:7 Ethyl acetate/Hexane) showed no SM remaining. The resulting solution was filtered through a celite-pad and the filtrate was diluted with water (50 mL) and ethyl acetate (50 mL). The combined organics were dried over Na₂SO₄, filtered and concentrated. The resulting residue was purified by normal phase chromatography, eluting with (30:70) ethyl acetate/hexane to yield 2-chloro-6-methyl-1-tosyl-4-(tributylstannyl)-1,6-dihydro-7H-pyrrolo[2,3-c] pyridin-7-one (2.5 g, 28%) as an off-white solid.

¹H NMR: (400 MHz, DMSO) δ 8.14 (d, J=8 Hz, 2H), 7.48 (d, J=8 Hz, 2H), 7.22 (s, 1H), 6.58 (s, 1H), 3.45 (s, 3H), 2.40 (s, 3H), 1.50-1.43 (m, 6H), 1.32-1.22 (m, 6H), 1.11-1.06 (m, 6H), 0.87 (t, J=8 Hz, 9H).

### Preparation 36: 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one

2-Chloro-6-methyl-1-tosyl-4-(tributylstannyl)-1,6-dihydro-7H-pyrrolo[2,3-c] pyridin-7-one (2.6 g, 4.15 mmol) and 4-bromo-5-(2,6-dimethylphenoxy)-1-methylpyridin-2(1H)-one (1.27 g, 4.15 mmol) were dissolved in toluene (24 mL). Reaction mixture was purged with argon for 30 min. Tetrakis (0.47 g, 0.41 mmol) was added to the reaction mixture and resulting mixture allowed to stir at 120° C. for 3 h. TLC (0.5:9.5 MeOH/DCM) after 3 h showed SM was consumed. The reaction mixture was diluted with water (50 mL) and extracted by ethyl acetate (50 mL). The combined organics were dried over Na₂SO₄, filtered and evaporated. The residue was purified by reverse phase chromatography, eluting with (60:40) acetonitrile/water to yield 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.95 g, 46%) as an off-white solid.

LCMS t_{R}: (Water, Acidic, 4.0 min): 2.171 min, m/z=563.9 [M+H]⁺.

¹H NMR: (400 MHz, DMSO) δ 8.14 (d, J=8.0 Hz, 2H), 7.80 (s, 1H), 7.47 (d, J=8.0 Hz, 2H), 7.11-7.02 (m, 3H), 6.78 (s, 1H), 6.68 (s, 1H), 6.47 (s, 1H), 3.52 (s, 3H), 3.33 (s, 3H), 2.39 (s, 3H), 2.05 (s, 6H).

### Preparation 37: 4-(5-(2,6-dimethylphenoxy)-1-methyl-2-oxo-1,2-dihydropyridin-4-yl)-2-(1-isopropyl-3-methyl-1H-pyrazol-4-yl)-6-methyl-1,6-dihydro-7H-pyrrolo[2,3-c] pyridin-7-one

2-Chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) was dissolved in Dioxane (1.5 mL) under argon. 1-1sopropyl-3-methyl-4-(4,4,5,5-tetramethyl-1,3,2-dioxaborolan-2-yl)-1H-pyrazole (0.13 g, 0.53 mmol) was added to the reaction mixture followed by sodium carbonate (0.084 g, 0.79 mmol) and water (0.3 mL) at room temperature. The suspension was degassed for 30 min. PdCl₂(dppf).DCM (0.021 g, 0.026 mmol) was added to the reaction mixture. The dark solution was heated at 140° C. for 1 h. TLC (9.5:0.5 DCM:methanol) showed no SM remaining. The reaction mixture was cooled to 0° C. Sodium hydroxide (0.053 g, 1.33 mmol) was added to the reaction mixture. The dark solution was allowed at stir at 140° C. for 40 min. TLC (9.5:0.5 DCM:methanol) showed complete deprotection. The resulting solution was directly concentrated underreduced pressure and ethyl acetate was added. The mixture was filtered and the filtrate was evaporated to an oil. The crude material was purified by prep HPLC purification using Instrument: A, Column: B; eluted with a gradient of 0.05% ammonium hydroxide solution in water and acetonitrile. Lyophilised fractions gave off-white solid (0.010 g, 8.0%)

LCMS t_{R} (Water, Acidic, 4.0 min): 1.630 min, m/z=498.1 [M+H]⁺

HPLC t_{R} (Waters Alliance e2695 with 2998 detector, Basic, 17.0 min): 6.763 min

¹H NMR: (400 MHz, DMSO) δ 12.01 (s, 1H), 8.36 (s, 1H), 7.46 (s, 1H), 7.11-7.04 (m, 2H), 6.66 (s, 1H), 6.53 (s, 1H), 6.34 (s, 1H), 5.76 (s, 1H), 4.43-4.37 (m, 1H), 3.59 (s, 3H), 3.32 (s, 3H), 2.32 (s, 3H), 2.10 (s, 6H), 1.43 (d, J=6.8 Hz, 6H).

### Example 7: 2-(1,3-dimethyl-1H-pyrazol-5-yl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

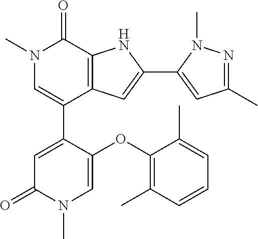

### Preparation 38: 2-(1,3-dimethyl-1H-pyrazol-5-yl)-4-(5-(2,6-dimethylphenoxy)-1-methyl-2-oxo-1,2-dihydropyridin-4-yl)-6-methyl-1,6-dihydro-7H-pyrrolo [2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 1,3-dimethyl-5-(4,4,5,5-tetramethyl-1,3,2-dioxaborolan-2-yl)-1H-pyrazole (0.088 g, 0.53 mmol) was reacted to give crude material. The resulting residue was purified by prep HPLC purification using Instrument: A, Column: C; eluted with a gradient of 0.05% ammonium hydroxide solution in water and acetonitrile. Lyophilised fractions gave white solid (0.027 g, 22%).

LCMS tR (Waters, Acidic, 4.0 min): 1.529 min, m/z=470.0 [M+H]⁺

HPLC tR (Waters Alliance e2695 with 2998 detector, Basic, 17.0 min): 6.376 min

1H NMR: (400 MHz, DMSO) δ 12.46 (s, 1H), 7.52 (s, 1H), 7.09-7.06 (m, 3H), 6.67 (s, 1H), 6.57 (s, 2H), 6.54 (s, 1H), 3.88 (s, 3H), 3.60 (s, 3H), 3.32 (s, 3H), 2.16 (s, 3H), 2.08 (s, 6H).

### Example 8: 4-(5-(2,6-dimethylphenoxy)-1-methyl-2-oxo-1,2-dihydropyridin-4-yl)-6-methyl-2-phenyl-1,6-dihydro-7H-pyrrolo[2,3-c] pyridin-7-one

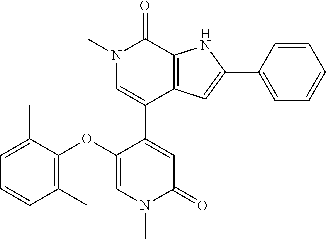 [SMILES: Cc1cccc(C)c1Oc1cn(C)c(=O)cc1-c1cn(C)c(=O)c2[nH]c(-c3ccccc3)cc12]

### Preparation 39: 4-(5-(2,6-dimethylphenoxy)-1-methyl-2-oxo-1,2-dihydropyridin-4-yl)-6-methyl-2-phenyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and phenylboronic acid (0.064 g, 0.53 mmoL) was reacted to give crude material. The resulting residue was purified by prep HPLC purification using Instrument: B, Column: A; eluted with a gradient of 0.05% ammonium hydroxide solution in water and acetonitrile. Lyophilised fractions gave off white solid (0.032 g, 27%)

LCMS t_{R} (Waters, Acidic, 4.0 min): 1.78 min, m/z=452.0 [M+H]⁺

HPLC t_{R} (Waters Alliance e2695 with 2998 detector, Basic, 17.0 min): 7.641 min

¹H NMR: (400 MHz, DMSO) δ 12.47 (s, 1H), 7.95 (d, J=7.6 Hz, 2H), 7.51 (s, 1H), 7.42 (apparent t, J=7.4 Hz, 2H), 7.32 (apparent t, J=7.2 Hz, 1H), 7.11-7.02 (m, 3H), 6.81 (s, 1H), 6.67 (s, 1H), 6.56 (s, 1H), 3.61 (s, 3H), 3.33 (s, 3H), 2.10 (s, 6H)

### Example 9: 4-(5-(2,6-dimethylphenoxy)-1-methyl-2-oxo-1,2-dihydropyridin-4-yl)-6-methyl-2-(pyridin-3-yi)-1,6-dihydro-7H-pyrrolo[2,3-c] pyridin-7-one.TFA

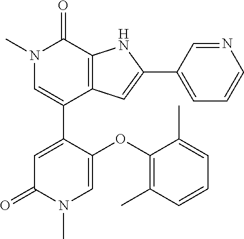 [SMILES: Cc1cccc(C)c1Oc1cn(C)c(=O)cc1-c1cn(C)c(=O)c2[nH]c(-c3cccnc3)cc12]

### Preparation 40: 4-(5-(2,6-dimethylphenoxy)-1-methyl-2-oxo-1,2-dihydropyridin-4-yl)-6-methyl-2-(pyridin-3-yl)-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and pyridin-3-ylboronic acid (0.065 g, 0.399 mmol) was reacted to give crude material. The resulting residue was purified by prep HPLC purification using Instrument: A, Column: C; eluted with a gradient of 0.05% Trifluoro acetic acid in water and acetonitrile. Lyophilised fractions gave brown sticky solid (0.010 g, 10%)

LCMS t_{R} (Water, Acidic, 4.0 min): 1.350 min, m/z=453.0 [M+H]⁺

HPLC t_{R} (Waters Alliance e2695 with 2998 detector, Basic, 17.0 min): 6.290 min

¹H NMR: (400 MHz, DMSO) δ 12.68 (s, 1H), 9.16 (s, 1H), 8.52 (d, J=5.6 Hz, 1H), 8.37 (d, J=8 Hz, 1H), 7.74-7.68 (m, 1H), 7.53 (s, 1H), 7.49-7.46 (m, 1H), 7.23 (s, 1H), 7.10 (d, J=4.4 Hz, 2H), 6.98 (s, 2H), 3.61 (s, 3H), 3.34 (s, 3H), 2.10 (s, 6H).

### Example 10: 4-(5-(2,6-dimethylphenoxy)-1-methyl-2-oxo-1,2-dihydropyridin-4-yl)-6-methyl-2-(pyridin-4-yl)-1,6-dihydro-7H-pyrrolo[2,3-c] pyridin-7-one

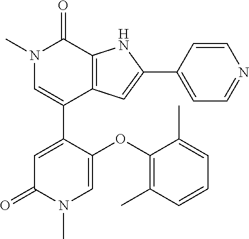 [SMILES: Cc1cccc(C)c1Oc1cn(C)c(=O)cc1-c1cn(C)c(=O)c2[nH]c(-c3ccncc3)cc12]

### Preparation 41: 4-(5-(2,6-dimethylphenoxy)-1-methyl-2-oxo-1,2-dihydropyridin-4-yl)-6-methyl-2-(pyridin-4-yl)-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and Pyridin-4-ylboronic acid (0.065 g, 0.53 mmol) was reacted to give crude material. The resulting residue was purified by prep HPLC purification using Instrument: A, Column: C; eluted with a gradient of 0.05% ammonium hydroxide solution in water and acetonitrile. Lyophilised fractions gave off white solid. (0.018 g, 13%)

LCMS t_{R} (Waters, Acidic, 4.0 min): 1.245 min, m/z=453.1 [M+H]⁺

HPLC t_{R} (Waters Alliance e2695 with 2998 detector, Basic, 17.0 min): 6.278 min

¹H NMR: (400 MHz, DMSO) δ 12.78 (s, 1H), 8.59 (d, J=5.6 Hz 2H), 7.96 (d, J=5.6 Hz 2H), 7.54 (s, 1H), 7.10-7.02 (m, 4H), 6.69 (s, 1H), 6.56 (s, 1H), 3.62 (s, 3H), 3.34 (s, 3H), 2.10 (s, 6H).

### Example 11: 4-(5-(2,6-dimethylphenoxy)-1-methyl-2-oxo-1,2-dihydropyridin-4-yl)-2-(1-isopropyl-1H-pyrazol-4-yl)-6-methyl-1,6-dihydro-7H-pyrrolo[2,3-c] pyridin-7-one

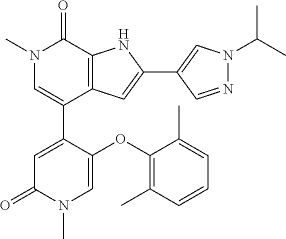 [SMILES: Cc1cccc(C)c1Oc1cn(C)c(=O)cc1-c1cn(C)c(=O)c2[nH]c(-c3cnn(C(C)C)c3)cc12]

### Preparation 42: 4-(5-(2,6-dimethylphenoxy)-1-methyl-2-oxo-1,2-dihydropyridin-4-yl)-2-(1-isopropyl-1H-pyrazol-4-yl)-6-methyl-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 1-isopropyl-4-(4,4,5,5-tetramethyl-1,3,2-dioxaborolan-2-yl)-1H-pyrazole (0.125 g, 0.53 mmol) was reacted to give crude material. The resulting residue was purified by prep HPLC purification using Instrument: A, Column: C; eluted with a gradient of 0.05% ammonium hydroxide solution in water and acetonitrile. Lyophilised fractions gave off white solid (0.026 g, 21%)

LCMS t_{R} (Waters, Acidic, 4.0 min): 1.622 min, m/z=484.1 [M+H]⁺

HPLC t_{R} (Waters Alliance e2695 with 2998 detector, Basic, 17.0 min): 6.702 min

¹H NMR: (400 MHz, DMSO) δ 12.22 (s, 1H), 8.37 (s, 1H), 8.00 (s, 1H), 7.48 (s, 1H), 7.12-7.03 (m, 3H), 6.67 (s, 1H), 6.54 (d, J=6.4 Hz, 2H), 4.48 (m, 1H), 3.58 (s, 3H), 2.50 (s, 3H), 2.08 (s, 6H), 1.44 (d, J=6.4 Hz, 6H).

### Example 12: 2-(1,3-dimethyl-1H-pyrazol-4-yl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

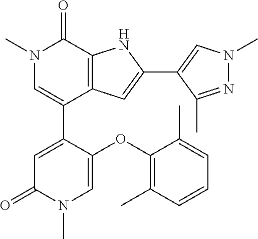

### Preparation 43: 2-(1,3-dimethyl-1H-pyrazol-4-yl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 1,3-dimethyl-4-(4,4,5,5-tetramethyl-1,3,2-dioxaborolan-2-yl)-1H-pyrazole (0.118 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.0185 g, 15%)

LCMS t_{R} (Water, Acidic, 4.0 min): 1.474 min, m/z=470.0 [M+H]⁺

HPLC t_{R} (Waters Alliance e2695 with 2998 detector, Basic, 17.0 min): 6.119 min

¹H NMR: (400 MHz, DMSO) δ 12.09 (s, 1H), 8.20 (s, 1H), 7.47 (s, 1H), 7.11-7.04 (m, 3H), 6.66 (s, 1H), 6.52 (s, 1H), 6.33 (s, 1H), 3.78 (s, 3H), 3.59 (s, 3H), 3.32 (s, 3H), 2.30 (s, 3H), 2.09 (s, 6H).

### Example 13: 4-(5-(2,6-dimethylphenoxy)-1-methyl-2-oxo-1,2-dihydropyridin-4-yl)-6-methyl-2-(1-methyl-1H-pyrrol-3-yl)-1,6-dihydro-7H-pyrrolo[2,3-c] pyridin-7-one

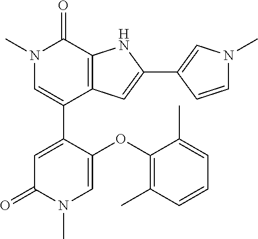

### Preparation 44: 4-(5-(2,6-dimethylphenoxy)-1-methyl-2-oxo-1,2-dihydropyridin-4-yl)-6-methyl-2-(1-methyl-1H-pyrrol-3-yl)-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 1-methyl-3-(4,4,5,5-tetramethyl-1,3,2-dioxaborolan-2-yl)-1H-pyrrole (0.110 g, 0.53 mmol) was reacted to give crude material. The resulting residue was purified by prep HPLC purification using Instrument: A, Column: C; eluted with a gradient of 0.05% ammonium hydroxide solution in water and acetonitrile. Lyophilised fractions gave off white solid. (0.00671 g, 6%)

LCMS t_{R} (Waters, Acidic, 4.0 min): 1.646 min, m/z=455.1 [M+H]⁺

HPLC t_{R} (Waters Alliance e2695 with 2998 detector, Basic, 17.0 min): 6.866 min

¹H NMR: (400 MHz, DMSO) δ 11.99 (s, 1H), 7.45 (s, 1H), 7.37 (s, 1H), 7.11 (d, J=7.2 Hz, 2H), 7.06 (m, 1H), 6.72 (s, 1H), 6.64 (s, 1H), 6.53 (s, 2H), 6.37 (s, 1H), 3.62 (s, 3H), 3.57 (s, 3H), 3.32 (s, 3H), 2.10 (s, 6H)

### Example 14: 4-(5-(2,6-dimethylphenoxy)-1-methyl-2-oxo-1,2-dihydropyridin-4-yl)-2-(2-fluorophenyl)-6-methyl-1,6-dihydro-7H-pyrrolo[2,3-c] pyridin-7-one

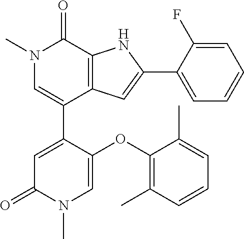 [SMILES: Cc1cccc(C)c1Oc1cn(C)c(=O)cc1-c1cn(C)c(=O)c2[nH]c(-c3ccccc3F)cc12]

### Preparation 45: 4-(5-(2,6-dimethylphenoxy)-1-methyl-2-oxo-1,2-dihydropyridin-4-yl)-2-(2-fluorophenyl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2-fluorophenyl) boronic acid (0.074 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.040 g, 33%)

LCMS t_{R} (Water, Acidic, 4.0 min): 1.830 min, m/z=470.0 [M+H]⁺

HPLC t_{R} (Waters Alliance e2695 with 2998 detector, Basic, 17.0 min): 7.739 min

¹H NMR: (400 MHz, DMSO) δ 12.49 (s, 1H), 8.11-8.07 (m, 1H), 7.54 (s, 1H), 7.40-7.26 (m, 3H), 7.10 (d, J=8.0 Hz, 2H), 7.06-7.02 (m, 1H), 6.80 (d, J=4.0 Hz, 1H), 6.67 (s, 1H), 6.53 (s, 1H), 3.62 (s, 3H), 3.33 (s, 3H), 2.09 (s, 6H)

### Example 15: 4-(5-(2,6-dimethylphenoxy)-1-methyl-2-oxo-1,2-dihydropyridin-4-yl)-6-methyl-2-(o-tolyl)-1,6-dihydro-7H-pyrrolo[2,3-c] pyridin-7-one

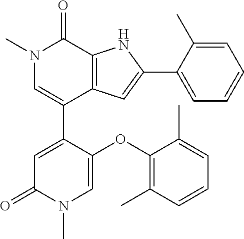 [SMILES: Cc1ccccc1-c1cc2c(-c3cc(=O)n(C)cc3Oc3c(C)cccc3C)cn(C)c(=O)c2[nH]1]

### Preparation 46: 4-(5-(2,6-dimethylphenoxy)-1-methyl-2-oxo-1,2-dihydropyridin-4-yl)-6-methyl-2-(o-tolyl)-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and o-tolyl boronic acid (0.0720 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.0245 g, 20%)

LCMS t_{R} (Water, Acidic, 4.0 min): 1.847 min, m/z=466.1 [M+H]+

HPLC t_{R} (Waters Alliance e2695 with 2998 detector, Basic, 17.0 min): 7.817 min

¹H NMR: (400 MHz, DMSO) δ 12.26 (s, 1H), 7.49 (m, 2H), 7.30-7.24 (m, 3H), 7.12-7.03 (m, 3H), 6.65 (s, 1H), 6.55 (s, 1H), 6.42 (s, 1H), 3.60 (s, 3H), 3.32 (s, 3H), 2.39 (s, 3H), 2.08 (s, 6H).

### Example 16: 4-(5-(2,6-dimethylphenoxy)-1-methyl-2-oxo-1,2-dihydropyridin-4-yl)-2-(2-fluoro-5-methylphenyl)-6-methyl-1,6-dihydro-7H-pyrrolo[2,3-c] pyridin-7-one

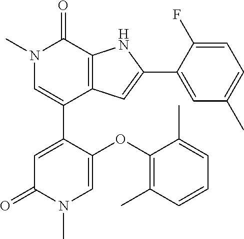

### Preparation 47: 4-(5-(2,6-dimethylphenoxy)-1-methyl-2-oxo-1,2-dihydropyridin-4-yl)-2-(2-fluoro-5-methylphenyl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2-fluoro-5-methylphenyl) boronic acid (0.082 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.076 g, 57%)

LCMS t_{R} (Water, Acidic, 4.0 min): 1.950 min, m/z=484.0 [M+H]⁺

HPLC t_{R} (Waters Alliance e2695 with 2998 detector, Basic, 17.0 min): 8.203 min

¹H NMR: (400 MHz, DMSO) δ 12.43 (s, 1H), 7.97 (d, J=7.2 Hz, 1H), 7.53 (s, 1H), 7.22-7.18 (m, 2H), 7.11-7.02 (m, 3H), 6.79 (d, J=2.4 Hz, 1H), 6.66 (s, 1H), 6.53 (s, 1H), 3.61 (s, 3H), 3.24 (s, 3H), 2.32 (s, 3H), 2.09 (s, 6H)

### Example 17: 4-(5-(2,6-dimethylphenoxy)-1-methyl-2-oxo-1,2-dihydropyridin-4-yl)-2-(2,6-dimethylpyridin-4-yl)-6-methyl-1,6-dihydro-7H-pyrrolo[2,3-c] pyridin-7-one.TFA

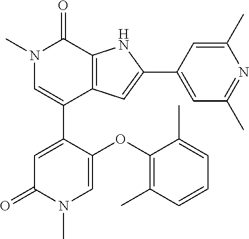

### Preparation 48: 4-(5-(2,6-dimethylphenoxy)-1-methyl-2-oxo-1,2-dihydropyridin-4-yl)-2-(2,6-dimethylpyridin-4-yl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2,6-dimethylpyridin-4-yl) boronic acid (0.080 g, 0.53 mmol) was reacted to give crude material. The resulting residue was purified by prep HPLC purification using Instrument: A, Column: A; eluted with a gradient of 0.05% Trifluoro Acetic acid solution in water and acetonitrile. Lyophilised fractions gave off white solid. (0.0278 g, 22%)

LCMS t_{R} (Waters, Acidic, 4.0 min): 1.280 min, m/z=481.1 [M+H]⁺

HPLC t_{R} (Waters Alliance e2695 with 2998 detector, Acidic, 17.0 min): 4.953 min

¹H NMR: (400 MHz, DMSO) δ 8.25 (br s, 2H), 7.58 (s, 1H), 7.39 (br s, 1H), 7.10 (d, J=7.2 Hz, 3H), 7.06-7.03 (m, 1H), 6.73 (s, 1H), 6.58 (s, 1H), 3.62 (s, 3H), 3.32 (s, 3H), 2.09 (s, 6H), 1.23 (s, 6H).

### Example 18: 4-(5-(2,6-dimethylphenoxy)-1-methyl-2-oxo-1,2-dihydropyridin-4-yl)-2-(5-fluoro-2-methylphenyl)-6-methyl-1,6-dihydro-7H-pyrrolo[2,3-c] pyridin-7-one

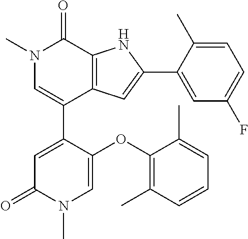 [SMILES: Cc1ccc(F)cc1-c1cc2c(-c3cc(=O)n(C)cc3Oc3c(C)cccc3C)cn(C)c(=O)c2[nH]1]

### Preparation 49: 4-(5-(2,6-dimethylphenoxy)-1-methyl-2-oxo-1,2-dihydropyridin-4-yl)-2-(5-fluoro-2-methylphenyl)-6-methyl-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2,6-dimethylpyridin-4-yl) boronic acid (0.080 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.044 g, 34.42%)

LCMS t_{R} (Waters, Acidic, 4.0 min): 1.901 min, m/z=484.1 [M+H]⁺

HPLC t_{R} (Waters Alliance e2695 with 2998 detector, basic, 17.0 min): 7.948 min

¹H NMR: (400 MHz, DMSO) δ 12.36 (s, 1H), 7.53 (s, 1H), 7.38-7.32 (m, 2H), 7.15-7.02 (m, 4H), 6.66 (s, 1H), 6.55 (s, 1H), 6.51 (d, J=2.4, 1H), 3.61 (s, 3H), 3.32 (s, 3H), 2.38 (s, 3H), 2.08 (s, 6H).

### Example 19: 4-(5-(2,6-dimethylphenoxy)-1-methyl-2-oxo-1,2-dihydropyridin-4-yl)-2-(2,5-dimethylphenyl)-6-methyl-1,6-dihydro-7H-pyrrolo[2,3-c] pyridin-7-one

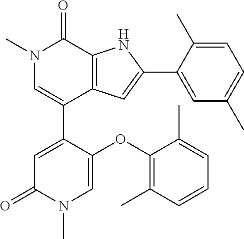

### Preparation 50: 4-(5-(2,6-dimethylphenoxy)-1-methyl-2-oxo-1,2-dihydropyridin-4-yl)-2-(2,5-dimethylphenyl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2,5-dimethylphenyl) boronic acid (0.079 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.026 g, 20%).

LCMS t_{R} (Water, Acidic, 4.0 min): 1.986 min, m/z=480.1 [M+H]⁺

HPLC t_{R} (Waters Alliance e2695 with 2998 detector, basic, 17.0 min): 8.314 min

¹H NMR: (400 MHz, DMSO) δ 12.21 (s, 1H), 7.51 (s, 1H), 7.34 (s, 1H), 7.18 (d, J=7.6 Hz, 1H), 7.12-7.03 (m, 4H), 6.65 (s, 1H), 6.55 (s, 1H), 6.41 (d, J=1.6 Hz, 1H), 3.60 (s, 3H), 3.32 (s, 3H), 2.34 (s, 3H), 2.29 (s, 3H), 2.09 (s, 6H).

### Example 20: 2-(5-chloro-2-methylphenyl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

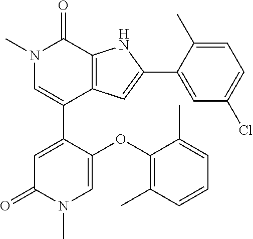 [SMILES: Cc1ccc(Cl)cc1-c1cc2c(-c3cc(=O)n(C)cc3Oc3c(C)cccc3C)cn(C)c(=O)c2[nH]1]

### Preparation 51: 2-(5-chloro-2-methylphenyl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (5-chloro-2-methylphenyl) boronic acid (0.090 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.020 g, 17%).

LCMS t_{R} (Waters, Acidic, 4.0 min): 1.990 min, m/z=501.6 [M+H]⁺

HPLC t_{R} (Waters Alliance e2695 with 2998 detector, basic, 17.0 min): 8.516 min

¹H NMR: (400 MHz, DMSO) δ 12.39 (s, 1H), 7.62-7.53 (m, 3H), 7.34 (s, 2H), 7.11-7.02 (m, 2H), 6.66 (s, 1H), 6.55-6.50 (m, 2H), 3.60 (s, 3H), 3.32 (s, 3H), 2.38 (s, 3H), 2.08 (s, 6H)

### Example 21: 4-(5-(2,6-dimethylphenoxy)-1-methyl-2-oxo-1,2-dihydropyridin-4-yl)-6-methyl-2-(2-methyl-5-(trifluoromethyl) phenyl)-1,6-dihydro-7H-pyrrolo[2,3-c] pyridin-7-one

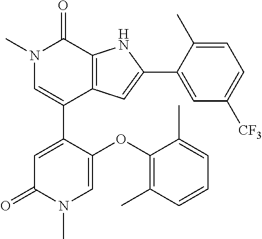 [SMILES: Cc1ccc(C(F)(F)F)cc1-c1cc2c(-c3cc(=O)n(C)cc3Oc3c(C)cccc3C)cn(C)c(=O)c2[nH]1]

### Preparation 52: 4-(5-(2,6-dimethylphenoxy)-1-methyl-2-oxo-1,2-dihydropyridin-4-yl)-6-methyl-2-(2-methyl-5-(trifluoromethyl) phenyl)-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2-methyl-5-(trifluoromethyl) phenyl) boronic acid (0.108 g, 0.53 mmoL) was reacted to give crude material. The resulting residue was purified by prep HPLC purification using Instrument: A, Column: A; eluted with a gradient of 0.05% ammonium hydroxide solution in water and acetonitrile. Lyophilised fractions gave off white solid. (0.032 g, 22%)

LCMS t_{R} (Waters, Acidic, 4.0 min): 2.055 min, m/z=534.0 [M+H]⁺

HPLC t_{R} (Waters Alliance e2695 with 2998 detector, Basic, 17.0 min): 8.702 min

¹H NMR: (400 MHz, DMSO) δ 12.51 (s, 1H), 7.85 (s, 1H), 7.64 (d, J=8 Hz, 1H), 7.55 (d, J=9.6 Hz, 2H), 7.11 (d, J=8.0 Hz, 2H), 7.05 (d, J=6.4 Hz, 1H), 6.67 (s, 1H), 6.56 (s, 2H), 3.61 (s, 3H), 3.30 (s, 3H), 2.32 (s, 3H), 2.08 (s, 6H)

### Example 22: 4-(5-(2,6-dimethylphenoxy)-1-methyl-2-oxo-1,2-dihydropyridin-4-yl)-6-methyl-2-(pyrimidin-5-yl)-1,6-dihydro-7H-pyrrolo[2,3-c] pyridin-7-one

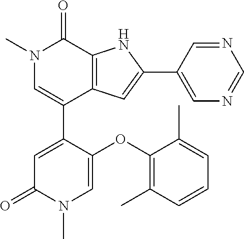 [SMILES: Cc1cccc(C)c1Oc1cn(C)c(=O)cc1-c1cn(C)c(=O)c2[nH]c(-c3cncnc3)cc12]

### Preparation 53: 4-(5-(2,6-dimethylphenoxy)-1-methyl-2-oxo-1,2-dihydropyridin-4-yl)-6-methyl-2-(pyrimidin-5-yl)-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and pyrimidin-5-ylboronic acid (0.066 g, 0.53 mmol) was reacted to give crude material. The resulting residue was purified by prep HPLC purification using Instrument: A, Column: C; eluted with a gradient of 0.05% Trifluoro Acetic acid solution in water and acetonitrile. Lyophilised fractions gave off white solid. (0.023 g, 20%)

LCMS t_{R} (Waters, Acidic, 4.0 min): 1.441 min, m/z=454.0 [M+H]⁺

HPLC t_{R} (Waters Alliance e2695 with 2998 detector, Basic, 17.0 min): 5.974 min

¹H NMR: (400 MHz, DMSO) δ 12.83 (s, 1H), 9.36 (s, 2H), 9.11 (s, 1H), 7.55 (s, 1H), 7.11 (m, 3H), 7.05 (d, J=6.4 Hz, 1H), 6.70 (s, 1H), 6.58 (s, 1H), 3.62 (s, 3H), 3.43 (s, 3H), 2.09 (s, 6H)

### Example 23: 4-(5-(2,6-dimethylphenoxy)-1-methyl-2-oxo-1,2-dihydropyridin-4-yl)-6-methyl-2-(p-tolyl)-1,6-dihydro-7H-pyrrolo[2,3-c] pyridin-7-one

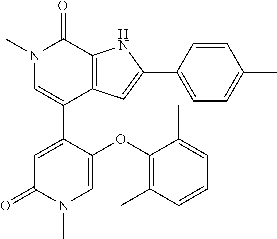

### Preparation 54: 4-(5-(2,6-dimethylphenoxy)-1-methyl-2-oxo-1,2-dihydropyridin-4-yl)-6-methyl-2-(p-tolyl)-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and p-tolyl boronic acid (0.072 g, 0.53 mmol) was reacted to give crude material. The resulting residue was purified by prep HPLC purification using Instrument: B, Column: A; eluted with a gradient of 0.05% ammonium hydroxide solution in water and acetonitrile. Lyophilised fractions gave off white solid (0.023 g, 19%).

LCMS t_{R} (Water, Acidic, 4.0 min): 1.917 min, m/z=466.0 [M+H]⁺

HPLC t_{R} (Waters Alliance e2695 with 2998 detector, basic, 17.0 min): 8.151 min

¹H NMR: (400 MHz, DMSO) δ 12.39 (s, 1H), 7.84 (d, J=8.0 Hz, 2H), 7.50 (s, 1H), 7.23 (d, J=8.0 Hz, 2H), 7.10 (d, J=7.2 Hz, 2H), 7.06-7.02 (m, 1H), 6.75 (s, 1H), 6.67 (s, 1H), 6.55 (s, 1H), 3.60 (s, 3H), 3.33 (s, 3H), 2.32 (s, 3H), 2.10 (s, 6H).

### Example 24: 4-(4-(5-(2,6-dimethylphenoxy)-1-methyl-2-oxo-1,2-dihydropyridin-4-yl)-6-methyl-7-oxo-6,7-dihydro-1H-pyrrolo[2,3-c] pyridin-2-yl) benzonitrile

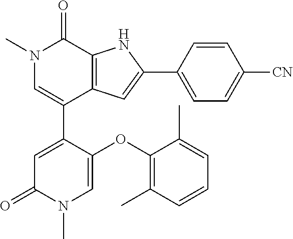 [SMILES: Cc1cccc(C)c1Oc1cn(C)c(=O)cc1-c1cn(C)c(=O)c2[nH]c(-c3ccc(C#N)cc3)cc12]

### Preparation 55: 4-(4-(5-(2,6-dimethylphenoxy)-1-methyl-2-oxo-1,2-dihydropyridin-4-yl)-6-methyl-7-oxo-6,7-dihydro-1H-pyrrolo[2,3-c]pyridin-2-yl) benzonitrile

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 4-cyanophenyl) boronic acid (0.078 g, 0.53 mmol) was reacted to give crude material. The resulting residue was purified by prep HPLC purification using Instrument: B, Column: A; eluted with a gradient of 0.05% ammonium hydroxide solution in water and acetonitrile. Lyophilised fractions gave off white solid (0.004 g, 4%).

LCMS t_{R} (Waters, Acidic, 4.0 min): 2.145 min, m/z=477.2 [M+H]⁺

HPLC t_{R} (Waters Alliance e2695 with 2998 detector, basic, 17.0 min): 7.449 min

¹H NMR: (400 MHz, DMSO) δ 12.70 (br s, 1H), 8.15 (d, J=7.6 Hz, 2H), 7.82 (d, J=7.6 Hz, 2H), 7.46 (s, 1H), 7.11-6.98 (m, 4H), 6.66 (s, 1H), 6.58 (s, 1H), 3.59 (s, 3H), 3.34 (s, 3H), 2.09 (s, 6H).

### Example 25: 4-(5-(2,6-dimethylphenoxy)-1-methyl-2-oxo-1,2-dihydropyridin-4-yl)-2-(4-fluorophenyl)-6-methyl-1,6-dihydro-7H-pyrrolo[2,3-c] pyridin-7-one

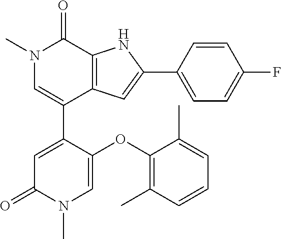 [SMILES: Cc1cccc(C)c1Oc1cn(C)c(=O)cc1-c1cn(C)c(=O)c2[nH]c(-c3ccc(F)cc3)cc12]

### Preparation 56: 4-(5-(2,6-dimethylphenoxy)-1-methyl-2-oxo-1,2-dihydropyridin-4-yl)-2-(4-fluorophenyl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (4-fluorophenyl) boronic acid (0.074 g, 0.53 mmoL) was reacted to give crude material. The resulting residue was purified by prep HPLC purification using Instrument: A, Column: C; eluted with a gradient of 0.05% ammonium hydroxide solution in water and acetonitrile. Lyophilised fractions gave white solid. (0.022 g, 22%)

LCMS t_{R} (Waters Acquity UPLC with QDA mass Detector, Acidic, 4.0 min): 1.82 min, m/z=470.0 [M+H]⁺

HPLC t_{R} (Waters Alliance e2695 with 2998 detector, Basic, 17.0 min): 7.780 min

¹H NMR: (400 MHz, DMSO) δ 12.51 (s, 1H), 8.02-7.99 (m, 2H), 7.51 (s, 1H), 7.26 (apparent t, J=8.8 Hz, 2H), 7.11-7.02 (m, 3H), 6.79 (s, 1H), 6.67 (s, 1H), 6.56 (s, 1H), 3.60 (s, 3H), 3.57 (s, 3H), 2.09 (s, 6H)

### Example 26: 4-(5-(2,6-dimethylphenoxy)-1-methyl-2-oxo-1,2-dihydropyridin-4-yl)-6-methyl-2-(4-(methylsulfonyl) phenyl)-1,6-dihydro-7H-pyrrolo[2,3-c] pyridin-7-one

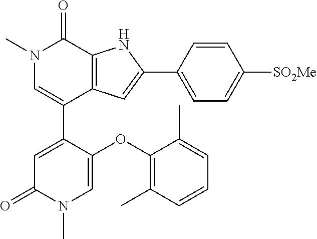 [SMILES: Cc1cccc(C)c1Oc1cn(C)c(=O)cc1-c1cn(C)c(=O)c2[nH]c(-c3ccc(S(C)(=O)=O)cc3)cc12]

### Preparation 57: 4-(5-(2,6-dimethylphenoxy)-1-methyl-2-oxo-1,2-dihydropyridin-4-yl)-6-methyl-2-(4-(methylsulfonyl) phenyl)-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 4,4,5,5-tetramethyl-2-(4-(methyl sulfonyl) phenyl)-1,3,2-dioxaborolane (0.150 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave white solid. (0.0235 g, 17%).

LCMS t_{R} (Waters, Acidic, 4.0 min): 1.627 min, m/z=529.9 [M+H]⁺

HPLC t_{R} (Waters Alliance e2695 with 2998 detector, basic, 17.0 min): 6.634 min

¹H NMR: (400 MHz, DMSO) δ 12.74 (br s, 1H), 8.23 (d, J=8.0 Hz, 2H), 7.91 (d, J=7.6 Hz, 2H), 7.49 (s, 1H), 7.11-6.99 (m, 4H), 6.67 (s, 1H), 6.58 (s, 1H), 3.60 (s, 3H), 3.34 (s, 3H), 3.24 (s, 3H), 2.10 (s, 6H).

### Example 27: 4-(5-(2,6-dimethylphenoxy)-1-methyl-2-oxo-1,2-dihydropyridin-4-yl)-6-methyl-2-(pyridazin-4-yl)-1,6-dihydro-7H-pyrrolo[2,3-c] pyridin-7-one

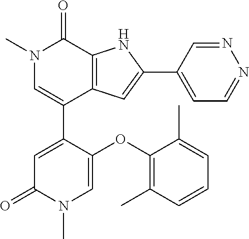 [SMILES: Cc1cccc(C)c1Oc1cn(C)c(=O)cc1-c1cn(C)c(=O)c2[nH]c(-c3ccnnc3)cc12]

### Preparation 58: 4-(tributyl stannyl) Pyridazine

Pyridazine (3.0 g, 37.4 mmol) was dissolved in THF (30.0 mL) under argon. LDA (2M, 18.0 ml, 37.4 mmol in THF) was added to the reaction mixture drop wise at −78° C. The reaction solution was stirred at −78° C. for 30 min. Tributyl(chloro)stannane (13.47 g, 41.4 mmol) was added to the reaction mixture. Reaction mixture was warmed to RT and stirred for 4 h. TLC (5.0:5.0 Hexane:Ethyl acetate) showed no SM remaining. Reaction mixture was quenched with cold water and the mixture was partitioned between water and ethyl acetate. Separated and organic layer concentrated under high vacuum. The product was purified by flash chromatography on silica gel eluting with Hexane/Ethyl acetate gradient (0-10%). Fractions corresponding to product were combined and concentrated to give brown oil (1.5 g, 11%)

LCMS t_{R} (Water, Acidic, 4.0 min): 2.915 min, m/z=370.7 [M+H]⁺

### Preparation 59: 4-(5-(2,6-dimethylphenoxy)-1-methyl-2-oxo-1,2-dihydropyridin-4-yl)-6-methyl-2-(pyridazin-4-yl)-1,6-dihydro-7H-pyrrolo[2,3-c] pyridin-7-one

2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.150 g, 0.266 mmol) was dissolved in Dioxane (3.0 mL) under argon. The suspension was degassed for 30 min. 4-(tributyl stannyl) Pyridazine (0.295 g, 0.799 mmol) was added to the reaction mixture followed by TEA (0.074 g, 0.532 mmol). The suspension was degassed for 10 min. Bis(triphenylphosphine)palladium chloride (0.037 g, 0.053 mmol) added to the reaction mixture. The reaction solution was heated at 160° C. for 2 h. TLC (9.0:1.0 DCM: Methanol) showed no SM remaining. Sodium hydroxide (0.053 g, 1.33 mmol) was added to the reaction mixture. The dark solution was allowed at stir at 140° C. for 40 min. TLC (9.0:1.0 DCM: Methanol) showed no SM remaining. The reaction mixture was partitioned between water and DCM. Separated and organic layer concentrated under high vacuum. The product was purified by reverse phase purification using Instrument: A, Column: B; eluted with a gradient of 0.05% ammonium hydroxide solution in water and acetonitrile. Lyophilised fractions gave white solid. (0.006 g, 4.9%)

LCMS t_{R} (Water, Acidic, 4.0 min): 1.400 min, m/z=454.0 [M+H]⁺

HPLC t_{R} (Waters Alliance e2695 with 2998 detector, Basic, 17.0 min): 5.695 min

1H NMR: (400 MHz, DMSO) δ 13.01 (s, 1H), 9.81 (s, 1H), 9.32 (d, J=5.2 Hz, 1H), 8.25 (d, J=3.2 Hz, 1H), 8.07 (s, 1H), 7.55 (s, 1H), 7.18-7.02 (m, 3H), 6.71 (s, 1H), 6.57 (s, 1H), 3.62 (s, 3H), 3.34 (s, 3H), 2.18 (s, 6H)

### Example 28: 4-(5-(2,6-dimethylphenoxy)-1-methyl-2-oxo-1,2-dihydropyridin-4-yl)-6-methyl-2-(4-(trifluoromethyl) phenyl)-1,6-dihydro-7H-pyrrolo[2,3-c] pyridin-7-one

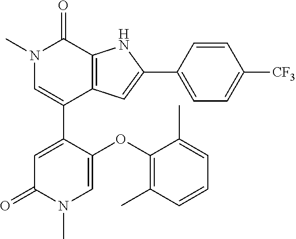 [SMILES: Cc1cccc(C)c1Oc1cn(C)c(=O)cc1-c1cn(C)c(=O)c2[nH]c(-c3ccc(C(F)(F)F)cc3)cc12]

### Preparation 60: 4-(5-(2,6-dimethylphenoxy)-1-methyl-2-oxo-1,2-dihydropyridin-4-yl)-6-methyl-2-(4-(trifluoromethyl) phenyl)-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (4-(trifluoromethyl) phenyl) boronic acid (0.101 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave white solid. (0.0247 g, 18%).

LCMS t_{R} (Waters, Acidic, 4.0 min): 2.050 min, m/z=520.0 [M+H]⁺

HPLC t_{R} (Waters Alliance e2695 with 2998 detector, basic, 17.0 min): 8.568 min

¹H NMR: (400 MHz, DMSO) δ 11.05 (brs, 1H), 8.19 (d, J=8.0 Hz, 2H), 7.77 (d, J=8.0 Hz, 2H), 7.54 (s, 1H), 7.11-6.98 (m, 4H), 6.69 (s, 1H), 6.57 (s, 1H), 3.61 (s, 3H), 3.34 (s, 3H), 2.06 (s, 6H).

### Example 29: 4-(4-(5-(2,6-dimethylphenoxy)-1-methyl-2-oxo-1,2-dihydropyridin-4-yl)-6-methyl-7-oxo-6,7-dihydro-1H-pyrrolo[2,3-c] pyridin-2-yl) benzamide

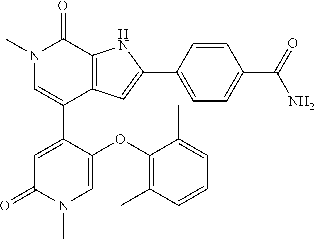 [SMILES: Cc1cccc(C)c1Oc1cn(C)c(=O)cc1-c1cn(C)c(=O)c2[nH]c(-c3ccc(C(N)=O)cc3)cc12]

### Preparation 61: 4-(4-(5-(2,6-dimethylphenoxy)-1-methyl-2-oxo-1,2-dihydropyridin-4-yl)-6-methyl-7-oxo-6,7-dihydro-1H-pyrrolo[2,3-c]pyridin-2-yl) benzamide

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (4-carbamoylphenyl) boronic acid (0.088 g, 0.53 mmol) was reacted to give crude material. The resulting residue was purified by prep HPLC purification using Instrument: C, Column: B; eluted with a gradient of 0.05% ammonium hydroxide solution in water and acetonitrile. Lyophilised fractions gave off white solid (0.014 g, 11%).

LCMS t_{R} (Waters, Acidic, 4.0 min): 1.452 min, m/z=494.9 [M+H]⁺

HPLC t_{R} (Waters Alliance e2695 with 2998 detector, basic, 17.0 min): 5.865 min

¹H NMR: (400 MHz, DMSO) δ 12.58 (s, 1H), 8.02 (d, J=12.8 Hz, 2H), 7.92 (s, 2H), 7.52 (s, 1H), 7.38 (s, 1H), 7.07 (d, J=14.8 Hz, 2H), 6.93 (s, 3H), 6.68 (s, 1H), 6.56 (s, 1H), 3.61 (s, 3H), 3.33 (s, 3H), 2.10 (s, 6H).

### Example 30: 4-(5-(2,6-dimethylphenoxy)-1-methyl-2-oxo-1,2-dihydropyridin-4-yl)-6-methyl-2-(1H-1,2,3-triazol-5-yl)-1,6-dihydro-7H-pyrrolo[2,3-c] pyridin-7-one

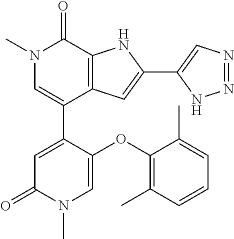 [SMILES: Cc1cccc(C)c1Oc1cn(C)c(=O)cc1-c1cn(C)c(=O)c2[nH]c(-c3cnn[nH]3)cc12]

### Preparation 62: 4-(5-(2,6-dimethylphenoxy)-1-methyl-2-oxo-1,2-dihydropyridin-4-yl)-6-methyl-2-(2-(tetrahydro-2H-pyran-2-yl)-2H-1,2,3-triazol-4-y)-1,6-dihydro-7H-pyrrolo[2,3-c]pyridin-7-one

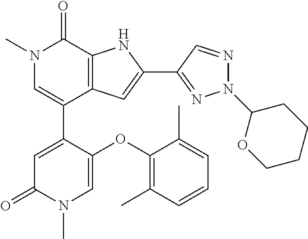 [SMILES: Cc1cccc(C)c1Oc1cn(C)c(=O)cc1-c1cn(C)c(=O)c2[nH]c(-c3cnn(C4CCCCO4)n3)cc12]

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 2-(tetrahydro-2H-pyran-2-yl)-4-(4,4,5,5-tetramethyl-1,3,2-dioxaborolan-2-yl)-2H-1,2,3-triazole (0.103 g, 0.53 mmol) was reacted to give crude material (0.22 g, crude)

LCMS t_{R} (Waters, Acidic, 4.0 min): 1.750 min, m/z=527.0 [M+H]⁺

### Preparation 63: 4-(5-(2,6-dimethylphenoxy)-1-methyl-2-oxo-1,2-dihydropyridin-4-yl)-6-methyl-2-(2H-1,2,3-triazol-4-yl)-1,6-dihydro-7H-pyrrolo[2,3-c] pyridin-7-one

To 4-(5-(2,6-dimethylphenoxy)-1-methyl-2-oxo-1,2-dihydropyridin-4-yl)-6-methyl-2-(2-(tetrahydro-2H-pyran-2-yl)-2H-1,2,3-triazol-4-yl)-1,6-dihydro-7H-pyrrolo[2,3-c] pyridin-7-one (0.22 g, 0.26 mmol, crude) was added aq. HCl (10 mL). The suspension was heated at 70° C. for 2 h. TLC (9.5:0.5 DCM:methanol) showed no SM remaining. The reaction mixture was cooled to 0° C. The resulting solution was partitioned between water and dichloromethane, separated and organic fraction concentrated under reduced pressure. The mixture was filtered; filtrate was evaporated to give an oil. The product was purified by reverse phase chromatography eluting with acetonitrile/water gradient (0-42%). Fractions corresponding to product were combined and lyophilised to give off-white solid (0.045 g, 25%)

LCMS t_{R} (Waters, Acidic, 4.0 min): 1.420 min, m/z=443.0 [M+H]⁺

HPLC t_{R} (Waters Alliance e2695 with 2998 detector, Basic, 17.0 min): 3.871 min

¹H NMR: (400 MHz, DMSO) δ 15.26 (s, 1H), 12.59 (s, 1H), 8.40 (s, 1H), 7.54 (s, 1H), 7.11 (d, J=7.6 Hz, 2H), 7.06-7.03 (m, 1H), 6.80 (s, 1H), 6.68 (s, 1H), 6.53 (s, 1H), 3.61 (s, 3H), 3.33 (s, 3H), 2.10 (s, 6H).

### Example 31: 4-(5-(2,6-dimethylphenoxy)-1-methyl-2-oxo-1,2-dihydropyridin-4-yl)-6-methyl-2-(2-methylthiazol-5-yl)-1,6-dihydro-7H-pyrrolo[2,3-c] pyridin-7-one

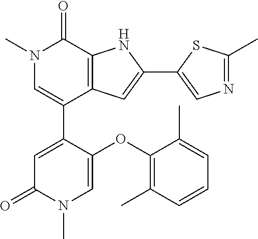

### Preparation 64: 4-(5-(2,6-dimethylphenoxy)-1-methyl-2-oxo-1,2-dihydropyridin-4-yl)-6-methyl-2-(2-methylthiazol-5-yl)-1,6-dihydro-7H-pyrrolo[2,3-c]pyridin-7-one

2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) was dissolved in Dioxane (1.5 mL) under argon. 2-methyl-5-(4,4,5,5-tetramethyl-1,3,2-dioxaborolan-2-yl) thiazole (0.119 g, 0.53 mmol) was added to the reaction mixture followed by potassium phosphate (0.169 g, 0.79 mmol) and water (0.3 mL) at room temperature. The suspension was degassed for 30 min. Xphos-Pd-G3 (0.031 g, 0.037 mmol) was added to the reaction mixture. The reaction dark solution was heated at 120° C. for 16 h. TLC (9.5:0.5 DCM: methanol) showed no SM remaining. The reaction mixture was cooled to 0° C. Sodium hydroxide (0.031 g, 0.79 mmol) was added to the reaction mixture. The reaction dark solution was allowed to stir at 120° C. for 6 h. TLC (9.5:0.5 DCM:methanol) showed no SM remaining. The resulting solution was directly concentrated under reduced pressure and ethyl acetate added. The mixture was filtered; filtrate was evaporated to an oil. The product was purified by Prep HPLC Purification using Instrument: A, Column: C; eluted with a gradient of 0.05% ammonium hydroxide solution in water and acetonitrile. Lyophilised fractions gave white solid. (0.027 g, 22%).

LCMS t_{R} (Waters, Acidic, 4.0 min): 1.587 min, m/z=472.9 [M+H]⁺

HPLC t_{R} (Waters Alliance e2695 with 2998 detector, basic, 17.0 min): 6.624 min

¹H NMR: (400 MHz, DMSO) δ 12.71 (s, 1H), 8.23 (s, 1H), 7.53 (s, 1H), 7.11-7.02 (m, 3H), 6.67 (s, 1H), 6.52 (d, J=4.4 Hz, 2H), 3.60 (s, 3H), 3.32 (s, 3H), 2.67 (s, 3H), 2.09 (s, 6H).

### Example 32: 4-(5-(2,6-dimethylphenoxy)-1-methyl-2-oxo-1,2-dihydropyridin-4-yl)-6-methyl-2-(1H-pyrazol-5-yl)-1,6-dihydro-7H-pyrrolo [2,3-c] pyridin-7-one

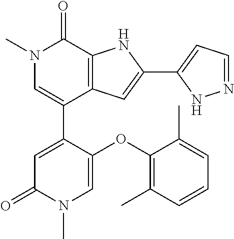 [SMILES: Cc1cccc(C)c1Oc1cn(C)c(=O)cc1-c1cn(C)c(=O)c2[nH]c(-c3ccn[nH]3)cc12]

### Preparation 65: 4-(5-(2,6-dimethylphenoxy)-1-methyl-2-oxo-1,2-dihydropyridin-4-yl)-6-methyl-2-(1H-pyrazol-5-yl)-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 5-(4,4,5,5-tetramethyl-1,3,2-dioxaborolan-2-yl)-1H-pyrazole (0.103 g, 0.53 mmol) was reacted to give crude material. The resulting residue was purified by prep HPLC purification using Instrument: B, Column: C; eluted with a gradient of 0.05% ammonium hydroxide solution in water and acetonitrile. Lyophilised fractions gave brown solid (0.019 g, 16%)

LCMS t_{R} (Waters, Acidic, 4.0 min): 1.480 min, m/z=442.1 [M+H]⁺

HPLC t_{R} (Waters Alliance e2695 with 2998 detector, basic, 17.0 min): 6.043 min

¹H NMR: (400 MHz, DMSO) δ 11.70 (m, 2H), 7.70 (s, 1H), 7.52 (s, 1H), 7.11 (d, J=7.2 Hz, 2H), 7.08-7.02 (m, 1H), 6.98 (d, J=1.2 Hz, 1H), 6.76 (s, 1H), 6.67 (s, 1H), 6.56 (s, 1H), 3.60 (s, 3H), 3.33 (s, 3H), 2.10 (s, 6H).

### Example 33: 4-(5-(2,6-dimethylphenoxy)-1-methyl-2-oxo-1,2-dihydropyridin-4-yl)-6-methyl-2-(1-methyl-1H-1,2,3-triazol-5-yl)-1,6-dihydro-7H-pyrrolo[2,3-c] pyridin-7-one

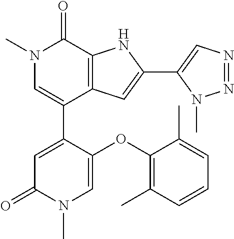 [SMILES: Cc1cccc(C)c1Oc1cn(C)c(=O)cc1-c1cn(C)c(=O)c2[nH]c(-c3cnnn3C)cc12]

### Preparation 66: 4-(5-(2,6-dimethylphenoxy)-1-methyl-2-oxo-1,2-dihydropyridin-4-yl)-6-methyl-2-(1-methyl-1H-1,2,3-triazol-5-yl)-1,6-dihydro-7H-pyrrolo[2,3-c] pyridin-7-one

2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) was dissolved in 1,4-dioxane (1.5 mL) under argon. 1-methyl-5-(tributyl stannyl)-1H-1,2,3-triazole (0.118 g, 0.53 mmol) was added to the reaction mixture followed by triethylamine (0.03 mL, 0.53 mmol) at room temperature. The suspension was degassed for 30 min. PdCl₂(PPh₃)₂(0.037 g, 0.052 mmol) was added to the reaction mixture. The dark solution was heated at 160° C. for 1 h. TLC (9.5:0.5 DCM:methanol) showed no SM remaining. The reaction mixture was cooled to 0° C. Sodium hydroxide (0.053 g, 1.33 mmol) was added to the reaction mixture. The dark solution was allowed at stir at 140° C. for 40 min. TLC (9.5:0.5 DCM:methanol) showed no SM remaining. The resulting solution was directly concentrated under reduced pressure and ethyl acetate was added. The mixture was filtered; filtrate was evaporated to an oil. The crude material was purified by reverse phase column chromatography and product was eluted at 20% acetonitrile in water. Fractions corresponding to product were combined and lyophilised to give off-white solid (0.024 g, 21%)

LCMS t_{R} (Waters, Acidic, 4.0 min): 1.442 min, m/z=456.9 [M+H]⁺

HPLC t_{R} (Waters Alliance e2695 with 2998 detector, Basic, 17.0 min): 5.891 min

¹H NMR: (400 MHz, DMSO) δ 12.76 (s, 1H), 8.09 (s, 1H), 7.44 (s, 1H), 7.06-7.02 (m, 3H), 6.84 (s, 1H), 6.70 (s, 1H), 6.34 (s, 1H), 4.23 (s, 3H), 3.32 (s, 3H), 3.26 (s, 3H), 2.09 (s, 6H).

### Example 34: 4-(5-(2,6-dimethylphenoxy)-1-methyl-2-oxo-1,2-dihydropyridin-4-yl)-2-(2-fluoro-5-(trifluoromethyl) phenyl)-6-methyl-1,6-dihydro-7H-pyrrolo[2,3-c] pyridin-7-one

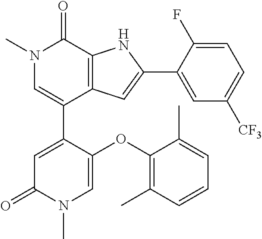 [SMILES: Cc1cccc(C)c1Oc1cn(C)c(=O)cc1-c1cn(C)c(=O)c2[nH]c(-c3cc(C(F)(F)F)ccc3F)cc12]

### Preparation 67: 4-(5-(2,6-dimethylphenoxy)-1-methyl-2-oxo-1,2-dihydropyridin-4-yl)-2-(2-fluoro-5-(trifluoromethyl) phenyl)-6-methyl-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2-fluoro-5-(trifluoromethyl) phenyl) boronic acid (0.110 g, 0.53 mmol) was reacted to give crude material. The resulting residue was purified by prep HPLC purification using Instrument: A, Column: B; eluted with a gradient of 0.05% ammonium hydroxide solution in water and acetonitrile. Lyophilised fractions gave white solid (0.031 g, 22%).

LCMS t_{R} (Waters, Acidic, 4.0 min): 2.069 min. m/z=538.0 [M+H]⁺

HPLC t_{R} (Waters Alliance e2695 with 2998 detector, basic, 17.0 min): 8.640 min

¹H NMR: (400 MHz, DMSO) δ 12.81 (br s, 1H), 8.63 (br s, 1H), 7.76-7.55 (m, 3H), 7.08-6.91 (m, 4H), 6.67-6.53 (m, 2H), 3.61 (s, 3H), 3.33 (s, 3H), 2.08 (s, 6H).

### Example 35: 4-(4-(5-(2,6-dimethylphenoxy)-1-methyl-2-oxo-1,2-dihydropyridin-4-yl)-6-methyl-7-oxo-6,7-dihydro-1H-pyrrolo[2,3-c] pyridin-2-yl) benzoic acid

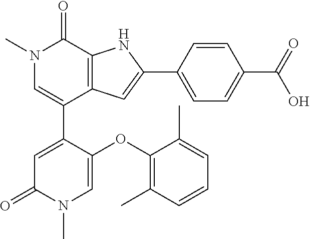 [SMILES: Cc1cccc(C)c1Oc1cn(C)c(=O)cc1-c1cn(C)c(=O)c2[nH]c(-c3ccc(C(=O)O)cc3)cc12]

### Preparation 68: 4-(4-(5-(2,6-dimethylphenoxy)-1-methyl-2-oxo-1,2-dihydropyridin-4-yl)-6-methyl-7-oxo-6,7-dihydro-1H-pyrrolo[2,3-c] pyridin-2-yl) benzoic acid

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (4-(ethoxy carbonyl) phenyl) boronic acid (0.103 g, 0.53 mmol) was reacted to give crude material. The resulting residue was purified by prep HPLC purification using Instrument: A, Column: A; eluted with a gradient of 0.05% ammonium hydroxide solution in water and acetonitrile. Lyophilised fractions gave white solid (0.00599 g, 5%).

LCMS t_{R} (Waters, Acidic, 4.0 min): 1.580 min, m/z=495.9 [M+H]⁺

HPLC t_{R} (Waters Alliance e2695 with 2998 detector, basic, 17.0 min): 3.898 min

¹H NMR: (400 MHz, DMSO) δ 12.46 (brs, 2H), 7.87 (s, 4H), 7.51 (s, 1H), 7.11-7.04 (m, 3H), 6.81 (s, 1H), 6.68 (s, 1H), 6.56 (s, 1H), 3.60 (s, 3H), 3.33 (s, 3H), 2.10 (s, 6H).

### Example 36: 2-(1,5-dimethyl-1H-pyrazol-4-yl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

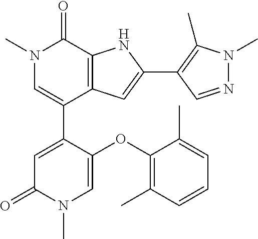

### Preparation 69: 2-(1,5-dimethyl-1H-pyrazol-4-yl)-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 1,5-dimethyl-4-(4,4,5,5-tetramethyl-1,3,2-dioxaborolan-2-yl)-1H-pyrazole (0.118 g, 0.53 mmol) was reacted to give crude material. The resulting residue was purified by prep HPLC purification using Instrument: C, Column: B; eluted with a gradient of 0.1% formic acid solution in water and acetonitrile. Lyophilised fractions gave brown solid (0.023 g, 19%).

LCMS t_{R} (Waters, Acidic, 4.0 min): 1.506 min, m/z=470.1 [M+H]⁺

HPLC t_{R} (Waters Alliance e2695 with 2998 detector, basic, 17.0 min): 6.205 min

¹H NMR: (400 MHz, DMSO) δ 12.15 (s, 1H), 7.92 (s, 1H), 7.48 (s, 1H), 7.11-7.02 (m, 3H), 6.66 (s, 1H), 6.53 (s, 1H), 6.32 (s, 1H), 3.77 (s, 3H), 3.59 (s, 3H), 3.32 (s, 3H), 2.40 (s, 3H), 2.09 (s, 6H).

### Example 37: 2-(2,6-difluorophenyl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

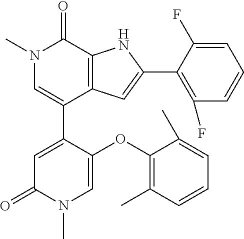 [SMILES: Cc1cccc(C)c1Oc1cn(C)c(=O)cc1-c1cn(C)c(=O)c2[nH]c(-c3c(F)cccc3F)cc12]

### Preparation 70: 2-(2,6-difluorophenyl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and (2,6-difluorophenyl) boronic acid (0.084 g, 0.53 mmol) was reacted to give crude material. The resulting residue was purified by prep HPLC purification using Instrument: C, Column: B; eluted with gradient of 0.05% Ammonium hydroxide solution in water and acetonitrile. Lyophilised gave off white solid. (0.005 g, 4%)

LCMS t_{R} (Waters, Acidic, 4.0 min): 1.790 min, m/z=487.9 [M+H]⁺

HPLC t_{R} (Waters Alliance e2695 with 2998 detector, basic, 17.0 min): 7.643 min

¹H NMR: (400 MHz, DMSO) δ 12.40 (brs, 1H), 7.95 (d, J=7.6 Hz, 1H), 7.54-7.40 (m, 2H), 7.34-7.22 (m, 1H), 7.11-7.03 (m, 3H), 6.67-6.53 (m, 3H), 3.61 (s, 3H), 3.36 (s, 3H), 2.08 (s, 6H).

### Example 38: 4-(5-(2,6-dimethylphenoxy)-1-methyl-2-oxo-1,2-dihydropyridin-4-yl)-6-methyl-2-(6-methyl pyrimidin-4-yl)-1,6-dihydro-7H-pyrrolo[2,3-c] pyridin-7-one

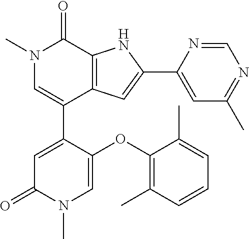

### Preparation 71: 4-methyl-6-(Trimethyl stannyl) pyrimidine

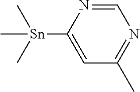

Toluene (10 mL, 20V) was degassed under argon for 30 min. 4-bromo-6-methylpyrimidine (0.50 g, 2.90 mmol) and Hexamethylditin (1.20 mL, 5.81 mmol) was added to the reaction mixture followed by Tetrakis (0.167 g, 0.145 mmol) at room temperature. The resulting mixture was allowed to stir at 100° C. for 4 h. TLC (3:7 Ethyl acetate/Hexane) showed no SM remaining. The resulting solution was filtered through celite-pad and filtrate was directly concentrated under vacuum to afford brown sticky solid (1 g, quantitative).

LCMS t_{R} (Waters, Acidic, 4.0 min): 0.890 min, m/z=259 [M+H]⁺.

### Preparation 72: 4-bromo-6-methyl-2-(6-methylpyrimidin-4-yl)-1-tosyl-1, 6-dihydro-7H-pyrrolo [2, 3-c] pyridin-7-one

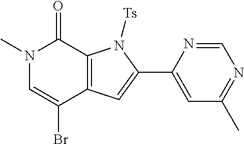

4-bromo-2-iodo-6-methyl-1-tosyl-1, 6-dihydro-7H-pyrrolo [2, 3-c] pyridin-7-one (0.65 g, 1.29 mmol) and 4-methyl-6-(Trimethyl stannyl) pyrimidine (1.0 g, 3.87 mmol) was dissolved in DMF (20 mL) under argon. Lithium chloride (0.082 g, 1.93 mmol) was added to the reaction mixture followed by Copper(I) iodide (0.074 g, 0.38 mmol) at room temperature. The suspension was degassed by using argon for 30 min. Tetrakis (0.074 g, 0.064 mmol) was added to the reaction mixture and resulting reaction mixture was allowed to stir at 80° C. for 16 h. TLC (3:7 Ethyl acetate/Hexane) showed no SM remaining. The reaction mixture was diluted with water (50 mL) and extracted by ethyl acetate (50 mL×3). The combined organics were dried over sodium sulphate, filtered and evaporated. The residue was purified by normal phase chromatography, eluting with (60:40) ethyl acetate/Hexane. Solvent reduction gave brown viscous (0.16 g, 9%).

LCMS t_{R}: (Waters, Acidic, 4.0 min): 1.945 min. m/z=472.7 [M+H]⁺.

### Preparation 73: 6-methyl-2-(6-methylpyrimidin-4-yl)-4-(4,4,5,5-tetramethyl-1,3,2-dioxaborolan-2-yl)-1-tosyl-1,6-dihydro-7H-pyrrolo[2,3-c]pyridin-7-one

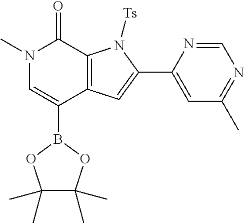

4-bromo-6-methyl-2-(6-methylpyrimidin-4-yl)-1-tosyl-1, 6-dihydro-7H-pyrrolo [2, 3-c] pyridin-7-one (0.12 g, 0.25 mmol) was dissolved in Dioxane (10 ml) under argon. The suspension was degassed by using argon for 30 min. Potassium acetate (0.049 g, 0.50 mmol) was added to the reaction mixture followed by Bis (pinacolato) diboron (0.19 g, 0.76 mmol).

Tris(dibenzylideneacetone)dipalladium (0.011 g, 0.012 mmol) and X-Phos (0.012 g, 0.025 mmol) were added to the reaction mixture. The reaction dark solution was heated at 90° C. for 12 h. TLC (5:5 Hexane:Ethyl acetate) showed no SM remaining. The reaction mixture was diluted with water (40 mL) and extracted by ethyl acetate (40 mL×3). The combined organics were dried over Na₂SO₄, filtered and evaporated to give brown viscous material. (0.13 g, Quantitative)

LCMS t_{R} (Waters, Acidic, 4.0 min): 1.477 min, m/z=438.7 [M+H]⁺ (boronic acid) and 2.232 min, m/z=520.8 [M+H]⁺ (boronic ester)

### Preparation 74: 4-(5-(2,6-dimethylphenoxy)-1-methyl-2-oxo-1,2-dihydropyridin-4-yl)-6-methyl-2-(6-methylpyrimidin-4-yl)-1,6-dihydro-7H-pyrrolo[2,3-c]pyridin-7-one

Following the procedure in preparation 43, 4-bromo-5-(2, 6-dimethylphenoxy)-1-methylpyridin-2(1H)-one (0.114 g, 0.37 mmol) and 6-methyl-2-(6-methylpyrimidin-4-yl)-4-(4,4,5,5-tetramethyl-1,3,2-dioxaborolan-2-yl)-1-tosyl-1,6-dihydro-7H-pyrrolo[2,3-c]pyridin-7-one (0.130 g, 0.29 mmol) was reacted to give crude material. The resulting residue was purified by prep HPLC purification using Instrument: C, Column: B; eluted with gradient of 0.05% Ammonium hydroxide solution in water and acetonitrile. Lyophilised gave off white solid (0.0044 g, 4%).

LCMS t_{R} (Waters, Acidic, 4.0 min): 1.497 min, m/z=468.1 [M+H]⁺

HPLC t_{R} (Waters Alliance e2695 with 2998 detector, basic, 17.0 min): 6.139 min

¹H NMR: (400 MHz, DMSO) δ 12.5 (s, 1H), 9.01 (s, 1H), 8.18 (s, 1H), 7.58 (s, 1H), 7.27 (s, 1H), 7.14-7.02 (m, 3H), 6.69 (s, 1H), 6.56 (s, 1H), 3.61 (s, 3H), 2.67 (s, 3H), 2.33 (s, 3H), 2.10 (s, 6H).

### Example 39: 4-(5-(2,6-dimethylphenoxy)-1-methyl-2-oxo-1,2-dihydropyridin-4-yl)-6-methyl-2-(pyrimidin-2-yl)-1,6-dihydro-7H-pyrrolo[2,3-c] pyridin-7-one

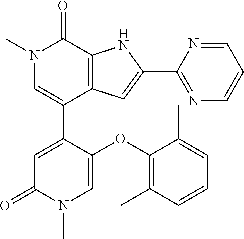 [SMILES: Cc1cccc(C)c1Oc1cn(C)c(=O)cc1-c1cn(C)c(=O)c2[nH]c(-c3ncccn3)cc12]

### Preparation 75: 4-bromo-6-methyl-2-(pyrimidin-2-yl)-1-tosyl-1, 6-dihydro-7H-pyrrolo [2, 3-c]

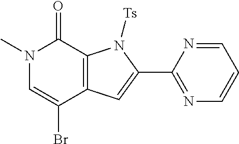 [SMILES: Cc1ccc(S(=O)(=O)n2c(-c3ncccn3)cc3c(Br)cn(C)c(=O)c32)cc1]

4-bromo-2-iodo-6-methyl-1-tosyl-1,6-dihydro-7H-pyrrolo[2,3-c] pyridin-7-one (0.55 g, 1.08 mmol) and 2-(tributyl stannyl) pyrimidine (0.80 g, 2.17 mmol) was dissolved in dioxane (22 mL) under argon. Copper(I) iodide (0.010 g, 0.054 mmol) was added to the reaction mixture at room temperature. The suspension was degassed by argon for 15 min. Tetrakis (0.062 g, 0.029 mmol) was added to the reaction mixture and resulting reaction mixture was allowed to stir at 80° C. for 12 h. TLC (3:7 Ethyl acetate/Hexane) showed no SM remaining. The reaction mixture was diluted with water (50 mL) and extracted by ethyl acetate (50 mL×3). The combined organics were dried over Na₂SO₄, filtered and evaporated. The residue was purified by normal phase chromatography, eluting with (40:60) ethyl acetate/Hexane. Solvent reduction gave brown solid. (0.15 g, 31%)

LCMS t_{R}: (Waters, Acidic, 4.0 min): 1.902 min, m/z=458.7 [M+H]⁺.

### Preparation 76: 6-methyl-2-(pyrimidin-2-yl)-4-(4, 4, 5, 5-tetramethyl-1, 3, 2-dioxaborolan-2-yl)-1-tosyl-1, 6-dihydro-7H-pyrrolo[2, 3-c]pyridin-7-one

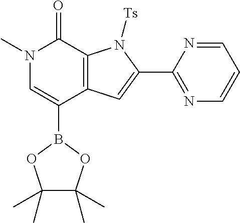 [SMILES: Cc1ccc(S(=O)(=O)n2c(-c3ncccn3)cc3c(B4OC(C)(C)C(C)(C)O4)cn(C)c(=O)c32)cc1]

Following the procedure in preparation 73, 4-bromo-6-methyl-2-(pyrimidin-2-yl)-1-tosyl-1, 6-dihydro-7H-pyrrolo [2, 3-c] pyridin-7-one (0.12 g, 0.26 mmol) was reacted to give title compound as brown viscous material. (0.20 g, Quantitative)

LCMS t_{R} (Waters, Acidic, 4.0 min): 1.422 min, m/z=424.8 [M+H]⁺ (boronic acid) and 2.236 min, m/z=506.9 [M+H]⁺ (boronic ester) Preparation 77: 4-(5-(2, 6-dimethylphenoxy)-1-methyl-2-oxo-1, 2-dihydropyridin-4-yl)-6-methyl-2-(pyrimidin-2-y)-1, 6-dihydro-7H-pyrrolo [2, 3-c] pyridin-7-one

Following the procedure in preparation 64, 4-bromo-5-(2, 6-dimethylphenoxy)-1-methylpyridin-2(1H)-one (0.15 g, 0.48 mmol) and 6-methyl-2-(pyrimidin-2-yl)-4-(4,4,5,5-tetramethyl-1,3,2-dioxaborolan-2-yl)-1-tosyl-1,6-dihydro-7H-pyrrolo[2,3-c] pyridin-7-one (0.20 g, 0.47 mmol) was reacted to give crude material. The resulting residue was purified by prep HPLC purification using Instrument: C, Column: B; eluted with gradient of 0.05% Ammonium hydroxide solution in water and acetonitrile. Lyophilised gave off white solid (0.021 g, 10%).

LCMS t_{R} (Waters, Acidic, 4.0 min): 1.535 min, m/z=454 [M+H]⁺

HPLC t_{R} (Waters Alliance e2695 with 2998 detector, basic, 17.0 min): 6.364 min

¹H NMR: (400 MHz, DMSO) δ 12.4 (br s, 1H), 8.86 (d, J=4.8 Hz, 2H), 7.57 (s, 1H), 7.40 (dd, J=4.8 Hz, J=10 Hz, 1H), 7.21 (s, 1H), 7.11-7.02 (m, 3H), 6.68 (s, 1H), 6.57 (s, 1H), 3.60 (s, 3H), 3.34 (s, 3H), 2.11 (s, 6H).

### Example 40: 2-(1,5-dimethyl-1H-pyrazol-4-yl)-4-(5-(2,6-dimethylphenoxy)-1-(oxetan-3-yl)-2-oxo-1,2-dihydropyridin-4-yl)-6-methyl-1,6-dihydro-7H-pyrrolo[2,3-c] pyridin-7-one

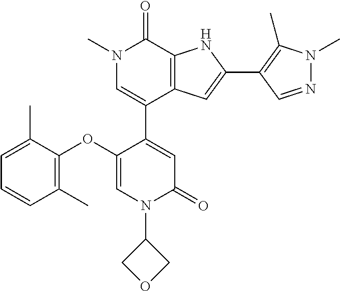 [SMILES: Cc1cccc(C)c1Oc1cn(C2COC2)c(=O)cc1-c1cn(C)c(=O)c2[nH]c(-c3cnn(C)c3C)cc12]

### Preparation 78: 4-bromo-2-(1,5-dimethyl-1H-pyrazol-4-yl)-6-methyl-1-tosyl-1,6-dihydro-7H-pyrrolo[2,3-c] pyridin-7-one

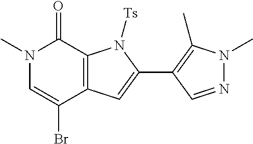

4-bromo-2-iodo-6-methyl-1-tosyl-1, 6-dihydro-7H-pyrrolo [2, 3-c] pyridin-7-one (1.5 g, 2.96 mmol) was dissolved in Dioxane (60 mL) under argon. 1,5-dimethyl-4-(4,4,5,5-tetramethyl-1,3,2-dioxaborolan-2-yl)-1H-pyrazole (1.31 g, 5.93 mmol) was added to the reaction mixture followed by potassium phosphate (01.25 g, 5.93 mmol) and water (40 mL) at room temperature. The suspension was degassed for 15 min. PdCl₂(dpp)DCM complex (0.169 g, 0.207 mmol) was added to the reaction mixture. The reaction dark solution was heated at 60° C. for up to 4 h. TLC (5:5 Hexane: ethyl acetate) showed no SM remaining. The reaction mixture was diluted with water (150 mL) and extracted by ethyl acetate (150 mL×3). The combined organics were dried over Na₂SO₄, filtered and evaporated. The residue was purified by normal phase chromatography, eluting with (50:50) ethyl acetate/Hexane. Solvent reduction gave brown solid (1.0 g, 70%).

LCMS t_{R}: (Waters, Acidic, 4.0 min): 1.789 min, m/z=474.7 [M+H]⁺

¹H NMR: (400 MHz, DMSO) δ 7.88 (s, 1H), 7.71-7.69 (d, J=8.4 Hz, 2H), 7.41-7.37 (m, 3H), 6.38 (s, 1H), 3.78 (s, 3H), 3.42 (s, 3H), 2.38 (s, 3H), 2.16 (s, 3H),

### Preparation 79: 2-(1,5-dimethyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c] pyridin-7-one

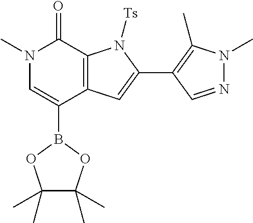

4-bromo-2-(1,5-dimethyl-1H-pyrazol-4-yl)-6-methyl-1-tosyl-1,6-dihydro-7H-pyrrolo[2,3-c]pyridin-7-one (1.0 g, 2.10 mmol) (0.83 g, 1.75 mmol) was dissolved in Dioxane (60 mL) under argon. The suspension was degassed by argon for 15 min. Potassium acetate (0.51 g, 5.25 mmol) was added followed by Bis (pinacolato) diboron (1.77 g, 7.0 mmol) to the reaction mixture and degassed for 15 min. Tris(dibenzylideneacetone) dipalladium (0) (0.080 g, 0.087 mmol) and X-Phos (0.083 g, 0.17 mmol) was added to the reaction mixture. The reaction dark solution was heated at 60° C. for 16 h. TLC (5:5 Hexane:Ethyl acetate) showed no SM remaining. The reaction mixture was diluted with water (50 mL) and extracted by ethyl acetate (50 mL×3). The combined organics layers were dried over Na₂SO₄, filtered and evaporated to give brown sticky solid (1.20 g, Quantitative)

LCMS t_{R} (Waters, Acidic, 4.0 min): 1.336 min, m/z=440.8 [M+H]⁺ (boronic acid) and 2.092 min, m/z=522.9 [M+H]⁺ (boronic ester)

### Preparation 80: 2-(1,5-dimethyl-1H-pyrazol-4-yl)-4-(5-(2,6-dimethylphenoxy)-1-(oxetan-3-yl)-2-oxo-1,2-dihydropyridin-4-yl)-6-methyl-1-tosyl-1,6-dihydro-7H-pyrrolo[2,3-c]pyridin-7-one

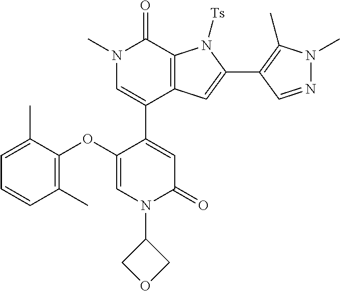 [SMILES: Cc1ccc(S(=O)(=O)n2c(-c3cnn(C)c3C)cc3c(-c4cc(=O)n(C5COC5)cc4Oc4c(C)cccc4C)cn(C)c(=O)c32)cc1]

4-bromo-5-(2, 6-dimethylphenoxy)-1-(oxetan-3-yl) pyridin-2(1H)-one (0.34 g, 0.99 mmol) was dissolved in Dioxane (20 mL) under argon. 2-(1,5-dimethyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c] pyridin-7-one (1.2 g, 2.29 mmol) was added to the reaction mixture followed by potassium phosphate (0.42 g, 1.99 mmol) and water (5 mL) at room temperature. The suspension was degassed for 15 min. Xphos-PdG₃ (0.084 g, 0.099 mmol) was added to the reaction mixture. The reaction dark solution was heated at 60° C. for 6 h. TLC (9.5:0.5 DCM:methanol) showed no SM remaining. The reaction mixture was diluted with water (150 mL) and extracted by ethyl acetate (150 mL×3). The combined organics were dried over Na₂SO₄, filtered and evaporated. The residue was purified by normal phase chromatography, eluting with (55:45) Acetonitrile/water. Fractions corresponding to product were combined and lyophilize to give brown solid (0.18 g, 19%)

LCMS t_{R} (Waters, Acidic, 4.0 min): 1.753 min, m/z=666.10 [M+H]⁺

### Preparation 81: 2-(1,5-dimethyl-1H-pyrazol-4-yl)-4-(5-(2,6-dimethylphenoxy)-1-(oxetan-3-yl)-2-oxo-1,2-dihydropyridin-4-yl)-6-methyl-1,6-dihydro-7H-pyrrolo[2,3-c] pyridin-7-one

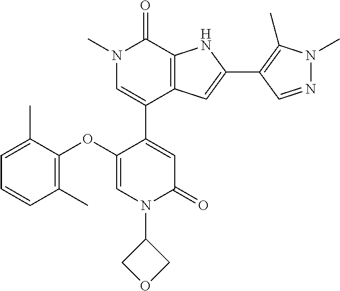 [SMILES: Cc1cccc(C)c1Oc1cn(C2COC2)c(=O)cc1-c1cn(C)c(=O)c2[nH]c(-c3cnn(C)c3C)cc12]

2-(1, 5-dimethyl-1H-pyrazol-4-yl)-4-(5-(2, 6-dimethylphenoxy)-1-(oxetan-3-yl)-2-oxo-1, 2-dihydropyridin-4-yl)-6-methyl-1-tosyl-1, 6-dihydro-7H-pyrrolo [2, 3-c] pyridin-7-one (0.180 g, 0.27 mmol) was dissolved in ethanol (18 mL). sodium hydroxide (0.075 g, 1.89 mmol) was added followed by water (2 mL) at room temperature. The resulting solution was heated at 60° C. for 3 h. TLC (9.5:0.5 DCM:methanol) showed no SM remaining. The resulting solution was directly concentrated to vacuum reduced pressure to a solid. The crude material was purified by reverse phase chromatography, eluting with (70:30) Acetonitrile/water. Fractions corresponding to product were combined and lyophilize to give off white solid. (0.057 g, 42%)

LCMS t_{R} (Waters, Acidic, 4.0 min): 1.513 min, m/z=512.1 [M+H]⁺

HPLC t_{R} (Waters Alliance e2695 with 2998 detector, basic, 17.0 min): 6.15 min

¹H NMR: (400 MHz, DMSO) δ 12.18 (s, 1H), 7.93 (s, 1H), 7.52 (s, 1H), 7.14-7.05 (m, 3H), 6.57 (s, 2H), 6.36 (s, 1H), 5.50 (t, J=6.8 Hz, 1H), 4.80 (t, J=7.2 Hz, 2H), 4.44 (t, J=6.8 Hz, 2H), 3.77 (s, 3H), 3.60 (s, 3H), 2.33 (s, 3H), 2.12 (s, 6H).

### Example 41: 4-(5-(2,6-dimethylphenoxy)-1-(oxetan-3-yl)-2-oxo-1,2-dihydropyridin-4-yl)-2-(2-fluorophenyl)-6-methyl-1,6-dihydro-7H-pyrrolo[2,3-c] pyridin-7-one

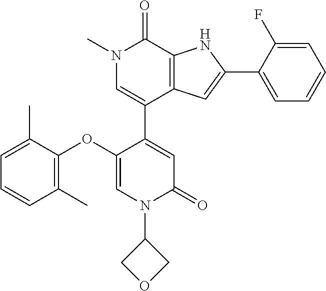 [SMILES: Cc1cccc(C)c1Oc1cn(C2COC2)c(=O)cc1-c1cn(C)c(=O)c2[nH]c(-c3ccccc3F)cc12]

### Preparation 82: 4-bromo-2-(2-fluorophenyl)-6-methyl-1-tosyl-1,6-dihydro-7H-pyrrolo[2,3-c]pyridin-7-one

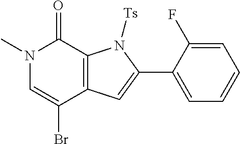 [SMILES: Cc1ccc(S(=O)(=O)n2c(-c3ccccc3F)cc3c(Br)cn(C)c(=O)c32)cc1]

Following the procedure in preparation 78, 4-bromo-2-iodo-6-methyl-1-tosyl-1, 6-dihydro-7H-pyrrolo [2, 3-c] pyridin-7-one (1.5 g, 2.96 mmol) was reacted to give title compound as brown solid (0.85 g, 60%)

LCMS t_{R}: (Waters, Acidic, 4.0 min): 2.327 min, m/z=474.7 [M+H]⁺

¹H NMR: (400 MHz, DMSO) δ 7.99 (s, 1H), 7.85 (d, J=8.4 Hz, 2H), 7.65-7.60 (m, 1H), 7.57-7.52 (m, 1H), 7.39 (d, J=8.4 Hz, 2H), 7.33 (d, J=8 Hz, 2H), 6.67 (s, 1H), 3.40 (s, 3H), 2.38 (s, 3H).

### Preparation 83: 2-(2-fluorophenyl)-6-methyl-4-(4,4,5,5-tetramethyl-1,3,2-dioxaborolan-2-yl)-1-tosyl-1,6-dihydro-7H-pyrrolo[2,3-c] pyridin-7-one

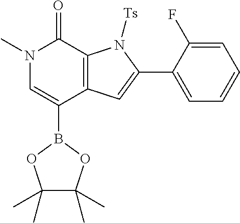 [SMILES: Cc1ccc(S(=O)(=O)n2c(-c3ccccc3F)cc3c(B4OC(C)(C)C(C)(C)O4)cn(C)c(=O)c32)cc1]

Following the procedure in preparation 79, 4-bromo-2-(2-fluorophenyl)-6-methyl-1-tosyl-1,6-dihydro-7H-pyrrolo[2,3-c] pyridin-7-one (0.8 g, 1.68 mmol) was reacted to give title compound as brown sticky solid (1.15 g, Quantitative).

LCMS t_{R} (Waters, Acidic, 4.0 min): 1.764 min, m/z=440.8 [M+H]⁺ (boronic acid) and 2.613 min, m/z=522.9 [M+H]⁺ (boronic ester)

### Preparation 84: 4-(5-(2,6-dimethylphenoxy)-1-(oxetan-3-yl)-2-oxo-1,2-dihydropyridin-4-yl)-2-(2-fluoro phenyl)-6-methyl-1-tosyl-1,6-dihydro-7H-pyrrolo[2,3-c]pyridin-7-one

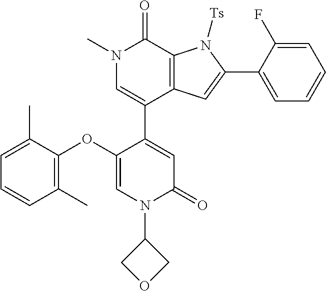 [SMILES: Cc1ccc(S(=O)(=O)n2c(-c3ccccc3F)cc3c(-c4cc(=O)n(C5COC5)cc4Oc4c(C)cccc4C)cn(C)c(=O)c32)cc1]

Following the procedure in preparation 80, 4-bromo-5-(2,6-dimethylphenoxy)-1-(oxetan-3-yl) pyridin-2(1H)-one (0.401 g, 1.14 mmol) and 2-(2-fluorophenyl)-6-methyl-4-(4,4,5,5-tetramethyl-1,3,2-dioxaborolan-2-yl)-1-tosyl-1,6-dihydro-7H-pyrrolo[2,3-c] pyridin-7-one (1.2 g, 2.29 mmol) was reacted to give title compound as brown liquid (0.18 g, 12%).

LCMS t_{R}: (Waters, Acidic, 4.0 min): 2.111 min, m/z=666.2 [M+H]⁺

¹H NMR: (400 MHz, DMSO) δ 7.82 (m, 2H), 7.53 (m, 2H), 7.37 (d, J=8 Hz, 2H), 7.32-7.29 (m, 3H), 7.13-7.08 (m, 3H), 6.71 (s, 1H), 6.55 (s, 1H), 6.52 (s, 1H), 5.58-5.54 (m, 1H), 4.78 (dd, J=7.2 Hz, J=14.4 Hz, 2H), 4.40 (dd, J=6.8 Hz, J=13.2 Hz, 2H), 3.50 (s, 3H), 2.39 (s, 3H), 2.04 (s, 6H).

### Preparation 85: 4-(5-(2,6-dimethylphenoxy)-1-(oxetan-3-yl)-2-oxo-1,2-dihydropyridin-4-yl)-2-(2-fluorophenyl)-6-methyl-1,6-dihydro-7H-pyrrolo[2,3-c] pyridin-7-one

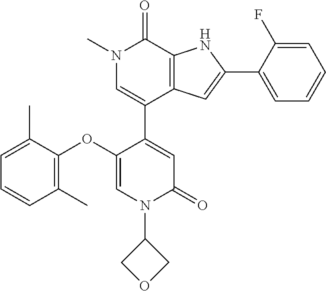 [SMILES: Cc1cccc(C)c1Oc1cn(C2COC2)c(=O)cc1-c1cn(C)c(=O)c2[nH]c(-c3ccccc3F)cc12]

Following the procedure in preparation 81, 4-(5-(2,6-dimethylphenoxy)-1-(oxetan-3-yl)-2-oxo-1,2-dihydropyridin-4-yl)-2-(2-fluorophenyl)-6-methyl-1-tosyl-1,6-dihydro-7H-pyrrolo[2,3-c] pyridin-7-one (0.180 g, 0.27 mmol) and sodium hydroxide (0.075 g, 1.89 mmol) were reacted to give the title compound (50.68 mg, 37%).

LCMS t_{R} (Water, basic, 17 min): 6.62 min, m/z=512.8 [M+H]⁺

HPLC t_{R} (Waters Alliance e2695 with 2998 detector, basic, 17.0 min): 7.597 min

¹H NMR: (400 MHz, DMSO) δ 12.24 (s, 1H), 8.07 (t, J=7.6 Hz, J=15.6 Hz, 1H), 7.52 (s, 1H), 7.41-7.39 (m, 1H), 7.33-7.29 (m, 2H), 7.20-7.06 (m, 3H), 6.83 (d, J=2.8 Hz, 1H), 6.61 (s, 1H), 6.58 (s, 1H), 5.52 (m, 1H), 4.83 (t, J=7.2 Hz, J=14.4 Hz, 2H), 4.46 (t, J=6.8 Hz, J=13.2 Hz, 2H), 3.63 (s, 3H), 2.14 (s, 6H).

### Example 42: 4-(1-(azetidin-3-yl)-5-(2,6-dimethylphenoxy)-2-oxo-1,2-dihydropyridin-4-yl)-2-(1-isopropyl-3-methyl-1H-pyrazol-4-yl)-6-methyl-1,6-dihydro-7H-pyrrolo[2,3-c]pyridin-7-one

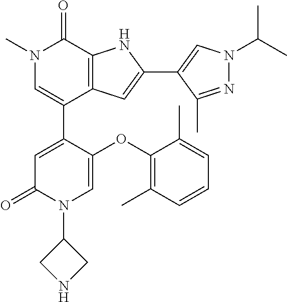 [SMILES: Cc1cccc(C)c1Oc1cn(C2CNC2)c(=O)cc1-c1cn(C)c(=O)c2[nH]c(-c3cn(C(C)C)nc3C)cc12]

### Preparation 86: 1-isopropyl-3-methyl-4-(4, 4, 5, 5-tetramethyl-1, 3, 2-dioxaborolan-2-yl)-1H-pyrazole and 1-isopropyl-5-methyl-4-(4,4,5,5-tetramethyl-1,3,2-dioxaborolan-2-yl)-1H-pyrazole

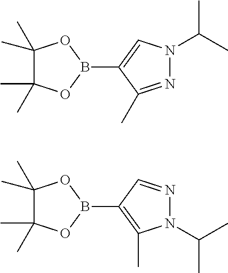

3-methyl-4-(4,4,5,5-tetramethyl-1,3,2-dioxaborolan-2-yl)-1H-pyrazole (5 g, 24.03 mmol) in acetonitrile (120 mL) was added Cesium carbonate (31.3 g, 96.12 mmol) under nitrogen at room temperature. Isopropyl iodide (5.04 g, 72.09 mmol) was added to the reaction mixture at room temperature. The resulting reaction mixture was allowed to stir at 90° C. for 12 h. TLC (5:5 Hexane: ethyl acetate) showed no SM remaining. The resulting solution was directly concentrated to vacuum reduced pressure and added ethyl acetate. The mixture was filtered; filtrate was evaporated to oil. The product was purified by flash chromatography on silica gel eluting with ethyl acetate/Hexane gradient (0-30%) to afford crude material as mixture of isomers (3.8 g, 60%). The crude material was used for the next step without further purification.

LCMS t_{R}: (Waters, Acidic, 4.0 min): 1.902 min & 1.951 min, m/z=250.8 [M+H]⁺

### Preparation 87: 4-bromo-2-(1-isopropyl-3-methyl-1H-pyrazol-4-yl)-6-methyl-1-tosyl-1,6-dihydro-7H-pyrrolo[2,3-c] pyridin-7-one and 4-bromo-2-(1-isopropyl-5-methyl-1H-pyrazol-4-yl)-6-methyl-1-tosyl-1,6-dihydro-7H-pyrrolo[2,3-c]pyridin-7-one

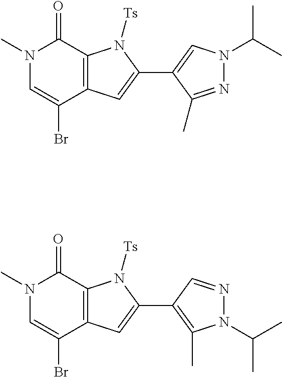

Following the procedure in preparation 78, 4-bromo-2-iodo-6-methyl-1-tosyl-1,6-dihydro-7H-pyrrolo[2,3-c] pyridin-7-one (2 g, 3.95 mmol) and 1-isopropyl-3-methyl-4-(4,4,5,5-tetramethyl-1,3,2-dioxaborolan-2-yl)-1H-pyrazole and 1-isopropyl-5-methyl-4-(4,4,5,5-tetramethyl-1,3,2-dioxaborolan-2-yl)-1H-pyrazole mixture of isomer (2.74 g, 10.99 mmol) was reacted to give title compound as mixture of isomer (1.5 g, 76%).

LCMS t_{R} (Waters, Acidic, 4.0 min): 1.975 min & 2.002 min, m/z=502.8 [M+H]⁺

¹H NMR: (400 MHz, DMSO) δ 7.88 (s, 1H), 7.77 (s, 1H), 7.63 (m, 2H), 7.36 (d, J=8 Hz, 2H), 7.41-7.38 (m, 1H), 3.94 (s, 1H), 3.50 (s, 3H), 2.37 (s, 3H), 2.05 (s, 3H), 1.50 (d, J=6.8 Hz, 6H).

### Preparation 88: 2-(1-isopropyl-3-methyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c] pyridin-7-one and 2-(1-isopropyl-5-methyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c]pyridin-7-one

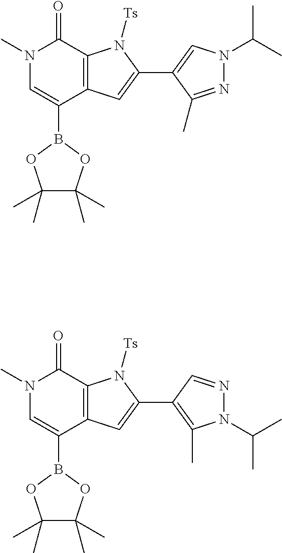

Following the procedure in preparation 79, 4-bromo-2-(1-isopropyl-3-methyl-1H-pyrazol-4-yl)-6-methyl-1-tosyl-1, 6-dihydro-7H-pyrrolo [2, 3-c] pyridin-7-one (1.5 g, 29.87 mmol) was reacted to give title compound as mixture of isomer as brown sticky solid (3 g, Quantitative).

LCMS t_{R} (Waters, Acidic, 4.0 min): 2.314 min & 2.346 min, m/z=551.0 [M+H]⁺ (boronic acid) and 1.501 min & 1.528 min, m/z=468.8 [M+H]⁺ (boronic ester) Preparation 89: Tert-butyl 3-(5-(2,6-dimethylphenoxy)-4-(2-(1-isopropyl-3-methyl-1H-pyrazol-4-yl)-6-methyl-7-oxo-1-tosyl-6,7-dihydro-1H-pyrrolo[2,3-c] pyridin-4-yl)-2-oxopyridin-1(2H)-yl) azetidine-1-carboxylate and tert-butyl 3-(5-(2,6-dimethylphenoxy)-4-(2-(1-isopropyl-5-methyl-1H-pyrazol-4-yl)-6-methyl-7-oxo-1-tosyl-6,7-dihydro-1H-pyrrolo[2,3-c]pyridin-4-yl)-2-oxopyridin-1(2H)-yl)azetidine-1-carboxylate

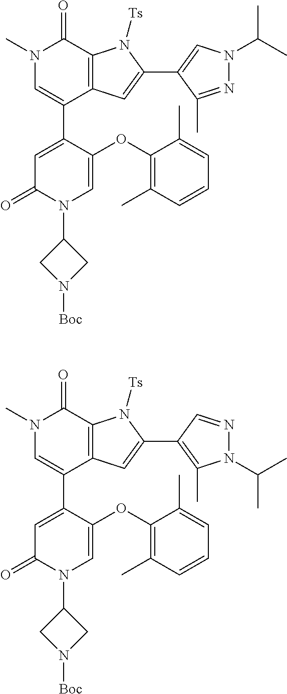

Following the procedure in preparation 80, Tert-butyl 3-(4-bromo-5-(2,6-dimethylphenoxy)-2-oxopyridin-1(2H)-yl) azetidine-1-carboxylate (0.70 g, 1.58 mmol) and 2-(1-isopropyl-3-methyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c] pyridin-7-one and 2-(1-isopropyl-5-methyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c]pyridin-7-one (2 g, 3.63 mmol) was reacted to give title compound as mixture of isomers as brown liquid (0.55 g, 20%).

LCMS t_{R} (Waters, Acidic, 4.0 min): 2.236 min & 2.267 min, m/z=793.2 [M+H]⁺

### Preparation 90: Tert-butyl 3-(5-(2,6-dimethylphenoxy)-4-(2-(1-isopropyl-3-methyl-1H-pyrazol-4-yl)-6-methyl-7-oxo-6,7-dihydro-1H-pyrrolo[2,3-c] pyridin-4-yl)-2-oxopyridin-1(2H)-yl) azetidine-1-carboxylate and tert-butyl 3-(5-(2,6-dimethylphenoxy)-4-(2-(1-isopropyl-5-methyl-1H-pyrazol-4-yl)-6-methyl-7-oxo-6,7-dihydro-1H-pyrrolo[2,3-c]pyridin-4-yl)-2-oxopyridin-1(2H)-yl)azetidine-1-carboxylate

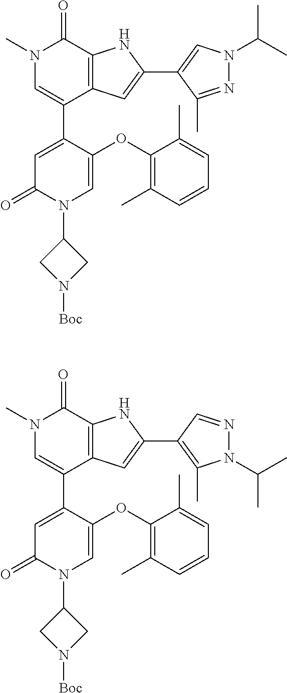

Following the procedure in preparation 81, Tert-butyl 3-(5-(2,6-dimethylphenoxy)-4-(2-(1-isopropyl-3-methyl-1H-pyrazol-4-yl)-6-methyl-7-oxo-1-tosyl-6,7-dihydro-1H-pyrrolo[2,3-c]pyridin-4-yl)-2-oxopyridin-1(2H)-yl) azetidine-1-carboxylate and tert-butyl 3-(5-(2,6-dimethylphenoxy)-4-(2-(1-isopropyl-5-methyl-1H-pyrazol-4-yl)-6-methyl-7-oxo-1-tosyl-6,7-dihydro-1H-pyrrolo[2,3-c]pyridin-4-yl)-2-oxopyridin-1(2H)-yl)azetidine-1-carboxylate (0.550 g, 0.69 mmol) was reacted to give crude material. The crude material was purified by prep HPLC purification using Instrument: A with Column: C with eluting 0.05% ammonium hydroxide in water and acetonitrile. Lyophilized gave yellow liquid of mixture of two isomers (0.38 g). The material was purified by SFS purification using Instrument PHP-04-Agilent 1260 Series infinity UV Detector and column CHIRALPAK IG, 250×10 mm, 5 μm eluting with methanol. Solvent reduction of separate fractions gave Tert-butyl 3-(5-(2,6-dimethylphenoxy)-4-(2-(1-isopropyl-3-methyl-1H-pyrazol-4-yl)-6-methyl-7-oxo-6,7-dihydro-1H-pyrrolo[2,3-c] pyridin-4-yl)-2-oxopyridin-1(2H)-yl) azetidine-1-carboxylate as off white solid (0.050 g, 15%) and Tert-butyl 3-(5-(2,6-dimethylphenoxy)-4-(2-(1-isopropyl-5-methyl-1H-pyrazol-4-yl)-6-methyl-7-oxo-6,7-dihydro-1H-pyrrolo[2,3-c] pyridin-4-yl)-2-oxopyridin-1(2H)-yl) azetidine-1-carboxylate (0.050 g, 15%).

Isomer-1: LCMS t_{R} (Waters, Acidic, 4.0 min): 1.991 min, m/z=639.2 [M+H]⁺

HPLC t_{R} (Waters Alliance e2695 with 2998 detector, basic, 17.0 min): 8.214 min

¹H NMR: (400 MHz, DMSO) δ 12.06 (s, 1H), 8.36 (s, 1H), 7.51 (s, 1H), 7.12 (d, J=7.2 Hz, 2H), 7.06-7.03 (m, 1H), 6.56 (s, 1H), 6.49 (s, 1H), 6.37 (d, J=2 Hz, 1H), 5.15 (m, 1H), 4.40 (m, 1H), 4.13-4.10 (m, 2H), 3.89 (m, 2H), 3.59 (s, 3H), 2.33 (s, 3H), 2.11 (s, 6H), 1.41 (d, J=6.8 Hz, 6H), 1.35 (s, 9H).

Isomer-2: LCMS t_{R} (Waters, Acidic, 4.0 min): 2.010 min, m/z=639.3 [M+H]⁺

HPLC t_{R} (Waters Alliance e2695 with 2998 detector, basic, 17.0 min): 8.263 min

¹H NMR: (400 MHz, DMSO) δ 12.17 (s, 1H), 7.98 (s, 1H), 7.52 (s, 1H), 7.12 (d, J=7.6 Hz, 2H), 7.05 (m, 1H), 6.57 (s, 1H), 6.49 (s, 1H), 6.36 (s, 1H), 5.17-5.14 (m, 1H), 4.59 (m, 1H), 4.11 (m, 2H), 3.88 (m, 2H), 3.59 (s, 3H), 2.42 (s, 3H), 2.11 (s, 6H), 1.41 (d, J=6.8 Hz, 6H), 1.35 (s, 9H).

### Preparation 91: 4-(1-(azetidin-3-yl)-5-(2,6-dimethylphenoxy)-2-oxo-1,2-dihydropyridin-4-yl)-2-(1-isopropyl-3-methyl-1H-pyrazol-4-yl)-6-methyl-1,6-dihydro-7H-pyrrolo[2,3-c] pyridin-7-one

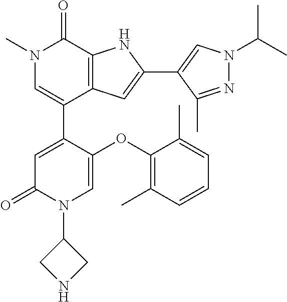 [SMILES: Cc1cccc(C)c1Oc1cn(C2CNC2)c(=O)cc1-c1cn(C)c(=O)c2[nH]c(-c3cn(C(C)C)nc3C)cc12]

Tert-butyl 3-(5-(2,6-dimethylphenoxy)-4-(2-(1-isopropyl-3-methyl-1H-pyrazol-4-yl)-6-methyl-7-oxo-6,7-dihydro-1H-pyrrolo[2,3-c] pyridin-4-yl)-2-oxopyridin-1(2H)-yl) azetidine-1-carboxylate (0.050 g, 0.078 mmol) was dissolved in DCM at room temperature under nitrogen. The resulting mixture was cooled at 0° C. and added TFA (0.50 mL, 10V). The reaction mixture allowed stirring at room temperature for 4 h. TLC (9:1 DCM: Methanol) showed no SM remaining. The resulting mixture was concentrated under vacuum to afford crude material. The crude material was triturated by diethyl ether to afford title compound as off white solid (0.022 g, 8%).

LCMS t_{R} (Water, Acidic, 4.0 min): 1.328 min, m/z=539.1 [M+H]⁺

HPLC t_{R} (Waters Alliance e2695 with 2998 detector, basic, 17.0 min): 7.298 min

¹H NMR: (400 MHz, DMSO) δ 12.01 (bs, 1H), 8.30 (s, 1H), 7.46 (s, 1H), 7.20-7.061 (m, 3H), 6.67 (s, 1H), 6.45 (s, 1H), 6.36 (s, 1H), 5.13-4.96 (m, 2H), 4.45-4.40 (m, 3H), 4.27-4.22 (m, 2H), 3.61 (s, 3H), 2.34 (s, 3H), 2.12 (s, 6H), 1.45 (d, J=6.4 Hz, 6H).

### Example: 43: 4-(1-(azetidin-3-yl)-5-(2,6-dimethylphenoxy)-2-oxo-1,2-dihydropyridin-4-yl)-2-(1-isopropyl-5-methyl-1H-pyrazol-4-yl)-6-methyl-1,6-dihydro-7H-pyrrolo[2,3-c] pyridin-7-one

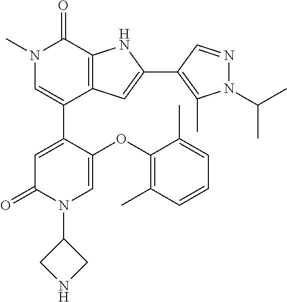 [SMILES: Cc1cccc(C)c1Oc1cn(C2CNC2)c(=O)cc1-c1cn(C)c(=O)c2[nH]c(-c3cnn(C(C)C)c3C)cc12]

### Preparation 92: 4-(1-(azetidin-3-yl)-5-(2,6-dimethylphenoxy)-2-oxo-1,2-dihydropyridin-4-yl)-2-(1-isopropyl-5-methyl-1H-pyrazol-4-yl)-6-methyl-1,6-dihydro-7H-pyrrolo[2,3-c] pyridin-7-one

Following the procedure in preparation 91, Tert-butyl 3-(5-(2,6-dimethylphenoxy)-4-(2-(1-isopropyl-5-methyl-1H-pyrazol-4-yl)-6-methyl-7-oxo-6,7-dihydro-1H-pyrrolo[2,3-c] pyridin-4-yl)-2-oxopyridin-1(2H)-yl) azetidine-1-carboxylate (0.050 g, 0.078 mmol) was reacted to give title compound as pale yellow solid (0.028 g, 10%).

LCMS t_{R} (Water, Acidic, 4.0 min): 1.332 min, m/z=539.1 [M+H]⁺

HPLC t_{R} (Waters Alliance e2695 with 2998 detector, basic, 17.0 min): 7.381 min

¹H NMR: (400 MHz, DMSO) δ 12.12 (bs, 1H), 8.68 (m, 1H), 7.93 (s, 1H), 7.47 (s, 1H), 7.21-7.04 (m, 3H), 6.67-6.34 (m, 2H), 5.40 (m, 1H), 5.15 (d, J=7.2 Hz, 2H), 4.95 (m, 1H), 4.60 (m, 1H), 4.25 (m, 1H), 3.61 (s, 3H), 2.43 (s, 3H), 2.12 (s, 6H), 1.44 (d, J=6.4 Hz, 6H)

### Example: 44: 4-(5-(2,6-dimethylphenoxy)-1-(oxetan-3-yl)-2-oxo-1,2-dihydropyridin-4-yl)-2-(1-isopropyl-3-methyl-1H-pyrazol-4-yl)-6-methyl-1-tosyl-1,6-dihydro-7H-pyrrolo[2,3-c] pyridin-7-one

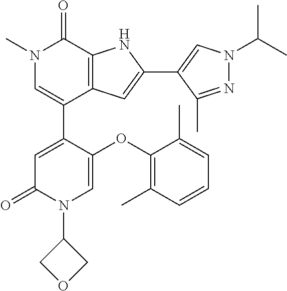 [SMILES: Cc1cccc(C)c1Oc1cn(C2COC2)c(=O)cc1-c1cn(C)c(=O)c2[nH]c(-c3cn(C(C)C)nc3C)cc12]

### Preparation 93: 4-(5-(2,6-dimethylphenoxy)-1-(oxetan-3-yl)-2-oxo-1,2-dihydropyridin-4-yl)-2-(1-isopropyl-3-methyl-1H-pyrazol-4-yl)-6-methyl-1-tosyl-1,6-dihydro-7H-pyrrolo[2,3-c]pyridin-7-one

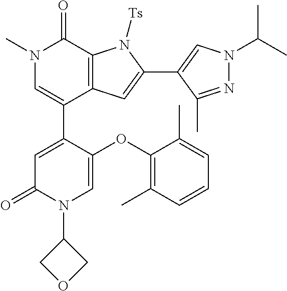 [SMILES: Cc1ccc(S(=O)(=O)n2c(-c3cn(C(C)C)nc3C)cc3c(-c4cc(=O)n(C5COC5)cc4Oc4c(C)cccc4C)cn(C)c(=O)c32)cc1]

Following the procedure in preparation 80, 4-bromo-5-(2,6-dimethylphenoxy)-1-(oxetan-3-yl) pyridin-2(1H)-one (0.50 g, 1.43 mmol) and 2-(1-isopropyl-3-methyl-1H-pyrazol-4-yl)-6-methyl-4-(4,4,5,5-tetramethyl-1,3,2-dioxaborolan-2-yl)-1-tosyl-1,6-dihydro-7H-pyrrolo[2,3-c]pyridin-7-one (1.57 g, 2.86 mmol) was reacted to give title compound as brown liquid (0.45 g, 24%).

LCMS t_{R} (Waters, Acidic, 4.0 min): 1.882 min & 1.909 min, m/z=694.2 [M+H]⁺

### Preparation 94: 4-(5-(2,6-dimethylphenoxy)-1-(oxetan-3-yl)-2-oxo-1,2-dihydropyridin-4-yl)-2-(1-isopropyl-3-methyl-1H-pyrazol-4-yl)-6-methyl-1,6-dihydro-7H-pyrrolo[2,3-c] pyridin-7-one

Following the procedure in preparation 81, 4-(5-(2,6-dimethylphenoxy)-1-(oxetan-3-yl)-2-oxo-1,2-dihydropyridin-4-yl)-2-(1-isopropyl-3-methyl-1H-pyrazol-4-yl)-6-methyl-1-tosyl-1,6-dihydro-7H-pyrrolo[2,3-c] pyridin-7-one (0.4 g, 0.57 mmol) was reacted to give crude material. The crude material was purified by prep HPLC purification using Instrument: A with Column: C with eluting 0.05% ammonium hydroxide in water and 20% a-line in acetonitrile.

Lyophilized gave yellow liquid of mixture of two isomers. The material was purified by SFS purification using Instrument PHP-04-Agilent 1260 Series infinity UV Detector and column CHIRALPAK IG, 250×10 mm, 5 μm eluting with 0.1% ammonia in heptane and IPA in acetonitrile. Solvent reduction of separate fractions gave title compound off white solid (0.012 g, 11%).

LCMS t_{R} (Waters, Acidic, 4.0 min): 1.994 min, m/z=540.4 [M+H]⁺

HPLC t_{R} (Waters Alliance e2695 with 2998 detector, basic, 17.0 min): 7.01 min

¹H NMR: (400 MHz, DMSO) δ 12.05 (s, 1H), 8.36 (s, 1H), 7.51 (s, 1H), 7.14-7.05 (m, 3H), 6.57 (d, J=4.8 Hz, 2H), 6.37 (s, 1H), 5.50 (m, 1H), 4.80 (dd, J=7.6 Hz, J=14.8 Hz, 2H), 4.46-4.38 (m, 3H), 3.59 (s, 3H), 2.33 (s, 3H), 2.12 (s, 6H), 1.41 (d, J=6.4 Hz, 6H).

### Example 45: 4-(5-(2,6-dimethylphenoxy)-1-methyl-2-oxo-1,2-dihydropyridin-4-yl)-6-methyl-2-(2-methylpyridin-4-yl)-1,6-dihydro-7H-pyrrolo[2,3-c]pyridin-7-one

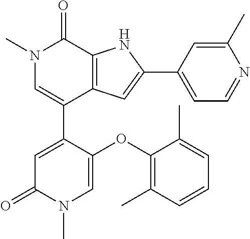

### Preparation 95: 4-bromo-2-chloro-7-methoxy-1-tosyl-1H-pyrrolo[2,3-c] pyridine

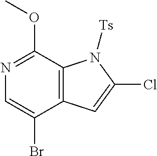 [SMILES: COc1ncc(Br)c2cc(Cl)n(S(=O)(=O)c3ccc(C)cc3)c12]

4-bromo-7-methoxy-1-tosyl-1H-pyrrolo [2, 3-c] pyridine (200 g, 526.3 mmol) was dissolved in dry THF (4000 mL, 20V) at room temperature. LDA (1M in THF) (684 mL, 684.2 mmol) was dropwise added to the reaction mixture at −78° C. for 30 min. The resulting reaction mixture was allowed to stir at −78° C. for 2 h. Hexachloroethane (199.3 g, 842 mmol) in dry THF (1000 mL) was dropwise added to the reaction mixture at −78° C. for 15 min. The resulting reaction mixture was allowed to stir at −78° C. to rt for 4 h. TLC (2:8 Ethyl acetate/Hexane) showed SM consumed. The reaction mixture was quenched in NH4Cl solution (3000 mL) aqueous was extracted with ethyl acetate (3×2000 mL). The Organic fraction was dried over Na2SO4, filtered and evaporated to yield 4-bromo-2-chloro-7-methoxy-1-tosyl-1H-pyrrolo[2,3-c] pyridine (160 g, 68%) as a White solid.

LCMS t_{R}: (Waters, Acidic, 4.0 min): 2.723 min, m/z=416.7 [M+H]+

1H NMR: (400 MHz, DMSO-d6) δ 8.08 (s, 1H), 7.96-7.94 (d, J=8.4 Hz, 2H), 7.54-7.52 (d, J=8.0 Hz, 2H), 7.05 (s, 1H), 3.87 (s, 3H), 2.42 (s, 3H).

### Preparation 96: 4-bromo-2-chloro-1-tosyl-1,6-dihydro-7H-pyrrolo[2,3-c]pyridin-7-one

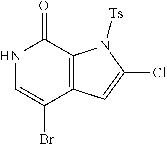 [SMILES: Cc1ccc(S(=O)(=O)n2c(Cl)cc3c(Br)c[nH]c(=O)c32)cc1]

4-bromo-2-chloro-7-methoxy-1-tosyl-1H-pyrrolo[2,3-c] pyridine (320 g, 772.9 mmol) was dissolved in Acetonitrile (3200 mL, 10V) at room temperature. Sodium iodide (173.8, 1159.4 mmol) was added to the reaction mixture and allowed stir at room temperature for 15 min. The resulting solution was cooled at 0° C. and dropwise added Trimethylsilyl chloride (147.2 mL, 1159.4 mmol). The resulting mixture was allowed to stir at ambient temperature for 1 h. Water (160 mL, 0.5V) was added to the reaction mixture and heated at 65° C. for 3 h. TLC (5.0:5.0 Hexane:Ethyl acetate) showed SM was consumed. The resulting mixture was quenched with ice-cold water (3200 mL) and stirred for 30 min. The resulting residue was filtered and triturated by n-hexane (320 mL) & diethyl ether (320 mL). The solid was dried overnight under vacuum at 45° C. to give white solid (295 g, 95%).

LCMS t_{R}: (Waters, Acidic, 4.0 min): 2.090 min, m/z=402.7 [M+H]⁺

¹H NMR (400 MHz, DMSO-d6) δ 11.66 (s, 1H), 8.12-8.10 (d, J=8.4 Hz, 2H), 7.50-7.48 (d, J=8.0 Hz, 2H), 7.45 (s, 1H), 6.79 (s, 1H), 2.41 (s, 3H)

### Preparation 97: 4-bromo-2-chloro-6-methyl-1-tosyl-1,6-dihydro-7H-pyrrolo[2,3-c]pyridin-7-one

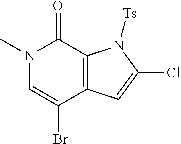 [SMILES: Cc1ccc(S(=O)(=O)n2c(Cl)cc3c(Br)cn(C)c(=O)c32)cc1]

4-bromo-2-chloro-1-tosyl-1,6-dihydro-7H-pyrrolo[2,3-c] pyridin-7-one (295 g, 737.6 mmol) was dissolved in DMF (5900 mL, 20V) at room temperature. Potassium carbonate (203 g, 1475 mmol) was portion wise added to the reaction mixture at 0° C. and the resulting solution was allowed to stir at same temperature for 30 min. Methyl iodide (69.2 mL, 1106.4 mmol) was dropwise added to the reaction mixture at 0° C. and allows to stir at room temperature for 4 h. TLC (5:5 Ethyl acetate/Hexane) after 4 h showed SM was consumed. The reaction mixture was quenched with ice cold water (5900 mL) to afford yellow coloured precipitate. The resulting precipitate was filtered and washed with water (3000 mL) and hexane (3000 mL). The solid was dried overnight under vacuum at 45° C. to give white solid (270 g, 88%)

LCMS t_{R}: (Waters, Acidic, 4.0 min): 2.243 min, m/z=416.7[M+H]⁺.

1H NMR: (400 MHz, DMSO-d6) δ 8.13 (d, J=8.4 Hz, 2H), 7.91 (s, 1H), 7.52-7.49 (d, J=8.0 Hz, 2H), 6.81 (s, 1H), 3.43 (s, 3H), 2.42 (s, 3H).

### Preparation 98: 2-chloro-6-methyl-1-(4-methylbenzenesulfonyl)-4-(4,4,5,5-tetramethyl-1,3,2-dioxaborolan-2-yl) pyrrolo [2,3-c] pyridin-7-one

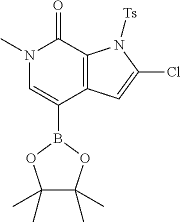 [SMILES: Cc1ccc(S(=O)(=O)n2c(Cl)cc3c(B4OC(C)(C)C(C)(C)O4)cn(C)c(=O)c32)cc1]

To a stirred mixture of 4-bromo-2-chloro-6-methyl-1-(4-methylbenzenesulfonyl) pyrrolo [2,3-c]pyridin-7-one (10.0 g, 24.1 mmol, 1.00 eq.) and bis(pinacolato)diboron (36.7 g, 144 mmol, 6.00 eq.) in THF (150 mL) were added KOAc (4.72 g, 48.1 mmol, 2.00 eq.) and Pd(PPh3)2Cl2 (1.69 g, 2.41 mmol, 0.100 eq.) at room temperature under nitrogen atmosphere. The resulting mixture was stirred for overnight at 60° C. under nitrogen atmosphere. The LCMS showed no SM remaining. The reaction was quenched with water. The resulting mixture was extracted with CH2Cl2 (3×150 mL). The combined organic layers were washed with brine, dried over anhydrous Na2SO4. After filtration, the filtrate was concentrated under reduced pressure. The residue was purified by silica gel column chromatography, eluted with PE/EA (3:1) to afford crude product as a yellow solid. The crude product was further purified by reversed phase flash chromatography with the following conditions: column, C18; mobile phase, MeCN in Water (0.1% FA), 50% to 90% gradient in 15 min; detector, UV 254 nm. This resulted in 2-chloro-6-methyl-1-(4-methylbenzenesulfonyl)-4-(4,4,5,5-tetramethyl-1,3,2-dioxaborolan-2-yl) pyrrolo[2,3-c]pyridin-7-one (4.30 g, 38.6%) as a light yellow solid

LCMS m/z=463 [M+H]⁺

1H NMR (300 MHz, DMSO-d6) δ 8.14-8.08 (m, 2H), 7.82 (s, 1H), 7.52-7.47 (m, 2H), 6.84 (s, 1H), 3.48 (s, 3H), 2.42 (s, 3H), 1.29 (s, 12H).

### Preparation 99: 4-[2-chloro-6-methyl-1-(4-methylbenzenesulfonyl)-7-oxopyrrolo [2,3-c]pyridin-4-yl]-5-(2,6-dimethylphenoxy)-1-methylpyridin-2-one

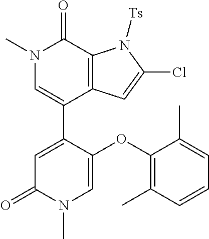 [SMILES: Cc1ccc(S(=O)(=O)n2c(Cl)cc3c(-c4cc(=O)n(C)cc4Oc4c(C)cccc4C)cn(C)c(=O)c32)cc1]

To a stirred mixture of 2-chloro-6-methyl-1-(4-methylbenzenesulfonyl)-4-(4,4,5,5-tetramethyl-1,3,2-dioxaborolan-2-yl)pyrrolo [2,3-c]pyridin-7-one (1.50 g, 3.24 mmol, 1.00 eq.) and 4-bromo-5-(2,6-dimethylphenoxy)-1-methylpyridin-2-one (1.20 g, 3.89 mmol, 1.20 eq.) in DME (1.50 mL) and H₂O (0.30 mL) was added Na₂CO₃ (690 mg, 6.48 mmol, 2.00 eq.) and Pd(dppf)Cl₂·CH₂Cl₂ (260 mg, 0.324 mmol, 0.10 eq.) at room temperature under nitrogen atmosphere. The resulting mixture was stirred overnight at 60° C. under nitrogen atmosphere. The LCMS showed no SM remaining. The mixture was allowed to cool down to room temperature, and then quenched with water. The resulting mixture was extracted with EtOAc (3×30 mL). The combined organic layers were washed with brine, dried over anhydrous Na2SO4. After filtration, the filtrate was concentrated under reduced pressure. The residue was purified by silica gel column chromatography, eluted with EtOAc/MeOH (12:1) to afford 4-[2-chloro-6-methyl-1-(4-methylbenzenesulfonyl)-7-oxopyrrolo [2,3-c]pyridin-4-yl]-5-(2,6-dimethylphenoxy)-1-methylpyridin-2-one (910 mg, 50%) as a yellow solid.

LCMS: m/z=564 [M+H]⁺

### Preparation 100: 4-{2-chloro-6-methyl-7-oxo-1H-pyrrolo[2,3-c]pyridin-4-yl}-5-(2,6-dimethyphenoxy)-1-methylpyridin-2-one

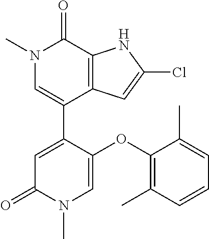 [SMILES: Cc1cccc(C)c1Oc1cn(C)c(=O)cc1-c1cn(C)c(=O)c2[nH]c(Cl)cc12]

To the mixture of 4-[2-chloro-6-methyl-1-(4-methylbenzenesulfonyl)-7-oxopyrrolo[2,3-c]pyridin-4-yl]-5-(2,6-dimethylphenoxy)-1-methylpyridin-2-one (910 mg, 1.61 mmol, 1.00 eq.) in 1,4-dioxane (5.0 mL) and H2O (1.0 mL) was added NaOH (645 mg, 16.1 mmol, 10.0 eq.). The mixture was stirred for 1 h at 60° C. The LCMS showed no SM remaining. The mixture was allowed to cool down to room temperature. The reaction was quenched with water at room temperature. The resulting mixture was extracted with EtOAc (3×30.0 mL). The combined organic layers were washed with brine, dried over anhydrous Na2SO4. After filtration, the filtrate was concentrated under reduced pressure. The residue was purified by silica gel column chromatography, eluted with EtOAc/MeOH (12:1) to afford 4-{2-chloro-6-methyl-7-oxo-1H-pyrrolo[2,3-c] pyridin-4-yl}-5-(2,6-dimethylphenoxy)-1-methylpyridin-2-one (328 mg, 49.7%) as a yellow solid.

LCMS: m/z=410 [M+H]⁺

### Preparation 101: 5-(2,6-dimethylphenoxy)-1-methyl-4-[6-methyl-2-(2-methypyridin-4-yl)-7-oxo-1H-pyrrolo [2,3-c] pyridin-4-yl] pyridin-2-one

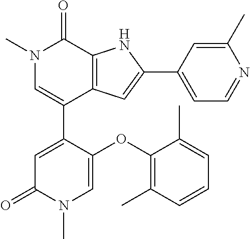

To a stirred mixture of 4-{2-chloro-6-methyl-7-oxo-1H-pyrrolo [2,3-c] pyridin-4-yl}-5-(2,6-dimethylphenoxy)-1-methylpyridin-2-one (60.0 mg, 0.146 mmol, 1.00 equiv) and 2-methylpyridin-4-ylboronic acid (40.1 mg, 0.292 mmol, 2.00 equiv) in DME (1.00 mL) and H2O (0.20 mL) were added K2CO3 (40.5 mg, 0.292 mmol, 2.00 equiv) and XPhos Pd G3 (12.4 mg, 0.0150 mmol, 0.100 equiv) at room temperature under nitrogen atmosphere. The resulting mixture was stirred for 4 h at 100° C. The reaction was quenched with water. The resulting mixture was extracted with EtOAc (3×30 mL). The combined organic layers were washed with brine and water, dried over anhydrous Na₂SO₄. After filtration, the filtrate was concentrated under reduced pressure. The crude product (60.0 mg) was purified by Prep-HPLC (Instrument C; Colum D), eluted with a gradient of 0.1% FA solution in water and acetonitrile, lyophilisation give white solid (40.0 mg). The product was further treated with HCl (g) in MeOH (1.00 mL, 4.0 N), followed by lyophilisation. This resulted in 5-(2,6-dimethylphenoxy)-1-methyl-4-[6-methyl-2-(2-methylpyridin-4-yl)-7-oxo-1H-pyrrolo[2,3-c]pyridin-4-yl]pyridin-2-one hydrochloride (35.0 mg, 48%) as a yellow solid.

LCMS tR (Shimadzu LMCS-2020, A, 2.80 min): 1.11 min, m/z=467.05 [M+H]⁺

1H NMR (400 MHz, DMSO-d6) δ 13.21 (bs, 1H), 8.77 (d, J=6.4 Hz, 1H), 8.53 (d, J=1.9 Hz, 1H), 8.44 (dd, J=6.4, 1.9 Hz, 1H), 7.61 (s, 1H), 7.53 (d, J=2.3 Hz, 1H), 7.14-7.08 (m, 2H), 7.07-7.01 (m, 1H), 6.77 (s, 1H), 6.65 (s, 1H), 3.63 (s, 3H), 3.37 (s, 3H), 2.73 (s, 3H), 2.09 (s, 6H).

### Example 46: 2-(2,5-difluorophenyl)-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one

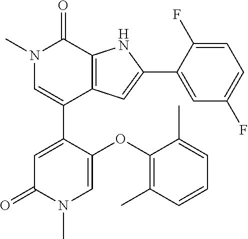 [SMILES: Cc1cccc(C)c1Oc1cn(C)c(=O)cc1-c1cn(C)c(=O)c2[nH]c(-c3cc(F)ccc3F)cc12]

### Preparation 102: 2-(2,5-difluorophenyl)-4-(5-(2,6-dimethylphenoxy)-1-methyl-2-oxo-1,2-dihydropyridin-4-yl)-6-methyl-1,6-dihydro-7H-pyrrolo[2,3-c]pyridin-7-one

Following the procedure in preparation 101, 4-{2-chloro-6-methyl-7-oxo-1H-pyrrolo[2,3-c]pyridin-4-yl}-5-(2,6-dimethylphenoxy)-1-methylpyridin-2-one (70.0 mg, 0.171 mmol, 1.00 equiv) and 2,5-difluorophenylboronic acid (53.9 mg, 0.342 mmol, 2.00 equiv) was reacted to give title compound (33.0 mg, 39.6%) as a white solid after purification by Prep-HPLC (Instrument C; Column A), eluted with a gradient of 10 mmol/L NH4HCO3 solution in water and acetonitrile.

LCMS tR (Shimadzu LMCS-2020, B, 2.80 min): 1.834 min, m/z=488.20 [M+H]⁺

1H NMR (300 MHz, DMSO-d6) δ 12.56 (s, 1H), 8.11-7.97 (m, 1H), 7.54 (s, 1H), 7.45-7.31 (m, 1H), 7.30-7.14 (m, 1H), 7.14-6.98 (m, 3H), 6.88 (d, J=3.8 Hz, 1H), 6.67 (s, 1H), 6.53 (s, 1H), 3.62 (s, 3H), 3.32 (s, 3H), 2.09 (s, 6H).

### Example 47: 2-(2,4-difluorophenyl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

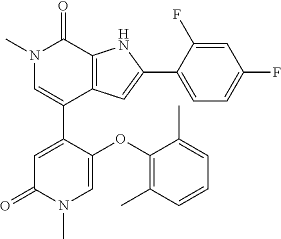 [SMILES: Cc1cccc(C)c1Oc1cn(C)c(=O)cc1-c1cn(C)c(=O)c2[nH]c(-c3ccc(F)cc3F)cc12]

### Preparation 103: 2-(2,4-difluorophenyl)-4-(5-(2,6-dimethylphenoxy)-1-methyl-2-oxo-1,2-dihydropyridin-4-yl)-6-methyl-1,6-dihydro-7H-pyrrolo[2,3-c] pyridin-7-one

Following the procedure in preparation 101, 4-{2-chloro-6-methyl-7-oxo-1H-pyrrolo [2,3-c]pyridin-4-yl}-5-(2,6-dimethylphenoxy)-1-methylpyridin-2-one (70.0 mg, 0.171 mmol, 1.00 equiv) and 2,4-difluorophenylboronic acid (53.9 mg, 0.342 mmol, 2.00 equiv) was reacted to give title compound (26.0 mg, 31.1%) as a white solid after purification by Prep-HPLC (Instrument C; Column A), eluted with a gradient of 0.1% NH₃ solution in water and acetonitrile.

LCMS t_{R} (Shimadzu LMCS-2020, B, 3.00 min): 1.835 min, m/z=488.25 [M+H]⁺

¹H NMR (300 MHz, DMSO-d⁶) δ 12.48 (s, 1H), 8.25-8.00 (m, 1H), 7.54 (s, 1H), 7.47-7.33 (m, 1H), 7.21 (t, J=8.0 Hz, 1H), 7.15-6.99 (m, 3H), 6.76 (d, J=3.6 Hz, 1H), 6.67 (s, 1H), 6.53 (s, 1H), 3.62 (s, 3H), 3.33 (s, 3H), 2.09 (s, 6H).

### Example 48: 2-{4-[5-(2,6-dimethylphenoxy)-1-methyl-2-oxopyridin-4-yl]-6-methyl-7-oxo-1H-pyrrolo[2,3-c]pyridin-2-yl}benzonitrile

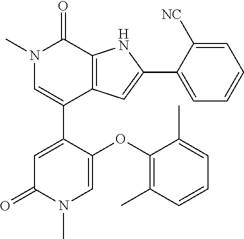 [SMILES: Cc1cccc(C)c1Oc1cn(C)c(=O)cc1-c1cn(C)c(=O)c2[nH]c(-c3ccccc3C#N)cc12]

### Preparation 104: 2-{4-[5-(2,6-dimethylphenoxy)-1-methyl-2-oxopyridin-4-yl]-6-methyl-7-oxo-1H-pyrrolo[2,3-c]pyridin-2-yl}benzonitrile

Following the procedure in preparation 101, 4-{2-chloro-6-methyl-7-oxo-1H-pyrrolo[2,3-c]pyridin-4-yl}-5-(2,6-dimethylphenoxy)-1-methylpyridin-2-one (100 mg, 0.244 mmol, 1.00 equiv) and 2-cyanophenylboronic acid (71.7 mg, 0.488 mmol, 2.00 equiv) was reacted to give title compound (20.0 mg, 17.2%) as a white solid after purification by Prep-HPLC (Instrument C; Column D), eluted with a gradient of 0.1% FA solution in water and acetonitrile.

LCMS t_{R} (Shimadzu LMCS-2020, B, 3.00 min): 1.675 min. m/z=477.30 [M+H]1

¹H NMR (300 MHz, DMSO-d⁶) b 12.73 (s, 1H), 7.95 (d, J=8.0 Hz, 2H), 7.83-7.72 (m, 1H), 7.61-7.50 (m, 2H), 7.14-6.99 (m, 3H), 6.97 (s, 1H), 6.67 (s, 1H), 6.56 (s, 1H), 3.62 (s, 3H), 3.32 (s, 3H), 2.07 (s, 6H).

### Example 49: 4-{4-[5-(2,6-dimethylphenoxy)-1-methyl-2-oxopyridin-4-yl]-6-methyl-7-oxo-1H-pyrrolo[2,3-c]pyridin-2-yl}-1-isopropylpyrazole-3-carbonitrile

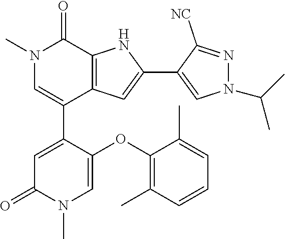 [SMILES: Cc1cccc(C)c1Oc1cn(C)c(=O)cc1-c1cn(C)c(=O)c2[nH]c(-c3cn(C(C)C)nc3C#N)cc12]

### Preparation 105: 4-bromo-1-isopropylpyrazole-3-carbonitrile

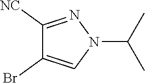

To a solution of 4-bromo-1H-pyrazole-3-carbonitrile (3.00 g, 17.4 mmol, 1.00 eq.) in DMF (20.0 mL) was added NaH (840 mg, 20.9 mmol, 1.20 eq., 60% wt in mineral oil). The mixture was stirred for 30 min at 0° C. followed by the addition of 2-bromopropane (2.45 g, 19.8 mmol, 1.14 eq.). The mixture was warmed up to room temperature and stirred overnight. The reaction was quenched with water. The resulting mixture was extracted with EtOAc (3×30 mL). The combined organic layers were washed with brine, dried over Na2SO4. After filtration, the filtrate was concentrated under reduced pressure. The residue was purified by silica gel column chromatography, eluted with PE/EA (5:1) to afford 4-bromo-1-isopropylpyrazole-3-carbonitrile (2.00 g, 54%) as a light yellow solid.

LCMS: m/z=216 [M+H]+

1H NMR (300 MHz, Chloroform-d) δ 7.52 (s, 1H), 4.61-4.43 (m, 1H), 1.51 (d, J=6.7 Hz, 6H).

### Preparation 106: 3-cyano-1-isopropylpyrazol-4-ylboronic acid

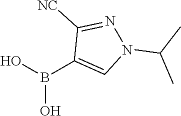

To a solution of 4-bromo-1-isopropylpyrazole-3-carbonitrile (500 mg, 2.34 mmol, 1.00 eq.) in THF (5.00 mL) was added n-BuLi (1.31 mL, 3.27 mmol, 1.40 eq., 2.50 M/L in Hexane) at −78° C. under nitrogen atmosphere. The reaction mixture was stirred for 30 min at −78° C. and followed by the addition of 2-isopropoxy-4,4,5,5-tetramethyl-1,3,2-dioxaborolane (869 mg, 4.67 mmol, 2.00 eq.). The mixture was allowed to warm up to room temperature. The reaction mixture was quenched with sat. NH4Cl. The resulting mixture was extracted with EtOAc (3×20 mL). The combined organic layers were washed with water and brine, dried over anhydrous Na2SO4. After filtration, the filtrate was concentrated under reduced pressure. The residue was purified by silica gel column chromatography, eluted with PE/EA (5:1) to afford 3-cyano-1-isopropylpyrazol-4-ylboronic acid (263 mg, 62.9%) as a light yellow solid.

LCMS: m/z=180 [M+H]⁺

### Preparation 107: 4-{4-[5-(2,6-dimethylphenoxy)-1-methyl-2-oxopyridin-4-yl]-6-methyl-7-oxo-1H-pyrrolo[2,3-c] pyridin-2-yl}-1-isopropylpyrazole-3-carbonitrile

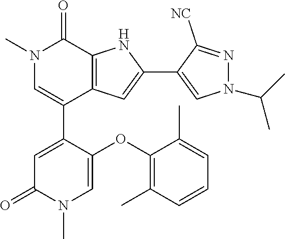 [SMILES: Cc1cccc(C)c1Oc1cn(C)c(=O)cc1-c1cn(C)c(=O)c2[nH]c(-c3cn(C(C)C)nc3C#N)cc12]

Following the procedure in preparation 101, 3-cyano-1-isopropylpyrazol-4-ylboronic acid (60.0 mg, 0.335 mmol, 1.00 eq.) and 4-{2-chloro-6-methyl-7-oxo-1H-pyrrolo[2,3-c]pyridin-4-yl}-5-(2,6-dimethylphenoxy)-1-methylpyridin-2-one (618 mg, 1.51 mmol, 4.50 eq.) was reacted to give title compound (22.0 mg, 12.9%) as a white solid after purification by Prep-HPLC (Instrument C; Column D), eluted with a gradient of 0.1% FA solution in water and acetonitrile.

LCMS t_{R} (Shimadzu LCMS-2020, A, 2.80 min): 1.599 min, m/z=509 [M+H]⁺

¹H NMR (300 MHz, Methanol-d⁴) δ 8.37 (s, 1H), 7.55 (s, 1H), 7.17-7.02 (m, 3H), 6.92 (s, 1H), 6.79 (s, 1H), 6.69 (s, 1H), 4.74-4.63 (m, 1H), 3.75 (s, 3H), 3.49 (s, 3H), 2.17 (s, 6H), 1.59 (d, J=6.7 Hz, 6H).

### Example 50: 4-[2-(2,3-difluorophenyl)-6-methyl-7-oxo-1H-pyrrolo[2,3-c]pyridin-4-yl]-5-(2,6-dimethylphenoxy)-1-methylpyridin-2-one

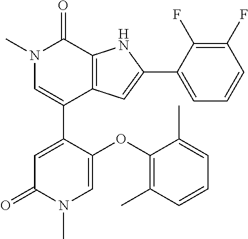 [SMILES: Cc1cccc(C)c1Oc1cn(C)c(=O)cc1-c1cn(C)c(=O)c2[nH]c(-c3cccc(F)c3F)cc12]

### Preparation 108: 4-[2-(2,3-difluorophenyl)-6-methyl-7-oxo-1H-pyrrolo[2,3-c]pyridin-4-yl]-5-(2,6-dimethylphenoxy)-1-methylpyridin-2-one

Following the procedure in preparation 101, 4-{2-chloro-6-methyl-7-oxo-1H-pyrrolo[2,3-c]pyridin-4-yl}-5-(2,6-dimethylphenoxy)-1-methylpyridin-2-one (70.0 mg, 0.171 mmol, 1.00 equiv) and 2,3-difluorophenylboronic acid (53.9 mg, 0.342 mmol, 2.00 equiv) was reacted to give title compound (31.0 mg, 37.1%) as a white solid after purification by Prep-HPLC (Instrument C; Column A), eluted with a gradient of 0.1% NH₃ solution in water and acetonitrile.

LCMS t_{R} (Shimadzu LMCS-2020, B, 3.00 min): 1.824 min, m/z=488 [M+H]⁺

¹H NMR (300 MHz, DMSO-d⁶) δ 12.59 (s, 1H), 8.00-7.80 (m, 1H), 7.55 (s, 1H), 7.45-7.35 (m, 1H), 7.35-7.25 (m, 1H), 7.18-6.99 (m, 3H), 6.84 (d, J=3.5 Hz, 1H), 6.67 (s, 1H), 6.54 (s, 1H), 3.62 (s, 3H), 3.32 (s, 3H), 2.10 (s, 6H).

### Example 51: 5-(2,6-dimethylphenoxy)-1-methyl-4-[6-methyl-2-(1-methyl-6-oxopyridin-3-yl)-7-oxo-1H-pyrrolo[2,3-c]pyridin-4-yl]pyridin-2-one

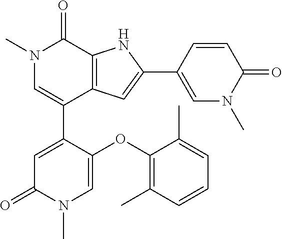 [SMILES: Cc1cccc(C)c1Oc1cn(C)c(=O)cc1-c1cn(C)c(=O)c2[nH]c(-c3ccc(=O)n(C)c3)cc12]

### Preparation 109: 5-(2,6-dimethylphenoxy)-1-methyl-4-[6-methyl-2-(1-methyl-6-oxopyridin-3-yl)-7-oxo-1H-pyrrolo[2,3-c]pyridin-4-yl]pyridin-2-one

Following the procedure in preparation 101, 4-{2-chloro-6-methyl-7-oxo-1H-pyrrolo[2,3-c]pyridin-4-yl}-5-(2,6-dimethylphenoxy)-1-methylpyridin-2-one (60.0 mg, 0.146 mmol, 1.00 equiv) and 1-methyl-5-(4,4,5,5-tetramethyl-1,3,2-dioxaborolan-2-yl)pyridin-2-one (51.6 mg, 0.219 mmol, 1.50 equiv) was reacted to give title compound (11.0 mg, 15.6%) as a white solid after purification by Prep-HPLC (Instrument C; Column C), eluted with a gradient of 0.1% FA solution in water and acetonitrile.

LCMS t_{R} (Shimadzu LMCS-2020, C, 2.80 min): 1.36 min, m/z=483.05 [M+H]⁺

¹H NMR (300 MHz, Methanol-d⁴) δ 8.27 (d, J=2.6 Hz, 1H), 8.08-7.95 (m, 1H), 7.61 (s, 1H), 7.18-7.04 (m, 3H), 6.85 (s, 1H), 6.76 (s, 1H), 6.71-6.64 (m, 2H), 3.74 (s, 3H), 3.67 (s, 3H), 3.48 (s, 3H), 2.17 (s, 6H).

### Example 52: 5-cyclopentyl-4-[2-(2,5-dimethylpyrazol-3-yl)-6-methyl-7-oxo-1H-pyrrolo[2,3-c] pyridin-4-yl]-1-methylpyridin-2-one

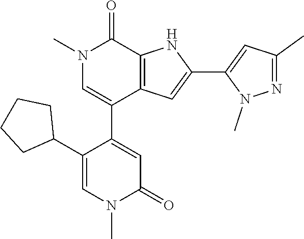 [SMILES: Cc1cc(-c2cc3c(-c4cc(=O)n(C)cc4C4CCCC4)cn(C)c(=O)c3[nH]2)n(C)n1]

### Preparation 110: 4-bromo-5-(cyclopent-1-en-1-yl)-1-methylpyridin-2-one

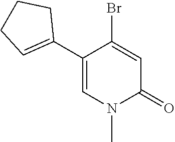

To a stirred mixture of 4-bromo-5-iodo-1-methylpyridin-2-one (5.80 g, 18.5 mmol, 1.00 equiv) and 2-(cyclopent-1-en-1-yl)-4,4,5,5-tetramethyl-1,3,2-dioxaborolane (4.34 g, 22.4 mmol, 1.21 equiv) in dioxane (20.0 mL) and H₂O (5.00 mL) were added Na₂CO₃ (2.04 g, 19.2 mmol, 1.04 equiv) and Pd(PPh₃)₄(7.90 g, 6.84 mmol, 0.370 equiv) at room temperature under nitrogen atmosphere. The resulting mixture was stirred overnight at 100° C. The resulting mixture was extracted with EtOAc (4×20.0 mL). The combined organic layers were washed with brine, dried over anhydrous Na₂SO₄. After filtration, the filtrate was concentrated under reduced pressure. The residue was purified by silica gel column chromatography, eluted with PE/EA (1:9) to afford 4-bromo-5-(cyclopent-1-en-1-yl)-1-methylpyridin-2-one (2.80 g, 59.6%) as a yellow solid.

LCMS: m/z=256 [M+H]⁺

### Preparation 111: 4-bromo-5-cyclopentyl-1-methylpyridin-2-one

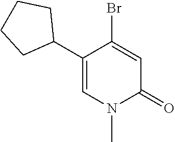

To a solution of 4-bromo-5-(cyclopent-1-en-1-yl)-1-methylpyridin-2-one (2.80 g, 11.0 mmol, 1.00 equiv) in MeOH (30.0 mL) was added Pt/C (4.30 g, 1.10 mmol, 5% wt). The mixture was stirred at room temperature for 4 h under hydrogen atmosphere. The resulting mixture was filtered, and filter cake was washed with MeOH. The filtrate was concentrated under reduced pressure. The residue was purified by reversed flash chromatography with the following conditions: column: C18; mobile phase, MeCN in Water (0.1% FA), 10% to 50% gradient in 10 min; detector, UV 254 nm. This resulted in 4-bromo-5-cyclopentyl-1-methylpyridin-2-one (1.98 g, 67.3%) as a brown solid.

LCMS: m/z=258 [M+H]⁺

### Preparation 112: 5-cyclopentyl-4-[2-(1-isopropylpyrazol-4-yl)-6-methyl-1-(4-methylbenzenesufonyl)-7-oxopyrrolo[2,3-c] pyridin-4-yl]-1-methylpyridin-2-one

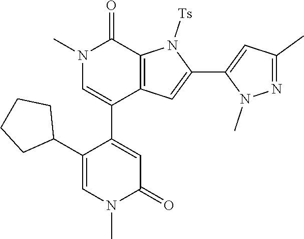 [SMILES: Cc1ccc(S(=O)(=O)n2c(-c3cc(C)nn3C)cc3c(-c4cc(=O)n(C)cc4C4CCCC4)cn(C)c(=O)c32)cc1]

To a stirred mixture of 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (450 mg, 0.972 mmol, 1.00 equiv) and 4-bromo-5-cyclopentyl-1-methylpyridin-2-one (373 mg, 1.46 mmol, 1.50 equiv) in DME (5.00 mL) and H₂O (1.00 mL) were added Na₂CO₃ (206 mg, 1.94 mmol, 2.00 equiv) and Pd(dppf)Cl₂CH₂Cl₂ (79.2 mg, 0.0970 mmol, 0.100 equiv) at room temperature under nitrogen atmosphere. The resulting mixture was stirred for 4 h at 60° C. The reaction was quenched with water. The resulting mixture was extracted with EtOAc (3×10.0 mL). The combined organic layers were washed with brine, dried over anhydrous Na₂SO₄. After filtration, the filtrate was concentrated under reduced pressure. The residue was purified by silica gel column chromatography, eluted with EA/MeOH (10:1) to afford 4-[2-chloro-6-methyl-1-(4-methylbenzenesulfonyl)-7-oxopyrrolo[2,3-c] pyridin-4-yl]-5-cyclopentyl-1-methylpyridin-2-one (375 mg, 75.3%) as a light yellow solid.

LCMS: m/z=572 [M+H]⁺

### Preparation 113: 5-cyclopentyl-4-[2-(2,5-dimethylpyrazol-3-yl)-6-methyl-7-oxo-1H-pyrrolo[2,3-c]pyridin-4-yl]-1-methylpyridin-2-one

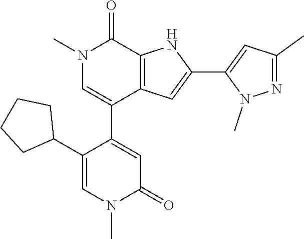 [SMILES: Cc1cc(-c2cc3c(-c4cc(=O)n(C)cc4C4CCCC4)cn(C)c(=O)c3[nH]2)n(C)n1]

To a stirred mixture of 5-cyclopentyl-4-[2-(2,5-dimethylpyrazol-3-yl)-6-methyl-1-(4-methylbenzenesulfonyl)-7-oxopyrrolo[2,3-c]pyridin-4-yl]-1-methylpyridin-2-one (60.0 mg, 0.105 mmol, 1.00 equiv) in dioxane (2.00 mL) and H₂O (0.400 mL) was added NaOH (42.0 mg, 1.05 mmol, 10.0 equiv) at room temperature. The resulting mixture was stirred for 4 h at 60° C. The reaction was quenched with water at room temperature. The resulting mixture was extracted with EtOAc (3×5.00 mL). The combined organic layers were washed with brine, dried over anhydrous Na₂SO₄. After filtration, the filtrate was concentrated under reduced pressure. The crude product was purified by Prep-HPLC (Instrument C; Column A), eluted with a gradient of 10 mmol/L NH₄HCO₃ solution in water and acetonitrile to afford 5-cyclopentyl-4-[2-(2,5-dimethylpyrazol-3-yl)-6-methyl-7-oxo-1H-pyrrolo[2,3-c] pyridin-4-yl]-1-methylpyridin-2-one (19.0 mg, 42.9%) as a white solid.

LCMS t_{R} (Shimadzu LMCS-2020, C, 2.80 min): 1.387 min, m/z=418.15 [M+H]⁺

¹H NMR (400 MHz, Chloroform-d) δ 11.41 (s, 1H), 7.23 (s, 1H), 6.87 (s, 1H), 6.53 (d, J=5.8 Hz, 2H), 6.23 (d, J=1.9 Hz, 1H), 3.94 (s, 3H), 3.69 (s, 3H), 3.63 (s, 3H), 2.83-2.70 (m, 1H), 2.30 (s, 3H), 1.86-1.70 (m, 4H), 1.54-1.41 (m, 2H), 1.41-1.18 (m, 2H).

### Example 53: 4-(5-cyclopentyl-1-methyl-2-oxo-1,2-dihydropyridin-4-yl)-2-(1-isopropyl-1H-pyrazol-4-yl)-6-methyl-1,6-dihydro-7H-pyrrolo[2,3-c]pyridin-7-one

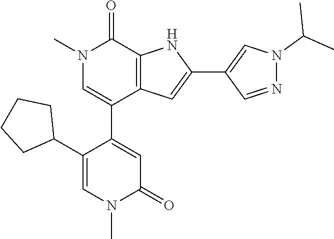 [SMILES: CC(C)n1cc(-c2cc3c(-c4cc(=O)n(C)cc4C4CCCC4)cn(C)c(=O)c3[nH]2)cn1]

### Preparation 114: 4-(5-cyclopentyl-1-methyl-2-oxo-1,2-dihydropyridin-4-yl)-2-(1-isopropyl-1H-pyrazol-4-yl)-6-methyl-1,6-dihydro-7H-pyrrolo[2,3-c]pyridin-7-one

Following the procedure in preparation 113, 5-cyclopentyl-4-[2-(1-isopropylpyrazol-4-yl)-6-methyl-1-(4-methylbenzenesulfonyl)-7-oxopyrrolo[2,3-c] pyridine-4-yl]-1-methylpyridin-2-one (170 mg, 0.290 mmol, 1.00 equiv) was reacted to give title compound (53.0 mg, 42.3%) as a white solid after purification by Prep-HPLC (Instrument C; Column A), eluted with a gradient of 0.05% NH₃ solution in water and acetonitrile.

LCMS t_{R} (Shimadzu LMCS-2020, D, 2.80 min): 1.492 min, m/z=432.10 [M+H]⁺

¹H NMR (400 MHz, DMSO-d⁶) δ 12.20 (bs, 1H), 8.36 (s, 1H), 8.01 (s, 1H), 7.68 (s, 1H), 7.17 (s, 1H), 6.25-6.20 (m, 2H), 4.51-4.40 (m, 1H), 3.55 (s, 3H), 3.50 (s, 3H), 2.77-2.74 (m, 1H), 1.73-1.39 (m, 4H), 1.24-1.20 (m, 10H).

### Example 54: 5-cyclopentyl-4-[2-(2,6-dimethylpyridin-4-yl)-6-methyl-7-oxo-1H-pyrrolo[2,3-c] pyridin-4-yl]-1-methylpyridin-2-one

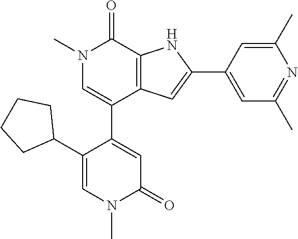 [SMILES: Cc1cc(-c2cc3c(-c4cc(=O)n(C)cc4C4CCCC4)cn(C)c(=O)c3[nH]2)cc(C)n1]

### Preparation 115: 5-cyclopentyl-4-[2-(2,6-dimethylpyridin-4-yl)-6-methyl-7-oxo-1H-pyrrolo[2,3-c] pyridin-4-yl]-1-methylpyridin-2-one

Following the procedure in preparation 113, 5-cyclopentyl-4-[2-(2,6-dimethylpyridin-4-yl)-6-methyl-1-(4-methylbenzenesulfonyl)-7-oxopyrrolo[2,3-c]pyridin-4-yl]-1-methylpyridin-2-one (170 mg, 0.292 mmol, 1.00 equiv) was reacted to give title compound (52.0 mg, 41.6%) as a white solid after purification by Prep-HPLC (Instrument C; Column A), eluted with a gradient of 10 mmol/L NH₄HCO₃ solution in water and acetonitrile.

LCMS t_{R} (Shimadzu LMCS-2020, E, 2.80 min): 1.470 min, m/z=429.10 [M+H]⁺

¹H NMR (400 MHz, DMSO-d⁶) δ 12.65 (s, 1H), 7.70-7.67 (m, 3H), 7.23 (s, 1H), 6.72 (s, 1H), 6.27 (s, 1H), 3.57 (s, 3H), 3.51 (s, 3H), 2.75-2.72 (m, 1H), 2.43 (s, 6H), 1.73-1.62 (m, 4H), 1.38-1.30 (m, 4H).

### Example 55: 5-cyclopentyl-1-methyl-4-{6-methyl-7-oxo-2-phenyl-1H-pyrrolo[2,3-c]pyridin-4-yl}pyridin-2-one

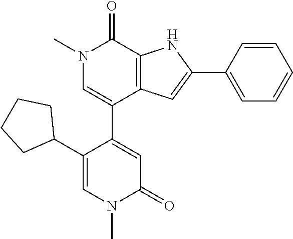 [SMILES: Cn1cc(C2CCCC2)c(-c2cn(C)c(=O)c3[nH]c(-c4ccccc4)cc23)cc1=O]

### Preparation 116: 5-cyclopentyl-1-methyl-4-{6-methyl-7-oxo-2-phenyl-1H-pyrrolo[2,3-c]pyridin-4-yl}pyridin-2-one

Following the procedure in preparation 113, 5-cyclopentyl-1-methyl-4-[6-methyl-1-(4-methylbenzenesulfonyl)-7-oxo-2-phenylpyrrolo[2,3-c] pyridin-4-yl]pyridin-2-one (150 mg, 0.271 mmol, 1.00 equiv) was reacted to give title compound (42.0 mg, 38.8%) as a white solid after purification by Prep-HPLC (Instrument C; Column A), eluted with a gradient of 0.05% NH₃ solution in water and acetonitrile.

LCMS t_{R} (Shimadzu LMCS-2020, D, 2.80 min): 1.444 min, m/z=400.10 [M+H]⁺

¹H NMR (300 MHz, DMSO-d⁶) δ 12.48 (s, 1H), 7.96-7.93 (m, 2H), 7.69 (s, 1H), 7.42-7.28 (s, 3H), 7.22 (s, 1H), 6.50 (s, 1H), 6.28 (s, 1H), 3.57-3.51 (m, 6H), 2.81-2.76 (m, 1H), 1.74-1.63 (m, 4H), 1.38 (s, 4H).

### Example 56: 5-ethynyl-4-[2-(1-isopropylpyrazol-4-yl)-6-methyl-7-oxo-1H-pyrrolo[2,3-c]pyridin-4-yl]-1-methylpyridin-2-one

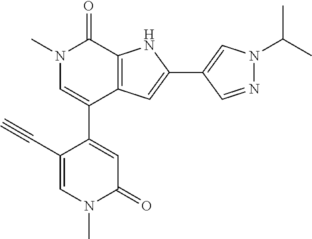 [SMILES: C#Cc1cn(C)c(=O)cc1-c1cn(C)c(=O)c2[nH]c(-c3cnn(C(C)C)c3)cc12]

### Preparation 117: 4-bromo-1-methyl-5-[2-(trimethylsilyl)ethynyl]pyridin-2-one

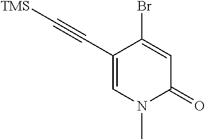

To a stirred mixture of 4-bromo-5-iodo-1-methylpyridin-2-one (17.0 g, 54.2 mmol, 1.00 equiv) and Et₃N (16.5 g, 162 mmol, 3.00 equiv) in THF (170 mL) were added Pd(dppf)Cl₂ (3.96 g, 5.42 mmol, 0.10 equiv) and Cul (1.03 g, 5.42 mmol, 0.10 equiv) at room temperature under nitrogen atmosphere. To the above mixture was added trimethylsilylacetylene (5.85 g, 59.7 mmol, 1.10 equiv) at room temperature. The resulting mixture was stirred for additional 2 h at room temperature. LCMS showed no starting material remaining. The resulting mixture was concentrated under reduced pressure. The residue was purified by silica gel column chromatography, eluted with PE/EA (6:1) to afford 4-bromo-1-methyl-5-[2-(trimethylsilyl)ethynyl]pyridin-2-one (6.60 g, 42.8%) as a brown solid.

LCMS: m/z=286 [M+H]⁺

### Preparation 118: 4-bromo-5-ethynyl-1-methylpyridin-2(1H)-one

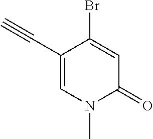

A mixture of 4-bromo-1-methyl-5-[2-(trimethylsilyl)ethynyl]pyridin-2-one (6.60 g, 23.2 mmol, 1.00 equiv) and K₂CO₃ (9.63 g, 69.7 mmol, 3.00 equiv) in MeOH (70 mL) was stirred for overnight at room temperature. The resulting mixture was filtered; the filter cake was washed with EtOAc (3×100 mL). The filtrate was concentrated under reduced pressure. The residue was purified by silica gel column chromatography, eluted with PE/EA (5:1) to afford 4-bromo-5-ethynyl-1-methylpyridin-2-one (2.10 g, 42.6%) as a brown solid.

LCMS: m/z=214 [M+H]⁺

### Preparation 119: 4-[2-chloro-6-methyl-1-(4-methylbenzenesulfonyl)-7-oxopyrrolo[2,3-c]pyridin-4-yl]-5-ethynyl-1-methylpyridin-2-one

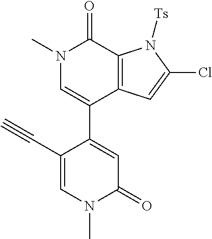 [SMILES: C#Cc1cn(C)c(=O)cc1-c1cn(C)c(=O)c2c1cc(Cl)n2S(=O)(=O)c1ccc(C)cc1]

To a mixture of 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (500 mg, 1.08 mmol, 1.00 equiv) and 4-bromo-5-ethynyl-1-methylpyridin-2-one (458 mg, 2.16 mmol, 2.00 equiv) in DME (4.0 mL) and DME (1.0 mL) was added K₂CO₃ (298 mg, 2.16 mmol, 2.00 equiv) and Pd(PPh₃)₄(125 mg, 0.108 mmol, 0.100 equiv) at room temperature under nitrogen atmosphere. The resulting mixture was stirred for 4 h at 60° C. The reaction was quenched with water at room temperature. The resulting mixture was extracted with EtOAc (4×20.0 mL). The combined organic layers were washed with brine (2×10.0 mL), dried over anhydrous Na₂SO₄. After filtration, the filtrate was concentrated under reduced pressure. The residue was purified by silica gel column chromatography, eluted with EtOAc/MeOH (10:1) to afford 4-[2-chloro-6-methyl-1-(4-methylbenzenesulfonyl)-7-oxopyrrolo[2,3-c] pyridin-4-yl]-5-ethynyl-1-methylpyridin-2-one (250 mg, 49.5%) as a light yellow solid.

LCMS: m/z=468 [M+H]⁺

### Preparation 120: 5-ethynyl-4-[2-(1-isopropylpyrazol-4-yl)-6-methyl-1-(4-methylbenzenesufonyl)-7-oxopyrrol[2,3-c] pyridin-4-yl]-1-methylpyridin-2-one

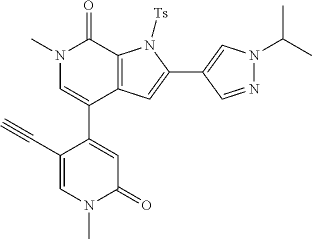 [SMILES: C#Cc1cn(C)c(=O)cc1-c1cn(C)c(=O)c2c1cc(-c1cnn(C(C)C)c1)n2S(=O)(=O)c1ccc(C)cc1]

To a mixture of 4-[2-chloro-6-methyl-1-(4-methylbenzenesulfonyl)-7-oxopyrrolo[2,3-c]pyridin-4-yl]-5-ethynyl-1-methylpyridin-2-one (250 mg, 0.534 mmol, 1.00 equiv) and 1-isopropylpyrazol-4-ylboronic acid (165 mg, 1.07 mmol, 2.00 equiv) in DME (2.00 mL) and H₂O (0.50 mL) was added Pd(PPh₃)₄(61.7 mg, 0.053 mmol, 0.100 equiv) and K₂CO₃ (148 mg, 1.07 mmol, 2.00 equiv) at room temperature under nitrogen atmosphere. The resulting mixture was stirred overnight at 80° C. The reaction was quenched with water at room temperature. The resulting mixture was extracted with EtOAc (4×20.0 mL). The combined organic layers were washed with brine (2×10.0 mL), dried over anhydrous Na₂SO₄. After filtration, the filtrate was concentrated under reduced pressure. The residue was purified by silica gel column chromatography, eluted with EtOAc/MeOH (10:1) to afford 5-ethynyl-4-[2-(1-isopropylpyrazol-4-yl)-6-methyl-1-(4-methylbenzenesulfonyl)-7-oxopyrrolo[2,3-c]pyridin-4-yl]-1-methylpyridin-2-one (90.0 mg, 31.1%) as a light yellow solid.

LCMS: m/z=542 [M+H]⁺

### Preparation 121: 5-ethynyl-4-[2-(1-isopropylpyrazol-4-yl)-6-methyl-7-oxo-1H-pyrrolo[2,3-c]pyridin-4-yl]-1-methylpyridin-2-one

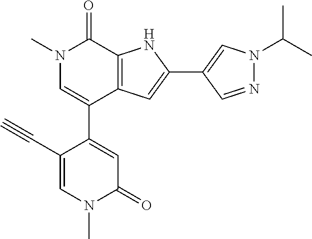 [SMILES: C#Cc1cn(C)c(=O)cc1-c1cn(C)c(=O)c2[nH]c(-c3cnn(C(C)C)c3)cc12]

A solution of 5-ethynyl-4-[2-(1-isopropylpyrazol-4-yl)-6-methyl-1-(4-methylbenzenesulfonyl)-7-oxopyrrolo[2,3-c] pyridin-4-yl]-1-methylpyridin-2-one (90.0 mg, 0.166 mmol, 1.00 equiv) and NaOH (66.4 mg, 1.66 mmol, 10.0 equiv) in MeOH (5.00 mL) and H₂O (1.00 mL) was stirred for 1 h at 40° C. The reaction was quenched with water at room temperature. The resulting mixture was extracted with EtOAc (4×20.0 mL). The combined organic layers were washed with brine (2×10.0 mL), dried over anhydrous Na₂SO₄. After filtration, the filtrate was concentrated under reduced pressure. The crude product was purified by Prep-HPLC (Instrument C; Column E), eluted with a gradient of 10 mmol/L NH₄HCO₃ solution in water and acetonitrile to afford 5-ethynyl-4-[2-(1-isopropylpyrazol-4-yl)-6-methyl-7-oxo-1H-pyrrolo[2,3-c]pyridin-4-yl]-1-methylpyridin-2-one (7.30 mg, 11.3%) as a white solid.

LCMS t_{R} (Shimadzu LMCS-2020, D, 2.80 min): 1.240 min, m/z=388.00 [M+H]⁺

¹H NMR (300 MHz, Methanol-d⁴) δ 8.14 (s, 1H), 8.09 (s, 1H), 7.91 (s, 1H), 7.50 (s, 1H), 6.69 (s, 1H), 6.50 (s, 1H), 4.61-4.52 (m, 1H), 3.86 (s, 3H), 3.54 (s, 3H), 3.47 (s, 1H), 1.54-1.28 (m, 6H).

### Example 57: 4-[2-(1-isopropylpyrazol-4-yl)-6-methyl-7-oxo-1H-pyrrolo[2,3-c]pyridin-4-yl]-1-methyl-5-(prop-1-yn-1-yl)pyridin-2-one

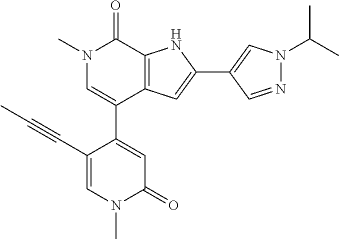 [SMILES: CC#Cc1cn(C)c(=O)cc1-c1cn(C)c(=O)c2[nH]c(-c3cnn(C(C)C)c3)cc12]

### Preparation 122: 4-bromo-1-methyl-5-(prop-1-yn-1-yl)pyridin-2-one

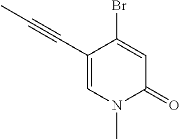

To a stirred mixture of 4-bromo-5-iodo-1-methylpyridin-2-one (5.00 g, 15.9 mmol, 1.00 equiv) and 2-butynoic acid (1.61 g, 19.1 mmol, 1.20 equiv) in DMSO (50.0 mL) were added DBU (7.27 g, 47.8 mmol, 3.00 equiv) and Pd(PPh₃)₂Cl₂ (560 mg, 0.796 mmol, 0.05 equiv) at room temperature under nitrogen atmosphere. The resulting mixture was stirred for 5 h at 110° C. under nitrogen atmosphere. The resulting mixture was filtered; the filter cake was washed with DMSO. The residue was purified by reversed-phase flash chromatography with the following conditions: C18; mobile phase, MeCN in Water (0.1% FA), 10% to 40% gradient in 15 min; detector, UV 254 nm. The resulting mixture was concentrated under reduced pressure. This resulted in 4-bromo-1-methyl-5-(prop-1-yn-1-yl) pyridin-2-one (1.70 g, 47.2%) as a yellow solid

LCMS: m/z=228 [M+H]⁺

### Preparation 123: 4-[2-chloro-6-methyl-1-(4-methylbenzenesulfonyl)-7-oxopyrrolo[2,3-c]pyridin-4-yl]-1-methyl-5-(prop-1-yn-1-yl) pyridin-2-one

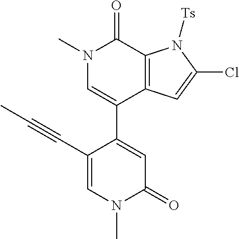 [SMILES: CC#Cc1cn(C)c(=O)cc1-c1cn(C)c(=O)c2c1cc(Cl)n2S(=O)(=O)c1ccc(C)cc1]

Following the procedure in preparation 119, 2-chloro-6-methyl-1-(4-methylbenzenesulfonyl)-4-(4,4,5,5-tetramethyl-1,3,2-dioxaborolan-2-yl)pyrrolo [2,3-c]pyridin-7-one (524 mg, 1.13 mmol, 1.00 equiv) and 4-bromo-1-methyl-5-(prop-1-yn-1-yl)pyridin-2-one (512 mg, 2.26 mmol, 2.00 equiv) was reacted to give title compound (470 mg, 86.1%) as a white solid.

LCMS: m/z=482 [M+H]⁺

### Preparation 124: 4-[2-(1-isopropylpyrazol-4-yl)-6-methyl-1-(4-methylbenzenesulfonyl)-7-oxopyrrolo[2,3-c] pyridin-4-yl]-1-methyl-5-(prop-1-yn-1-yl)pyridin-2-one

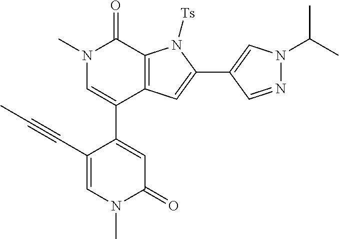 [SMILES: CC#Cc1cn(C)c(=O)cc1-c1cn(C)c(=O)c2c1cc(-c1cnn(C(C)C)c1)n2S(=O)(=O)c1ccc(C)cc1]

Following the procedure in preparation 112, 4-[2-chloro-6-methyl-1-(4-methylbenzenesulfonyl)-7-oxopyrrolo[2,3-c] pyridin-4-yl]-1-methyl-5-(prop-1-yn-1-yl)pyridin-2-one (250 mg, 0.519 mmol, 1.00 equiv) and 1-isopropylpyrazol-4-ylboronic acid (160 mg, 1.04 mmol, 2.00 equiv) was reacted to give title compound (100 mg, 34.7%) as a white solid.

LCMS: m/z=556 [M+H]⁺

### Preparation 125: 4-[2-(1-isopropylpyrazol-4-yl)-6-methyl-7-oxo-1H-pyrrolo[2,3-c]pyridin-4-y]-1-methyl-5-(prop-1-yn-1-yl)pyridin-2-one

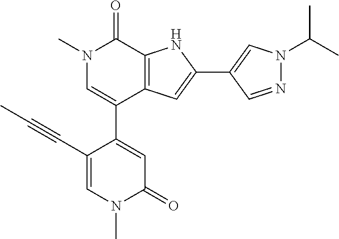 [SMILES: CC#Cc1cn(C)c(=O)cc1-c1cn(C)c(=O)c2[nH]c(-c3cnn(C(C)C)c3)cc12]

Following the procedure in preparation 113, 4-[2-(1-isopropylpyrazol-4-yl)-6-methyl-1-(4-methylbenzenesulfonyl)-7-oxopyrrolo[2,3-c] pyridin-4-yl]-1-methyl-5-(prop-1-yn-1-yl) pyridin-2-one (100 mg, 0.180 mmol, 1.00 equiv) was reacted to give title compound (8.40 mg, 11.6%) as an off-white solid after purification by Prep-HPLC (Instrument C; Column A), eluted with a gradient of 10 mmol/L NH₄HCO₃ solution in water and acetonitrile.

LCMS t_{R} (Shimadzu LMCS-2020, D, 2.80 min): 1.362 min, m/z=402.05 [M+H]⁺

¹H NMR (400 MHz, Methanol-d⁴) δ 8.15 (s, 1H), 7.92 (d, J=3.9 Hz, 2H), 7.50 (s, 1H), 6.68 (s, 1H), 6.50 (s, 1H), 4.67-4.50 (m, 1H), 3.69 (s, 3H), 3.61 (s, 3H), 1.83 (s, 3H), 1.53 (d, J=6.6 Hz, 6H).

### Example 58: 4-[2-(2,6-dimethylpyridin-4-yl)-6-methyl-1-(4-methylbenzenesulfonyl)-7-oxopyrrolo[2,3-c]pyridin-4-yl]-1-methyl-5-(prop-1-yn-1-yl)pyridin-2-one

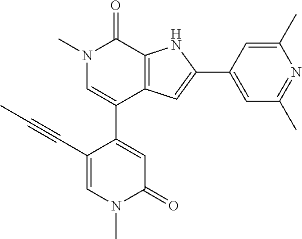 [SMILES: CC#Cc1cn(C)c(=O)cc1-c1cn(C)c(=O)c2[nH]c(-c3cc(C)nc(C)c3)cc12]

### Preparation 126: 4-[2-(2,6-dimethylpyridin-4-yl)-6-methyl-1-(4-methylbenzenesulfonyl)-7-oxopyrrolo[2,3-c]pyridin-4-yl]-1-methyl-5-(prop-1-yn-1-yl)pyridin-2-one

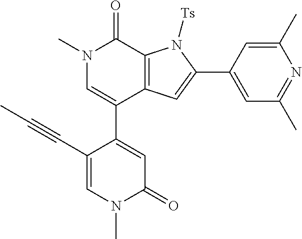 [SMILES: CC#Cc1cn(C)c(=O)cc1-c1cn(C)c(=O)c2c1cc(-c1cc(C)nc(C)c1)n2S(=O)(=O)c1ccc(C)cc1]

Following the procedure in preparation 112, 4-[2-chloro-6-methyl-1-(4-methylbenzenesulfonyl)-7-oxopyrrolo[2,3-c] pyridin-4-yl]-1-methyl-5-(prop-1-yn-1-yl)pyridin-2-one (270 mg, 0.560 mmol, 1.00 equiv) and 2,6-dimethylpyridin-4-ylboronic acid (169 mg, 1.12 mmol, 2.00 equiv) was reacted to give title compound (70.0 mg, 22.6%) as a white solid.

LCMS: m/z=552 [M+H]⁺

### Preparation 127: 4-[2-(2,6-dimethylpyridin-4-yl)-6-methyl-7-oxo-1H-pyrrolo[2,3-c]pyridin-4-yl]-1-methyl-5-(prop-1-yn-1-yl)pyridin-2-one

Following the procedure in preparation 113, 4-[2-(2,6-dimethylpyridin-4-yl)-6-methyl-1-(4-methylbenzenesulfonyl)-7-oxopyrrolo[2,3-c] pyridin-4-yl]-1-methyl-5-(prop-1-yn-1-yl)pyridin-2-one (70.0 mg, 0.127 mmol, 1.0 equiv) was reacted to give title compound (6.50 mg, 12.9%) as a yellow solid after purification by Prep-HPLC (Instrument C; Column F), eluted with a gradient of 0.1% FA solution in water and acetonitrile.

LCMS t_{R} (Shimadzu LMCS-2020, D, 2.80 min): 1.270 min, m/z=399.05 [M+H]⁺

¹H NMR (400 MHz, Methanol-d⁴) δ 7.92 (s, 1H), 7.61-7.43 (m, 3H), 6.95 (s, 1H), 6.66 (s, 1H), 3.69 (s, 3H), 3.61 (s, 3H), 2.55 (s, 6H), 1.81 (s, 3H).

### Example 59: 1-cyclopropyl-5-(2,6-dimethylphenoxy)-4-[2-(2,6-dimethylpyridin-4-yl)-6-methyl-7-oxo-1H-pyrrolo[2,3-c]pyridin-4-yl]pyridin-2-one

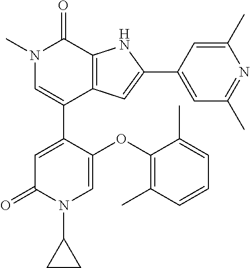

### Preparation 128: 2-bromo-5-(2,6-dimethylphenoxy)pyridine

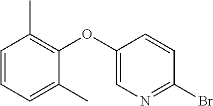 [SMILES: Cc1cccc(C)c1Oc1ccc(Br)nc1]

To a stirred mixture of 2-bromo-5-fluoropyridine (200 g, 1.14 mol, 1.00 equiv) and 2,6-dimethylphenol (138.9 g, 1.13 mol, 1.0 equiv) in DMSO (2.0 L) was added Cs2CO3 (407.3 g, 1.25 mol, 1.10 equiv) in portions at room temperature. The resulting mixture was stirred for 2 h at 120° C. Desired product could be detected by TLC. The reaction was quenched by the addition of Water/Ice (1.0 L) at room temperature. The resulting mixture was extracted with EtOAc (5×500 mL). The combined organic layers were washed with brine (3×500 mL), dried over anhydrous Na2SO4, filtered and concentrated under reduced pressure. The residue was purified by silica gel column chromatography, eluted with PE/EA (12:1) to afford 2-bromo-5-(2,6-dimethylphenoxy)pyridine (248 g, 78.46%) as an off-white solid.

LCMS: m/z=280.0 [M+H]⁺

¹H NMR (400 MHz, DMSO-d⁶) δ 8.03 (d, J=3.1 Hz, 1H), 7.55 (d, J=8.7 Hz, 1H), 7.20-7.11 (m, 3H), 7.04 (dd, J=8.7, 3.2 Hz, 1H), 2.07 (s, 6H).

### Preparation 129: 2-bromo-5-(2,6-dimethylphenoxy)-4-iodopyridine

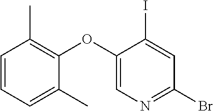 [SMILES: Cc1cccc(C)c1Oc1cnc(Br)cc1I]

To a solution of 2-bromo-5-(2,6-dimethylphenoxy)pyridine (248 g, 0.89 mol, 1.00 equiv) in THF (2.5 L) was added LDA solution (513 mL, 1.26 mol, 2.0 M/L in THF/Hexane) dropwise at −78° C. under N₂ atmosphere. The mixture was stirred at −78° C. for 40 min. To the above mixture was added a solution of I₂ (1.13 kg, 4.46 mol, 5.0 equiv) in THF (200 mL) dropwise. The mixture was stirred for another 15 min and then allowed to warm up to room temperature. The mixture was stirred for 1 h at room temperature. The reaction was quenched with aq. Na₂S2O₃ (600 mL). The mixture was extracted with EtOAc (3×500 mL). The combined organic phases were washed with brine (500 mL), dried over anhydrous Na₂SO₄, filtered and concentrated under vacuum. The residue was purified by silica gel column chromatography, eluted with PE/EA (10:1) to afford 2-bromo-5-(2,6-dimethylphenoxy)-4-iodopyridine (260 g, 72.17%) as a white solid.

LCMS: m/z=405.9 [M+H]⁺

¹H NMR (300 MHz, DMSO-d⁶) δ 8.24 (s, 1H), 7.27-7.12 (m, 4H), 2.07 (s, 6H).

### Preparation 130: 5-(2,6-dimethylphenoxy)-4-iodopyridin-2(1H)-one

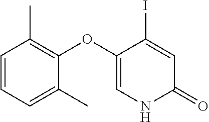 [SMILES: Cc1cccc(C)c1Oc1c[nH]c(=O)cc1I]

To a stirred solution of 2-bromo-5-(2,6-dimethylphenoxy)-4-iodopyridine (260 g, 0.64 mol, 1.00 equiv) in t-BuOH (2.6 L) was added KOH (361.0 g, 6.43 mol, 10.0 equiv) in portions at room temperature. The resulting mixture was stirred for 6 h at 120° C. in autoclave. The mixture was allowed to cool down to room temperature. The reaction was quenched with Water/Ice at room temperature. The aqueous layer was extracted with CH₂Cl₂ (3×500 mL). The combined organic layer was concentrated under reduced pressure. The residue was purified by silica gel column chromatography, eluted with PE/EA (1:4) to afford crude product. The crude product was further purified by trituration with aq. K₂CO₃ (2.0 L, 1.0 N) overnight. After filtration, the filter cake was dried to afford 5-(2,6-dimethylphenoxy)-4-iodo-1H-pyridin-2-one (90.4 g, 41.18%) as a white solid.

LCMS: m/z=341.9 [M+H]⁺

1H NMR (400 MHz, DMSO-d₆) δ 11.14 (s, 1H), 7.17-7.07 (m, 4H), 6.23 (s, 1H), 2.09 (s, 6H).

### Preparation 131: 1-cyclopropyl-5-(2,6-dimethylphenoxy)-4-iodopyridin-2-one

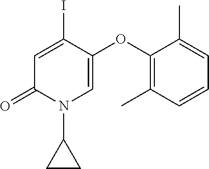 [SMILES: Cc1cccc(C)c1Oc1cn(C2CC2)c(=O)cc1I]

To a stirred mixture of 5-(2,6-dimethylphenoxy)-4-iodo-1H-pyridin-2-one (10.0 g, 29.3 mmol, 1.00 equiv) and cyclopropylboronic acid (5.54 g, 64.5 mmol, 2.20 equiv) in DCE (400 mL) were added Cu(OAc)₂ (5.70 g, 31.4 mmol, 1.07 equiv) and 2,2′-bipyridine (4.90 g, 31.4 mmol, 1.07 equiv) at room temperature under nitrogen atmosphere. The resulting mixture was stirred for 3 h at 70° C. The resulting mixture was extracted with CH₂Cl₂ (3×20 mL), dried over anhydrous Na₂SO₄, filtered and concentrated under reduced pressure. The residue was purified by silica gel column chromatography, eluted with PE/EA (1:1) to afford 1-cyclopropyl-5-(2,6-dimethylphenoxy)-4-iodopyridin-2-one (1.8 g, 16.11%) as a yellow oil.

LCMS: m/z=382 [M+H]⁺

### Preparation 132: 4-[2-chloro-6-methyl-1-(4-methylbenzenesulfonyl)-7-oxopyrrolo[2,3-c]pyridin-4-yl]-1-cyclopropyl-5-(2,6-dimethylphenoxy)pyridin-2-one

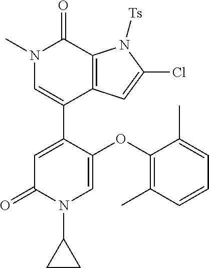 [SMILES: Cc1ccc(S(=O)(=O)n2c(Cl)cc3c(-c4cc(=O)n(C5CC5)cc4Oc4c(C)cccc4C)cn(C)c(=O)c32)cc1]

To a stirred mixture of 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (1.8 g, 3.89 mmol, 1.0 equiv) and 1-cyclopropyl-5-(2,6-dimethylphenoxy)-4-iodopyridin-2-one (2.22 g, 5.84 mmol, 1.5 equiv) in DME (20 mL) were added Na₂CO₃ (820 mg, 7.78 mmol, 2.0 equiv), Pd(dppf) C₂ (0.32 g, 0.389 mmol, 0.1 equiv) and H₂O (4 mL) at room temperature under nitrogen atmosphere. The resulting mixture was stirred overnight at 60° C. under nitrogen atmosphere. Desired product could be detected by LCMS. The resulting mixture was extracted with EtOAc (3×50 mL), dried over anhydrous Na₂SO₄, filtered and concentrated under reduced pressure. The residue was purified by silica gel column chromatography, eluted with EtOAc/MeOH (10:1) to afford 4-[2-chloro-6-methyl-1-(4-methylbenzenesulfonyl)-7-oxopyrrolo[2,3-c]pyridin-4-yl]-1-cyclopropyl-5-(2,6-dimethylphenoxy)pyridin-2-one (900 mg, 39.21%) as a yellow solid.

LCMS: m/z=590 [M+H]⁺

### Preparation 133: 4-{2-chloro-6-methyl-7-oxo-1H-pyrrolo[2,3-c]pyridin-4-yl}-1-cyclopropyl-5-(2,6-dimethylphenoxy)pyridin-2-one

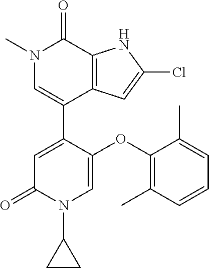 [SMILES: Cc1cccc(C)c1Oc1cn(C2CC2)c(=O)cc1-c1cn(C)c(=O)c2[nH]c(Cl)cc12]

To a stirred mixture of 4-[2-chloro-6-methyl-1-(4-methylbenzenesulfonyl)-7-oxopyrrolo[2,3-c]pyridin-4-yl]-1-cyclopropyl-5-(2,6-dimethylphenoxy)pyridin-2-one (900 mg, 1.53 mmol, 1.0 equiv) in dioxane (8.0 mL) and H₂O (2.0 mL) was added NaOH (610 mg, 15.3 mmol, 10.0 eq.).The resulting mixture was stirred for 2 h at 60° C. under nitrogen atmosphere. Desired product could be detected by LCMS. The resulting mixture was extracted with EtOAc (3×20 mL), dried over anhydrous Na₂SO₄, filtered and concentrated under reduced pressure. The residue was purified by silica gel column chromatography, eluted with EA/MeOH (10:1) to afford 4-{2-chloro-6-methyl-7-oxo-1H-pyrrolo[2,3-c]pyridin-4-yl}-1-cyclopropyl-5-(2,6-dimethylphenoxy)pyridin-2-one (300 mg, 45.12%) as a yellow solid.

LCMS: m/z=436 [M+H]⁺

### Preparation 134: 1-cyclopropyl-5-(2,6-dimethylphenoxy)-4-[2-(2,6-dimethypyridin-4-yl)-6-methyl-7-oxo-1H-pyrrolo[2,3-c] pyridin-4-y]pyridin-2-one

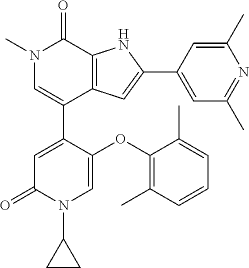

To a stirred mixture of 4-{2-chloro-6-methyl-7-oxo-1H-pyrrolo[2,3-c]pyridin-4-yl}-1-cyclopropyl-5-(2,6-dimethylphenoxy)pyridin-2-one (300 mg, 0.688 mmol, 1.0 equiv) and 2,6-dimethylpyridin-4-ylboronic acid (156 mg, 1.03 mmol, 1.50 equiv) in DME (5 mL) were added K₂CO₃ (190.23 mg, 1.376 mmol, 2 equiv), XPhos Pd G3 (58.25 mg, 0.069 mmol, 0.1 equiv) and H₂O (1.0 mL) at room temperature under nitrogen atmosphere. The resulting mixture was stirred overnight at 60° C. under nitrogen atmosphere. Desired product could be detected by LCMS. The resulting mixture was extracted with EtOAc (3×20 mL), dried over anhydrous Na₂SO₄, filtered and concentrated under reduced pressure. The crude product was purified by Prep-HPLC (Instrument C; Column G), eluted with a gradient of 0.1% FA solution in water and acetonitrile to afford the product, and the product was further treated with HCl in MeOH (2.0 mL, 4.0 M/L) and lyophilized to give 1-cyclopropyl-5-(2,6-dimethylphenoxy)-4-[2-(2,6-dimethylpyridin-4-yl)-6-methyl-7-oxo-1H-pyrrolo[2,3-c]pyridin-4-yl]pyridin-2-one hydrochloride (46 mg, 12.31%) as a yellow solid.

LCMS^{a} t_{R} (Shimadzu LMCS-2020, D, 2.80 min): 1.41 min, m/z=507.1 [M+H]⁺

1H NMR (400 MHz, Methanol-d₄) δ 8.14 (s, 2H), 7.74 (s, 1H), 7.52 (s, 1H), 7.15-7.05 (m, 4H), 6.68 (s, 1H), 4.85 (s, 1H), 3.74 (s, 3H), 3.41 (s, 1H), 2.77 (s, 6H), 2.15 (s, 6H), 1.11 (s, 2H), 0.79 (s, 2H).

### Example 60: 2-(1,3-dimethyl-1H-pyrazol-5-yl)-6-methyl-4-(1-methyl-2-oxo-5-phenyl-1,2-dihydropyridin-4-yl)-1,6-dihydro-7H-pyrrolo[2,3-c] pyridin-7-one

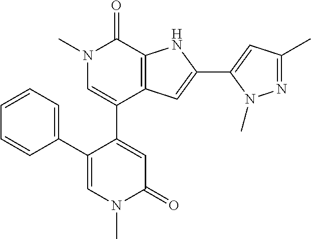 [SMILES: Cc1cc(-c2cc3c(-c4cc(=O)n(C)cc4-c4ccccc4)cn(C)c(=O)c3[nH]2)n(C)n1]

### Preparation 135: 4-bromo-1-methyl-5-phenylpyridin-2-one

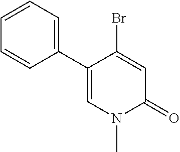

To a stirred mixture of 4-bromo-5-iodo-1-methylpyridin-2-one (5.00 g, 15.9 mmol, 1.00 equiv) and phenyl boronic acid (2.72 g, 22.3 mmol, 1.40 equiv) in DMF (30.0 mL) and H₂O (3.00 mL) was added Na₂CO₃ (3.38 g, 31.9 mmol, 2.00 equiv) and Pd(PPh₃)₄(920 mg, 0.796 mmol, 0.05 equiv) at room temperature under nitrogen atmosphere. The resulting mixture was stirred for 6 h at 100° C. The reaction was quenched with water. The resulting mixture was extracted with EtOAc (3×100 mL). The combined organic layers were washed with brine and water, dried over anhydrous Na₂SO₄. After filtration, the filtrate was concentrated under reduced pressure. The residue was purified by silica gel column chromatography, eluted with PE/EA (1:1) to afford 4-bromo-1-methyl-5-phenylpyridin-2-one (2.00 g, 47.6%) as a light yellow solid.

LCMS: m/z=266 [M+H]⁺

### Preparation 136: 2-chloro-6-methyl-4-(1-methyl-2-oxo-5-phenyl-1,2-dihydropyridin-4-yl)-1-tosyl-1,6-dihydro-7H-pyrrolo[2,3-c]pyridin-7-one

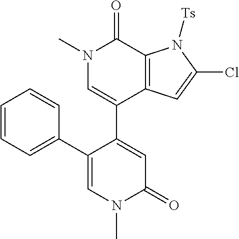 [SMILES: Cc1ccc(S(=O)(=O)n2c(Cl)cc3c(-c4cc(=O)n(C)cc4-c4ccccc4)cn(C)c(=O)c32)cc1]

To a stirred mixture of 2-chloro-6-methyl-1-(4-methylbenzenesulfonyl)-4-(4,4,5,5-tetramethyl-1,3,2-dioxaborolan-2-yl)pyrrolo[2,3-c]pyridin-7-one (800 mg, 1.73 mmol, 1.00 equiv) and 4-bromo-1-methyl-5-phenylpyridin-2-one (548 mg, 2.08 mmol, 1.20 equiv) in 1,4-dioxane (8.00 mL) and H₂O (2.00 mL) was added Pd(PPh₃)₄(200 mg, 0.173 mmol, 0.100 equiv) and Na₂CO₃ (366 mg, 3.46 mmol, 2.00 equiv) at room temperature under nitrogen atmosphere. The resulting mixture was stirred for 4 h at 60° C. The reaction was quenched with water at room temperature. The resulting mixture was extracted with EtOAc (4×20.0 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford 2-chloro-6-methyl-4-(1-methyl-2-oxo-5-phenyl-1,2-dihydropyridin-4-yl)-1-tosyl-1,6-dihydro-7H-pyrrolo[2,3-c]pyridin-7-one (500 mg, 56.5%) as a light yellow solid.

LCMS: m/z=520 [M+H]⁺

### Preparation 137: 4-{2-chloro-6-methyl-7-oxo-1H-pyrrolo[2,3-c]pyridin-4-yl}-5-phenyl-1H-pyridin-2-one

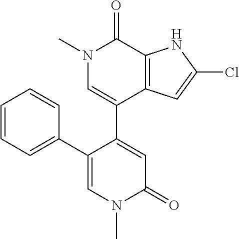 [SMILES: Cn1cc(-c2ccccc2)c(-c2cn(C)c(=O)c3[nH]c(Cl)cc23)cc1=O]

To a mixture of 4-[2-chloro-6-methyl-1-(4-methylbenzenesulfonyl)-7-oxopyrrolo[2,3-c]pyridin-4-yl]-1-methyl-5-phenylpyridin-2-one (470 mg, 0.904 mmol, 1.00 equiv) in 1,4-dioxane (5.00 mL) and H₂O (1.00 mL) was added NaOH (362 mg, 9.04 mmol, 10.0 equiv) at room temperature. The resulting mixture was stirred for 1 h at 60° C. The reaction was quenched with water. The resulting mixture was extracted with EtOAc (4×20.0 mL). The combined organic layers were washed with brine, dried over anhydrous Na₂SO₄. After filtration, the filtrate was concentrated under reduced pressure. The residue was purified by silica gel column chromatography, eluted with EtOAc/MeOH (10:1) to afford 4-{2-chloro-6-methyl-7-oxo-1H-pyrrolo[2,3-c]pyridin-4-yl}-5-phenyl-1H-pyridin-2-one (300 mg, 94.4%) as a white solid.

LCMS: m/z=366 [M+H]⁺

### Preparation 138: 2-(1,3-dimethyl-1H-pyrazol-5-yl)-6-methyl-4-(1-methyl-2-oxo-5-phenyl-1,2-dihydropyridin-4-yl)-1,6-dihydro-7H-pyrrolo[2,3-c]pyridin-7-one

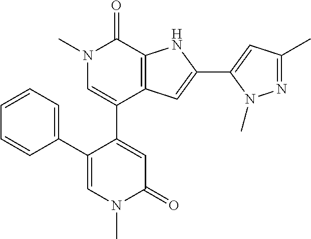 [SMILES: Cc1cc(-c2cc3c(-c4cc(=O)n(C)cc4-c4ccccc4)cn(C)c(=O)c3[nH]2)n(C)n1]

A mixture of 4-{2-chloro-6-methyl-7-oxo-1H-pyrrolo[2,3-c]pyridin-4-yl}-1-methyl-5-phenylpyridin-2-one (60.0 mg, 0.164 mmol, 1.00 equiv) and 2,5-dimethylpyrazol-3-ylboronic acid (34.4 mg, 0.246 mmol, 1.50 equiv) in DME (1.00 mL) and H₂O (0.200 mL) was added K₂CO₃ (45.3 mg, 0.328 mmol, 2.00 equiv) and XPhos Pd G3 (13.9 mg, 0.016 mmol, 0.100 equiv) at room temperature under nitrogen atmosphere. The resulting mixture was stirred for 1 h at 60° C. The reaction was quenched with water at room temperature. The resulting mixture was extracted with EtOAc (4×20.0 mL). The combined organic layers were washed with brine, dried over anhydrous Na₂SO₄. After filtration, the filtrate was concentrated under reduced pressure. The crude product was purified by Prep-HPLC (Instrument C; Column D), eluted with a gradient of 0.1% FA solution in water and acetonitrile to afford 4-[2-(2,5-dimethylpyrazol-3-yl)-6-methyl-7-oxo-1H-pyrrolo[2,3-c] pyridin-4-yl]-1-methyl-5-phenylpyridin-2-one (35.0 mg, 50.2%) as a white solid.

LCMSⁱ t_{R} (Shimadzu LMCS-2020, C, 2.80 min): 1.274 min, m/z=426.10 [M+H]⁺

¹H NMR (300 MHz, DMSO-d⁶) δ 12.18 (s, 1H), 8.14 (s, 1H), 7.88 (s, 1H), 7.36-7.08 (m, 5H), 6.49-6.46 (m, 2H), 5.89 (d, J=2.1 Hz, 1H), 3.63-3.54 (m, 3H), 3.41 (s, 6H), 2.11 (s, 3H).

### Example 61: 4-[2-(1-isopropylpyrazol-4-yl)-6-methyl-7-oxo-1H-pyrrolo[2,3-c]pyridin-4-yl]-1-methyl-5-phenylpyridin-2-one

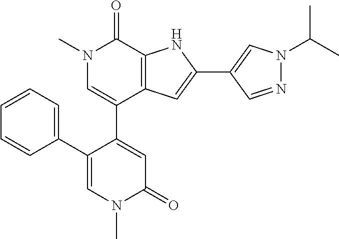 [SMILES: CC(C)n1cc(-c2cc3c(-c4cc(=O)n(C)cc4-c4ccccc4)cn(C)c(=O)c3[nH]2)cn1]

### Preparation 139: 4-[2-(1-isopropylpyrazol-4-yl)-6-methyl-7-oxo-1H-pyrrolo[2,3-c]pyridin-4-yl]-1-methyl-5-phenylpyridin-2-one

Following the procedure in preparation 138, 4-{2-chloro-6-methyl-7-oxo-1H-pyrrolo[2,3-c]pyridin-4-yl}-1-methyl-5-phenylpyridin-2-one (60.0 mg, 0.164 mmol, 1.00 equiv) and 1-isopropylpyrazol-4-ylboronic acid (37.9 mg, 0.246 mmol, 1.50 equiv) was reacted to give title compound (31.0 mg, 43.0%) as a white solid after purification by Prep-HPLC (Instrument C; Column A), eluted with a gradient of 0.1% FA solution in water and acetonitrile.

LCMS^{j} t_{R} (Shimadzu LMCS-2020, C, 2.80 min): 1.393 min, m/z=440.2 [M+H]⁺

¹H NMR (300 MHz, DMSO-d⁶) δ 12.01 (s, 1H), 8.24 (s, 1H), 7.86 (s, 2H), 7.19-7.18 (m, 4H), 7.13-7.11 (m, 1H), 7.01 (s, 1H), 6.48 (s, 1H), 6.05 (s, 1H), 4.48-4.41 (m, 1H), 3.58 (s, 3H), 3.42 (s, 3H), 1.42-1.40 (m, 6H).

### Example 62: 4-[2-(2,6-dimethylpyridin-4-yl)-6-methyl-7-oxo-1H-pyrrolo[2,3-c]pyridin-4-yl]-1-methyl-5-phenylpyridin-2-one

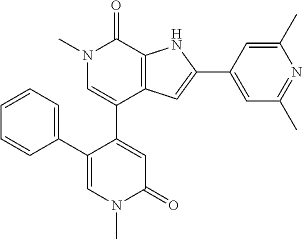 [SMILES: Cc1cc(-c2cc3c(-c4cc(=O)n(C)cc4-c4ccccc4)cn(C)c(=O)c3[nH]2)cc(C)n1]

### Preparation 140: 4-[2-(2,6-dimethylpyridin-4-yl)-6-methyl-7-oxo-1H-pyrrolo[2,3-c]pyridin-4-yl]-1-methyl-5-phenylpyridin-2-one

Following the procedure in preparation 138, 4-{2-chloro-6-methyl-7-oxo-1H-pyrrolo[2,3-c] pyridin-4-yl}-1-methyl-5-phenylpyridin-2-one (60.0 mg, 0.164 mmol, 1.00 equiv) and 2,6-dimethylpyridin-4-ylboronic acid (37.1 mg, 0.246 mmol, 1.50 equiv) was reacted to give title compound (33.0 mg, 46.1%) as a yellow solid after purification by Prep-HPLC (Instrument C; Column C), eluted with a gradient of 0.1% NH₃·H₂O solution in water and acetonitrile.

LCMS^{k} t_{R} (Shimadzu LMCS-2020, C, 2.80 min): 1.012 min. m/z=437.05 [M+H]⁺

¹H NMR (400 MHz, DMSO-d⁶) δ 12.43 (s, 1H), 7.88 (s, 1H), 7.49 (s, 2H), 7.22-7.15 (m, 5H), 7.11-7.07 (m, 1H), 6.50-6.48 (m, 2H), 3.56 (s, 3H), 3.46 (s, 3H), 2.41 (s, 6H).

### Example 63: 1-methyl-4-{6-methyl-7-oxo-2-phenyl-1H-pyrrolo[2,3-c]pyridin-4-yl}-5-phenylpyridin-2-one

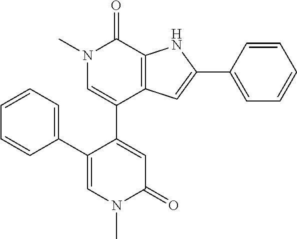 [SMILES: Cn1cc(-c2ccccc2)c(-c2cn(C)c(=O)c3[nH]c(-c4ccccc4)cc23)cc1=O]

### Preparation 141: 1-methyl-4-{6-methyl-7-oxo-2-phenyl-1H-pyrrolo[2,3-c]pyridin-4-yl}-5-phenylpyridin-2-one

Following the procedure in preparation 138, 4-{2-chloro-6-methyl-7-oxo-1H-pyrrolo[2,3-c] pyridin-4-yl}-1-methyl-5-phenylpyridin-2-one (60.0 mg, 0.164 mmol, 1.00 equiv) and phenyl boronic acid (30.0 mg, 0.246 mmol, 1.50 equiv) was reacted to give title compound (30.0 mg, 44.9%) as a white solid after purification by Prep-HPLC (Instrument C; Column A), eluted with a gradient of 0.1% FA solution in water and acetonitrile.

LCMS^{l} t_{R} (Shimadzu LMCS-2020, C, 2.80 min): 1.564 min, m/z=408.10 [M+H]⁺

¹H NMR (300 MHz, DMSO-d⁶) δ 12.20 (s, 1H), 7.87 (s, 1H), 7.77-7.75 (m, 2H), 7.38-7.15 (m, 8H), 7.10-7.06 (m, 1H), 6.50 (s, 1H), 6.22 (s, 1H), 3.56-3.48 (m, 6H).

### Example 64: 4-(5-(2,6-dimethylphenoxy)-1-methyl-2-oxo-1,2-dihydropyridin-4-yl)-6-methyl-2-(3-methyl-1-(oxetan-3-yl)-1H-pyrazol-4-yl)-1,6-dihydro-7H-pyrrolo[2,3-c]pyridin-7-one

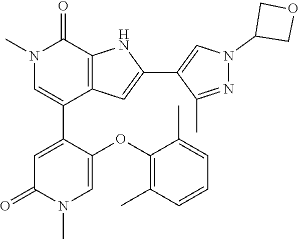 [SMILES: Cc1cccc(C)c1Oc1cn(C)c(=O)cc1-c1cn(C)c(=O)c2[nH]c(-c3cn(C4COC4)nc3C)cc12]

### Preparation 142: 3-methyl-1-(oxetan-3-yl)-4-(4, 4, 5, 5-tetramethyl-1, 3, 2-dioxaborolan-2-yl)-1H-pyrazole

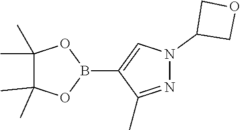 [SMILES: Cc1nn(C2COC2)cc1B1OC(C)(C)C(C)(C)O1]

Sodium hydride (60% w/w, 0.480 g, 12.01 mmol) was suspended in DMF (15 mL) under nitrogen and was cooled at 0° C. in an ice bath. 3-methyl-4-(4,4,5,5-tetramethyl-1,3,2-dioxaborolan-2-yl)-1H-pyrazole (1.0 g, 4.80 mmol) was dissolved in DMF (5 mL) and drop wise added over a period of 30 min (evolution of gas was observed and formation of an exotherm raised the reaction temperature to 0° C.). The reaction mixture was stirred at 0° C. for 30 min. 3-iodooxetane (2.65 g, 14.41 mmol) was drop wise added to the reaction mixture at 0° C. over a period of 5 min. The resulting grey suspension gradually warmed to room temperature and stirred at room temperature for 16 h. TLC (9:1 DCM/Methanol) showed no SM remaining. The reaction mixture was cooled at 0° C. and slowly added water (100 mL). The mixture was stirred 5 min before separating the phases. The aqueous phase was extracted with ethyl acetate (3×50 mL). The combined organics were washed (brine), dried (Na₂SO₄), filtered and evaporated to brown oil. The residue was purified by normal phase chromatography, eluting with (60:40) ethyl acetate/hexane. Solvent reduction gave yellow oil (0.38 g, 30%).

¹H NMR: (400 MHz, DMSO) δ 7.92 (s, 1H), 5.48 (m, 1H), 4.90-4.84 (m, 4H), 2.32 (s, 3H), 1.24 (s, 12H).

### Preparation 143: 4-(5-(2,6-dimethylphenoxy)-1-methyl-2-oxo-1,2-dihydropyridin-4-yl)-6-methyl-2-(3-methyl-1-(oxetan-3-yl)-1H-pyrazol-4-yl)-1,6-dihydro-7H-pyrrolo[2,3-c] pyridin-7-one

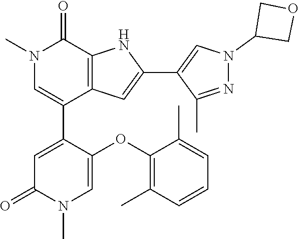 [SMILES: Cc1cccc(C)c1Oc1cn(C)c(=O)cc1-c1cn(C)c(=O)c2[nH]c(-c3cn(C4COC4)nc3C)cc12]

Following the procedure in preparation 37, 2-chloro-4-(5-(2,6-dimethylphenoxy)-1-methyl-2-oxo-1,2-dihydropyridin-4-yl)-6-methyl-1-tosyl-1,6-dihydro-7H-pyrrolo[2,3-c] pyridin-7-one (0.15 g, 0.26 mmol) and 3-methyl-1-(oxetan-3-yl)-4-(4,4,5,5-tetramethyl-1,3,2-dioxaborolan-2-yl)-1H-pyrazole (0.140 g, 0.53 mmol) was reacted to give crude material. The resulting residue was purified by prep HPLC purification using Instrument: A, Column: C; eluted with a gradient of 0.05% Ammonium Hydroxide solution in water and acetonitrile. Lyophilised gave white solid off white sticky (0.0024 g, 2%).

LCMS t_{R} (Waters, Acidic, 4.0 min): 1.488 min. m/z=512.1 [M+H]⁺

HPLC t_{R} (Waters Alliance e2695 with 2998 detector, basic, 17.0 min): 5.932 min

¹H NMR: (400 MHz, DMSO) δ 12.12 (s, 1H), 8.45 (s, 1H), 7.48 (s, 1H), 7.01 (d, J=7.2 Hz, 2H), 7.06-7.04 (m, 1H), 6.66 (s, 1H), 6.52 (s, 1H), 6.38 (s, 1H), 5.50 (t, J=6.8 Hz, 1H), 4.93 (m, 2H), 4.87 (d, J=6.4 Hz, 2H), 3.59 (s, 3H), 3.31 (s, 3H) 2.37 (s, 3H), 2.09 (s, 6H)

### Example 65: 4-(5-(2,6-dimethylphenoxy)-1-methyl-2-oxo-1,2-dihydropyridin-4-yl)-6-methyl-2-(1-((methylsulfonyl)methyl)-1H-pyrazol-4-yl)-1,6-dihydro-7H-pyrrolo[2,3-c]pyridin-7-one

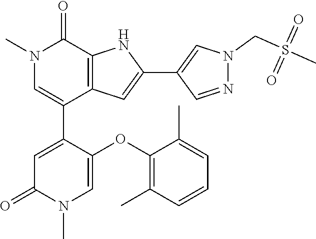

### Preparation 144: 4-(5-(2,6-dimethylphenoxy)-1-methyl-2-oxo-1,2-dihydropyridin-4-yl)-6-methyl-2-(1-((methylsulfonyl)methyl)-1H-pyrazol-4-yl)-1,6-dihydro-7H-pyrrolo[2,3-c] pyridin-7-one

Following the procedure in preparation 101, 2-chloro-4-(5-(2,6-dimethylphenoxy)-1-methyl-2-oxo-1,2-dihydropyridin-4-yl)-6-methyl-1,6-dihydro-7H-pyrrolo [2,3-c] pyridin-7-one (0.20 g, 0.488 mmol) and 1-((methyl sulfonyl) methyl)-4-(4,4,5,5-tetramethyl-1,3,2-dioxaborolan-2-yl)-1H-pyrazole (0.55 g, 1.95 mmol) was reacted to give title compound (0.025 g, 10%) as an off-white solid after purification by flash chromatography in revere phase using Biotage select with UV detector, silica: C18 silica 50 μm eluting with (60:40) acetonitrile/water.

LCMS t_{R} (Waters, Acidic, 17.0 min): 8.303 min, m/z=534.7 [M+H]⁺.

HPLC t_{R} (Waters Alliance e2695 with 2998 detector, Acidic, 17.0 min): 5.84 min

¹H NMR: (400 MHz, DMSO) δ 12.44 (s, 1H), 8.44 (s, 1H), 8.22 (s, 1H), 7.49 (d, J=7.6 Hz, 1H), 7.11 (d, J=7.2 Hz, 2H), 7.05 (m, 1H), 6.64 (m, 2H), 6.55 (s, 1H), 5.80 (s, 2H), 3.59 (s, 3H), 3.31 (s, 3H), 3.04 (s, 3H), 2.09 (s, 6H)

### Primary Activity

Exemplary compounds of the disclosure are active against BRD4 BD2 and selective over BRD4 BD1. BRD4 is a representative example of the BET family, as the binding sites of all BET family members are structurally similar. The half maximal inhibitory concentration (IC50) of Examples 1 to 65 of the compounds is described herein against BRD4 BD1 and BD2 and the fold selectivity calculated (IC50 BD1/IC50 BD2). IC50s and fold selectivity's are determined as described below and are represented in Table 1.

### Bromodomain Assay Procedure

NanoBRET assay was carried out according to the manufacturer's suggested protocol (Promega, Madison, WI). HEK293 cells were transfected using NanoLuc-BRD4-BD1 or NanoLuc-BRD4-BD2 fusion vectors and incubated at 37° C. in an atmosphere of 5% CO2 for 20-24 hours. The transfected cells were then dispensed into 96-well plates using 90 μl cell suspension per well containing 2×10⁵ cells/ml in Opti-MEM and 1x final concentration of tracer. 90 μl per well of cell suspension without tracer was also dispensed into at least 3 wells as “No tracer control samples” for background correction. Serially diluted test compounds were prepared at 1000× concentration in DMSO and further diluted to 10× concentration in Opti-MEM. 10 μl per well of the serially diluted 10× test compound was added to the 96-well plates containing cells with 1x tracer. Plates were then incubated at 37° C.+5% C₀₂ incubator for 2 hours. Immediately prior to BRET measurements, a 3x solution consisting of 1:166 dilution of Nano-Glo® Substrate plus a 1:500 dilution of Extracellular NanoLuc Inhibitor in Opti-MEM was prepared and 50 μl per well was added to the cells. Donor emission (450 nm) and acceptor emission (610 nm) were measured using PHERAstar (BMG LabTech). For data analysis, the raw BRET ratio was generated and converted to milliBRET units with background correction using the formula: [(Acceptorₛₐₘₚₗₑ/Donorₛₐₘₚₗₑ)−(Acceptor_{no tracer control}/Donor_{no tracer control})]×1000. The mBU data was plotted as a function of compound concentration and IC50s for BRET assay were determined by nonlinear regression analysis of concentration response curves using the GraphPad Prism software.

### Fold Selectivity Calculated (IC50 BD1/IC50 BD2).

Compounds of the disclosure were compared to Comparative Example A:

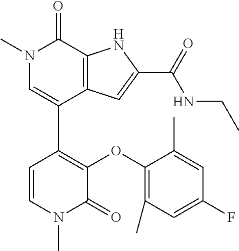 [SMILES: CCNC(=O)c1cc2c(-c3ccn(C)c(=O)c3Oc3c(C)cc(F)cc3C)cn(C)c(=O)c2[nH]1]

TABLE 1
IC50s of Examples 1-59 from BRD4 BD1 and BD2

| | | | Fold |
|---|---|---|---|
| | BRD4 | BRD4 | selectivity |
| | BD1 | BD2 | BRD4 |
| Example | IC50 | IC50 | BD1/BD2 |
| Comparative Example A | # | +++ | xx |
| Example 1 | # | ++++ | xxx |
| Example 2 | # | ++++ | xx |
| Example 3 | # | ++++ | xxx |
| Example 4 | # | ++ | x |
| Example 5 | # | +++ | xx |
| Example 6 | ## | ++++ | xxx |
| Example 7 | # | ++++ | xxx |
| Example 8 | # | +++ | xx |
| Example 9 | # | ++ | x |
| Example 10 | # | ++++ | xxx |
| Example 11 | ### | ++++ | xx |
| Example 12 | ## | ++++ | xx |
| Example 13 | # | ++++ | xxx |
| Example 14 | # | ++++ | xxx |
| Example 15 | # | ++ | x |
| Example 16 | # | ++++ | xxx |
| Example 17 | # | ++++ | xxx |
| Example 18 | # | +++ | xx |
| Example 19 | # | ++ | x |
| Example 20 | # | ++ | x |
| Example 21 | # | + | — |
| Example 22 | # | +++ | xx |
| Example 23 | # | ++ | x |
| Example 24 | # | ++ | x |
| Example 25 | # | ++++ | xxx |
| Example 26 | # | ++++ | xx |
| Example 27 | # | +++ | xx |
| Example 28 | # | +++ | xx |
| Example 29 | # | ++++ | xxx |
| Example 30 | # | +++ | xx |
| Example 31 | ### | ++++ | xx |
| Example 32 | # | +++ | xx |
| Example 33 | # | + | x |
| Example 34 | # | ++++ | xxx |
| Example 35 | # | + | — |
| Example 36 | # | ++++ | xx |
| Example 37 | # | +++ | xx |
| Example 38 | # | ++++ | xxx |
| Example 39 | # | +++ | xx |
| Example 40 | ### | ++++ | x |
| Example 41 | # | +++ | xx |
| Example 42 | # | +++ | xx |
| Example 43 | # | + | x |
| Example 44 | # | ++++ | xx |
| Example 45 | ## | ++++ | x |
| Example 46 | # | ++++ | xxx |
| Example 47 | # | ++++ | xxx |
| Example 48 | # | ++++ | xxx |
| Example 49 | ### | ++++ | xx |
| Example 50 | # | ++++ | xxx |
| Example 51 | # | +++ | xx |
| Example 52 | ### | ++++ | x |
| Example 53 | ### | ++++ | xx |
| Example 54 | ### | ++++ | xx |
| Example 55 | ### | ++++ | x |
| Example 56 | ### | ++++ | x |
| Example 57 | ### | ++++ | x |
| Example 58 | # | ++++ | xxx |
| Example 59 | # | ++++ | xxx |
| Example 60 | # | ++++ | xxx |
| Example 61 | # | ++++ | xxx |
| Example 62 | ### | ++++ | xxx |
| Example 63 | # | ++++ | xxx |
| Example 64 | ## | ++++ | xx |
| Example 65 | # | ++++ | x |

Key
+ BRD4 BD2 IC50 >1 μM
++ BRD4 BD2 IC50 >0.2 μM and ≤1 μM
+++ BRD4 BD2 IC50 >0.05 μM and ≤0.2 μM
++++ BRD4 BD2 IC50 ≤0.05 μM
# BRD4 BD1 IC50 >1 μM
## BRD4 BD1 IC50 >0.5 μM and ≤1 μM
### BRD4 BD1 IC50 >0.05 μM and ≤0.5 μM
— Fold ≥0 and ≤2
x Fold >2 and ≤50
xx Fold >50 and ≤200
xxx Fold >200

In some embodiments BET BOII selective protein inhibitors exhibit one, two or more of the following characteristics +++ or ++++ BRD4 BD2 IC50, # BRD4 BD1 IC50 and xx or xxx Fold selectivity. In some embodiments BET BOII selective protein inhibitors exhibit one, two or more of the following characteristics +++ or ++++ BRD4 BD2 IC50, # or ## BRD4 BD1 IC50 and xx or xxx Fold selectivity. In an embodiment BET BDII selective protein inhibitors exhibit greater than about 200 Fold selectivity for BDII over BDI. In some embodiments some BET BDII selective protein inhibitors exhibit greater than about 250 Fold selectivity, greater than about 300 Fold selectivity, greater than about 350 Fold selectivity greater than about 400 Fold selectivity, greater than about 500 Fold selectivity, greater than about 600 Fold selectivity, greater than about 700 Fold selectivity, greater than about 800 Fold selectivity, greater than about 900 Fold selectivity, or greater than about 1000 Fold selectivity for BDII over BDI depending on the structure. In some embodiments BET BDII selective protein inhibitors exhibit the following characteristics +++ or ++++ BRD4 BD2 IC50 and xx or xxx Fold selectivity. Preferably, BET BDII selective protein inhibitors exhibit an IC50 of <0.05 μM for BRD4 BDII and a Fold selectivity of >200 for BDII over BDI. Generally BET BDII selective protein inhibitors with an IC50 of <0.05 μM for BRD4 BD II and a Fold selectivity of >200 for BDII over BDI are particularly promising drug candidates, but compounds having a lower activity and/or selectivity may in one or more embodiments be useful in particular contexts. In addition to the compounds showing activity and selectivity other factors in selecting promising drug candidates can include for example, plasma stability, clearance, pK, and bioavailability. For drug candidates for oral delivery a higher bioavailability can translate into a lower dosage and potentially fewer side effects e.g., in the alimentary canal.

### Mouse Plasma Stability

Exemplary compounds of the disclosure are stable upon incubation in mouse plasma. Stability is expressed as a % remaining after 120 minutes. The experimental methods and results (Table 3) are provided hereinafter.

### Mouse Plasma Stability Assay Procedure

The stock solution of compound (10 or 50 mM) was prepared in DMSO. From the stock solution, a working solution of compound (500 uM) was prepared in DMSO (100%). To 735 μL of mouse plasma, 15 μL of working solution of compound was added—resulting in a final concentration of 10 μM (2% DMSO) for the compound. The sample was then incubated at 37° C. Aliquot was withdrawn at time-points—0, 15, 30, 60, 90 and 120 mins. The reaction was stopped by using chilled acetonitrile containing internal standard (IS). The samples were centrifuged and the supernatants analyzed using LC-MS/MS. The percent remaining of the compound at each time point was calculated with respect to the control sample (0 min time-point).

Compounds of the disclosure were compared to Comparative Example A (see above) and Comparative Example B:

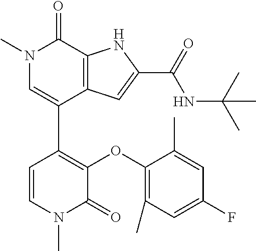 [SMILES: Cc1cc(F)cc(C)c1Oc1c(-c2cn(C)c(=O)c3[nH]c(C(=O)NC(C)(C)C)cc23)ccn(C)c1=O]

TABLE 2
Mouse plasma stability of Example compounds

| | Mouse Plasma |
|---|---|
| | Stability |
| | % remaining at |
| Example | 120 minutes |
| Comparative Example A | 7 |
| Comparative Example B | 100 |
| Example 1 | 95 |
| Example 2 | 100 |
| Example 3 | 77 |
| Example 7 | 84 |
| Example 11 | 98 |
| Example 14 | 99 |
| Example 17 | 42 |

In one or more embodiments, BET BDII selective protein inhibitors exhibit a mouse plasma stability of greater than about 70%, greater than about 75%, greater than about 80%, greater than about 85%, greater than about 90%, or greater than about 95% at 120 minutes. In an embodiment, BET BDII selective protein inhibitors exhibit a mouse plasma stability of about 90% or greater at 120 minutes. BET BDII selective protein inhibitors with a mouse plasma stability of about 90% or greater than 90% at 120 minutes are particularly promising drug candidates, but compounds having a lower mouse plasma stability may in some embodiments be useful in particular contexts. Thus, in one or more embodiments it may be that in one or more embodiments, some compounds have a mouse plasma stability of about 90% or greater than 90% at 120 minutes in addition to having good activity and a selectivity of greater than about 200 Fold.

### Intrinsic Clearance in Mouse Liver Microsomes

BET protein inhibitors with a low rate of clearance in mouse liver microsomes are promising oral drug candidates. Some of the exemplary compounds of the current disclosure have low clearance in mouse liver microsomes, the rate of which is expressed in ml/min/g liver. The experimental methods and results (Table 3) are provided hereinafter.

### Intrinsic Clearance in Mouse Liver Microsomes Assay Procedure 1

A 50 mM stock solution (in DMSO) was prepared for the compound. From the stock solution, a working solution of 0.5 mM was prepared by diluting the compound in DMSO. This concentration of working solution was prepared considering a final concentration of 1 uM with 0.1% DMSO. The compound (1 uL of working solution) was spiked in PBS with pH 7.4 (22 uL) at a concentration of 1 uM. Subsequently, 110 uL of 10 mM NADPH was added (as a co-factor). The sample was incubated at 37° C. for 15 min. Following this, pre-warmed mouse liver microsomes (27.5 uL; final protein conc. 0.5 mg/mL) were added. The samples were then incubated at 37° C. Aliquots of samples were withdrawn at 0, 5, 15, 30, 45 and 60 min. The reaction was stopped by using chilled acetonitrile containing internal standard. The samples were centrifuged and the supernatants analysed by LC-MS/MS. The percent compound remaining at each time point was calculated with respect to that of the 0 min sample. The data were then analysed to calculate half-life and intrinsic clearance (CLint). Note that control samples were run without NADPH and blank samples were prepared using DMSO without the test compound.

TABLE 3
Mouse Intrinsic clearance of Example compounds

| | Mouse Microsomal |
|---|---|
| | Stability |
| Example | ml/min/g liver |
| Comparative Example A | 0.9 |
| Comparative Example B | 5.1 |
| Example 1 | 0.7 |
| Example 2 | 0.9 |
| Example 3 | 0.5 |
| Example 4 | 0.6 |
| Example 5 | 0.6 |

In one or more embodiments, BET BDII selective protein inhibitors exhibit a mouse microsomal stability of <about 5, <about 4, <about 3, <about 2, or <about 1 ml/min/g liver. In an embodiment, BET BDII selective protein inhibitors exhibit a mouse microsomal stability of <2 about ml/min/g liver. BET BDII selective protein inhibitors with a mouse microsomal stability of <about 2 ml/min/g liver are particularly promising drug candidates, but compounds having a lower mouse microsomal stability may in some embodiments be useful in particular contexts. Thus, it may be that in one or more embodiments, some compounds have a mouse microsomal stability of <about 2 ml/min/g liver and or having a mouse plasma stability of about 90% or greater than 90% at 120 minutes in addition to having good activity and a selectivity of greater than about 200 Fold.

### Intrinsic Clearance in Rat Liver Microsomes Assay Procedure

A 50 mM stock solution (in DMSO) was prepared for the compound. From the stock solution, a working solution of 0.5 mM was prepared by diluting the compound in DMSO. This concentration of working solution was prepared considering a final concentration of 1 uM with 0.1% DMSO. The compound (1 uL of working solution) was spiked in PBS with pH 7.4 (22 uL) at a concentration of 1 uM. Subsequently, 110 uL of 10 mM NADPH was added (as a co-factor). The sample was incubated at 37° C. for 15 min. Following this, pre-warmed rat liver microsomes (27.5 uL; final protein conc. 0.5 mg/mL) were added. The samples were then incubated at 37° C. Aliquots of samples were withdrawn at 0, 5, 15, 30, 45 and 60 min. The reaction was stopped by using chilled acetonitrile containing internal standard. The samples were centrifuged and the supernatants analysed by LC-MS/MS. The percent compound remaining at each time point was calculated with respect to that of the 0 min sample. The data were then analysed to calculate half-life. Note that control samples were run without NADPH and blank samples were prepared using DMSO without the test Compound.

TABLE 4
Rat Intrinsic clearance of Example compounds

| | Rat Microsomal Stability |
|---|---|
| Example | T½ (min) |
| Example 6 | >60 |
| Example 7 | >60 |
| Example 8 | >60 |
| Example 9 | >60 |
| Example 10 | 44 |
| Example 11 | >60 |
| Example 12 | >60 |
| Example 13 | >60 |
| Example 14 | >60 |
| Example 15 | 23 |
| Example 16 | >60 |
| Example 17 | >60 |
| Example 18 | >60 |
| Example 19 | >60 |
| Example 20 | >60 |
| Example 21 | >60 |
| Example 22 | >60 |
| Example 23 | >60 |
| Example 24 | >60 |
| Example 25 | >60 |
| Example 26 | >60 |
| Example 28 | >60 |
| Example 29 | >60 |
| Example 30 | >60 |
| Example 31 | >60 |
| Example 32 | >60 |
| Example 33 | >60 |
| Example 35 | >60 |
| Example 36 | >60 |

BET BDII selective protein inhibitors exhibit a rat microsomal stability of >about 20 minutes, >about 20 minutes, >about 30 minutes, >about 40 minutes, >about 50 minutes, >about 60 minutes half-life. In an embodiment, BET BDII selective protein inhibitors exhibit a rat microsomal stability of >about 30 minutes half-life. BET BDII selective protein inhibitors with a rat microsomal stability of >about 30 minutes half-life are particularly promising drug candidates, but compounds having a lower rat microsomal stability may in some embodiments be useful in particular contexts. Thus, it may be that in one or more embodiments, some compounds have a rat microsomal stability of >about 30 minutes half-life and or have a mouse plasma stability of about 90% or greater than 90% at 120 minutes and or have a mouse microsomal stability of <about 2 ml/min/g liver in addition to having good activity and a selectivity of greater than about 200 Fold.

### Inhibition of IL-17A and IL-22 Release in Human PBMCs

### Assay Procedure

1. Cryopreserved human peripheral blood mononuclear cells (PBMCs) were obtained.
2. On the day of experiment, PBMCs were thawed in RPMI-1640 medium supplemented with 10% fetal bovine serum (FBS).
3. Compounds were serially diluted from 10 mM in DMSO and further diluted in assay media to 100× the required final concentration, of which 2 μl was added per well to a round bottom 96-well tissue culture plate. Final DMSO concentration per well was 0.1%.
4. CD2, CD3 and CD28 antibodies coated beads from T cell Activation/Expansion Kit (Cat. No. 130-091-441, Miltenyi Biotec) were added to the PBMCs at a bead-to-cell ratio of 1:2.
5. PBMCs along with the beads were seeded at 2×10⁵ cells/well in the round bottom 96-well plate containing different compounds and controls in a total volume of 200 μl.
6. The cells were cultured for 72 hrs at 37° C., 5% CO₂.
7. Supernatant was collected and IL-17A and IL-22 are analysed by ELISA according to manufacturer's protocol using kits from Invitrogen.
8. Concentration of the cytokines, interpolated from the standard curve, were normalized to percent inhibition values using assay maximum and minimum controls. IC50 values were plotted using nonlinear regression curve using GraphPad Prism.

Certain compounds of the present disclosure were tested according to this assay and were observed to cause a reduction in IL-17A and IL-22 concentrations. IL-17A and IL-22 are biomarkers associated with inflammation and thus the reduction indicates that the compounds of the invention have potential to be useful as anti-inflammatory drugs.

Balb/c mouse pharmacokinetic parameters of selected Example compounds after oral (PO) and intravenous (IV) dosing

### Assay Procedure

Compounds were formulated in 5% DMSO, 40% PEG-400, 55% Milli-Q water at a concentration of 0.5 mg/mL for 5 mg/kg and 3 mg/mL for 30 mg/kg PO dosing. Two male Balb/c mice per arm were dosed with up to 4 compounds in cassette mode or three male Balb/c mice per arm were dosed individually as indicated in Table 9, with each compound being dosed at the concentration indicated in Table 9. Blood was sampled at various timepoints and plasma isolated. 10 μL of plasma with 5 μL of blank Balb/c mouse plasma were added to 200 μL of acetonitrile containing IS mixture. The samples were vortexed for 30 s. After centrifugation at 4 degree Celsius, 3900 rpm for 15 min, the supernatant was diluted 3 times with water. 20 μL of diluted supernatant was injected into the LC/MS/MS system for quantitative analysis and concentration of analyte determined by comparison with matrix matched standard curve. WinNonlin (Phoenix™, version 8.3) or other similar software was used for pharmacokinetic calculations.

TABLE 5
Balb/c mouse pharmacokinetic parameters after
oral (PO) dosing of Example compounds

| | Mouse PO | | | |
|---|---|---|---|---|
| | Dose | CMax | Bioavailability | Casette/ |
| Example | (mg/kg) | (ng/mL) | (%) | Individual |
| 47 | 5 | 718 | 100 | Casette |
| 49 | 5 | 19.3 | 7.81 | Casette |
| 50 | 5 | 293 | 73 | Casette |
| 51 | 5 | 24.4 | 6.83 | Casette |
| 59 | 30 | 5460 | 100* | Individual |
| 60 | 5 | 631 | 59.9 | Casette |
| 61 | 5 | 622 | 51.2 | Casette |
| 62 | 5 | 1290 | 88.7 | Casette |
| 63 | 5 | 1590 | 86.5 | Casette |
| 64 | 30 | 1010 | 12.5 | Individual |

*Mean bioavailability >100% due to last IV data point being at 8 h.

In one or more embodiments BET BDII selective protein inhibitors exhibit a bioavailability of >about 12%, >about 20%, >about 25%, >about 30%, >about 40%, >about 50%, >about 60%>about 70%, >about 80%, >about 90%, or >about 95%. In an embodiment, BET BDII selective protein inhibitors exhibit a bioavailability of >about 25%. BET BDII selective protein inhibitors with a bioavailability of >about 55% are particularly desirable. BET BDII selective protein inhibitors with a bioavailability of >about 25% are promising and >about 55% particularly promising drug candidates, but compounds having a bioavailability of about 25% or less may in some embodiments be useful in particular contexts. Thus, it may be that in one or more embodiments, some compounds have a bioavailability of >about 25% and or have a mouse plasma stability of about 90% or greater than 90% at 120 minutes and or have a mouse microsomal stability of <about 2 ml/min/g liver and or have a rat microsomal stability of >about 30 minutes half-life and or have a IL-17 IC50 of <about 200 nM and or have a IL-22 IC50 of <about 20 nM in addition to having good activity and a selectivity of greater than about 200 Fold.

The data in Table 5 shows that compounds according to formula (XXXI), formula (XXXII), formula (XXXIII), formula (XXXV), formula (XXXVII) and formula (XXXVIII) have particularly high bioavailability.

1. A compound of formula (I), or a pharmaceutically acceptable salt or N-oxide thereof: 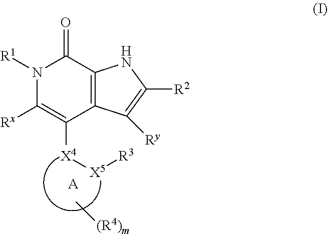
wherein:
Ring A is independently selected from phenyl, 5-membered heterocyclyl and 6-membered heterocyclyl, wherein X⁴ is independently selected from carbon and nitrogen and X⁵ is independently selected from carbon and nitrogen;
R¹ is independently selected from C₁-C₃-alkyl, C₁-C₃-fluoroalkyl, C₃-C₄-cycloalkyl and 4-membered heterocycloalkyl;
R² is independently selected from 5-membered heterocyclyl, 6-membered heterocyclyl and phenyl, each optionally substituted with from 1 to 4 R^{2a} groups;
R^{2a} is independently at each occurrence selected from ═O, ═S, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₆ cycloalkyl and 4- to 6-membered heterocyclyl;
R³ is independently selected from R^{3a}, OR^{3b}, and NR⁶R^{3b};
R^{3a} is independently selected from H, CN, C₁-C₄-alkyl, C₂-C₄-alkenyl, C₂-C₄-alkynyl, C₁-C₄-haloalkyl, C₂-C₄-haloalkenyl, and C₀-C₃-alkylene-R^{3c}; wherein R^{3c} is independently at each occurrence selected from C₃-C₈-cycloalkyl, C₅-C₈-cycloalkenyl, 5- to 8-membered heterocycloalkenyl, 3- to 8-membered heterocycloalkyl, phenyl and 5- or 6-membered heteroaryl; wherein where R^{3c} is cycloalkyl, heterocycloalkyl, cycloalkenyl, or heterocycloalkenyl, R^{3c} is optionally substituted with from 1 to 4 R⁸ groups and where R^{3c} is phenyl or heteroaryl, R^{3c} is optionally substituted with from 1 to 5 R⁹ groups;
R^{3b} is independently selected from C₁-C₄-alkyl, C₂-C₄-alkylene-O—C₁-C₄-alkyl, C₁-C₄-haloalkyl and C₀-C₃-alkylene-R^{3d}; wherein R^{3d} is independently at each occurrence selected from C₃-C₈-cycloalkyl, 3- to 8-membered heterocycloalkyl, phenyl and 5- or 6-membered heteroaryl; wherein where R^{3d} is cycloalkyl or heterocycloalkyl, R^{3d} is optionally substituted with from 1 to 4 R⁸ groups and where R^{3d} is phenyl or heteroaryl, R^{3d} is optionally substituted with from 1 to 5 R⁹ groups;
R⁴ is independently at each occurrence selected from ═O, ═S, halo, nitro, cyano, C₀-C₄-alkylene-NR⁵R⁶, C₀-C₄-alkylene-OR⁷, SR⁶, SOR⁶, C₀-C₄-alkylene-S(O)₂R⁶, SO₂NR⁶R⁶, C₀-C₄-alkylene-CO₂R⁶, C₀-C₄-alkylene-C(O)R⁶, C₀-C₄-alkylene-CONR⁶R⁶, C₁-C₄-alkyl, C₁-C₄-alkyl-S(O)₂R⁶, C₂-C₄-alkenyl, C₂-C₄-alkynyl, C₁-C₄-haloalkyl, cyclopropyl, cyclobutyl, and 4- to 6-membered heterocycloalkyl;
R⁵ is independently at each occurrence selected from H, C₁-C₄-alkyl, C(O)—C₁-C₄-alkyl and S(O)₂-C₁-C₄-alkyl; or R⁵ and R⁶, together with the nitrogen atom to which they are attached form a C₅-C₈-heterocycloalkyl group optionally substituted with from 1 to 4 R⁸ groups;
R⁶ is independently at each occurrence selected from H and C₁-C₄-alkyl; or where two R⁶ groups are attached to the same nitrogen, those two R⁶ groups together with the nitrogen atom to which they are attached optionally form a C₅-C₈-heterocycloalkyl group optionally substituted with from 1 to 4 R⁸ groups;
R⁷ is independently at each occurrence selected from H, C₁-C₄-alkyl, C(O)—C₁-C₄-alkyl and C₁-C₄-haloalkyl;
R⁸ is independently at each occurrence selected from ═O, ═S, fluoro, nitro, cyano, NR⁵R⁶, OR⁷, SR⁶, SOR⁶, S(O)₂R⁶, SO₂NR⁶R⁶, C₀₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R⁹ is independently at each occurrence selected from halo, nitro, cyano, NR⁵R⁶, OR⁷, SR⁶, SOR⁶, S(O)₂R⁶, SO₂NR⁶R⁶, C₀₂R⁶, C(O)R⁶, CONR⁶R⁶, C₁-C₄-alkyl, C₂-C₄-alkenyl, C₂-C₄-alkynyl, C₁-C₄-haloalkyl and cyclopropyl;
R^{x} and R^{y} are each independently selected from H, halo, nitro, cyano, NR⁵R⁶, OR⁷, SR⁶, SOR⁶, S(O)₂R⁶, SO₂NR⁶R⁶, CO₂R⁶, C(O)R⁶, CONR⁶R⁶, C₁-C₄-alkyl, C₂-C₄-alkenyl, C₂-C₄-alkynyl, C₁-C₄-haloalkyl, C₃-C₄-cycloalkyl and 4-membered heterocycloalkyl;
m is an integer selected from 0, 1, 2, 3 and 4;
wherein any of the aforementioned alkyl, alkylene, alkenyl, or cyclopropyl groups is optionally substituted, where chemically possible, by 1 to 5 substituents which are each independently at each occurrence selected from the group consisting of: C₁-C₄-alkyl, oxo, fluoro, nitro, cyano, NR^{a}R^{b}, OR^{a}, SR^{a}, CO₂R^{a}, C(O)R^{a}, CONR^{a}R^{a}, S(O)R^{a} and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂-C₁-C₄-alkyl.

2. A compound of claim 1, wherein R^{x} and R^{y} are each H.

3. A compound of claim 1, wherein when Ring A is a 5 membered heterocyclyl it is not a pyrrolidone.

4. A compound of claim 1, wherein X⁴ is carbon.

5. A compound of claim 1, wherein R¹ is methyl.

6. A compound of claim 1, wherein R² is 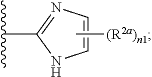
and wherein n1 is independently an integer selected from 0, 1, and 2.

7. A compound of claim 1, wherein R² is 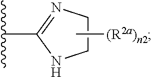
and wherein n2 is independently an integer selected from 0, 1, 2, and 3.

8. A compound of claim 1, wherein R² is 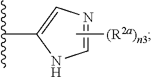
and wherein n3 is independently an integer selected from 0, 1, and 2.

9. A compound of claim 1, wherein R² is 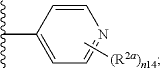
wherein n14 is independently an integer selected from 0, 1, 2, 3, and 4.

10. A compound of claim 1, wherein R² is 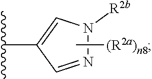
wherein n8 is independently an integer selected from 0, 1, 2, and 3; and R^{2b} is selected from H, C₁-C₄-alkyl, C₃-C₆ cycloalkyl and 4- to 6-membered heterocyclyl.

11. A compound of claim 1, wherein R² is 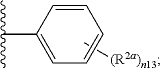
wherein n13 is independently an integer selected from 0, 1, 2, 3, 4, and 5.

12. A compound of claim 1, wherein R² is 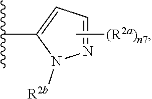
wherein n7 is independently an integer selected from 0, 1 and 2; and R^{2b} is selected from H, C₁-C₄-alkyl, C₃-C₆ cycloalkyl and 4- to 6-membered heterocyclyl.

13. A compound of claim 1, wherein Ring A is phenyl.

14. A compound of claim 1, wherein Ring A is pyridone.

15. A compound of claim 14, wherein Ring A is substituted on the nitrogen with 1 group selected from C₁-C₄-alkyl, cyclopropyl, cyclobutyl and 4-membered heterocycloalkyl.

16. A compound of claim 14, wherein Ring A is 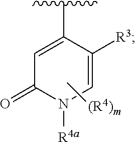
wherein R^{4a} is selected from H, C₁-C₄-alkyl, cyclopropyl and 4-membered heterocycloalkyl.

17. A compound of claim 16, wherein R^{4a} is selected from methyl, cyclopropyl, oxetane and azetidine.

18. A compound of claim 1, wherein Ring A is 5-membered heteroaryl.

19. A compound of claim 1, wherein R³ is R^{3a}.

20. A compound of claim 19, wherein R^{3a} is phenyl optionally substituted with from 1 to 3 R⁹ groups.

21. A compound of claim 1, wherein R³ is OR^{3b}.

22. A compound of claim 21, wherein R^{3b} is phenyl; optionally substituted with from 1 to 3 R⁹ groups.

23. A compound of claim 21, wherein R^{3b} is C₀-C₃-alkylene-R^{3d}; wherein R^{3d} is independently at each occurrence selected from C₃-C₆-cycloalkyl and 4- to 6-membered heterocycloalkyl; wherein R^{3d} is optionally substituted with from 1 to 4 R⁸ groups.

24. A compound of claim 1 selected from: 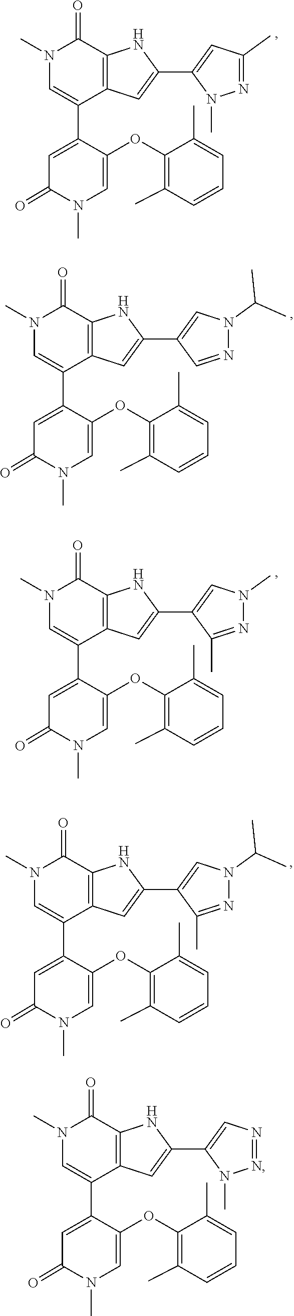 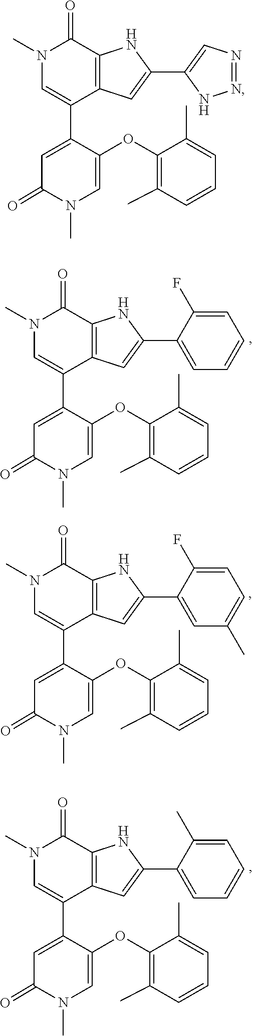 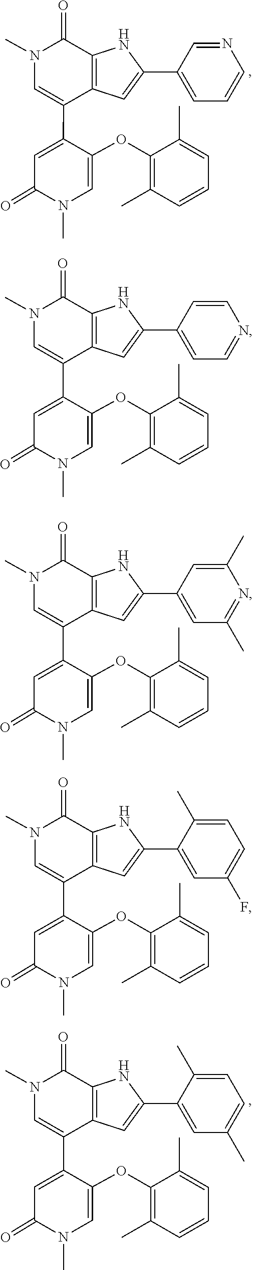 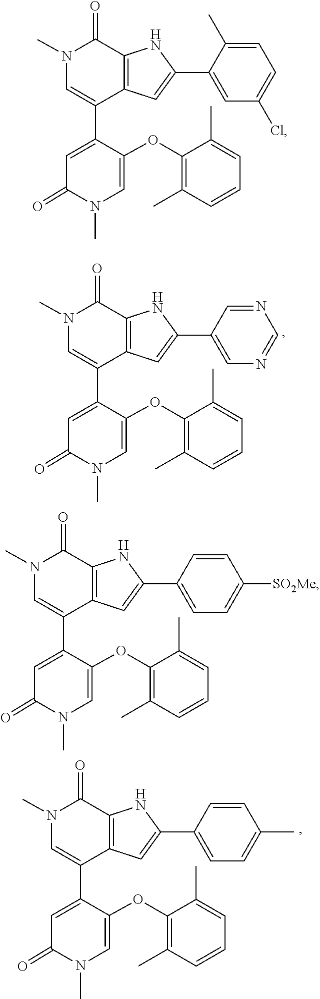 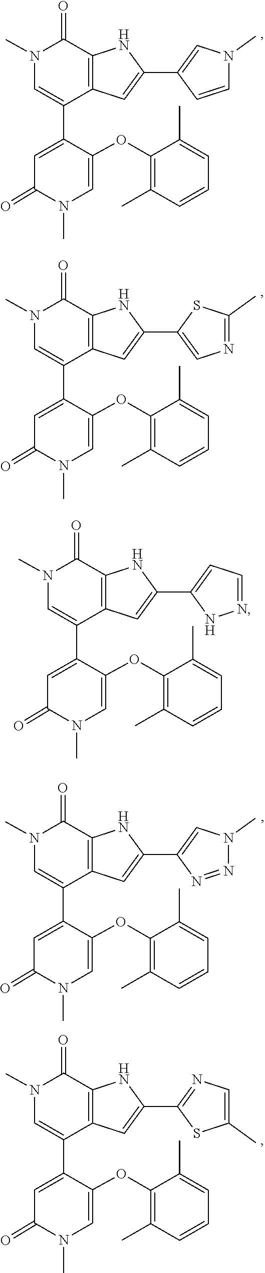 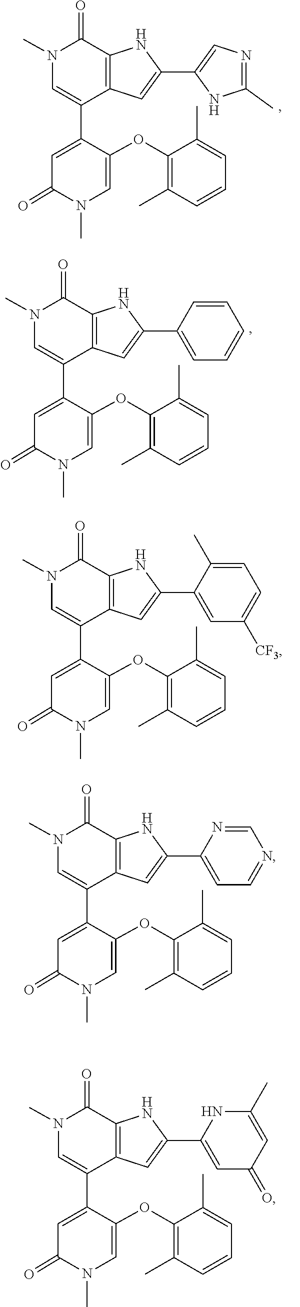 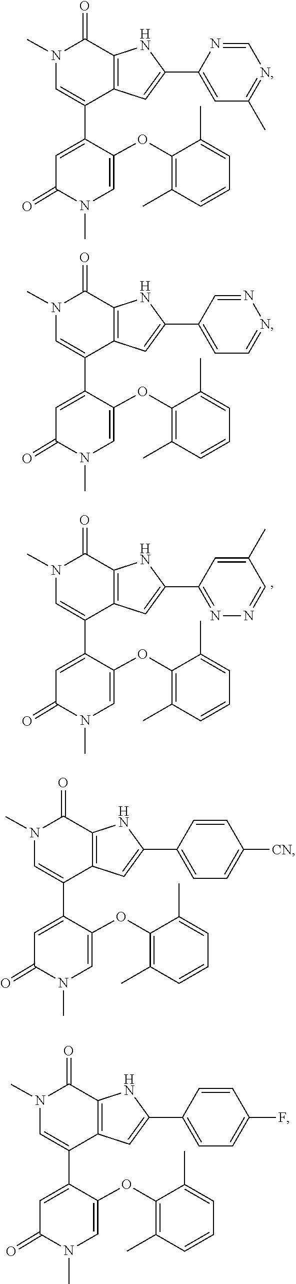 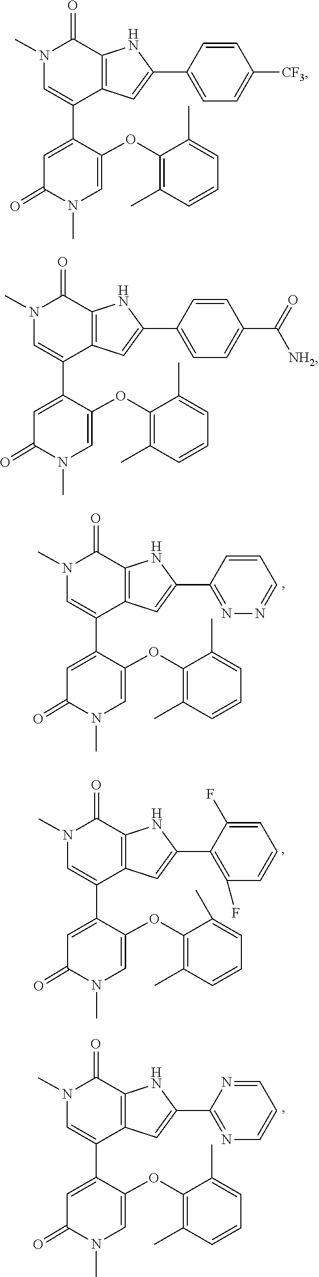 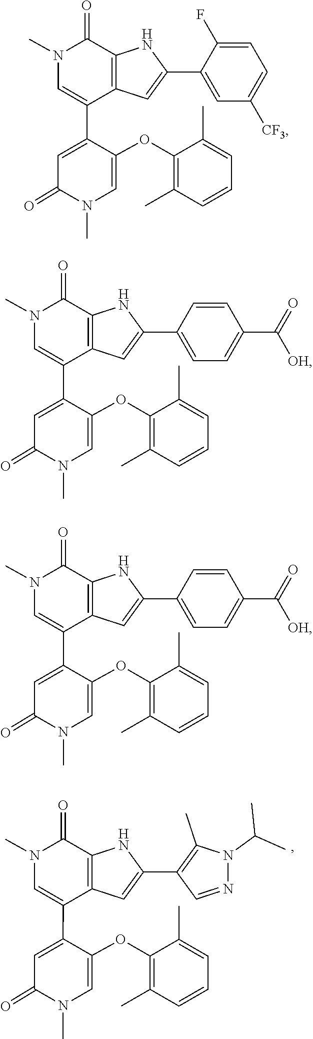 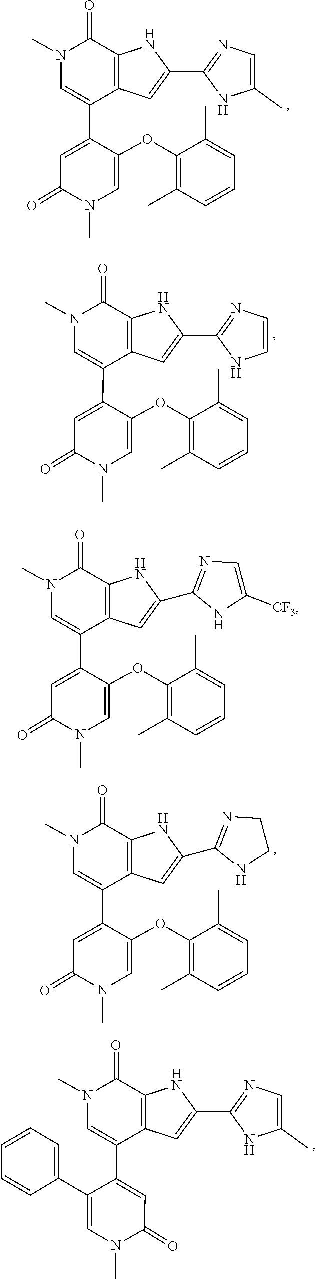 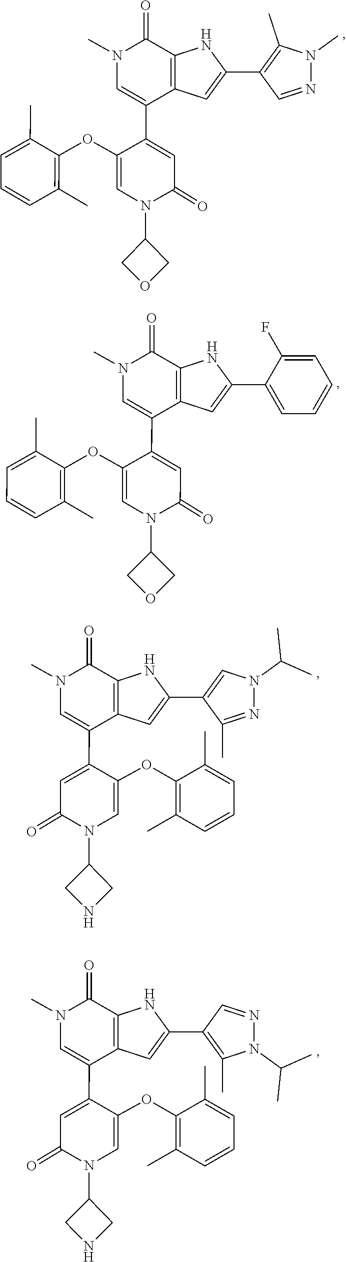 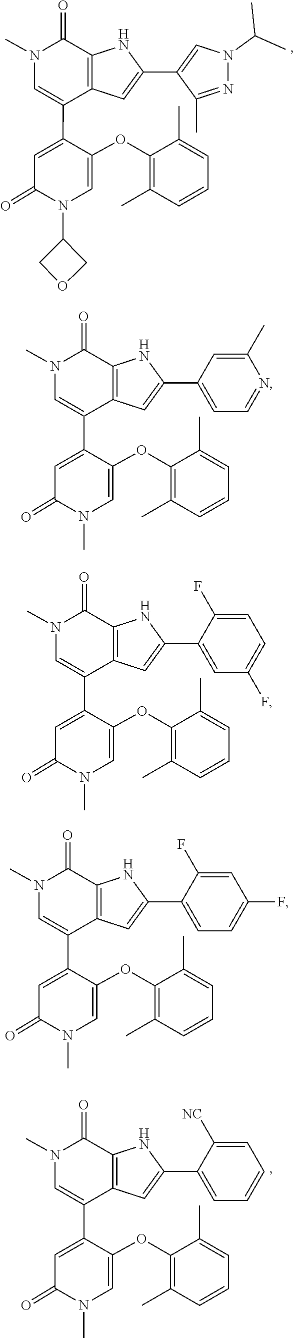 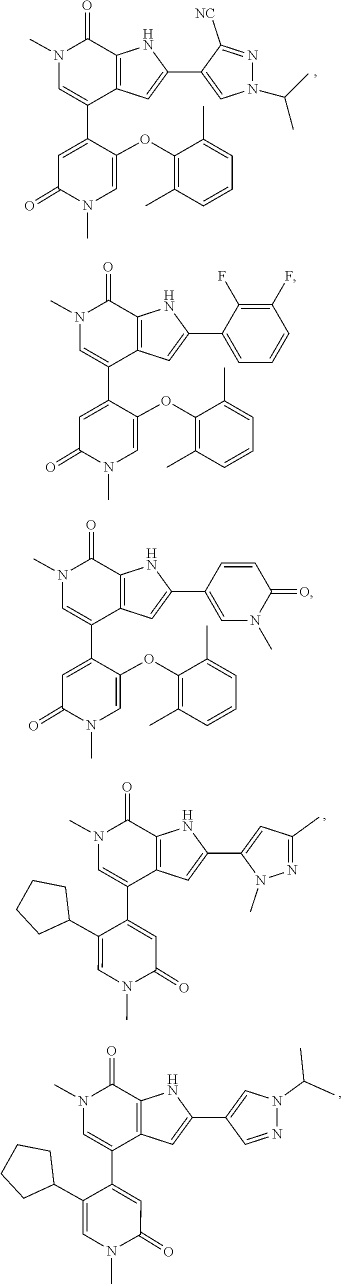 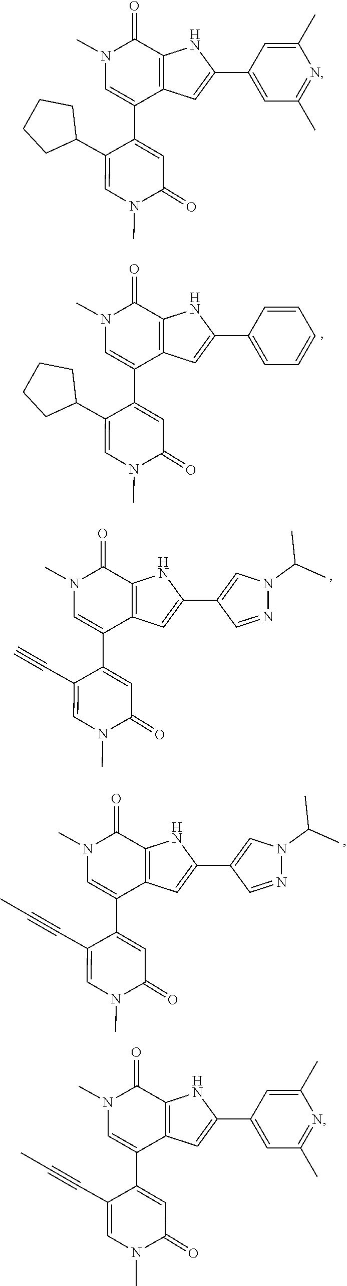 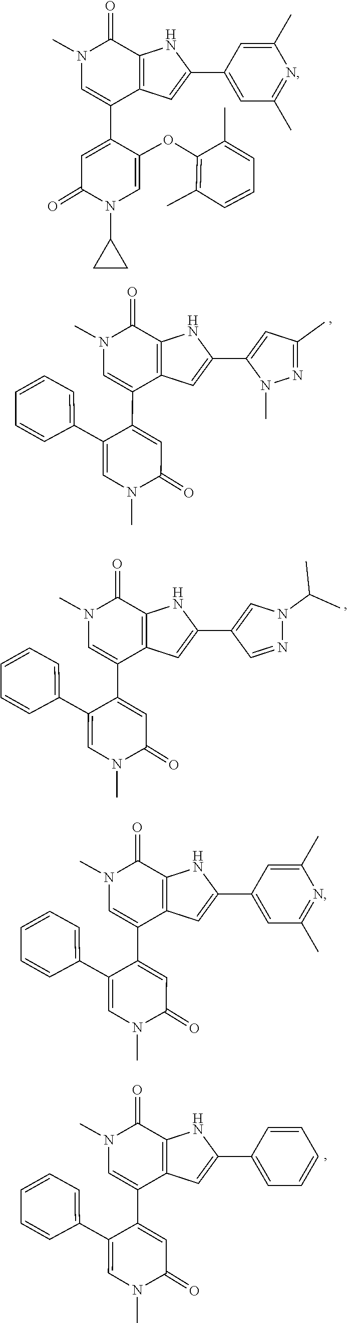 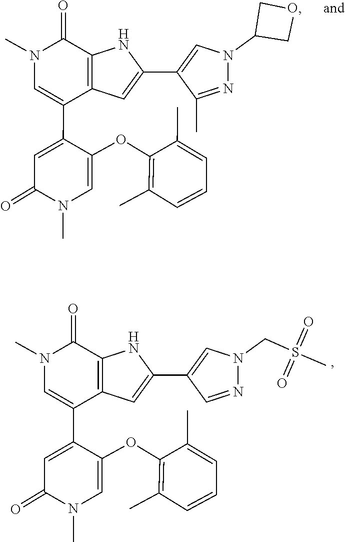
or a pharmaceutically acceptable salt or N-oxide thereof.

25. A pharmaceutical composition comprising a compound of claim 1, or a pharmaceutically acceptable salt or N-oxide thereof, and one or more pharmaceutically acceptable excipients.

26. (canceled)

27. A method of treating a disease or disorder selected from one or more of an inflammatory disease or disorder, an immune disease or disorder, and an autoimmune disease or disorder, comprising administering to a warm-blooded animal a therapeutically effective amount of a compound of claim 1, or a pharmaceutically acceptable salt or N-oxide thereof.

28. (canceled)
